(19) United States
(12) Patent Application Publication
(10) Pub. No.: US 2010/0233172 A1
(43) Pub. Date: Sep. 16, 2010

(54) METHODS OF INHIBITING QUIESCENT TUMOR PROLIFERATION

(75) Inventors: Roberto Weinmann, Princeton, NJ (US); Rameh Hafezi, Franklin Park, NJ (US); Arthur M.P. Doweyko, Vero Beach, FL (US); Ashok Ramesh Dongre, Newtown, PA (US); Deborah L. Roussell, Trenton, NJ (US); Robert F. Carney, Wilmington, DE (US)

Correspondence Address: Choate Hall & Stewart LLP/Infinity/BMS, Two International Place, Boston, MA, 02110, US

(73) Assignee: Bristol-Myers Squibb Company

(21) Appl. No.: 12/639,083

(22) Filed: Dec. 16, 2009

Related U.S. Application Data
(60) Provisional application No. 61/201,913, filed on Dec. 16, 2008.

(51) Int. Cl. A61K 39/395; A61P 35/00; A61K 31/713; A61K 38/16; A61K 31/5513; G01N 33/567; G01N 33/573; C12N 5/09; G01N 33/53

(52) U.S. Cl. 424/135.1; 424/174.1; 514/44 A; 514/12; 514/221; 435/7.23; 435/7.21; 435/7.4; 435/366; 435/7.1

(57) ABSTRACT

Improved compositions and methods are disclosed which are useful of the treatment and prevention of proliferative disorders, and methods of screening to identify compounds for such treatments.

This application claims benefit to provisional application U.S. Ser. No. 61/201,913 filed Dec. 16, 2008; under 35 U.S.C. 119(e). The entire teachings of the referenced application are incorporated herein by reference.

### BRIEF DESCRIPTION OF THE SEQUENCE LISTING

Incorporated herein by reference in its entirety is a Sequence Listing, comprising SEQ ID NO:1 through SEQ ID NO:114. The Sequence Listing is submitted herewith as an ASCII text file, and thus constitutes both the paper and computer readable form thereof. The Sequence Listing, in IBM/PC MS-DOS, ASCII text format, was first created on Dec. 14, 2009, and is 104 KB in size.

### FIELD OF THE INVENTION

Improved compositions and methods are disclosed which are useful of the treatment and prevention of proliferative disorders, and methods of screening to identify compounds for such treatments.

### BACKGROUND OF THE INVENTION

Farnesyltransferase inhibitors (FTIs) are novel chemotherapeutic agents vigorously pursued for their potential anticancer activity in humans (Cestac et al., Ann. Pharm. Fr., 63:76-84 (2005); Zhu et al., Curr. Opin. Investig. Drugs, 4:1428-1435 (2003)). BMS-214662, an imidazole-containing tetrahydrobenzodiazepine, is a small molecule inhibitor of farnesyltransferase (FT), (Hunt et al., J. Med. Chem., 43:3587-3595 (2000)). BMS-214662 showed remarkable anti-tumor activity in a significant number of tumor xenograft models, producing curative efficacy in many instances (Rose et al., Cancer Res., 61:7507-7517 (2001)). The regressions and cures described for several human tumor xenografts, following a relatively short course of treatment with BMS-214662, raised the question as to what mechanism underlay such rapid reduction of tumor volume. The unexpected rapid clearance contrasted with the cytostatic activity (Kohl et al., Proc. Natl. Acad. Sci. USA, 91:9141-9145 (1994); Nagasu et al., Cancer Res., 55:5310-5314 (1995)) or slow regressions in certain tumors (Kohl et al., Nat. Med., 1:792-797 (1995)) reported for other FTI agents (Kohl et al., Proc. Natl. Acad. Sci. USA, 91:9141-9145 (1994); Nagasu et al., Cancer Res., 55:5310-5314 (1995)).

As a solid tumor grows, the vascular development may not keep pace with the rapid proliferation of the malignant cell population. Consequently, solid tumor masses typically exhibit abnormal blood vessel networks that, unlike vessels in normal tissues, fail to provide adequate nutritional and oxygen support to all tumor cells for optimal growth. Solid tumors, therefore, comprise both proliferating (P) and quiescent (Q) tumor cells. In most solid tumors, Q cells constitute the majority of the total tumor cell population (Jackson, R. C., Adv. Enzyme Regul., 29:27-46 (1989)) and are thought to constitute a reservoir for the origin of new P populations. Since previous results indicated that BMS-214662 caused massive apoptosis in HCT-116 solid tumors (Rose et al., Cancer Res., 61:7507-7517 (2001)), the inventors hypothesized that BMS-214662 may bring about rapid tumor regressions by specifically targeting the bulk of the tumor mass, i.e., the tumor's Q population. Conventional chemotherapeutic agents act primarily on P cells and are not curative. Therefore the clinical utility of BMS-214662 in targeting the Q tumor cell population may be a significant contribution to more efficacious therapy.

The general pattern of sensitivity of tumor cell sub-populations (either P or Q) to cytotoxic or cytostatic compounds was also explored. The selective effects of BMS-214662 on Q cells in tissue culture and in mouse xenograft models are described. Surprisingly, this preferential activity of BMS-214662 does not translate to catastrophic toxicity in adult animals, where most somatic cells are non-proliferating (but are not cancer cells). The inventors explored possible treatment combinations for therapeutic use of BMS-214662, exploiting its potency and selectivity towards Q cells.

Furthermore, the inventors describe herein what is believed to be the first elucidation of the mechanism of action for the BMS-214662 compound—namely through modulation and/or agonism of the Apoptosis Inducing Factor, AIF, HOP protein, phosphatase 1G, PTPN6, HSP70, and/or other target proteins, as described herein.

AIF (gi|NP_{—}04199.1) is the causative protein responsible for caspase-independent apoptosis (Lorenzo et al., Cell. Death Differ., 6:516-524 (1999); Susin et al., J. Exp. Med., 189:381-393 (1999); Susin et al., Nature, 397:441-446 (1999); and Susin et al., J. Exp. Med., 186:25-37 (1999)). When a cell receives an apoptotic insult, it has been shown that AIF translocates from the mitochondria to the cell nucleus where it induces programmed cell death (Lorenzo et al., Drug Resistance Updates, 10:235-255 (2007)).

HOP (gi|NP_{—}006810.1), also referred to as STI or the Hsp70-Hsp90 Organizing Protein, is a protein involved in heat shock response that acts as a scaffolding protein for the molecular chaperones Hsp70 and Hsp90 (Danial et al., Biochemica et Biophysica Acta, 1783:1003-1014 (2008)). HOP interacts with Hsp70 and Hsp90 though specific tetratricopeptide-repeat (TPR)-rich binding domains, and forms a Hsp70-HOP-Hsp90 chaperone heterocomplex. HOP is known to be localized predominately in the cytoplasm, but has been shown to shuttle between the nucleus and the cytoplasm (Longshaw et al., J. Cell. Sci., 117:701-710 (2004)). The cellular localization of HOP, in addition to its intereaction with Hsp90, appears to be regulated by phosphorylation (Longshaw et al.; Danial et al.).

Phosphatase 1G (gi|NP_{—}02698.1) is also referred to as PPM16, and is a member of the PP2C family of phosphatases which are known to function as negative regulators of cell stress response pathways. PPM16 specifically, has been shown to be involved in chromatin dephosphorylation in response to DNA damage (Kimura et al., J. Cell. Biol., 175(3):389-400 (2006). More recently, PPM16 has also been shown to dephosphorylate substrates required for formation of the spliceosome. In addition, it has also been shown to be a novel regulator of p21(Cip1/WAF1) protein stability via the Ala signaling pathway (Suh et al., BBRC, 386(3):467-70 (2009)).

Protein tyrosine phosphatase, non-receptor type 6 isoform 2 (gi|NP_{—}002822.2) is also referred to as PTPN6. The protein encoded by this gene is a member of the protein tyrosine phosphatase (PTP) family. PTPs are known to be signaling molecules that regulate a variety of cellular processes including cell growth, differentiation, mitotic cycle, and oncogenic transformation. N-terminal part of this PTP contains two tandem Src homolog (SH2) domains, which act as protein phospho-tyrosine binding domains, and mediate the interaction of this PTP with its substrates. This PTP is expressed primarily in hematopoietic cells, and functions as an important regulator of multiple signaling pathways in hematopoietic cells. This PTP has been shown to interact with, and dephosphorylate a wide spectrum of phospho-proteins involved in hematopoietic cell signaling. Multiple alternatively spliced variants of this gene, which encode distinct isoforms, have been reported.

While AIF is known to be associated with apoptosis, it has heretofore been unknown that agonism of AIF, HOP, phosphatase 1G, and/or PTPN6 can result in the induction of apoptosis in quiescent cells, specifically, and preferably quiescent cancer cells. The induction of apoptosis in quiescent cells represents a critical missing link in the treatment of cancer. Because a majority of the cells in any given tumor are in a quiescent state, current therapeutic cancer treatment regimens target only the minority proliferating population of cells and thus, fail to induce apoptosis in the quiescent state. Having a therapeutic regimen that targets the quiescent population of a tumor would satisfy an unmet need in the art.

Accordingly, there remains a need in the art to identify enhanced methods for treating cancer, particularly those that target the quiescent tumor cell population. It is an object of the invention to provide efficacious treatment regimens for treating cancer and other proliferative disorders through agonism of AIF, HOP, and/or phosphatase 1G.

### SUMMARY OF THE INVENTION

The present invention provides a method for treating proliferative disease comprising administering to a mammal in need thereof a compound that agonizes AIF, either directly or indirectly. In one aspect, the proliferative disease is one or more cancerous solid tumors. In another aspect, the proliferative disease is one or more refractory tumors. In another aspect, the proliferative disease is a leukemia. Preferably, said treatment method results in the induction of apoptosis in quiescent cells, quiescent tumor cells, tumor stem cells, and/or quiescent stem cells.

The present invention provides a method for treating proliferative disease comprising administering to a mammal in need thereof a compound that agonizes AIF, either directly or indirectly, wherein said compound agonizes the apoptotic activity of AIF. In one aspect, the proliferative disease is one or more cancerous solid tumors. In another aspect, the proliferative disease is one or more refractory tumors. In another aspect, the proliferative disease is a leukemia. Preferably, said treatment method results in the induction of apoptosis in quiescent cells, quiescent tumor cells, tumor stem cells, and/or quiescent stem cells.

The present invention provides a method for treating proliferative disease comprising administering to a mammal in need thereof a compound that agonizes AIF, either directly or indirectly, wherein said compound agonizes the transport of AIF from the mitochondria to the cell nucleus. In one aspect, the proliferative disease is one or more cancerous solid tumors. In another aspect, the proliferative disease is one or more refractory tumors. In another aspect, the proliferative disease is a leukemia. Preferably, said treatment method results in the induction of apoptosis in quiescent cells, quiescent tumor cells, tumor stem cells, and/or quiescent stem cells.

The present invention provides a method for treating proliferative disease comprising administering to a mammal in need thereof a compound that agonizes HOP, either directly or indirectly. In one aspect, the proliferative disease is one or more cancerous solid tumors. In another aspect, the proliferative disease is one or more refractory tumors. In another aspect, the proliferative disease is a leukemia. Preferably, said treatment method results in the induction of apoptosis in quiescent cells, quiescent tumor cells, tumor stem cells, and/or quiescent stem cells.

The present invention provides a method for treating proliferative disease comprising administering to a mammal in need thereof a compound that agonizes HOP, either directly or indirectly, wherein said compound agonizes the ability of HOP to transport proteins to the nucleus, or agonizes the ability to facilitate Hsp70-Hsp90 molecular chaperone complex to transport proteins to the nucleus. In one aspect, the proliferative disease is one or more cancerous solid tumors. In another aspect, the proliferative disease is one or more refractory tumors. In another aspect, the proliferative disease is a leukemia. Preferably, said treatment method results in the induction of apoptosis in quiescent cells, quiescent tumor cells, tumor stem cells, and/or quiescent stem cells.

The present invention provides a method for treating proliferative disease comprising administering to a mammal in need thereof a compound that agonizes HOP, either directly or indirectly, wherein said compound agonizes the ability of HOP to transport proteins to the nucleus, or agonizes the ability of the Hsp70-Hsp90 molecular chaperone complex to transport proteins to the nucleus, wherein at least one of those proteins is AIF. In one aspect, the proliferative disease is one or more cancerous solid tumors. In another aspect, the proliferative disease is one or more refractory tumors. In another aspect, the proliferative disease is a leukemia. Preferably, said treatment method results in the induction of apoptosis in quiescent cells, quiescent tumor cells, tumor stem cells, and/or quiescent stem cells.

The present invention provides a method for treating proliferative disease comprising administering to a mammal in need thereof a compound that agonizes phophatase 1G, either directly or indirectly. In one aspect, the proliferative disease is one or more cancerous solid tumors. In another aspect, the proliferative disease is one or more refractory tumors. In another aspect, the proliferative disease is a leukemia. Preferably, said treatment method results in the induction of apoptosis in quiescent cells, quiescent tumor cells, tumor stem cells, and/or quiescent stem cells.

The present invention provides a method for treating proliferative disease comprising administering to a mammal in need thereof a compound that agonizes protein tyrosine phosphatase non-receptor type 6 isoform 2, either directly or indirectly. In one aspect, the proliferative disease is one or more cancerous solid tumors. In another aspect, the proliferative disease is one or more refractory tumors. In another aspect, the proliferative disease is a leukemia. Preferably, said treatment method results in the induction of apoptosis in quiescent cells, quiescent tumor cells, tumor stem cells, and/or quiescent stem cells.

The present invention also provides a method for identifying a compound that is useful for treatment of proliferative disease comprising incubating quiescent cells, quiescent tumor cells, tumor stem cells, and/or quiescent stem cells with a test compound, determining whether apoptosis is induced in said cells, and confirming that said compound agonizes a member of the group consisting of: AIF; HOP; the ability of HOP to facilitate the Hsp70-Hsp90 molecular chaperone complex to transport proteins to the nucleus; Hsp70-Hsp90 molecular chaperone complex to transport proteins to the nucleus; protein tyrosine phosphatase non-receptor type 6 isoform 2; and phosphatase 1G.

The present invention also provides a method for identifying a compound that is useful for the treatment of proliferative diseases comprising incubating a test compound with AIF, identifying those compounds that bind to AIF, and determining whether incubation of said AIF-binding compounds are capable of inducing apoptosis in quiescent cells, quiescent tumor cells, tumor stem cells, and/or quiescent stem cells. Such binding may be detected using assays otherwise known in the art, including, but not limited to, calorimetry, changes in AIF conformation, gel shift assays, binding assays, detection of chemical shifts in NMR, and competition experiments using labeled compounds (radiolabeled, chemically labeled, fluorescently labeled, enzymatically labeled, etc.) known to bind AIF, such as any one of the compounds disclosed herein.

The present invention also provides a method for identifying a compound that is useful for treatment of proliferative disease comprising incubating a test compound with HOP, identifying those compounds that bind to HOP, and determining whether incubation of said HOP-binding compounds are capable of inducing apoptosis in quiescent cells, quiescent tumor cells, tumor stem cells, and/or quiescent stem cells. Such binding may be detected using assays otherwise known in the art, including, but not limited to, calorimetry, changes in HOP conformation, gel shift assays, binding assays, detection of chemical shifts in NMR, and competition experiments using labeled compounds (radiolabeled, chemically labeled, fluorescently labeled, enzymatically labeled, etc.) known to bind HOP, such as any one of the compounds disclosed herein.

The present invention also provides a method for identifying a compound that is useful for treatment of proliferative disease comprising incubating a test compound with phosphatase 1G, identifying those compounds that bind to phosphatase 1G, and determining whether incubation of said phosphatase 1G-binding compounds are capable of inducing apoptosis in quiescent cells, quiescent tumor cells, tumor stem cells, and/or quiescent stem cells. Such binding may be detected using assays otherwise known in the art, including, but not limited to, calorimetry, changes in phosphatase 1G conformation, gel shift assays, binding assays, detection of chemical shifts in NMR, and competition experiments using labeled compounds (radiolabeled, chemically labeled, fluorescently labeled, enzymatically labeled, etc.) known to bind phosphatase 1G, such as any one of the compounds disclosed herein.

The present invention also provides a method for identifying a compound that is useful for treatment of proliferative disease comprising incubating a test compound with protein tyrosine phosphatase non-receptor type 6 isoform 2, identifying those compounds that bind to protein tyrosine phosphatase non-receptor type 6 isoform 2, and determining whether incubation of said protein tyrosine phosphatase non-receptor type 6 isoform 2-binding compounds are capable of inducing apoptosis in quiescent cells, quiescent tumor cells, tumor stem cells, and/or quiescent stem cells. Such binding may be detected using assays otherwise known in the art, including, but not limited to, calorimetry, changes in protein tyrosine phosphatase non-receptor type 6 isoform 2 conformation, gel shift assays, binding assays, detection of chemical shifts in NMR, and competition experiments using labeled compounds (radiolabeled, chemically labeled, fluorescently labeled, enzymatically labeled, etc.) known to bind protein tyrosine phosphatase non-receptor type 6 isoform 2, such as any one of the compounds disclosed herein.

The present invention also provides a method for identifying a compound useful for treatment of proliferative diseases comprising incubating a cell with a compound, wherein said cell is capable of expressing AIF either endogenously or recombinately, and wherein said cell is further incubated with a labeled antibody specific to AIF either prior to, during, or after incubation with said compound, and determining whether said compound increases the frequency or amount of AIF that is accumulated, localized, or translocated to the nucleus or modulates the biological activity of AIF, relative to a control cell that has not been exposed to said test compound. Preferably, said cells are quiescent cells, quiescent tumor cells, tumor stem cells, and/or quiescent stem cells. Preferably, such a compound specifically induces apoptosis in quiescent cells.

The present invention also provides a method for identifying a compound that is useful for treatment of proliferative disease comprising incubating a cell with a compound, wherein said cell is capable of expressing HOP either endogenously or recombinately, and wherein said cell is further incubated with a labeled antibody specific to HOP either prior to, during, or after incubation with said compound, and determining whether said compound increases the frequency or amount of AIF or HOP that is accumulated, localized, or translocated to the nucleus or modulates the biological activity of HOP, relative to a control cell that has not been exposed to said test compound. Preferably, said cells are quiescent cells, quiescent tumor cells, tumor stem cells, and/or quiescent stem cells. Preferably, such a compound specifically induces apoptosis in quiescent cells.

The present invention also provides a method for identifying a compound that is useful for treatment of proliferative disease comprising incubating a cell with a compound, wherein said cell is capable of expressing phosphatase 1G either endogenously or recombinately, and wherein said cell is further incubated with a labeled antibody specific to phosphatase 1G either prior to, during, or after incubation with said compound, and determining whether said compound increases the frequency or amount of phosphatase 1G that is accumulated, localized, or translocated to the nucleus or modulates the biological activity of phosphatase 1G, relative to a control cell that has not been exposed to said test compound. Preferably, said cells are quiescent cells, quiescent tumor cells, tumor stem cells, and/or quiescent stem cells. Preferably, such a compound specifically induces apoptosis in quiescent cells.

The present invention also provides a method for identifying a compound that is useful for treatment of proliferative disease comprising incubating a cell with a compound, wherein said cell is capable of expressing protein tyrosine phosphatase non-receptor type 6 isoform 2 either endogenously or recombinately, and wherein said cell is further incubated with a labeled antibody specific to protein tyrosine phosphatase non-receptor type 6 isoform 2 either prior to, during, or after incubation with said compound, and determining whether said compound increases the frequency or amount of protein tyrosine phosphatase non-receptor type 6 isoform 2 that is accumulated, localized, or translocated to the nucleus or modulates the biological activity of protein tyrosine phosphatase non-receptor type 6 isoform 2, relative to a control cell that has not been exposed to said test compound. Preferably, said cells are quiescent cells, quiescent tumor cells, tumor stem cells, and/or quiescent stem cells. Preferably, such a compound specifically induces apoptosis in quiescent cells.

The present invention also provides a method for identifying a compound that is useful for treatment of proliferative disease comprising incubating a cell with a compound, wherein said cell is capable of expressing HOP either endogenously or recombinately, and wherein said cell is further incubated with a labeled antibody specific to a protein capable of being accumulated, localized, or translocated to the nucleus (e.g., AIF, phosphatase 1G, protein tyrosine phosphatase non-receptor type 6 isoform 2, etc.), either prior to, during, or after incubation with said compound, and determining whether said compound increases the frequency or amount of said protein that is accumulated, localized, or translocated to the nucleus, relative to a control cell that has not been exposed to said test compound. Preferably, said cells are quiescent cells, quiescent tumor cells, tumor stem cells, and/or quiescent stem cells. Preferably, such a compound specifically induces apoptosis in quiescent cells.

The present invention provides a method for treating proliferative disease comprising administering to a mammal in need thereof a compound that modulates AIF, stress-induced-phosphoprotein 1 (Hsp70/Hsp90-organizing protein), protein phosphatase 1G, protein tyrosine phosphatase non-receptor type 6 isoform 2, and/or tubulin, preferably in quiescent cells, quiescent tumor cells, tumor stem cells, and/or quiescent stein cells.

In one aspect, the proliferative disease is one or more cancerous solid tumors. In another aspect, the proliferative disease is one or more refractory tumors. In another aspect, the proliferative disease is a leukemia. Preferably, said treatment method results in the induction of apoptosis in quiescent cells, quiescent tumor cells, tumor stem cells, and/or quiescent stem cells.

The present invention also encompasses methods of treating proliferative disorders using therapeutically effective amounts of an anti-proliferative compound with an AIF agonist modulator compound.

The present invention also encompasses methods of treating proliferative disorders using therapeutically effective amounts of an anti-proliferative compound with a HOP agonist modulator compound.

The present invention also encompasses methods of treating proliferative disorders using therapeutically effective amounts of an anti-proliferative compound with a phosphatase 1G agonist modulator compound.

The present invention also encompasses methods of treating proliferative disorders using therapeutically effective amounts of an anti-proliferative compound with a protein tyrosine phosphatase non-receptor type 6 isoform 2 agonist modulator compound.

The present invention also encompasses methods of treating proliferative disorders using therapeutically effective amounts of an anti-proliferative compound with a Hsp70 antagonist modulator compound which inhibits the ability of Hsp70 to bind to and sequester AIF, thus resulting in an effective, agonism-like effect of AIF and thus leading to caspase-independent, apoptosis.

The present invention is also directed to a method of inducing apoptosis in a cell comprising administering a pharmaceutically acceptable amount of a compound according to formula I,

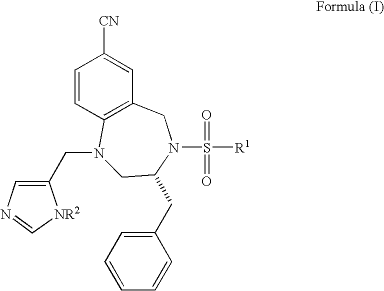

wherein R¹ is selected from the group consisting of:

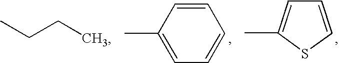

wherein R² is either H or CH₃, and wherein said cell is selected from the group consisting of: quiescent cells, quiescent tumor cells, tumor stem cells, and quiescent stem cells.

As used herein the terms “modulate” or “modulates” refer to an increase or decrease in the amount, quality or effect of DNA, RNA, or protein, or the increase or decrease of a particular biological activity.

A “modulator” of AIF, HOP, phosphatase 1G, protein tyrosine phosphatase non-receptor type 6 isoform 2, Hsp70, and/or Other Target Protein for the purposes of the present invention, may be a small molecule, antibody, domain antibody, single-chain antibody, an antibody fragment, an RNAi molecule directed against the encoding nucleotide sequence of any of these proteins, an adnectin, antisense molecules directed against the encoding nucleotide sequence of any of these proteins, or any other molecule, protein, nucleic acid that is capable of agonizing the biological activity of any of these proteins, either directly or indirectly, such that said modulator results in apoptosis, preferably in quiescent cells.

For the purposes of the present invention, the phrase “AIF agonist” or “agonist of AIF” or “agonize AIF” means not only agonize the biochemical activity of AIF, but also an indirect activity that may result in increased AIF activity as evidenced by an increase in apoptosis in non-proliferating cancer cells, quiescent cells, tumor stem cells, and/or quiescent tumor cells. For example, AIF agonism may be observed by agonizing the ability of HOP to accumulate, localize, or translocate AIF to the nucleus. Alternatively, AIF agonism may be observed by inhibiting the ability of Hsp70 to sequester AIF, thus making it available for translocation into the nucleus resulting in induction of apoptosis. Other examples of AIF agonism are disclosed herein.

For the purposes of the present invention, the phrase “agonism of” HOP, phosphatase 1G, protein tyrosine phosphatase non-receptor type 6 isoform 2, Hsp70, and/or Other Target Protein means modulation of the biochemical, enzymatic, translocation, protein activation, or other functional activity, in which an increased level of apoptosis is observed, which may or may not be attributable to increased translocation of AIF into the nucleus, increased availability of AIF, increased availability of AIF free from Hsp70 sequestration, direct agonism of AIF biological activity via agonism of one or more of the proteins described herein, or indirect agonism of AIF via agonism of one or more of the proteins described herein.

“Other Target Protein” means any other protein that has been shown to bind to the BMS-214662 compound aside from AIF, HOP, phosphatase 1G, protein tyrosine phosphatase non-receptor type 6 isoform 2, and/or Hsp70, including but not limited to, tubulin, HSP60, adenyl cyclase AP, pyruvate kinase, alpha-enolase, 5′ methylthioadenosine phosphorylase, 14-3-3 Protein Sigma, Nit protein 2 (Nit-2), Prolyl-4-hydroxylase-beta, eukaryotic translation elongation factor 1 alpha, and/or the proteins outlined in FIG. 7, or as described elsewhere herein. Such Other Target Proteins may result in agonism of AIF, directly, or indirectly, when bound to a compound of the present invention, or may result in apoptosis through a caspase-independent mechanism when bound to a compound of the present invention, or alternatively, may result in apoptosis through a caspase-dependent mechanism when bound to a compound of the present invention.

### BRIEF DESCRIPTION OF THE DRAWINGS

The file of this patent contains at least one Figure executed in color. Copies of this patent with color Figure(s) will be provided by the Patent and Trademark Office upon request and payment of the necessary fee.

FIG. 1. Flow cytometry analysis of HCT-116 cells treated with BMS-214662. Untreated HCT-116 cells in P or in Q state (Panel a) were analyzed by flow cytometry. The y axis represents cell numbers and the x axis DNA content. HCT-116 Q cells treated with BMS-214662 3 μM for 4 hr (Panel b, lower line), and washed and chased with spent medium without compound for 20 hrs (Panel b, top line) were analyzed by flow cytometry for activated caspase 3 as indicated in the Examples described herein. The x axis indicates the level of activated caspase while the y axis represents percentage of total fluorescence adjusted to 100%. In panels c and d HCT 116 P and Q, respectively were treated with BMS-214662 at 3 μM for 4 hours followed by a 20 hour chase in the presence of BrdU and analyzed by flow cytometry after reacting with BrdU and p85 PARP antibodies. The x axis indicates the level of BrdU incorporation, while the y axis indicates the p85 cleaved PARP cells. The lower right grouping of dots represent BrdU incorporating cells (33.1% for P, 9.9% for Q), the upper left grouping of dots represent Q apoptotic cells (4.5% for P, 23.2% for Q), the upper right grouping of dots represents BrdU incorporating cell that are apoptotic (7.7% in P, 4.2% in Q) while the lower left grouping of dots represents single cells with background, low level staining (35% in P, 39.4% in Q).

FIG. 2. Selective targeting of quiescent tumor cells by BMS-214662 and of proliferating cells by paclitaxel and ixabepilone in vitro. Colony forming ability of P (diamonds) or Q (squares) tumor cells following treatment with BMS-214662, paclitaxel or ixabepilone for 16 hr was determined as described herein. Surviving fraction was calculated based on the number of cells from untreated controls that grew into a colony (taken as 1 with >50% of the plated cells forming colonies). a: HCT-116 colon treated cells, b: HT29 colon cells, c: patient 7 ovarian cancer, d: K562 CML cells (not from colony assay but direct cell counts); e and f represent colony Ruining HCT-116 cells treated with paclitaxel and ixabepilone, respectively.

FIG. 3. Generic structure for BMS-214662 and related FTIs. Data for the substituents at the R¹ and R² positions are displayed in Table 1.

FIG. 4. Combination of hormonal therapy with BMS-214662. Panel a and b show the results of analyzing by flow cytometry cells of MDA-PCa2b prostate tumors grown in nude mice before and after castration, respectively. BrdU incorporating cells (P) are indicated in the upper grouping of dots, while the BrdU non-incorporating/non-proliferating, Q cells are in the lower grouping of dots. Panel c shows the analysis of tumor growth in nude mice with (diamonds) and without (open circles) oral treatment (daily gavage) with BMS-214662 (indicated by the small triangles between days 5 and 14) in intact animals. Castrated animals one day after castration (indicated by X) with (circles) and without BMS-214662 treatment (triangles). Panel d shows results obtained against MCF-7 human breast xenografts in nude mice either intact (diamonds), treated with BMS-214662 alone (blue triangles), after removal of the estradiol pellets (diamonds) or after tamoxifen treatment (days 10-48, 3 times a week), with (lower grouping of circles) or without (upper grouping of circles) addition of BMS-214662, which was given orally daily in two cycles (indicated by vertical arrows at the bottom) and results for 8 mice per group are presented.

FIG. 5. Combination chemotherapy of BMS-214662 with cytotoxic agents against HCT-116 tumor xenografts in nude mice. Panels a and b. Nude mice with established HCT-116 tumors (˜200 mg size) were subjected to N treatment, either with vehicle (open circles), paclitaxel alone (20 mg/kg, squares), BMS-214662 alone (80 mg/kg, panel a or 40 mg/kg, panel b; circles) or in combination (lower grouping of circles). Paclitaxel and/or BMS-214662 were administered once a week for four weeks, and results for 8 mice per group are averaged. Panel c. Nude mice with established HCT-116/VM46 MDR resistant tumors (˜200 mg size) acted as controls (open circles), or were subjected to IV treatment with ixabepilone at 15 mg/kg (squares) administered every 4 days, three times (MTD, vertical arrows) or BMS-214662 was administered by itself daily by gavage at a dose of 400 mg/kg (squares). Combination of ixabepilone at 6 mg/kg with the same regimen, followed 24 hr later by BMS-214662 daily as indicated by the dashed blue line at 300 mg/kg (open diamonds) or 400 mg/kg (filled squares) are indicated and resulted in 3/7 cures. Panel d and e. Nude mice with established HCT-116 tumors of ˜300 mg size were subjected to IV treatment with CPT-11 (30 mg/kg, orange circles) and/or BMS-214662 either at 80 mg/kg (panel d) or 60 mg/kg (panel e) alone (light triangles) or in combination (diamonds) were administered once a week for three weeks. The sequence of treatment was CPT-11 (at MTD) followed 24 hr later by BMS-214662, as indicated by the brown triangles.

FIG. 6. BMS-214662 exposure required to kill 50% of P (a) and Q (b) HCT116 tumor cells. HCT-116 IC₅₀s for cell killing using the colony forming assay were determined after different times of exposure to BMS-214662, followed by washing off the drug and plotted against the exposure. The exposure achieved in clinical trials after 1 and 24 hr infusions were used to establish the exposure achievable in humans (hatched bar) (see Ryan et al., Clin. Cancer Res., 10:2222-2230 (2004); Papadimitrakopoulou et al., Clin. Cancer Res., 11:4151-4159 (2005); Tabernero et al., J. Clin. Oncol., 23:2521-2533 (2005); Cortes et al., J. Clin. Oncol., 23:2805-2812 (2005); and Dy et al., Clin. Cancer Res., 11:1877-1883 (2005)).

FIG. 7. Shows a polyacrylamide gel of proteins from an early cross-linking experiment using HCT116 quiescent cell extracts and BMS-214662 (referred to in FIG. 14). As shown, bands consistent with several forms of AIF were observed (i.e., bands with molecular weight of 57, 62, and 67 kDa). Other proteins identified from these bands that co-migrated with the different faints of AIF are also indicated.

FIG. 8. Shows a polyacrylamide gel of proteins from an early affinity capture experiment in either the presence or absence of BMS-236724, a biotinylated analogue of BMS-214662, using HCT116 quiescent cell extracts. BMS-236724 has farnesyl transferase inhibitory activity and retains some of the pro-apoptotic activity of the BMS-214662 compound. As shown, bands consistent with several forms of AIF were observed.

FIG. 9. Panels A and B show a polyacrylamide gel of proteins from an early cross-linking experiment with BMS-540864 in either the presence of absence of competitor compounds BMS-214662 or BMS-225975, using cell extracts from either HCT-116 quiescent cells (“HCT-116 Q”) or HCT-116 proliferating cells (“HCT-116 P”) relative to cell extracts from non-treated HCT-116 cells (“Control”). Panel C shows a polyacrylamide gel of proteins from an early cross-linking experiment in either the presence of BMS-214662, using cell extracts from either quiescent (“HCT-116/r Q”) or proliferating forms (“HCT-116/r P”) of a resistant strain of HCT-116 cells. As shown, the protein banding patterns observed for the BMS-214662 resistant HCT-116 cells were more similar to the proliferating HC-116 P cells regardless of whether these cells were proliferating or quiescent. These results further support the connection between AIF and the pro-apoptotic effects of BMS-214662 because the AIF bands are not observed in the resistant HCT-116 strain nor in the proliferating, BMS-214662 sensitive HCT-116 strain, but rather only in the quiescent HCT-116 strain.

FIG. 10A. Shows a general schematic illustrating the method used to isolate and identify proteins that bound to BMS-214662.

FIG. 10B. Shows a specific schematic illustrating the specific steps used to isolate proteins that bound to BMS-214662 and analyze them on polyacrylamide gels.

FIG. 11. Shows a silver stained polyacrylamide gel of the peptides that eluted from the monomeric avidin bead columns shown in FIGS. 10A-B.

FIG. 12. Shows the sequence of the AIF protein (gi|NP_{—}004199.1; SEQ ID NO:1), as well as denoting the specific peptide fragments that were identified by LC/LC/MS/MS from the tryptic digests of the protein present in specific gel slices from SDS-PAGE of the BMS-844472 bound fraction. The peptides denoted in bold was identified in the first analysis of gel slices shown in FIG. 8; the peptides denoted in single underlining was identified in the B gel slice in a second experiment; the peptide denoted in italics was identified in B slices in both experiments as well as in an early cross-linking experiment; and the number sign (“#”) indicates the cleavage site of the extramitochondrial protein, and the asterisk (“*”) indicates start of the reported 3D structure of AIF (shown in FIG. 13).

FIG. 13. Shows a molecular 3-dimensional model of the AIF protein with the BMS-214662 compound (shown in pink). As shown, the AIF protein has a deep binding pocket in which the BMS-214662 compound may be bound.

FIG. 14. Shows a molecular 3-dimensional model of the AIF protein with the BMS-214662 compound illustrating the stabilizing interaction between the imidazole and the arginine at position 450 of the AIF binding pocket (left panel). The model also demonstrates the loss of the stabilizing interaction when the BMS-214662 compound is substituted with the BMS-225975 compound, an N-methylated analogue that does not induce apoptosis.

FIG. 15. Shows a sequence alignment between the human AIF protein (SEQ ID NO:1), the mouse AIF orthologue (SEQ ID NO:2), and Benzene 1,2-dioxygenase system ferredoxin—NAD(+) reductase subunit (gi|1168642; SEQ ID NO:3). As shown, a strong conservation exists between both proteins, however, there are some changes in the proximity of the predicted BMS-214662 contact sites, which could lead to a difference in substrate specificity. Such changes may explain the differential therapeutic window observed with the BMS-214662 compound in mice and humans. A summary of these peptide fragments, the ions observed from the LC/LC/MS/MS spectra, in addition to the frequency of observed fragments is provided in Table 3.

FIG. 16. Shows one model of the mechanism of action for BMS-214662 in agonizing the pro-apoptotic activity of AIF. Proliferative cell condition is represented by “P”; Quiescent cell condition is represented by “Q”; and AIF is represented by the structures shown with an asterisk “*”. According to the model, conditions of limiting nutrients or reduced mitochondrial function as is present for cells in a quiescent state result in conformation changes in the AIF protein that makes it more accessible to the protease calpain, which results in proteolytic cleavage and the subsequent release of AIF from the inner mitochondrial membrane. BMS-214662 binds to AIF and facilitates its release from the inner mitochondrial membrane into the cytosol by stabilizing the structure of AIF that is sensitive to calpain activity. Once in the cytoplasm, it would be bound by the Hsp70-HOP-Hsp90 complex and shuttled to the nucleus.

FIG. 17. Shows a second model of the mechanism of action for BMS-214662 in agonizing the release of AIF from the Hsp70-HOP-Hsp90 complex. In this model, AIF is released into the cytoplasm and bound by Hsp70-HOP-Hsp90 in an inactive form on account of HSP70 being known to inhibit the proapoptotic function of AIF. An alternative or additional activity of BMS-214662 would be related to the release from the Hsp70-HOP-Hsp90 complex to act in the nucleus in combination with DNAse-G in the initiation of the apoptotic process, as shown. The cleavage of PARP (polyADPribose polymerase) in turn frees polyADP ribopolymers which in turn feedback for more AIF release. AIF is represented by the structures shown with an asterisk “*”.

FIG. 18. Shows the sequence of the HOP protein (gi|NP_{—}006810.1; (SEQ ID NO:3), as well as denoting the specific peptide fragments that were shown to bind to BMS-214662 and/or biotinylated analogues thereof. The peptides denoted in bold, italics, single underlining, and double underlining, were identified in one, two, three, and four binding column experiments, respectively. Overall, the peptides that bound to BMS-214662 and/or biotinylated analogues thereof represented approximately 50% of the HOP protein. A summary of these peptide fragments, the ions observed from the LC/LC/MS/MS spectra, in addition to the frequency of observed fragments is provided in Table 4.

FIGS. 19A-C. Shows the observed LC/LC/MS/MS spectra for fragment 1 of AIF that bound to BMS-214662 and/or biotinylated analogues thereof (SEQ ID NO:7).

FIG. 20. Shows a summary of the ions observed from the LC/LC/MS/MS spectra for fragment 1 of AIF that bound to BMS-214662 and/or biotinylated analogues thereof (SEQ ID NO:7).

FIGS. 21A-C. Shows the observed LC/LC/MS/MS spectra for fragment 2 of AIF that bound to BMS-214662 and/or biotinylated analogues thereof (SEQ ID NO:8).

FIG. 22. Shows a summary of the ions observed from the LC/LC/MS/MS spectra for fragment 2 of AIF that bound to BMS-214662 and/or biotinylated analogues thereof (SEQ ID NO:8).

FIGS. 23A-C. Shows the observed LC/LC/MS/MS spectra for fragment 1 of HOP that bound to BMS-214662 and/or biotinylated analogues thereof (SEQ ID NO:9).

FIG. 24. Shows a summary of the ions observed from the LC/LC/MS/MS spectra for fragment 1 of HOP that bound to BMS-214662 and/or biotinylated analogues thereof (SEQ ID NO:9).

FIGS. 25A-C. Shows the observed LC/LC/MS/MS spectra for fragment 2 of HOP that bound to BMS-214662 and/or biotinylated analogues thereof (SEQ ID NO:10).

FIG. 26. Shows a summary of the ions observed from the LC/LC/MS/MS spectra for fragment 2 of HOP that bound to BMS-214662 and/or biotinylated analogues thereof (SEQ ID NO:10).

FIGS. 27A-C. Shows the observed LC/LC/MS/MS spectra for fragment 3 of HOP that bound to BMS-214662 and/or biotinylated analogues thereof (SEQ ID NO:11).

FIG. 28. Shows a summary of the ions observed from the LC/LC/MS/MS spectra for fragment 3 of HOP that bound to BMS-214662 and/or biotinylated analogues thereof (SEQ ID NO:11).

FIGS. 29A-C. Shows the observed LC/LC/MS/MS spectra for fragment 4 of HOP that bound to BMS-214662 and/or biotinylated analogues thereof (SEQ ID NO:12).

FIG. 30. Shows a summary of the ions observed from the LC/LC/MS/MS spectra for fragment 4 of HOP that bound to BMS-214662 and/or biotinylated analogues thereof (SEQ ID NO:12).

FIGS. 31A-C. Shows the observed LC/LC/MS/MS spectra for fragment 5 of HOP that bound to BMS-214662 and/or biotinylated analogues thereof (SEQ ID NO:13).

FIG. 32. Shows a summary of the ions observed from the LC/LC/MS/MS spectra for fragment 5 of HOP that bound to BMS-214662 and/or biotinylated analogues thereof (SEQ ID NO:13).

FIGS. 33A-C. Shows the observed LC/LC/MS/MS spectra for fragment 6 of HOP that bound to BMS-214662 and/or biotinylated analogues thereof (SEQ ID NO:14).

FIG. 34. Shows a summary of the ions observed from the LC/LC/MS/MS spectra for fragment 6 of HOP that bound to BMS-214662 and/or biotinylated analogues thereof (SEQ ID NO:14).

FIGS. 35A-C. Shows the observed LC/LC/MS/MS spectra for fragment 7 of HOP that bound to BMS-214662 and/or biotinylated analogues thereof (SEQ ID NO:15).

FIG. 36. Shows a summary of the ions observed from the LC/LC/MS/MS spectra for fragment 7 of HOP that bound to BMS-214662 and/or biotinylated analogues thereof (SEQ ID NO:15).

FIGS. 37A-C. Shows the observed LC/LC/MS/MS spectra for fragment 8 of HOP that bound to BMS-214662 and/or biotinylated analogues thereof (SEQ ID NO:16).

FIG. 38. Shows a summary of the ions observed from the LC/LC/MS/MS spectra for fragment 8 of HOP that bound to BMS-214662 and/or biotinylated analogues thereof (SEQ ID NO:16).

FIGS. 39A-C. Shows the observed LC/LC/MS/MS spectra for fragment 9 of HOP that bound to BMS-214662 and/or biotinylated analogues thereof (SEQ ID NO:17).

FIG. 40. Shows a summary of the ions observed from the LC/LC/MS/MS spectra for fragment 9 of HOP that bound to BMS-214662 and/or biotinylated analogues thereof (SEQ ID NO:17).

FIGS. 41A-C. Shows the observed LC/LC/MS/MS spectra for fragment 10 of HOP that bound to BMS-214662 and/or biotinylated analogues thereof (SEQ ID NO:18).

FIG. 42. Shows a summary of the ions observed from the LC/LC/MS/MS spectra for fragment 10 of HOP that bound to BMS-214662 and/or biotinylated analogues thereof (SEQ ID NO:18).

FIGS. 43A-C. Shows the observed LC/LC/MS/MS spectra for fragment 11 of HOP that bound to BMS-214662 and/or biotinylated analogues thereof (SEQ ID NO:19).

FIG. 44. Shows a summary of the ions observed from the LC/LC/MS/MS spectra for fragment 11 of HOP that bound to BMS-214662 and/or biotinylated analogues thereof (SEQ ID NO:19).

FIGS. 45A-C. Shows the observed LC/LC/MS/MS spectra for fragment 12 of HOP that bound to BMS-214662 and/or biotinylated analogues thereof (SEQ ID NO:20).

FIG. 46. Shows a summary of the ions observed from the LC/LC/MS/MS spectra for fragment 12 of HOP that bound to BMS-214662 and/or biotinylated analogues thereof (SEQ ID NO:20).

FIGS. 47A-C. Shows the observed LC/LC/MS/MS spectra for fragment 13 of HOP that bound to BMS-214662 and/or biotinylated analogues thereof (SEQ ID NO:21).

FIG. 48. Shows a summary of the ions observed from the LC/LC/MS/MS spectra for fragment 13 of HOP that bound to BMS-214662 and/or biotinylated analogues thereof (SEQ ID NO:21).

FIGS. 49A-C. Shows the observed LC/LC/MS/MS spectra for fragment 14 of HOP that bound to BMS-214662 and/or biotinylated analogues thereof (SEQ ID NO:22).

FIG. 50. Shows a summary of the ions observed from the LC/LC/MS/MS spectra for fragment 14 of HOP that bound to BMS-214662 and/or biotinylated analogues thereof (SEQ ID NO:22).

FIGS. 51A-C. Shows the observed LC/LC/MS/MS spectra for fragment 15 of HOP that bound to BMS-214662 and/or biotinylated analogues thereof (SEQ ID NO:23).

FIG. 52. Shows a summary of the ions observed from the LC/LC/MS/MS spectra for fragment 15 of HOP that bound to BMS-214662 and/or biotinylated analogues thereof (SEQ ID NO:23).

FIGS. 53A-C. Shows the observed LC/LC/MS/MS spectra for fragment 16 of HOP that bound to BMS-214662 and/or biotinylated analogues thereof (SEQ ID NO:24).

FIG. 54. Shows a summary of the ions observed from the LC/LC/MS/MS spectra for fragment 16 of HOP that bound to BMS-214662 and/or biotinylated analogues thereof (SEQ ID NO:24).

FIGS. 55A-C. Shows the observed LC/LC/MS/MS spectra for fragment 17 of HOP that bound to BMS-214662 and/or biotinylated analogues thereof (SEQ ID NO:25).

FIG. 56. Shows a summary of the ions observed from the LC/LC/MS/MS spectra for fragment 17 of HOP that bound to BMS-214662 and/or biotinylated analogues thereof (SEQ ID NO:25).

FIGS. 57A-C. Shows the observed LC/LC/MS/MS spectra for fragment 18 of HOP that bound to BMS-214662 and/or biotinylated analogues thereof (SEQ ID NO:26).

FIG. 58. Shows a summary of the ions observed from the LC/LC/MS/MS spectra for fragment 18 of HOP that bound to BMS-214662 and/or biotinylated analogues thereof (SEQ ID NO:26).

FIG. 59. Shows the sequence of the phosphatase 1G protein (gi|NP_{—}817092.1; SEQ ID NO:5), as well as denoting the specific peptide fragments that were shown to bind to BMS-214662 and/or biotinylated analogues thereof. A summary of these peptide fragments, the ions observed from the LC/LC/MS/MS spectra, in addition to the frequency of observed fragments is provided in Table 5.

FIGS. 60A-C. Shows the observed LC/LC/MS/MS spectra for fragment 1 of phosphatase 1G that bound to BMS-214662 and/or biotinylated analogues thereof (SEQ ID NO:27).

FIG. 61. Shows a summary of the ions observed from the LC/LC/MS/MS spectra for fragment 1 of phosphatase 1G that bound to BMS-214662 and/or biotinylated analogues thereof (SEQ ID NO:27).

FIGS. 62A-C. Shows the observed LC/LC/MS/MS spectra for fragment 2 of phosphatase 1G that bound to BMS-214662 and/or biotinylated analogues thereof (SEQ ID NO:28).

FIG. 63. Shows a summary of the ions observed from the LC/LC/MS/MS spectra for fragment 2 of phosphatase 1G that bound to BMS-214662 and/or biotinylated analogues thereof (SEQ ID NO:28).

FIGS. 64A-C. Shows the observed LC/LC/MS/MS spectra for fragment 3 of phosphatase 1G that bound to BMS-214662 and/or biotinylated analogues thereof (SEQ ID NO:29).

FIG. 65. Shows a summary of the ions observed from the LC/LC/MS/MS spectra for fragment 3 of phosphatase 1G that bound to BMS-214662 and/or biotinylated analogues thereof (SEQ ID NO:29).

FIGS. 66A-C. Shows the observed LC/LC/MS/MS spectra for fragment 4 of phosphatase 1G that bound to BMS-214662 and/or biotinylated analogues thereof (SEQ ID NO:30).

FIG. 67. Shows a summary of the ions observed from the LC/LC/MS/MS spectra for fragment 4 of phosphatase 1G that bound to BMS-214662 and/or biotinylated analogues thereof (SEQ ID NO:30).

FIG. 68. Shows the sequence of the PTPN6 (gi|NP_{—}536858.1; SEQ ID NO:6), as well as denoting the specific peptide fragments that were shown to bind to BMS-214662 and/or biotinylated analogues thereof. A summary of these peptide fragments, the ions observed from the LC/LC/MS/MS spectra, in addition to the frequency of observed fragments is provided in Table 6.

FIGS. 69A-C. Shows the observed LC/LC/MS/MS spectra for fragment 1 of PTPN6 that bound to BMS-214662 and/or biotinylated analogues thereof (SEQ ID NO:31).

FIG. 70. Shows a summary of the ions observed from the LC/LC/MS/MS spectra for fragment 1 of PTPN6 that bound to BMS-214662 and/or biotinylated analogues thereof (SEQ ID NO:31).

FIGS. 71A-C. Shows the observed LC/LC/MS/MS spectra for fragment 2 of PTPN6 that bound to BMS-214662 and/or biotinylated analogues thereof (SEQ ID NO:32).

FIG. 72. Shows a summary of the ions observed from the LC/LC/MS/MS spectra for fragment 2 of PTPN6 that bound to BMS-214662 and/or biotinylated analogues thereof (SEQ ID NO:32).

FIGS. 73A-C. Shows the observed LC/LC/MS/MS spectra for fragment 3 of PTPN6 that bound to BMS-214662 and/or biotinylated analogues thereof (SEQ ID NO:33).

FIG. 74. Shows a summary of the ions observed from the LC/LC/MS/MS spectra for fragment 3 of PTPN6 that bound to BMS-214662 and/or biotinylated analogues thereof (SEQ ID NO:33).

FIGS. 75A-C. Shows the observed LC/LC/MS/MS spectra for fragment 4 of PTPN6 that bound to BMS-214662 and/or biotinylated analogues thereof (SEQ ID NO:34).

FIG. 76. Shows a summary of the ions observed from the LC/LC/MS/MS spectra for fragment 4 of PTPN6 that bound to BMS-214662 and/or biotinylated analogues thereof (SEQ ID NO:34).

FIGS. 77A-C. Shows the observed LC/LC/MS/MS spectra for fragment 5 of PTPN6 that bound to BMS-214662 and/or biotinylated analogues thereof (SEQ ID NO:35).

FIG. 78. Shows a summary of the ions observed from the LC/LC/MS/MS spectra for fragment 5 of PTPN6 that bound to BMS-214662 and/or biotinylated analogues thereof (SEQ ID NO:35).

FIGS. 79A-C. Shows the observed LC/LC/MS/MS spectra for fragment 6 of PTPN6 that bound to BMS-214662 and/or biotinylated analogues thereof (SEQ ID NO:36).

FIG. 80. Shows a summary of the ions observed from the LC/LC/MS/MS spectra for fragment 6 of PTPN6 that bound to BMS-214662 and/or biotinylated analogues thereof (SEQ ID NO:36).

FIG. 81. Shows the sequence of an isoform of the Hsp70 protein (gi|NP_{—}005336.2; SEQ ID NO:37), as well as denoting the specific peptide fragments of this isoform that were shown to bind to BMS-214662 and/or biotinylated analogues thereof. A summary of these peptide fragments, the ions observed from the LC/LC/MS/MS spectra, in addition to the frequency of observed fragments is provided in Table 7.

FIG. 82. Shows the sequence of an isoform of the Hsp70 protein (gi|NP_{—}068814.2; SEQ ID NO:49), as well as denoting the specific peptide fragments of this isoform that were shown to bind to BMS-214662 and/or biotinylated analogues thereof. A summary of these peptide fragments, the ions observed from the LC/LC/MS/MS spectra, in addition to the frequency of observed fragments is provided in Table 8.

FIG. 83. Shows the sequence of an isoform of the Hsp70 protein (gi|NP_{—}002145.3; SEQ ID NO:55), as well as denoting the specific peptide fragments of this isoform that were shown to bind to BMS-214662 and/or biotinylated analogues thereof. A summary of these peptide fragments, the ions observed from the LC/LC/MS/MS spectra, in addition to the frequency of observed fragments is provided in Table 9.

FIG. 84. Shows the sequence of an isoform of the Hsp70 protein (gi|NP_{—}005338.1; SEQ ID NO:57), as well as denoting the specific peptide fragments of this isoform that were shown to bind to BMS-214662 and/or biotinylated analogues thereof. A summary of these peptide fragments, the ions observed from the LC/LC/MS/MS spectra, in addition to the frequency of observed fragments is provided in Table 10.

FIG. 85. Shows the sequence of an isoform of the Hsp70 protein (gi|NP_{—}002146.2; SEQ ID NO:62), as well as denoting the specific peptide fragments of this isoform that were shown to bind to BMS-214662 and/or biotinylated analogues thereof. A summary of these peptide fragments, the ions observed from the LC/LC/MS/MS spectra, in addition to the frequency of observed fragments is provided in Table 11.

FIG. 86. Shows the sequence of an isoform of the Hsp70 protein (gi|NP_{—}006588.1; SEQ ID NO:67), as well as denoting the specific peptide fragments of this isoform that were shown to bind to BMS-214662 and/or biotinylated analogues thereof. A summary of these peptide fragments, the ions observed from the LC/LC/MS/MS spectra, in addition to the frequency of observed fragments is provided in Table 12.

FIG. 87. Shows the sequence of an isoform of the Hsp70 protein (gi|NP_{—}004125.3; SEQ ID NO:76), as well as denoting the specific peptide fragments of this isoform that were shown to bind to BMS-214662 and/or biotinylated analogues thereof. A summary of these peptide fragments, the ions observed from the LC/LC/MS/MS spectra, in addition to the frequency of observed fragments is provided in Table 13.

FIG. 88. Shows the sequence of the prolyl-4-hydroxylase, beta protein (gi|NP_{—}000909.2; SEQ ID NO:78), as well as denoting the specific peptide fragments of this isoform that were shown to bind to BMS-214662 and/or biotinylated analogues thereof. A summary of these peptide fragments, the ions observed from the LC/LC/MS/MS spectra, in addition to the frequency of observed fragments is provided in Table 14.

FIG. 89. Shows the sequence of the pyruvate kinase 3 protein (gi|NP_{—}002645.3; SEQ ID NO:84), as well as denoting the specific peptide fragments of this isoform that were shown to bind to BMS-214662 and/or biotinylated analogues thereof. A summary of these peptide fragments, the ions observed from the LC/LC/MS/MS spectra, in addition to the frequency of observed fragments is provided in Table 15.

FIG. 90. Shows the sequence of the enolase 1 protein (gi|NP_{—}001419.1; SEQ ID NO:100), as well as denoting the specific peptide fragments of this isoform that were shown to bind to BMS-214662 and/or biotinylated analogues thereof. A summary of these peptide fragments, the ions observed from the LC/LC/MS/MS spectra, in addition to the frequency of observed fragments is provided in Table 16.

FIG. 91. Shows the sequence of the adenylate cyclase protein (gi|NP_{—}006358.1; SEQ ID NO:112), as well as denoting the specific peptide fragments of this isoform that were shown to bind to BMS-214662 and/or biotinylated analogues thereof. A summary of these peptide fragments, the ions observed from the LC/LC/MS/MS spectra, in addition to the frequency of observed fragments is provided in Table 17.

### DETAILED DESCRIPTION OF THE INVENTION

In accordance with the present invention, the inventors have identified for the first time, the association between agonizing AIF, HOP, phosphatase 1G, protein tyrosine phosphatase non-receptor type 6 isoform 2, Hsp70, and/or Other Target Protein, or in antagonizing the ability of Hsp70 to sequester AIF, with the benefit of selectively inducing apoptosis in quiescent, non-proliferating cells. This association was elucidated based upon the identification of the mechanism of action of BMS-214662.

In the studies described herein, BMS-214662 caused rapid regressions of large tumors in a number of xenograft models, in many instances producing curative efficacy. Described originally as a farnesyltransferase inhibitor (FTI), this level of activity was not consistent with the properties reported for other FTIs. This observation suggested that BMS-214662 is selectively targeting the quiescent cells, since they constitute most of the solid tumors mass, and contain variable proportions of proliferating cells. Cells made quiescent by nutrient deprivation were shown to be one to two orders of magnitude more sensitive to apoptosis induced by BMS-214662 than cells actively proliferating. The inventors demonstrate that combining BMS-214662 with agents that either block proliferation in several tumor cell types (i.e., enrich the quiescent G0/G1 state), or are cytotoxic to proliferating cells under a variety of conditions, resulted in therapeutic synergy. The synergy was observed both in vitro and in vivo, in multiple tumor xenograft models, and in combination with a variety of agents that target proliferating cells. Most existing anticancer drugs, including the cytostatic FTIs, show a preferential cytoxic or cytostatic activity against proliferating cells. Because of the differential selectivity of BMS-214662 for quiescent cells, these drug effects can be observed at clinically achievable concentrations. These findings impact the scope of clinical development of BMS-214662 to realize its full potential for human benefit. The results described herein also show the feasibility of using this model to further discover novel agents targeting quiescent tumor cells and cancer stem cells, providing a new strategy for the development of anti-cancer therapies.

### BMS-214662 Selectively Targets Quiescent Cells

Our previous report (Rose et al., Cancer Res., 61:7507-7517 (2001)) demonstrated that BMS-214662 differed sharply from other FTIs, such as SCH66336 (Schering-Plough), L-744832 (Merck), R-115777 (Johnson & Johnson), BIM-46228, FTI-277, B-956 and FTI-2153. These compounds acted as cytostatic drugs and produced non-curative regressions in transgenic tumor models or xenografts of human tumors (Nagasu et al., Cancer Res., 55:5310-531 (1995); Kohl et al., Nat. Med., 1:792-797 (1995); Prevost et al., Int. J. Cancer, 83:283-287 (1999); Prevost et al., Int. J. Cancer, 91:718-722 (2001); Sun et al., Cancer Res., 59:4919-4926 (1999); Barrington et al., Mol. Cell. Biol., 18: 85-92 (1998); Norgaard et al., Clin. Cancer Res., 5:35-42 (1999); Liu et al., Cancer Res., 58:4947-4956 (1998); and End et al., Cancer Res., 61:131-137 (2001)). BMS-214662 displayed both potent tumor regression and curative activity accompanied by extensive apoptosis in such models (Rose et al., Cancer Res., 61:7507-7517 (2001)). Since a majority of the cells in a solid tumor are in a Q cell state (Jackson, R. C., Adv. Enzyme Regul., 29:27-46 (1989)), these observations suggested that the target for BMS-214662 may include the non-proliferating, i.e., Q, cell subpopulation. To investigate this hypothesis, the inventors determined the cytotoxicity of BMS-214662 in vitro versus HCT-116 cells in P and Q phases.

### Proliferation Analysis of HCT116 Cells In Vitro

To model the nutrient deprivation status of solid tumors in vitro the inventors kept cells in culture without re-feeding for 4 days and drug was provided in spent media for the fifth day. These Q cells were still alive and fully capable of resuming growth on re-feeding, for weeks after Q cells were induced.

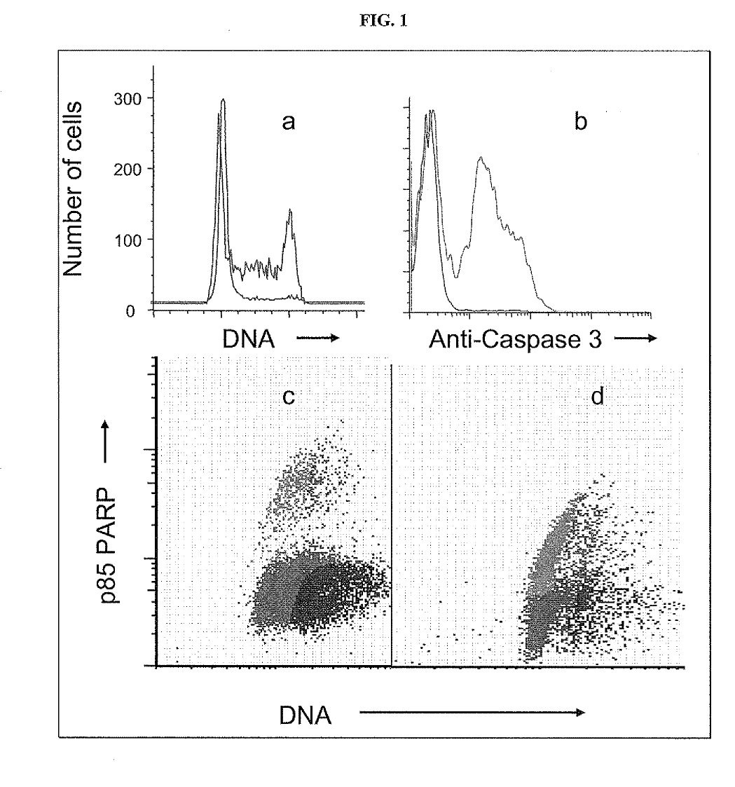

The P (S and G2/M) and Q (G0/G1) character of HCT-116 cells in culture for 3 and 5 days was confirmed by cell cycle analysis using flow cytometry (P cells were in G1=29.7%; S=50.7% and G2=18.6%, Q cells were in G1=77.2%; S=17.4% and G2=2.8%, FIG. 1a) and confirmed by BrdU labeling studies (FIG. 1, panels c and d). P cells were dramatically reduced in the 5 day Q cell population and accumulate in G0/G1, as shown by DNA content and reduced BrdU incorporation (FIGS. 1a and d).

A short (4 hr) incubation with BMS-214662 (3 uM) followed by a 20 hour chase resulted in a significant increase in the number of pre-apoptotic cells in the Q cell population, as measured by detection of activated caspase 3 (FIG. 1b), activated caspase 9 (not shown) or by cleaved poly-ADP-ribose polymerase (p85 PARP, FIG. 1d), a marker for an irreversible apoptosis step (collectively referred to as “caspase-dependent apoptosis”). In contrast, P cells remained abundant after BMS-214662 treatment, since this short exposure and low dose were insufficient to activate the apoptosis pathway as detected by PARP cleavage (FIG. 1c).

Reduced toxicity of BMS-214662 in P cells could be due to differential permeability. The inventors examined the RAS farnesylation state in P cells treated with BMS-214662 for 24 hours, and found that the unmodified form accumulated. The inhibitory activity of BMS-214662 on FT in P cells excludes lack of drug penetration as an argument for the lack of cytotoxic activity (data not shown). In contrast, in the Q cells the level of farnesylated RAS remained unchanged during the 24 hour treatment. The inventors believe this stability was due to lack of turnover of the RAS protein in the absence of cell proliferation, and not to lack of penetration of the compound, since BMS-214662 efficiently induced effector caspase 3 activation, PARP cleavage and apoptosis in these Q cells.

### Quantitative Analysis of Cell Survival in the Presence of BMS-214662

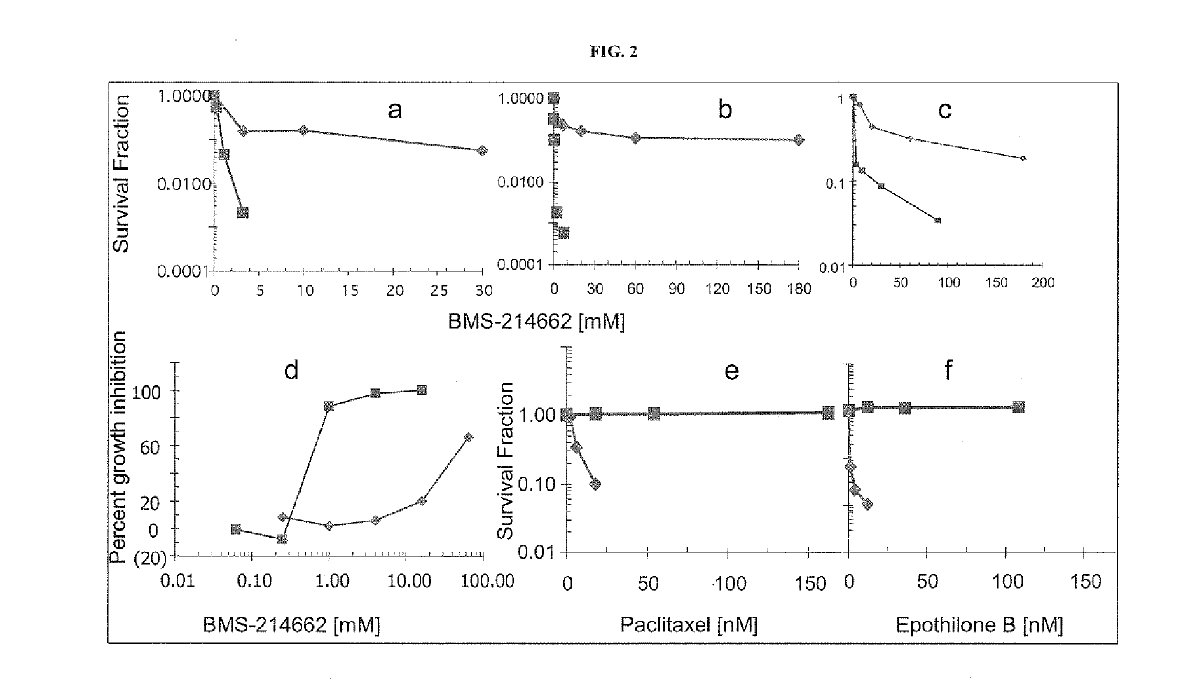

The potency with which BMS-214662 killed tumor cells was evaluated by a clonogenic assay of the surviving cell population following treatment. BMS-214662 killed both P and Q HCT-116 human colon carcinoma cells (35 and 0.3 μM, respectively; FIG. 2a). Selected examples of 3 other human cell lines tested, including colon (HT-29), ovarian (pat-7) and chronic myelogenous leukemia (CML, displayed as percent inhibition) K-562 are shown in FIGS. 2b, 2d and 2e, respectively. HT-29 colon cells in the Q phase were the most sensitive to BMS-214662, with a selectivity ratio (IC90 for P/Q) of 160, while the selectivity ratios in the K562 line was variable, with ratios ranging from 5 to 68, depending on experimental conditions.

### BMS-214662 is Unique Among Anti-Cancer Drugs in Killing Quiescent Cells

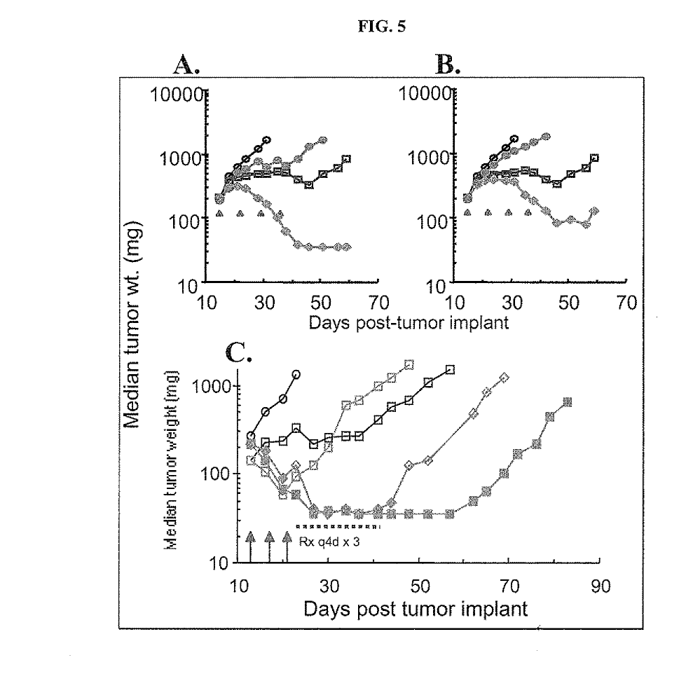

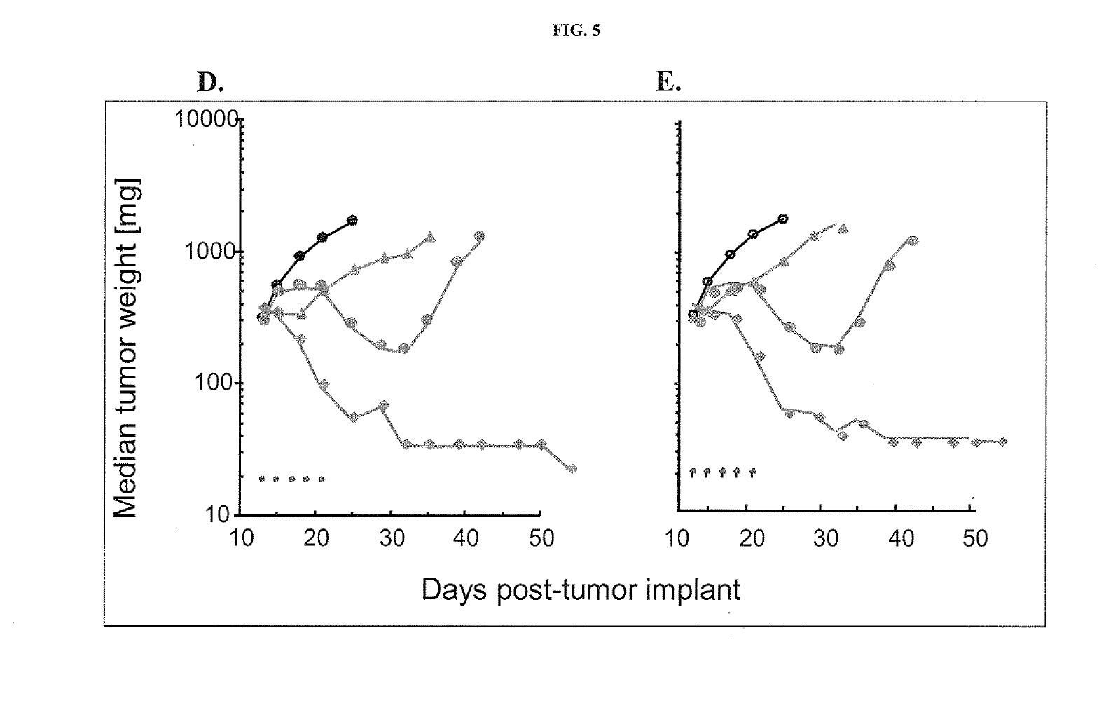

Most cytotoxic anti-cancer drugs work by affecting DNA synthesis or cell division, and therefore target P cells while leaving Q cells unaffected. In our assay system the cancer agents paclitaxel (IC90=17.8 nM, (FIG. 2e), ixabepilone (IC90=1.3 nM, FIG. 2f) and 5-fluorouracil (5FU, not shown) are preferentially cytotoxic to P tumor cells. The relative sensitivities of P versus Q cells (>83-fold more sensitive to ixabepilone for P cells, Q cell values not reached for paclitaxel) for these classic cytotoxic compounds were expected, and are the opposite of the effect of BMS-214662.

### Structural Basis for Quiescent Cell Selectivity of Tetrahydrobenzodiazepine FTIs, and its Relationship to Anti-Tumor Efficacy

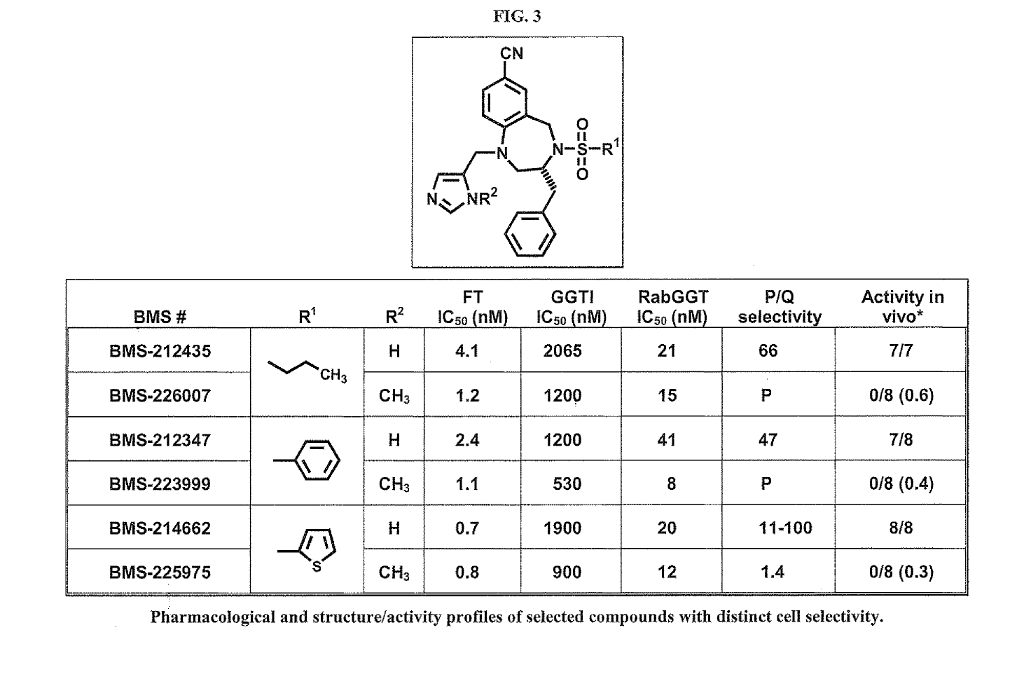

In an effort to understand the structural basis for the selectivity of BMS-214662 to Q cells, and its relationship to in viva anti-tumor efficacy, the inventors tested several related tetrahydrobenzodiazepine compounds (FIG. 3) in these assays.

Table 1 summarizes the structures tested and the results obtained. Prenyltransferase inhibitory activity against FT, geranylgeranyl transferase I (GGTI), (which shares its α subunit with FT), and the more distantly related Rab geranylgeranyl transferase (GGTII, previously associated with proapoptotic activity (Lackner et al., Cancer Cell., 4:325-36 (2005))), have been tested in vitro. Activities in cellular assays, P versus Q cell population selectivity data and efficacies in vivo against HCT-116 xenografts are also shown.

P versus Q cell selectivity results were best typified by BMS-225975 and BMS-214662. This otherwise identical pair of compounds has either a hydrogen atom (BMS-214662) or a methyl group (BMS-225975) on the π nitrogen of the imidazole. Both compounds showed similar potent inhibition of FT, GGTII and cell proliferation in vitro, but differed dramatically in their tumor regressing activity in vivo (Manne et al., Cancer Res., 64:3974-3980 (2004)) and selectivity, i.e., BMS-225975 is almost equally active on P and Q cells (ratio 0.7). Similar P/Q activity ratios were obtained for SCH66336 and R-115777 (0.74 and 0.83, respectively) as well as for other cytostatic FTIs (Lombardo et al., Bioorg. Med. Chem. Lett., 15:1895-1899 (2005)). On the other hand, BMS-212435 and BMS-212347 resemble BMS-214662 in their potent activity in vivo against HCT-116 human colon tumor xenografts and also exhibited selective targeting of Q tumor cells in vitro. While the simplest interpretation of this data indicates an important role for the H moiety, analysis of structure/activity relationships of additional compounds suggested additional chemical complexity.

The compounds described above have similar metabolism and pharmacokinetic properties, excluding these factors as potential explanations for the lack of antitumor activity in vivo or in tissue culture. Thus, Q cell selectivity of these compounds, correlated with in vivo anti-tumor activity in the HCT116 xenograft model, and not with potency against FT, GGT I or GGTII.

Synergistic Combination of BMS-214662 with Anti-Proliferative Agents In Vitro

The mechanistic studies presented above showing the selective cytotoxicity of BMS-214662 to Q tumor cells raised the prospect of combination therapy with existing anticancer drugs (which target P tumor cells, FIG. 2). The inventors investigated the effects of BMS-214662 in combination with paclitaxel, using clonogenic cell survival assays. Results analyzed using an isobologram plot indicate that combination of paclitaxel with BMS-214662 in vitro yielded a synergistic cytotoxic effect. Synergism was only observed when cells were treated with the two agents simultaneously, or when paclitaxel treatment preceded BMS-214662 addition. When BMS-214662 was administered 20 hr prior to paclitaxel, antagonism was observed. These results suggested that an accumulation of cells in the Q fraction by paclitaxel resulted in more efficacious treatment. The mechanism underlying the dependency of synergism on treatment sequence has been suggested to be related to cell cycle effects of BMS-214662. This result suggests a clear strategy to follow in the clinical application of BMS-214662.

### Quiescent Cells Constitute a Major Fraction of Solid Tumors, and are Sensitive to BMS-214662 In Vivo

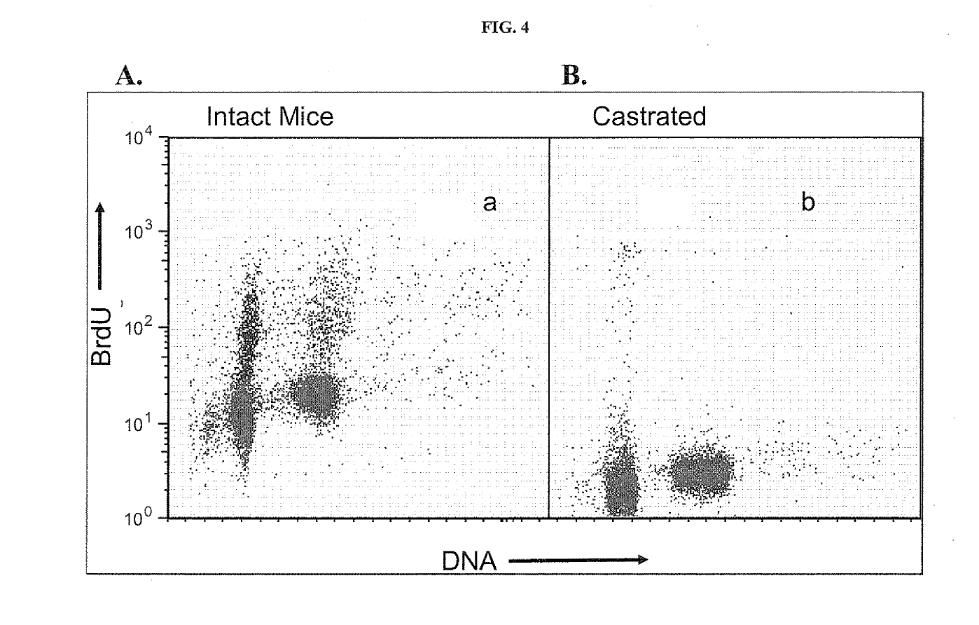

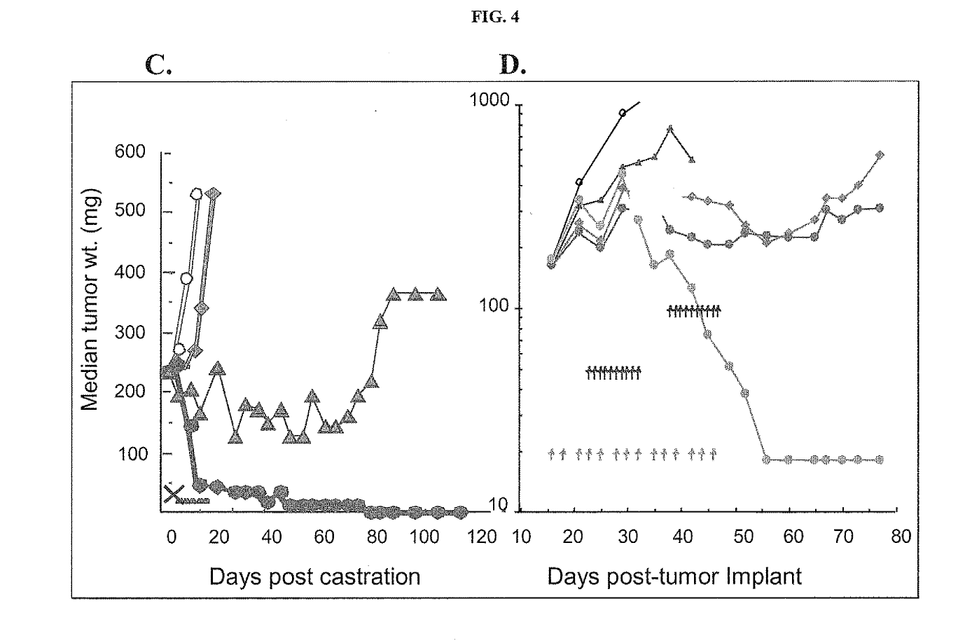

Hormonal deprivation by castration has been demonstrated to result in a transient but reproducible arrest of both prostate and mammary tumor growth. To evaluate and experimentally manipulate with hormones the population of Q cells in tumors, the inventors analyzed xenografts of MDA-PCa2b, an androgen-dependent line derived from a prostate cancer patient (Navone et al., Clin. Cancer Res., 3:2493-2500 (1997)) and MCF7, an estrogen dependent breast cancer line. Nude mice bearing MDA-PCa2b xenograft tumors were infused with BrdU for 24 hr. Their tumors were removed, dissociated and analyzed by flow cytometry. In a 120 mg MDA-PCa2b tumor, only about 40% of constituted P cells while the remaining 60% did not incorporate BrdU, reflecting their Q character (FIG. 4a). Although the shorter 24 hour measurement of BrdU incorporation may slightly overestimate the number of Q cells, similar results were observed for xenografts of HCT-116, San-1 (salivary gland) and Pat 21 (ovarian, results not shown), and results obtained with longer incorporation experiments (Jackson, R. C., Adv. Enzyme Regul., 29:27-46 (1989)), support the fact that Q cells predominate in tumors.

In the MDA-PCa2b established tumors one day after castration, the rapid disappearance of the P cells (FIG. 4a, right panel) was paralleled by a prolonged stasis, and tumor size remained relatively constant until “hormone independent” growth 70-80 days later (FIG. 4b). In the absence of castration, administration of BMS-214662 for 12 days had no significant effect on tumor growth. However, treatment with the compound one day after castration resulted in a dramatic reduction of tumor load, presumably by targeting the now Q tumor cell population in this xenograft model (FIG. 4b).

Xenografts of the mammary cell line MCF-7 provided a second hormonally regulated tumor model. Similar to many human breast cancers, MCF-7 requires estrogen for growth and anti-estrogens, such as tamoxifen, inhibit MCF-7 xenograft growth. The low host estrogen levels in mice require supplementing with an estrogen pellet for effective tumor growth. In a manner analogous to the prostate tumor example above, stasis of MCF-7 tumors is observed on removal of the estrogen pellet or on administration of tamoxifen. Again, while treatment with BMS-214662 alone was ineffective, combination of tamoxifen followed by BMS-214662 resulted in reduction of tumor size to undetectable levels (FIG. 4c, tumor cures in 3 of 8 mice). In conclusion, induction of stasis and/or elimination of the P cell population, resulted in significant potentiation of the anti-tumor activity of these treatment combinations with BMS-214662.

Synergistic Interaction of BMS-214662 with Cytotoxic Agents In Vivo

The inventors next investigated the value of combination therapies in vivo using existing or novel cytotoxic agents that preferentially kill P cells, such as paclitaxel, ixabepilone and CPT-11 (FIGS. 2, c and f and FIG. 5). To provide a clinically relevant assessment of the anti-tumor effects of combining paclitaxel with BMS-214662, both agents were administered intravenously (IV). The levels of w BMS-214662 in this study were relatively low (40-80 mg/kg) and were chosen in order to mimic the plasma exposure that was achieved in patients in phase 1 studies after a 1 hr infusion (˜30 μM×hr) (Ryan et al., Clin. Cancer Res., 10:2222-2230 (2004); Papadimitrakopoulou et al., Clin. Cancer Res., 11:4151-4159 (2005); Tabernero et al., J. Clin. Oncol., 23:2521-2533 (2005)). Clear synergy of BMS-214662 with paclitaxel resulting in tumor shrinkage were observed at both doses of BMS-214662 in combination with 20 mg/kg paclitaxel given weekly×4 (paclitaxel was given 3 hr prior to BMS-214662, FIGS. 5a and b). This synergistic interaction was seen not only in tumor growth delay, but also in terms of the more clinically relevant measures of tumor response, namely, partial and complete tumor regression/response, (PR and CR, respectively) and in tumor cures.

Therapeutic synergism was also clearly demonstrated by the combination of BMS-214662 and ixabepilone in vivo. The inventors chose the multidrug resistant human colon carcinoma xenograft HCT/VM46, against which both compounds have modest anti-tumor activity as single agents (FIG. 5c, 1.6 and 1.1 LCK, respectively) but did not produce any tumor cures. However, in combination (ixabepilone followed 24 hr later by BMS-214662), a highly significant increase in tumor growth delay (3.7 LCK) and curative effects were observed in 3 of 7 mice (FIG. 5c). When BMS-214662 treatment was administered 24 hr prior to ixabepilone, no therapeutic synergism was observed, with the combination performing only as well as ixabepilone given alone (results not shown). Therefore, the consistently observed synergy of the combinations is highly sequence dependent, in vitro and in vivo.

CPT-11, a topoisomerase I inhibitor, selectively targets P cells that are undergoing active DNA synthesis. Mice bearing advanced (300 mg) HCT-116 xenografts were treated with CPT-11 followed one hr later by BMS-214662. CPT-11 was administered IV at or near its MTD of 30 mg/kg/injection(inj) whilst BMS-214662 was given at two different dose levels: 60 and 80 mg/kg/inj, IV. The combination produced significantly higher PR and CR rates as compared to single agents (FIGS. 5d and e). Thus, the inventors conclude that treatments that eliminate P cell populations, whether hormonal manipulations or cytotoxic agents, followed by treatment with BMS-214662, resulted in sequence dependent synergism. This synergism increased the efficacy and reduced the dose of BMS-214662 required, when compared to its single agent activity.

### Achievable Levels

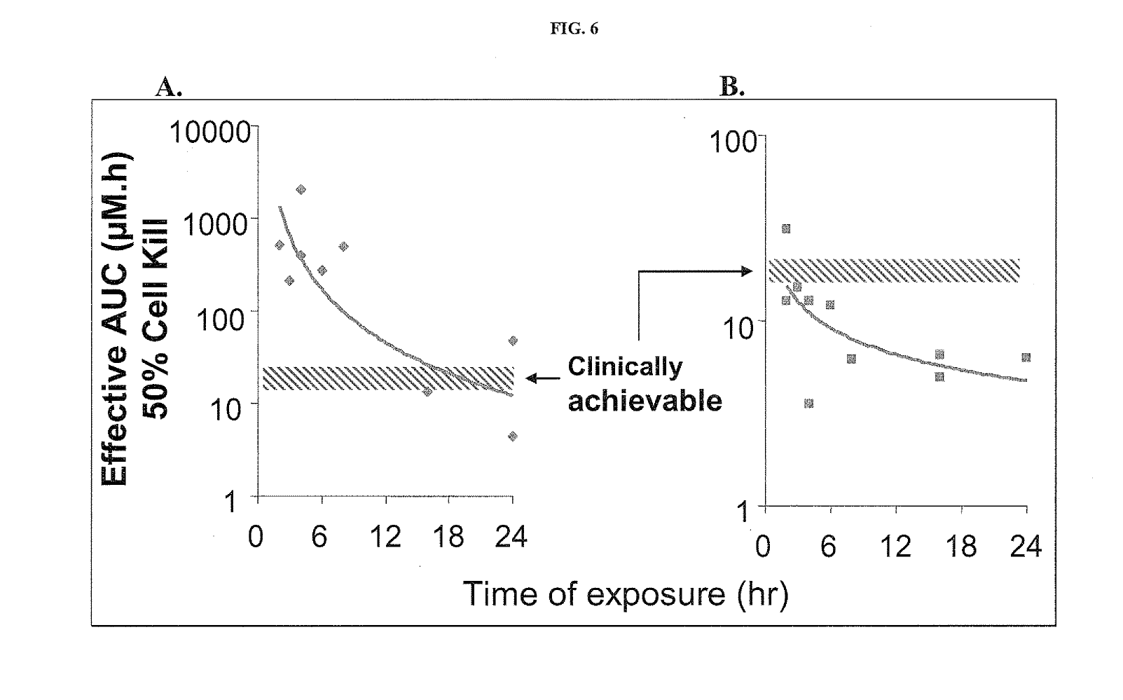

To investigate the possible clinical impact of doses of BMS-214662 the inventors performed a pharmacokinetic analysis in P and Q cell populations. The inventors determined the concentrations of BMS-214662 and exposure times required to detect changes in cell viability by our colony forming assay (FIG. 6). The levels of BMS-214662 exposure achieved in phase I studies in humans (22-26) are also presented. For P cells, the inventors found that effective cell killing requires a concentration of BMS-214662 which exceeds the levels achievable in humans, while in Q cells, the levels of BMS-214662 required for killing 50% of the cells can be readily achieved.

### Mechanism of Action of BMS-214662

The development of chemical proteomic approaches has allowed us to identify targets for drugs of unknown MOA. The approach consists of identifying which points on the drug of interest can be readily modified (using SAR studies) to attach long arms of a molecule that can be linked to a solid support. Developments in a form of monomeric avidin that gently releases bound biotin molecules at 2 mM biotin has allowed derivatives of biotin to be attached on one end to avidin solid supports and to molecules of interest at the other end, with linkers of different lengths. Gentle elution allowed for the purification of bound proteins 5-100,000 fold and then followed by their subsequent identification by MS/MS.

Previously, several experiments were run to identify the target of BMS-214662. All of these experiments were unable to give a clear result. There may have been several reasons for this result, which include continuity of resources, technical developments in both detection and analytical procedures and unified focus. The strategies used included affinity chromatography of a biotinylated compound on monomeric avidin agarose, cross linking with an azido derivatized compound, competition with inactive compounds, and protein size fractionation, in addition to extensive transcriptional profiling.

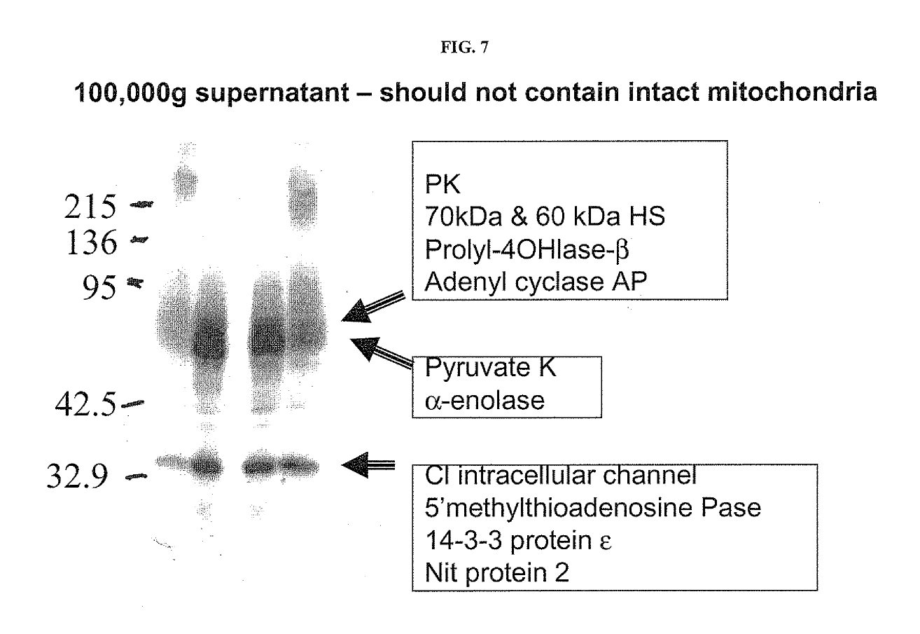

Affinity capture experiments were repeated which led to the identification of HOP and AIF as proteins that specifically bound to BMS-214662. Subsequent molecular modeling demonstrated that BMS-214662 could bind to a deep pocket within the AIF protein. In addition, the HOP molecular chaperone phosphoprotein was found to bind to the BMS-214662 affinity matrix. Results of these experiments on ALL cells are summarized in FIGS. 7 to 15 and Table 2.

The demonstration that BMS-214662 could bind to a pocket within the AIF protein in-silico, binding to the deep pocket within the AIF protein, should make competition with other molecules rather complex. In addition, it is proposed that inactive competitors may bind to AIF, but not be effectors for the proapoptotic effect of AIF.

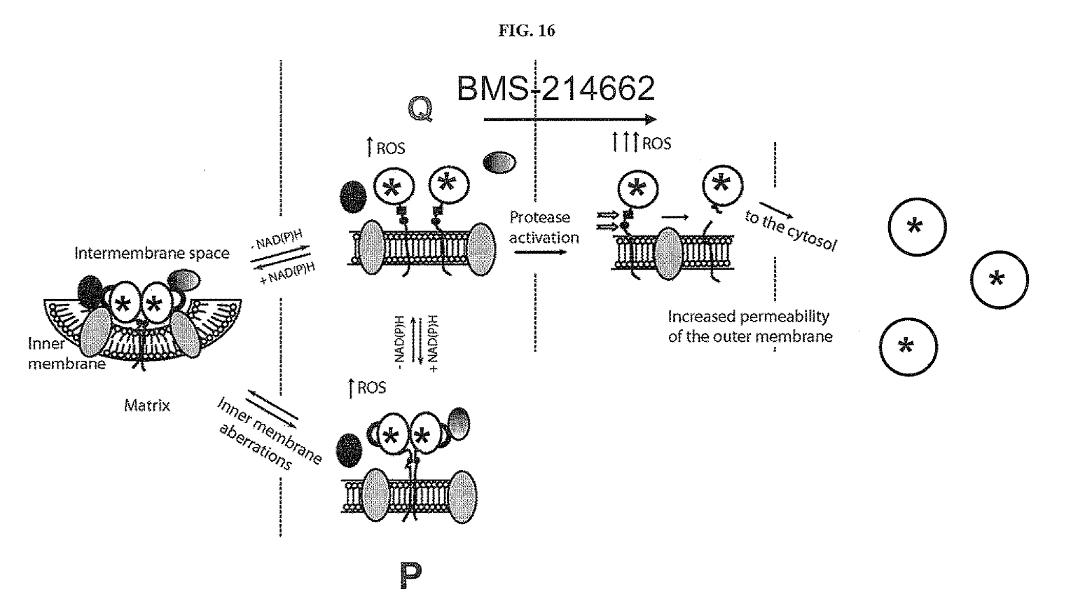

One explanation for the agonizing effect on the pro-apoptotic activity of activated AIF by BMS-214662 is that BMS-214662 may be accelerating the escape of precursor AIF from mitochondria to the nucleus after cleavage of the 100 amino terminal amino acids of the protein, or altering the tertiary structure or folding of the protein to accelerate its nuclear translocation. According to this model (shown in FIG. 16), conditions of limiting nutrients or reduced mitochondrial function as is present for cells in a quiescent state result in conformation changes in the AIF protein that makes it more accessible to the protease calpain, which results in proteolytuc cleavage and the subsequent release of AIF from the inner mitochondrial membrane. BMS-214662 binds to AIF and is believed to facilitate its release from the inner mitochondrial membrane into the cytosol by stabilizing the structure of AIF that is sensitive to calpain activity. Once in the cytoplasm, it would be bound by the Hsp70-HOP-Hsp90 complex and shuttled to the nucleus. Based upon the short time frame in which BMS-214662 induces apoptosis in quiescent cells (less than 2 hours), it seems unlikely that transcriptional changes caused by BMS-214662, if any, would result in the observed activity.

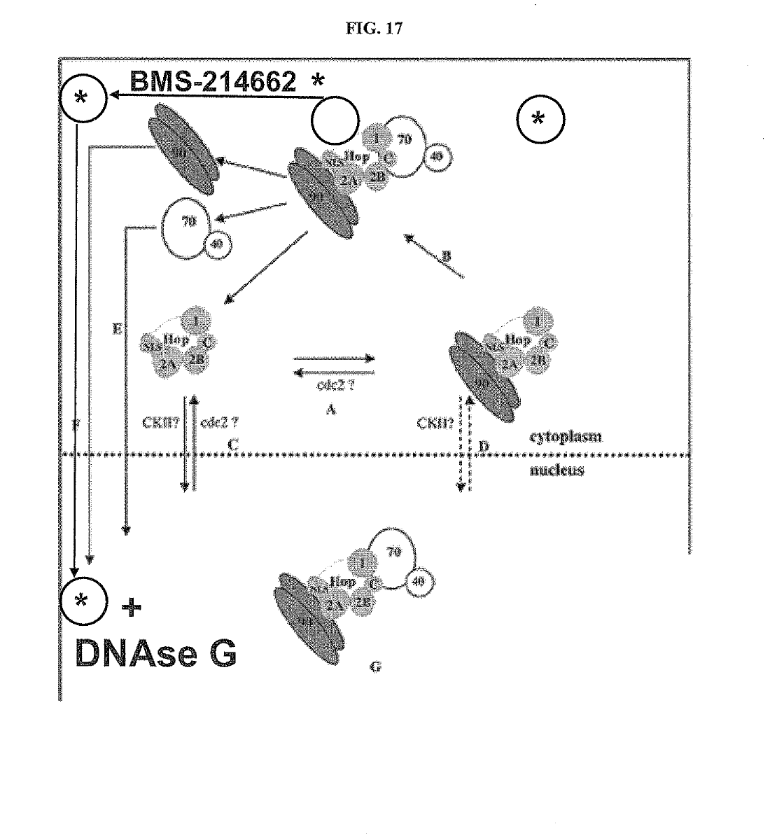

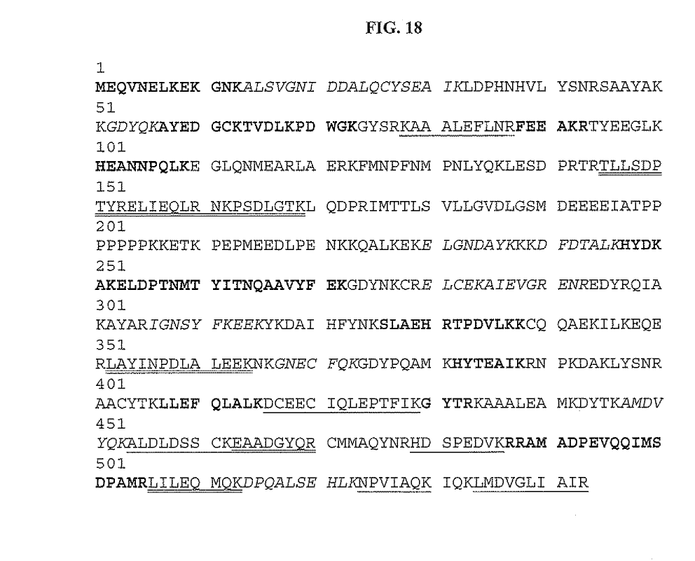

Another alternative explanation is that BMS-214662 agonizes HOP in such a way that it increases the frequency by which it accumulates, localizes, or translocats to the nucleus, and that such an increased frequency results in an increased level of AIF being transported into the nucleus, either directly by HOP, or indirectly via the Hsp70-HOP-Hsp90 complex. According to this model (shown in FIG. 17), the mechanism of action for BMS-214662 in agonizing the release of AIF from the Hsp70-HOP-Hsp90 complex. In this model, AIF is released into the cytoplasm and bound by Hsp70-HOP-Hsp90 in an inactive form on account of HSP70 being known to inhibit the proapoptotic function of AIF. An alternative or additional activity of BMS-214662 would be related to the release from the Hsp70-HOP-Hsp90 complex to act in the nucleus in combination with DNAse-G in the initiation of the apoptotic process, as shown. The cleavage of PARP (polyADPribose polymerase) in turn frees polyADP ribopolymers which in turn feedback for more AIF release.

As shown in FIG. 17, the possible links between the subcellular localization of the phosphoprotein Hop and its functionality as a Hsp70/Hsp90 scaffolding protein are shown. Hop exists on its own or in complex with Hsp90, in the cytoplasm under normal conditions. This may be regulated by phosphorylation, with cdc2 kinase phosphorylation of Hop disrupting its interaction with Hsp90 (cdc2 arrow; A). Interaction of Hop with Hsp90 is known to facilitate a number of other interactions, of which the most well established one is the interaction of the Hop-Hsp90 complex with Hsp70 (associated with its co-chaperone Hsp40 and substrate) in order to facilitate substrate transfer from Hsp70 to Hsp90 (13). This multichaperone complex then dissociates, freeing its various components (including AIF, for example). Hop is known to translocate to the nucleus (C) under stressful conditions, and its localization may be regulated by phosphorylation, with CKII phosphorylation possibly promoting nuclear localization (CKII arrow) and cdc2 kinase phosphorylation possibly promoting cytoplasmic retention (cdc2 arrow). It is speculated that Hop may also be capable of moving into the nucleus in concert with Hsp90 (D) as a complex (arrows shown in dotted lines) by either the putative NLS (222-239), or through the functioning of multiple NLSs, and possibly also promoted by CKII phosphorylation (CKII dotted arrow). It is already known that both Hsp70 (together with Hsp40) and Hsp90 translocate into the nucleus under heat shock (E and F respectively). Within the nucleus Hop may have a number of functions, including its basic function of interacting with Hsp70 and/or Hsp90 to form nuclear complexes (G). The 1, 2A, 213, C and NLS annotations on Hop refer to its TPR1, TPR2A and TPR2B domains, C-terminal domain, and nuclear localization signal sequence, respectively. Hsp40, Hsp70 and Hsp90 are labeled as 40, 70 and 90, respectively.

A third explanation for the ability of BMS-214662 to inhibit quiescent, non-proliferating tumor cells implicates cytoplasmic factors, including, but not limited to Hsp-70. In this model, the BMS 214662 agonist activity towards AIF may also occur in whole or in part through disruption of AIF binding to cytoplasmic factors. Such disruption would result in release of AIF from the bound cytoplasmic factor and permit AIF localization into the nucleus where it could facilitate chromatin condensation and induction of apoptosis. Such a direct interaction between AIF and cytoplasmic HSP70 has been reported and the cellular consequences of the interaction elucidated (Powers et al., FEBS Lett., 581:3758-3769 (2007); Morano, K. A., Ann. NY Acad. Sci., 1113:1-14 (2007); Ravagnan et al., Nat. Cell Biol., 3:839-843 (2001); Gurbuxani et al., Oncogene, 22:6669-6678 (2003); Ruchalsk et al., J. Biol. Chem., 281:7873-7880 (2006); Schmitt et al., Can. Res., 66:4191-4197 (2006); Park et al., Autrophagy, 4:364-367 (2008); Jolly et al., J. Natl. Cancer Inst., 92:1564-1572 (2000); Mosser et al., Oncogene, 23:2907-2918 (2004)). Indeed, transfection of HSP70 into MEF cells has been shown to sequester AIF protein in the cytoplasm, to prevent AIF localization to the nucleus, and reduce to AIF induction of caspase independent apoptosis (Ravagnan et al., Nat. Cell. Biol., 3:839-843 (2001); Gurbuxani et al., Oncogene, 22:6669-6678 (2003)). In addition, knockdown of HSP70 levels by antisense was shown to increase AIF localization to the nucleus, and induce AIF caspase independent apoptosis (Ruchalsk et al., J. Biol. Chem., 281:7873-7880 (2006); Ravagnan et al., Nat. Cell Biol., 3:839-843 (2001)). Through deletion mapping, the AIF and HSP70 domains required for this interaction have been defined (Ravagnan et al., Nat. Cell Biol., 3:839-843 (2001); Gurbuxani et al., Oncogene, 22:6669-6678 (2003); Ruchalsk et al., J. Biol. Chem., 281:7873-7880 (2006); Schmitt et al., Can. Res., 66:4191-4197 (2006)). In a parallel pathway of caspase dependent apoptosis, HSP70 binding to APAF-1 prevents and inhibits apoptosis (Ravagnan et al., Nat. Cell Biol., 3:839-843 (2001); Park et al., Autrophagy, 4:364-367 (2008)). These activities of HSP70 are thought to be safety mechanisms to prevent cell death in response to transient cellular stresses in normal cells, whereas in some cancer cells the same mechanism is activated to provide a proliferative advantage to cancer cells, allowing them to escape cell death processes.

Normally, HSP70 expression is transcriptionally upregulated only in response to cellular stress. In cancer cells, levels of the inducible form of HSP70 protein are elevated and may function to provide protection from cell death process such as autophagy and apoptosis (Park et al., Autrophagy, 4:364-367 (2008); Jolly et al., J. Natl. Cancer Inst., 92:1564-1572 (2000); Mosser et al., Oncogene, 23:2907-2918 (2004); Ciocca, D. R. et al., Cell Stress Chaperones, 10:86-103 (2005); Kaur, J. et al., Int. J. Cancer, 63:774-779 (1995); Hantschel, M. et al., Cell Stress Chaperones, 5:438-442 (2000)). Cancer cells treated with cytotoxic drugs additionally show stress induced increases in the levels of HSP70 as a stress and/or resistance mechanism response (Pocaly et al., Leukemia, 21:93-101 (2007)). Interestingly, stem cells have been reported to express inducible HSP70 in the absence of stress, a potential survival mechanism for these progenitor cells (Geraci et al., Cell. Death Differ., 13:1057-1063 (2006); and Lyngholm et al., Exp. Eye Res., 87:96-105 (2008)).

Disruption of the AIF-HSP70 cytoplasmic complex by BMS-214662 may occur through binding to either member of the complex. As a result, BMS-214662 may induce apoptosis through release of cytoplasmic bound AIF and subsequent localization of AIF to the nucleus where it facilitates chromatin condensation and induces apoptosis. Cancer cells would be predicted to be especially sensitive to such an activity of BMS-214662 because of their elevated levels of inducible HSP70. Elevation of HSP70 in response to stress associated with drug treatment, may additionally make cancer cells more sensitive to the combination of a cytotoxic drug with BMS-214662. Elevated levels of inducible HSP70 in stem cells, suggests that cancer stem cells may be sensitive to such BMS-214662 activity through AIF caspase independent apoptosis. It is possible that the quiescent, nonproliferating cell population of large tumors, which persist under stress conditions of hypoxia and reduced nutrient supply, may also have elevated levels of HSP70 and may thus also be sensitive to this BMS-214662 activity through an AIF-induced apoptosis mechanism.

### Discussion

In this report the inventors describe experiments that indicate that BMS-214662 preferentially targets the Q tumor cell population, a property that correlates with its anti-tumor activity as a single agent. Selective targeting and killing of Q cells is a novel and unique property of BMS-214662, since it has not been previously described for any anti-cancer agent currently in therapeutic use. The clinical implications of this finding are highly significant since previous oncology therapeutics have been directed towards P cells and the mechanisms responsible for promoting proliferation and eliciting cell division. Indeed, in our model the inventors show that conventional agents, including paclitaxel, CPT-11, and ixabepilone, exhibited preferential killing of P cells, as expected, while not affecting Q cells.

Remarkably, a close structural analog of BMS-214662, such as BMS-225975, behaves like many other cytostatic FTIs by showing potent inhibition of cell proliferation in vitro and cytostatic effects on tumor growth in vivo. However, BMS-225975 differs dramatically from BMS-214662 with its selective apoptotic potency and its tumor regressing activity in vivo (Manne et al., Cancer Res., 64:3974-3980 (2004)). Similar results were also shown for two more structurally related pairs of compounds. Our results therefore suggest that the potent anti-tumor regression activity and selectivity towards Q cells are closely intertwined.

### How Do Quiescent Cells Die?

In our work, clonogenic cell survival assays were used to demonstrate that BMS-214662 preferentially targeted Q tumor cells in an in vitro model of nutrient deprivation. Clonogenic assays are the gold standard for analysis of cell survival, and allowed us to establish that in Q cells BMS-214662 not only elicited some steps in the apoptosis process (DNA fragmentation by tunnel, cleavage of PARP, caspase activation) but affected the viability and functional capabilities of the treated cells.

Selective cell killing of Q cells by BMS-214662 was observed for many tumor cell types in our in vitro system, although the selectivity ratios varied considerably among the tumor cell lines tested, and may thus be an inherent property of the cell types.

Our results show that the pro-apoptotic activity of the compounds in table 1 against Q cells does not correlate with their potency against FT, GGT I or GGTII. Efforts to understand the mechanism of action for the proapoptotic activity identified GGTII as an apoptotic target for some BMS FTIs (Lackner et al., Cancer Cell., 4:325-336 (2005)). However, this is probably not the basis of selectivity for Q cells, since N-methyl versions of several of the FTIs tested retain potent GGTII activity and yet lack selectivity for Q cells (see Table 1). The inventors suggest that a general pro-apoptotic activity of BMS-214662 and its analogues at high concentrations in vitro may be affecting both P and Q cells and may be related to GGTII inhibitory activity, as previously suggested (Lackner et al., Cancer Cell., 4:325-336 (2005)). In contrast, the selective activity on Q cells, which occurs at lower concentrations and is described here for the first time, may be affecting different pathways and is reflected in the potency of the compounds in mouse xenograft tumor models. In summary, the compounds with preferential activity on Q cells and with anti-tumor activity in xenografts demonstrated FT, GGTI and GGTII enzyme inhibition comparable to compounds that lacked Q cell selectivity and had low in vivo tumor efficacy. All analogs that demonstrated selectivity for Q cells were also found to be active in the in vivo tumor xenograft models.

So is FT activity even required for the Q cell selectivity? The existence of compounds lacking FT activity that display reasonable Q selectivity ratios suggests that it is not essential (F. L, and M-L. W., unpublished). The in vitro and in vivo paradigms described here provide the biological tools and model compounds for the discovery and analysis of newer chemical entities selectively targeting Q cells.

### Therapeutic Implications

Selective toxicity of Q tumor cells at lower concentrations of BMS-214662 is a unique feature of this compound and raises the prospect of synergistic combination therapy with existing anticancer drugs targeting P tumor cells.

In xenograft models, synergistic combination therapy of BMS-214662 in vivo with cytostatic agents that inhibit cell proliferation and/or block cell progression in G0/G1, like hormonal agents, or with cytotoxic agents that preferentially kill P cells, such as paclitaxel, ixabepilone and CPT-11 was highly efficacious and dependent on sequence of administration. In all cases tested, synergistic interaction was observed not only in tumor growth delay, but also in terms of the more clinically relevant measures of tumor response, namely, PR and CR and in tumor cures in mice. This efficacy can be attributed to the high proportion of Q cells (56-80%) in tumors and the enhanced sensitivity of Q cells to the compound.

The clinical development of BMS-214662 as a single agent has resulted in recommended doses of 209 mg/m² and 275 mg/m² in the 1 and 24 hr infusion schedules, and achieved an AUC (area under the curve) of ˜30 uM×hr (Ryan et al., Clin. Cancer Res., 10:2222-2230 (2004); Papadimitrakopoulou et al., Clin. Cancer Res., 11:4151-4159 (2005); Tabernero et al., J. Clin. Oncol., 23: 2521-2533 (2005)). Our work shows these are well above the concentrations that are required to demonstrate single agent activity vs. Q cells in vitro (FIG. 6) and that are efficacious when achieved in mice in optimally sequenced combinations (FIG. 5a). The data presented here clearly demonstrates that concentrations readily achievable in clinical trials suffice to kill Q cells in vitro and in vivo, but may be insufficient to kill P cells, given the lengthy exposure required.

In fact, apoptosis was detected in inflammatory breast cancer lesions of patients treated in sequence with paclitaxel and BMS-214662, by in situ analysis (Tabernero et al., J. an. Oncol., 23: 2521-2533 (2005)), although the P/Q status was not established in the affected cells. In a trial of single agent in heavily pretreated acute myelogenous leukemia patients, evidence of response was found in 17% of the patients (Cortes et al., J. Clin. Oncol., 23:2805-2812 (2005)). The efficacy of a combination of BMS-214662 with carboplatin and TAXOL® was suggested by the results obtained in a phase 1 study (Dy et al., Clin. Cancer Res., 11:1877-1883 (2005)) and was well tolerated.

In traditional cancer treatments, one explanation for the failure to cure with cytotoxic drugs has been proposed to be due to recurrence from a residual Q cell population. In CML mouse models and in CML patients, recurrence of the disease upon discontinuation of treatment occurs through a reservoir of cancer stem cells that fails to respond to imatinib (Graham et al., Blood, 99:319-325 (2002)). One of the characteristics of cancer stem cells, as identified by specific cell surface markers and by label dilution and proliferation experiments, is their Q state (Graham et al., Blood, 99:319-325 (2002)). The unique property of BMS-214662 in targeting Q cells has been exploited to inhibit these hematopoietic cancer stem cells from mice in vivo and from human in vitro, in combination with dasatinib or imatinib (Cong et al., ASH, 2861 (2005); and Mhairi et al., ASH, 693 (2005)). Whether this exciting approach will extend to other cancer stem cells or is restricted to the Q cells in liquid tumors is a matter of intense investigation at this time. Surprisingly, the selectivity to Q cells may not be general, in terms of affecting all Q cells in an organism, as toxicity studies have found limited toxicity in animals and humans, especially in the effective range required for the quiescent cell activity. These results suggest promise for further development of BMS-214662 in cancer treatment.

The phrase “agonizes AIF” and “AIF agonist” includes increasing the biological activity of AIF, in general, which may include, but is not limited to increasing the amount of AIF in the active, mature form; increasing the amount of AIF that accumulates, localizes, or translocates to the nucleus; and increasing the ability of AIF to induce apoptosis, either directly or indirectly.

The phrase “agonizes HOP” and “HOP agonist” includes increasing the biological activity of HOP, in general, which may include, but is not limited to increasing the amount of HOP in its active, phosphorylated form; increasing the amount or ability of HOP to form a heterocomplex with Hsp70 and/or Hsp90; increasing the frequency by which HOP is able to translocate proteins to the nucleus; increasing frequency by which HOP, in conjunction with the Hsp70-Hsp90 complex, is able to translocate proteins to the nucleus; and increasing the frequency, ability, or activity of HOP in facilitating protein translocation to the nucleus, preferably proteins that can induce apoptosis, such as AIF, either directly or indirectly.

The compounds of the present invention are useful in the treatment of a variety of cancers, including (but not limited to) the following; carcinoma, including that of the bladder, breast, colon, kidney, liver, lung, including small cell lung cancer, ovary, prostate, testes, pancreas, esophagus, stomach, gall bladder, cervix, thyroid and skin, including squamous cell carcinoma; hematopoietic tumors of lymphoid lineage, including leukemia, acute lymphocytic leukemia, acute lymphoblastic leukemia, B-cell lymphoma, T-cell lymphoma, Hodgkins lymphoma, non-Hodgkins lymphoma, hairy cell lymphoma, and Burketts lymphoma; hematopoietic tumors of myeloid lineage, including acute and chronic myelogenous leukemias, myelodysplastic syndrome and promyelocytic leukemia; tumors of the central and peripheral nervous system, including astrocytoma, neuroblastoma, glioma, and schwannomas; tumors of mesenchymal origin, including fibrosarcoma, rhabdomyosarcoma, and osteosarcoma; other tumors, including melanoma, xenoderma pigmentosum, keratoactanthoma, seminoma, thyroid follicular cancer and teratocarcinoma.

By the administration of a composition having one (or a combination) of the compounds of this invention, development of tumors in a mammalian host is reduced, or tumor burden is reduced, or tumor regression is produced.

The compounds of the present invention may also inhibit tumor angiogenesis, thereby affecting the growth of tumors. Such anti-angiogenesis properties of the compounds described herein may also be useful in the treatment of certain forms of blindness related to retinal vascularization.

The compounds of the present invention may also be useful in the treatment of diseases other than cancer that may be associated with signal transduction pathways operating through ras, e.g., neurofibromatosis, atherosclerosis, pulmonary fibrosis, arthritis, psoriasis, glomerulonephritis, restenosis following angioplasty or vascular surgery, hypertrophic scar formation, polycystic kidney disease and endotoxic shock.

The compounds of the present invention may induce or inhibit apoptosis, a physiological cell death process critical for normal development and homeostasis. Alterations of apoptotic pathways contribute to the pathogenesis of a variety of human diseases. Compounds described herein, as modulators of apoptosis, will be useful in the treatment of a variety of human diseases with aberrations in apoptosis including cancer (particularly, but not limited to follicular lymphomas, carcinomas with p53 mutations, hormone dependent tumors of the breast, prostrate and ovary, and precancerous lesions such as familial adenomatous polyposis), viral infections (including but not limited to herpesvirus, poxvirus, Epstein-Barr virus, Sindbis virus and adenovirus), autoimmune diseases (including but not limited to systemic lupus erythematosus, immune mediated glomerulonephritis, rheumatoid arthritis, psoriasis, inflammatory bowl diseases and autoimmune diabetes mellitus), neurodegenerative disorders (including but not limited to Alzheimer's disease, AIDS-related dementia, Parkinson's disease, amyotrophic lateral sclerosis, retinitis pigmentosa, spinal muscular atrophy and cerebellar degeneration), AIDS, myelodysplastic syndromes, aplastic anemia, ischemic injury associated myocardial infarctions, stroke and reperfusion injury, arrhythmia, atherosclerosis, toxin-induced or alcohol induced liver diseases, hematological diseases (including but not limited to chronic anemia and aplastic anemia), degenerative diseases of the musculoskeletal system (including but not limited to osteoporosis and arthritis), aspirin-sensitive rhinosinusitis, cystic fibrosis, multiple sclerosis, kidney diseases, and cancer pain.

The compounds of the present invention may also be useful in combination with known anti-cancer and cytotoxic agents and treatments, including radiation. If formulated as a fixed dose, such combination products employ the compounds of this invention within the dosage range described below and the other pharmaceutically active agent within its approved dosage range. The compounds of the present invention may be used sequentially with known anticancer or cytotoxic agents and treatment, including radiation when a combination formulation is inappropriate.

The present invention provides methods for the treatment of a variety of other cancers, including, but not limited to, the following: carcinoma including that of the bladder (including accelerated and metastatic bladder cancer), breast, colon (including colorectal cancer), kidney, liver, lung (including small and non-small cell lung cancer and lung adenocarcinoma), ovary, prostate, testes, genitourinary tract, lymphatic system, rectum, larynx, pancreas (including exocrine pancreatic carcinoma), esophagus, stomach, gall bladder, cervix, thyroid, and skin (including squamous cell carcinoma); hematopoietic tumors of lymphoid lineage including leukemia, acute lymphocytic leukemia, acute lymphoblastic leukemia, B-cell lymphoma, T-cell lymphoma, Hodgkins lymphoma, non-Hodgkins lymphoma, hairy cell lymphoma, histiocytic lymphoma, and Burketts lymphoma; hematopoietic tumors of myeloid lineage including acute and chronic myelogenous leukemias, myelodysplastic syndrome, myeloid leukemia, and promyelocytic leukemia; tumors of the central and peripheral nervous system including astrocytoma, neuroblastoma, glioma, and schwannomas; tumors of mesenchymal origin including fibrosarcoma, rhabdomyosarcoma, and osteosarcoma; other tumors including melanoma, xenoderma pigmentosum, keratoactanthoma, seminoma, thyroid follicular cancer, and teratocarcinoma; melanoma, unresectable stage III or IV malignant melanoma, squamous cell carcinoma, small-cell lung cancer, non-small cell lung cancer, glioma, gastrointestinal cancer, renal cancer, ovarian cancer, liver cancer, colorectal cancer, endometrial cancer, kidney cancer, prostate cancer, thyroid cancer, neuroblastoma, pancreatic cancer, glioblastoma multiforme, cervical cancer, stomach cancer, bladder cancer, hepatoma, breast cancer, colon carcinoma, and head and neck cancer, gastric cancer, germ cell tumor, bone cancer, bone tumors, adult malignant fibrous histiocytoma of bone; childhood malignant fibrous histiocytoma of bone, sarcoma, pediatric sarcoma, sinonasal natural killer, neoplasms, plasma cell neoplasm; myelodysplastic syndromes; neuroblastoma; testicular germ cell tumor, intraocular melanoma, myelodysplastic syndromes; myelodysplastic/myeloproliferative diseases, synovial sarcoma, chronic myeloid leukemia, acute lymphoblastic leukemia, philadelphia chromosome positive acute lymphoblastic leukemia (Ph+ ALL), multiple myeloma, acute myelogenous leukemia, chronic lymphocytic leukemia, mastocytosis and any symptom associated with mastocytosis, and any metastasis thereof. In addition, disorders include urticaria pigmentosa, mastocytosises such as diffuse cutaneous mastocytosis, solitary mastocytoma in human, as well as dog mastocytoma and some rare subtypes like bullous, erythrodermic and teleangiectatic mastocytosis, mastocytosis with an associated hematological disorder, such as a myeloproliferative or myelodysplastic syndrome, or acute leukemia, myeloproliferative disorder associated with mastocytosis, mast cell leukemia, in addition to other cancers. Other cancers are also included within the scope of disorders including, but are not limited to, the following: carcinoma, including that of the bladder, urothelial carcinoma, breast, colon, kidney, liver, lung, ovary, pancreas, stomach, cervix, thyroid, testis, particularly testicular seminomas, and skin; including squamous cell carcinoma; gastrointestinal stromal tumors (“GIST”); hematopoietic tumors of lymphoid lineage, including leukemia, acute lymphocytic leukemia, acute lymphoblastic leukemia, B-cell lymphoma, T-cell lymphoma, Hodgkins lymphoma, non-Hodgkins lymphoma, hairy cell lymphoma and Burketts lymphoma; hematopoietic tumors of myeloid lineage, including acute and chronic myelogenous leukemias and promyelocytic leukemia; tumors of mesenchymal origin, including fibrosarcoma and rhabdomyosarcoma; other tumors, including melanoma, seminoma, tetratocarcinoma, neuroblastoma and glioma; tumors of the central and peripheral nervous system, including astrocytoma, neuroblastoma, glioma, and schwannomas; tumors of mesenchymal origin, including fibrosarcoma, rhabdomyosarcoma, and osteosarcoma; and other tumors, including melanoma, xenoderma pigmentosum, keratoactanthoma, seminoma, thyroid follicular cancer, teratocarcinoma, chemotherapy refractory non-seminomatous germ-cell tumors, and Kaposi's sarcoma, and any metastasis thereof.

Most preferably, the invention is used to treat accelerated or metastatic cancers of the bladder, pancreatic cancer, prostate cancer, non-small cell lung cancer, colorectal cancer, and breast cancer.

In a preferred embodiment of this invention, a method is provided for the treatment of cancerous tumors. Advantageously, the method of this invention reduces the development of tumors, reduces tumor burden, or produces tumor regression in a mammalian host.

Methods for the safe and effective administration of most of these chemotherapeutic agents are known to those skilled in the art. In addition, their administration is described in the standard literature.

For example, the administration of many of the chemotherapeutic agents is described in the Physicians' Desk Reference (PDR), e.g., 1996 edition (Medical Economics Company, Montvale, N.J. 07645-1742, USA); the disclosure of which is incorporated herein by reference thereto.

The human AIF, HOP, phosphatase 1G, protein tyrosine phosphatase non-receptor type 6 isoform 2, Hsp70, and/or Other Target Protein polypeptides and/or peptides, or immunogenic fragments or oligopeptides thereof, can be used for screening therapeutic drugs or compounds in a variety of drug screening techniques. The fragment employed in such a screening assay may be free in solution, affixed to a solid support, borne on a cell surface, or located intracellularly. The reduction or abolition of activity of the formation of binding complexes between either protein and the agent being tested can be measured. Thus, the present invention provides a method for screening or assessing a plurality of compounds for their specific binding affinity with a AIF, HOP, phosphatase 1G, protein tyrosine phosphatase non-receptor type 6 isoform 2, Hsp70, and/or Other Target Protein polypeptide, or a bindable peptide fragment, of this invention, comprising providing a plurality of compounds, combining the AIF, HOP, phosphatase 1G, protein tyrosine phosphatase non-receptor type 6 isoform 2, Hsp70, and/or Other Target Protein polypeptide, or a bindable peptide fragment, with each of a plurality of compounds for a time sufficient to allow binding under suitable conditions and detecting binding of the AIF, HOP, phosphatase 1G, protein tyrosine phosphatase non-receptor type 6 isoform 2, Hsp70, and/or Other Target Protein polypeptide or peptide to each of the plurality of test compounds, thereby identifying the compounds that specifically bind to the AIF, HOP, phosphatase 1G, protein tyrosine phosphatase non-receptor type 6 isoform 2, Hsp70, and/or Other Target Protein polypeptide or peptide. Preferably, such a modulator compound agonizes the activity of AIF, HOP, phosphatase 1G, protein tyrosine phosphatase non-receptor type 6 isoform 2, Hsp70, and/or Other Target Proteinpolypeptide and results in the induction of apoptosis, preferably in quiescent cells.

Methods of identifying compounds that modulate the activity of the novel human AIF, HOP, phosphatase 1G, protein tyrosine phosphatase non-receptor type 6 isoform 2, Hsp70, and/or Other Target Protein polypeptides and/or peptides are provided by the present invention and comprise combining a potential or candidate compound or drug modulator of apoptotic and/or chaperone biological activity with an AIF, HOP, phosphatase 1G, protein tyrosine phosphatase non-receptor type 6 isoform. 2, Hsp70, and/or Other Target Protein polypeptide or peptide, for example, the AIF, HOP, phosphatase 1G, protein tyrosine phosphatase non-receptor type 6 isoform 2, Hsp70, and/or Other Target Protein amino acid sequence, and measuring an effect of the candidate compound or drug modulator on the biological activity of the AIF, HOP, phosphatase 1G, protein tyrosine phosphatase non-receptor type 6 isoform 2, Hsp70, and/or Other Target Protein polypeptide or peptide. Such measurable effects include, for example, physical binding interaction; the ability to cleave a suitable substrate; effects on native and cloned AIF, HOP, phosphatase 1G, protein tyrosine phosphatase non-receptor type 6 isoform 2, Hsp70, and/or Other Target Protein-expressing cell line; and effects of modulators or other apoptotic and/or chaperone-mediated physiological measures. Preferably, such a modulator compound agonizes the activity of AIF, HOP, phosphatase 1G, protein tyrosine phosphatase non-receptor type 6 isoform 2, Hsp70, and/or Other Target Protein polypeptide and results in the induction of apoptosis, preferably in quiescent cells.

Another method of identifying compounds that modulate the biological activity of the novel AIF, HOP, phosphatase 1G, protein tyrosine phosphatase non-receptor type 6 isoform 2, Hsp70, and/or Other Target Protein polypeptides of the present invention comprises combining a potential or candidate compound or drug modulator of a apoptotic and/or chaperone biological activity with a host cell that expresses the AIF, HOP, phosphatase 1G, protein tyrosine phosphatase non-receptor type 6 isoform 2, Hsp70, and/or Other Target Protein polypeptide and measuring an effect of the candidate compound or drag modulator on the biological activity of the AIF, HOP, phosphatase 1G, protein tyrosine phosphatase non-receptor type 6 isoform 2, Hsp70, and/or Other Target Protein polypeptide. The host cell can also be capable of being induced to express the AIF, HOP, phosphatase 1G, protein tyrosine phosphatase non-receptor type 6 isoform 2, Hsp70, and/or Other Target Protein polypeptide, e.g., via inducible expression. Physiological effects of a given modulator candidate on the AIF, HOP, phosphatase 1G, protein tyrosine phosphatase non-receptor type 6 isoform 2, Hsp70, and/or Other Target Protein polypeptide can also be measured. Thus, cellular assays for particular apoptotic and/or chaperone modulators may be either direct measurement or quantification of the physical biological activity of the AIF, HOP, phosphatase 1G, protein tyrosine phosphatase non-receptor type 6 isoform 2, Hsp70, and/or Other Target Protein polypeptide, or they may be measurement or quantification of a physiological effect. Such methods preferably employ a AIF, HOP, phosphatase 1G, protein tyrosine phosphatase non-receptor type 6 isoform 2, Hsp70, and/or Other Target Protein polypeptide as described herein, or an overexpressed recombinant AIF, HOP, phosphatase 1G, protein tyrosine phosphatase non-receptor type 6 isoform 2, Hsp70, and/or Other Target Proteinpolypeptide in suitable host cells containing an expression vector as described herein, wherein the AIF, HOP, phosphatase 1G, protein tyrosine phosphatase non-receptor type 6 isoform 2, Hsp70, and/or Other Target Protein polypeptide is expressed, overexpressed, or undergoes upregulated expression. Preferably, such a modulator compound agonizes the activity of AIF, HOP, phosphatase 1G, protein tyrosine phosphatase non-receptor type 6 isoform 2, Hsp70, and/or Other Target Protein polypeptide and results in the induction of apoptosis, preferably in quiescent cells.

Another aspect of the present invention embraces a method of screening for a compound that is capable of modulating the biological activity of a AIF, HOP, phosphatase 1G, protein tyrosine phosphatase non-receptor type 6 isoform 2, Hsp70, and/or Other Target Protein polypeptide, comprising providing a host cell containing an expression vector harboring a nucleic acid sequence encoding a AIF, HOP, phosphatase 1G, protein tyrosine phosphatase non-receptor type 6 isoform 2, Hsp70, and/or Other Target Protein polypeptide, or a functional peptide or portion thereof; determining the biological activity of the expressed AIF, HOP, phosphatase 1G, protein tyrosine phosphatase non-receptor type 6 isoform 2, Hsp70, and/or Other Target Protein polypeptide in the absence of a modulator compound; contacting the cell with the modulator compound and determining the biological activity of the expressed AIF, HOP, phosphatase 1G, protein tyrosine phosphatase non-receptor type 6 isoform 2, Hsp70, and/or Other Target Protein polypeptide in the presence of the modulator compound. In such a method, a difference between the activity of the AIF, HOP, phosphatase 10, protein tyrosine phosphatase non-receptor type 6 isoform 2, Hsp70, and/or Other Target Protein polypeptide in the presence of the modulator compound and in the absence of the modulator compound indicates a modulating effect of the compound. Preferably, such a modulator compound agonizes the activity of AIF, HOP, phosphatase 10, protein tyrosine phosphatase non-receptor type 6 isoform 2, Hsp70, and/or Other Target Protein polypeptide and results in the induction of apoptosis, preferably in quiescent cells.

Essentially any chemical compound can be employed as a potential modulator or ligand in the assays according to the present invention. Compounds tested as apoptotic and/or chaperone modulators can be any small chemical compound, or biological entity (e.g., protein, sugar, nucleic acid, lipid). Test compounds will typically be small chemical molecules and peptides. Generally, the compounds used as potential modulators can be dissolved in aqueous or organic (e.g., DMSO-based) solutions. The assays are designed to screen large chemical libraries by automating the assay steps and providing compounds from any convenient source. Assays are typically run in parallel, for example, in microtiter formats on microtiter plates in robotic assays. There are many suppliers of chemical compounds, including Sigma (St. Louis, Mo.), Aldrich (St. Louis, Mo.), Sigma-Aldrich (St. Louis, Mo.), Fluka Chemika-Biochemica Analytika (Buchs, Switzerland), for example. Also, compounds may be synthesized by methods known in the art.

High throughput screening methodologies are particularly envisioned for the detection of modulators of the novel AIF, HOP, phosphatase 1G, protein tyrosine phosphatase non-receptor type 6 isoform 2, Hsp70, and/or Other Target Protein polynucleotides and polypeptides described herein. Such high throughput screening methods typically involve providing a combinatorial chemical or peptide library containing a large number of potential therapeutic compounds (e.g., ligand or modulator compounds). Such combinatorial chemical libraries or ligand libraries are then screened in one or more assays to identify those library members (e.g., particular chemical species or subclasses) that display a desired characteristic activity. The compounds so identified can serve as conventional lead compounds, or can themselves be used as potential or actual therapeutics.

A combinatorial chemical library is a collection of diverse chemical compounds generated either by chemical synthesis or biological synthesis, by combining a number of chemical building blocks (i.e., reagents such as amino acids). As an example, a linear combinatorial library, e.g., a polypeptide or peptide library, is formed by combining a set of chemical building blocks in every possible way for a given compound length (i.e., the number of amino acids in a polypeptide or peptide compound). Millions of chemical compounds can be synthesized through such combinatorial mixing of chemical building blocks.

The preparation and screening of combinatorial chemical libraries is well known to those having skill in the pertinent art. Combinatorial libraries include, without limitation, peptide libraries (e.g., U.S. Pat. No. 5,010,175; Furka, Int. J. Pept. Prot. Res., 37:487-493 (1991); and Houghton et al., Nature, 354:84-88 (1991)). Other chemistries for generating chemical diversity libraries can also be used. Nonlimiting examples of chemical diversity library chemistries include, peptoids (PCT Publication No. WO 91/019735), encoded peptides (PCT Publication No. WO 93/20242), random bio-oligomers (PCT Publication No. WO 92/00091), benzodiazepines (U.S. Pat. No. 5,288,514), diversomers such as hydantoins, benzodiazepines and dipeptides (Hobbs et al., Proc. Natl. Acad. Sci. USA, 90:6909-6913 (1993)), vinylogous polypeptides (Hagihara et al., J. Amer. Chem. Soc., 114:6568 (1992)), nonpeptidal peptidomimetics with glucose scaffolding (Hirschmann et al., J. Amer. Chem. Soc., 114:9217-9218 (1992)), analogous organic synthesis of small compound libraries (Chen et al., J. Amer. Chem. Soc., 116:2661 (1994)), oligocarbamates (Cho et al., Science, 261:1303 (1993)), and/or peptidyl phosphonates (Campbell et al., J. Org. Chem., 59:658 (1994)), nucleic acid libraries (see Ausubel, Berger and Sambrook, all supra), peptide nucleic acid libraries (U.S. Pat. No. 5,539,083), antibody libraries (e.g., Vaughn et al., Nature Biotechnology, 14(3):309-314 (1996)) and PCT/US96/10287), carbohydrate libraries (e.g., Liang et al., Science, 274-1520-1522 (1996)) and U.S. Pat. No. 5,593,853), small organic molecule libraries (e.g., benzodiazepines, Baum, Chem. Eng. News, 33 (Jan. 18, 1993); and U.S. Pat. No. 5,288,514; isoprenoids, U.S. Pat. No. 5,569,588; thiazolidinones and metathiazanones, U.S. Pat. No. 5,549,974; pyrrolidines, U.S. Pat. Nos. 5,525,735 and 5,519,134; morpholino compounds, U.S. Pat. No. 5,506,337; and the like).

Devices for the preparation of combinatorial libraries are commercially available (e.g., 357 MPS, 390 MPS, Advanced Chem Tech, Louisville Ky.; Symphony, Rainin, Woburn, Mass.; 433A Applied Biosystems, Foster City, Calif.; 9050 Plus, Millipore, Bedford, Mass.). In addition, a large number of combinatorial libraries are commercially available (e.g., ComGenex, Princeton, N.J.; Asinex, Moscow, Russia; Tripos, Inc., St. Louis, Mo.; ChemStar, Ltd., Moscow, Russia; 3D Pharmaceuticals, Exton, Pa.; Martek Biosciences, Columbia, Md., and the like).

In one embodiment, the invention provides solid phase based in vitro assays in a high throughput format, where the cell or tissue expressing an ion channel is attached to a solid phase substrate. In such high throughput assays, it is possible to screen up to several thousand different modulators or ligands in a single day. In particular, each well of a microtiter plate can be used to perform a separate assay against a selected potential modulator, or, if concentration or incubation time effects are to be observed, every 5-10 wells can test a single modulator. Thus, a single standard microtiter plate can assay about 96 modulators. If 1536 well plates are used, then a single plate can easily assay from about 100 to about 1500 different compounds. It is possible to assay several different plates per day; thus, for example, assay screens for up to about 6,000-20,000 different compounds are possible using the described integrated systems.

In another of its aspects, the present invention encompasses screening and small molecule (e.g., drug) detection assays which involve the detection or identification of small molecules that can bind to a given protein, i.e., a AIF, HOP, phosphatase 1G, protein tyrosine phosphatase non-receptor type 6 isoform 2, Hsp70, and/or Other Target Protein polypeptide or peptide. Particularly preferred are assays suitable for high throughput screening methodologies.

In such binding-based detection, identification, or screening assays, a functional assay is not typically required. All that is needed is a target protein, preferably substantially purified, and a library or panel of compounds (e.g., ligands, drugs, small molecules) or biological entities to be screened or assayed for binding to the protein target. Preferably, most small molecules that bind to the target protein will modulate activity in some manner, due to preferential, higher affinity binding to functional areas or sites on the protein.

An example of such an assay is the fluorescence based thermal shift assay (3-Dimensional Pharmaceuticals, Inc., 3DP, Exton, Pa.) as described in U.S. Pat. Nos. 6,020,141 and 6,036,920 to Pantoliano et al.; see also, Zimmermann, J., Gen. Eng. News, 20(8) (2000)). The assay allows the detection of small molecules (e.g., drugs, ligands) that bind to expressed, and preferably purified, ion channel polypeptide based on affinity of binding determinations by analyzing thermal unfolding curves of protein-drug or ligand complexes. The drugs or binding molecules determined by this technique can be further assayed, if desired, by methods, such as those described herein, to determine if the molecules affect or modulate function or activity of the target protein.

To purify a AIF, HOP, phosphatase 1G, protein tyrosine phosphatase non-receptor type 6 isoform 2, Hsp70, and/or Other Target Protein polypeptide or peptide to measure a biological binding or ligand binding activity, the source may be a whole cell lysate that can be prepared by successive freeze-thaw cycles (e.g., one to three) in the presence of standard protease inhibitors. The AIF, HOP, phosphatase 1G, protein tyrosine phosphatase non-receptor type 6 isoform 2, Hsp70, and/or Other Target Protein polypeptide may be partially or completely purified by standard protein purification methods, e.g., affinity chromatography using specific antibody described infra, or by ligands specific for an epitope tag engineered into the recombinant AIF, HOP, phosphatase 1G, protein tyrosine phosphatase non-receptor type 6 isoform 2, Hsp70, and/or Other Target Protein polypeptide molecule, also as described herein. Binding activity can then be measured as described.

Compounds which are identified according to the methods provided herein, and which modulate or regulate the biological activity or physiology of the AIF, HOP, phosphatase 1G, protein tyrosine phosphatase non-receptor type 6 isoform 2, Hsp70, and/or Other Target Protein polypeptides according to the present invention are a preferred embodiment of this invention. It is contemplated that such modulatory compounds may be employed in treatment and therapeutic methods for treating a condition that is mediated by the novel AIF, HOP, phosphatase 1G, protein tyrosine phosphatase non-receptor type 6 isoform 2, Hsp70, and/or Other Target Protein polypeptides by administering to an individual in need of such treatment a therapeutically effective amount of the compound identified by the methods described herein.

In addition, the present invention provides methods for treating an individual in need of such treatment for a disease, disorder, or condition that is mediated by the AIF, HOP, phosphatase 1G, protein tyrosine phosphatase non-receptor type 6 isoform 2, Hsp70, and/or Other Target Protein polypeptides of the invention, comprising administering to the individual a therapeutically effective amount of the AIF, HOP, phosphatase 1G, protein tyrosine phosphatase non-receptor type 6 isoform 2, Hsp70, and/or Other Target Protein-modulating compound identified by a method provided herein.

The present invention contemplates the use of an AIF, HOP, phosphatase 1G, protein tyrosine phosphatase non-receptor type 6 isoform 2, Hsp70, and/or Other Target Protein protein and/or peptide sequence which is at least about 80%, 85%, 90%, 91%, 92%, 93%, 93.6%, 94%, 95%, 96%, 97%, 97.9%, 98%, 99%, 99.1%, 99.2%, 99.3%, 99.4%, 99.5%, 99.6%, 99.7%, 99.8%, or 99.9% identical to, for example, the wild-type sequences for methods to identify compounds that bind to the AIF, HOP, phosphatase 1G, protein tyrosine phosphatase non-receptor type 6 isoform 2, Hsp70, and/or Other Target Protein protein, and which preferably agonize their activity and induce apoptosis, preferably in quiescent cells.

Antisense oligonucleotides may be single or double stranded. Double stranded RNA's may be designed based upon the teachings of Paddison et al., Proc. Nat. Acad. Sci., 99:1443-1448 (2002); and International Publication Nos. WO 01/29058, and WO 99/32619; which are hereby incorporated herein by reference.

Double stranded RNA may also take the form of an RNA inhibitor (“RNAi”) such that they are competent for RNA interference. For example, anti-AIF, anti-HOP, and/or anti-phosphase 1G RNAi molecules may take the form of the molecules described by Mello et al., in PCT Publication No. WO 1999/032619; PCT Publication No. WO 2001/029058; U.S.S.N. 2003/0051263; U.S.S.N. 2003/0055020; U.S.S.N. 2003/0056235; U.S.S.N. 2004/265839; U.S.S.N. 2005/0100913; U.S.S.N. 2006/0024798; U.S.S.N. 2008/0050342; U.S.S.N. 2008/0081373; U.S.S.N. 2008/0248576; U.S.S.N. 2008/055443; U.S. Pat. Nos. 6,506,559, 7,282,564, 7,538,095, and/or 7,560,438. The teachings of these patent and patent applications are hereby incorporated herein by reference in their entirety.

For example, the anti-AIF, anti-HOP, and/or anti-phosphase 1G RNAi molecules may be double stranded RNA, and between about 25 to 400 nucleotides in length, and complementary to the encoding nucleotide sequence of AIF, HOP, and/or phosphase 1G. Such RNAi molecules may be about 20, about 25, about 30, about 35, about 45, and about 50 nucleotides in length. In this context, the term “about” is construed to be about 1, 2, 3, 4, 5, or 6 nucleotides longer in either the 5′ or 3′ direction, or both.

Alternatively, the anti-AIF, anti-HOP, and/or anti-phosphase 1G RNAi molecules of the present invention may take the form of double stranded RNAi molecules described by Kreutzer in European Patent EP 1144639, and European Patent EP1214945. The teachings of these patent and patent applications are hereby incorporated herein by reference in their entirety. Specifically, the anti-AIF, anti-HOP, and/or anti-phosphase 1G RNAi molecules of the present invention may be double stranded RNA that is complementary to the coding region of AIF, HOP, and/or phosphase 1G, and is between about 15 to about 49 nucleotides in length, and preferably between about 15 to about 21 nucleotides in length. In this context, the term “about” is construed to be about 1, 2, 3, 4, 5, 6, 7, 8, 9, or 10 nucleotides longer in either the 5′ or 3′ direction, or both. Such anti-AIF, anti-HOP, and/or anti-phosphase 1G molecules can be stabilized by chemical linkage of the single RNA strands.

Alternatively, the anti-AIF, anti-HOP, and/or anti-phosphase 1G RNAi molecules of the present invention may take the form be double stranded RNAi molecules described by Tuschl in European Patent EP1309726. The teachings of these patent and patent applications are hereby incorporated herein by reference in their entirety. Specifically, the anti-AIF, anti-HOP, and/or anti-phosphase 1G RNAi molecules of the present invention may be double stranded RNA that is complementary to the coding region of AIF, HOP, and/or phosphase 1G, and is between about 21 to about 23 nucleotides in length, and are either blunt ended or contain either one or more overhangs on the 5′ end or 3′ end of one or both of the strands with each overhang being about 1, 2, 3, 4, 5, 6, or more nucleotides in length. The ends of each strand may be modified by phosphorulation, hybroxylation, or other modifications. In addition, the internucleotide linkages of one or more of the nucleotides may be modified, and may contain 2′-OH. In this context, the term “about” is construed to be about 1, 2, 3, 4, 5, 6, 7, 8, 9, or 10 nucleotides longer in either the 5′ or 3′ direction, or both. Such anti-AIF, anti-HOP, and/or anti-phosphase 1G molecules can be stabilized by chemical linkage of the single RNA strands.

Alternatively, the anti-AIF, anti-HOP, and/or anti-phosphase 1G RNAi molecules of the present invention may take the form be double stranded RNAi molecules described by Tuschl in U.S. Pat. Nos. 7,056,704 and 7,078,196. The teachings of these patent and patent applications are hereby incorporated herein by reference in their entirety. Specifically, the anti-AIF, anti-HOP, and/or anti-phosphase 1G RNAi molecules of the present invention may be double stranded RNA that is complementary to the coding region of AIF, HOP, and/or phosphase 1G, and is between about 19 to about 25 nucleotides in length, and are either blunt ended or contain either one or more overhangs on the 5′ end or 3′ end of one or both of the strands with each overhang being about 1, 2, 3, 4, or 5 or more nucleotides in length. The ends of each strand may be modified by phosphorulation, hybroxylation, or other modifications. In addition, the internucleotide linkages of one or more of the nucleotides may be modified, and may contain 2′-OH. In this context, the term “about” is construed to be about 1, 2, 3, 4, 5, 6, 7, 8, 9, or 10 nucleotides longer in either the 5′ or 3′ direction, or both. Such anti-AIF, anti-HOP, and/or anti-phosphase 1G molecules can be stabilized by chemical linkage of the single RNA strands.

Additionally, the anti-AIF, anti-HOP, and/or anti-phosphase 1G RNAi molecules of the present invention may take the form be RNA molecules described by Crooke in U.S. Pat. Nos. 5,898,031, 6,107,094, 7,432,249, 7,432,250 and European Application No, EP0928290. The teachings of these patent and patent applications are hereby incorporated herein by reference in their entirety. Specifically, the anti-AIF, anti-HOP, and/or anti-phosphase 1G molecules may be single stranded RNA, containing a first segment having at least one ribofuranosyl nucleoside subunit which is modified to improve the binding affinity of said compound to the preselected RNA target when compared to the binding affinity of an unmodified oligoribonucleotide to the RNA target; and a second segment comprising at least four consecutive ribofuranosyl nucleoside subunits having 2′-hydroxyl moieties thereon; said nucleoside subunits of said oligomeric compound being connected by internucleoside linkages which are modified to stabilize said linkages from degradation as compared to phosphodiester linkages. Preferably, such RNA molecules are about 15 to 25 nucleotides in length, or about 17 to about 20 nucleotides in length. Preferably such molecules are competent to activate a double-stranded RNAse enzyme to effect cleavage of AIF, HOP, and/or phosphase 1G RNA. In this context, the term “about” is construed to be about 1, 2, 3, 4, 5, 6, 7, 8, 9, or 10 nucleotides longer in either the 5′ or 3′ direction, or both. Such anti-AIF, anti-HOP, and/or anti-phosphase 1G molecules can be stabilized by chemical linkage of the single RNA strands.

SiRNA reagents are specifically contemplated by the present invention. Such reagents are useful for inhibiting expression of the polynucleotides of the present invention and may have therapeutic efficacy. Several methods are known in the art for the therapeutic treatment of disorders by the administration of siRNA reagents. One such method is described by Tiscornia et al (Proc. Natl. Acad. Sci., 100(4):1844-1848 (2003)); WO 04/09769, filed Jul. 18, 2003; and Reich, S. J. et al., Mol. Vis., 9:210-216 (May 30, 2003), which are incorporated by reference herein in its entirety.

In order to facilitate a further understanding of the invention, the following examples are presented primarily for the purpose of illustrating more specific details thereof. The scope of the invention should not be deemed limited by the examples, but to encompass the entire subject matter defined by the claims.

### REFERENCES

1. Cestac, P. et al., Ann. Pharm. Fr., 63:76-84 (2005).
2. Zhu, K. et al., Curr. Opin. Investig. Drugs, 4:1428-1435 (2003).
3. Hunt, J. T. et al., J. Med. Chem., 43:3587-3595 (2000).
4. Rose, W. C. et al., Cancer Res., 61:7507-7517 (2001).
5. Kohl, N. E. et al., Proc. Natl. Acad. Sci. USA, 91:9141-9145 (1994).
6. Nagasu, T. et al., Cancer Res., 55:5310-5314 (1995).
7. Kohl, N. E. et al., Nat. Med., 1:792-797 (1995).
8. Jackson, R. C., Adv. Enzyme Regul., 29:27-46 (1989).
9. Prevost, G. P. et al., Int. J. Cancer, 83:283-287 (1999).
10. Prevost, G. P. et al., Int. J. Cancer, 91:718-722 (2001).
11. Sun, J. et al., Cancer Res., 59:4919-4926 (1999).
12. Barrington, R. E. et al., Mol. Cell. Biol., 18:85-92 (1998).
13. Norgaard, P. et al., Clin. Cancer Res., 5:35-42 (1999).
14. Liu, M. et al., Cancer Res., 58:4947-4956 (1998).
15. End, D. W. et al., Cancer Res., 61:131-137 (2001).
16. Lackner, M. R. et al., Cancer Cell., 4:325-336 (2005).
17. Manne, V. et al., Cancer Res., 64:3974-3980 (2004).
18. Lombardo, L. J. et al., Bioorg. Med. Chem. Lett., 15:1895-1899 (2005).
19. Navone, N. M. et al., Clin. Cancer Res., 3:2493-2500 (1997).
20. Marzo, I. et al., Leukemia, 18:1599-1604 (2004).
21. Gomez-Benito, M. et al., Mol. Pharmacol., 67:1991-1998 (2005).
22. Ryan, D. P. et al., Clin. Cancer Res., 10:2222-2230 (2004).
23. Papadimitrakopoulou, V. et al., Clin. Cancer Res., 11:4151-4159 (2005).
24. Tabernero, J. et al., J. Clin. Oncol., 23:2521-2533 (2005).
25. Cortes, J. et al., J. Clin. Oncol., 23:2805-2812 (2005).
26. Dy, G. K. et al., Clin. Cancer Res., 11:1877-1883 (2005).
27. Graham, S. M. et al., Blood, 99:319-325 (2002).
28. Peng, C. et al., ASH, 2861 (2005).
29. Copland, M. et al., ASH, 693 (2005).
30. Churbanona et al., J. Biol. Chem., 283:5622-5631 (2008).
31. Daniel et al., Biochim. Biophys. Acta., 1783(6):1003 (2008).
32. Powers et al., FEBS Lett., 581:3758-3769 (2007).
33. Murano, K. A., Ann. NY Acad. Sci., 1113:1-14 (2007).
34. Ravagnan et al., Nat. Cell Biol., 3:839-843 (2001).
35. Gurbuxani et al., Oncogene, 22:6669-6678 (2003).
36. Ruchalsk et al., J. Biol. Chem., 281:7873-7880 (2006).
37. Schmitt et al., Can. Res., 66:4191-4197 (2006).
38. Park et al., Autrophagy, 4:364-367 (2008).
39. Jolly et al., J. Natl. Cancer Inst., 92:1564-1572 (2000).
40. Mosser et al., Oncogene, 23:2907-2918 (2004).
41. Ciocca, D. R. et al., Cell Stress Chaperones, 10:86-103 (2005).
42. Kaur, J. et al., Int. J. Cancer, 63:774-779 (1995).
43. Hantschel, M. et al., Cell Stress Chaperones, 5:438-442 (2000).
44. Pocaly, M. et al., Leukemia, 21:93-101 (2007).
45. Geraci, F. et al., Cell. Death Differ., 13:1057-1063 (2006).
46. Lyngholm, M. et al., Exp. Eye Res., 87:96-105 (2008).

### TABLES

TABLE 1
Pharmacological and structure/activity profiles of selected FTI with distinct
cell selectivity.

| | | | FT | GGTI | RabGGT | P/Q | Activity |
|---|---|---|---|---|---|---|---|
| BMS # | R¹ | R² | IC₅₀ (nM) | IC₅₀ (nM) | IC₅₀ (nM) | selectivity | in vivo* |
| BMS- 212435 BMS- 226007 | 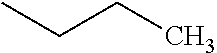 | H CH₃ | 4.1 2.1 | 2065 1200 | 21 15 | 66 P | 7/7 0/8 (0.6) |
| BMS- 212347 BMS- 223999 | 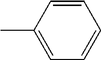 | H CH₃ | 2.4 1.1 | 1200 530 | 41 8 | 47 P | 7/8 0/8 (0.4) |
| BMS- 214662 BMS- 225975 | 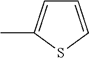 | H CH₃ | 0.7 0.8 | 1900 900 | 20 12 | 11-100 1.4 | 8/8 0/8 (0.3) |

Footnotes: The ratios of P/Q selectivity were derived from the IC90s on HCT116 cells, except for BMS-214662 where the range on a variety of cell types is presented. All in vivo tests (at MTD: 600 mg/kg) on HCT116 xenografts is expressed as cures, or as LCK (log cell kill) when no cures were achieved.

### Example 1

### Method of Demonstrating that BMS-214662 Selectively Targets Apoptosis of Quiescent Tumor Cells

### Materials and Methods

Drug Preparation. For in vitro studies, BMS-214662, BMS-225975, ixabepilone, paclitaxel, and 5-FU were dissolved in DMSO with dilutions made using either water or RPMI1640 (Invitrogen/GIBCO) plus 10% heat inactivated fetal bovine serum (Invitrogen/GIBCO). For in vivo studies the compounds were dissolved in ethanol followed by dilution with water to a final concentration of <10% ethanol.

Mice. CDF1 mice and Balb/c background athymic (nude) female mice approximately five weeks of age, were purchased from Harlan Sprague-Dawley (Indianapolis, Ind.). All procedures involving animals subjects were performed with approval from the Bristol-Myers Squibb Pharmaceutical Research Institute Animal Care and Use Committee (ACUC), which is fully accredited by the American Association for Accreditation of Laboratory Animal Care (AAALAC).

Tumors and Efficacy Testing. The human colon tumor line HCT-116, passaged subcutaneously (s.c.) in vivo at approximately two to three week intervals, was used and tested as reported earlier (Rose et al., Cancer Res., 61:7507-7517 (2001)). MDA-PCa2b was tested as described (Navone et al., Clin. Cancer Res., 3:2493-500 (1997)).

Tumor Cell Lines. Human carcinoma cell lines: HCT-116 (human colon carcinoma), Pat-7 (human ovarian carcinoma), HT29 (human colon carcinoma) and K562 (CML) were maintained in RPMI1640 supplemented with 10% fetal bovine serum. Proliferating cultures were set up by plating 3×10⁵ cells in 10 ml of RPMI medium in T75 flasks on day 0 and treated with compounds (dissolved in fresh medium) on day 2 or 3. Quiescent cultures were set up by plating 3×10⁵ cells in 10 ml of RPMI media in T75 flasks on day 0, changing medium on day 2, and treated with drugs dissolved in spent media on day 6 for 17 hr.

Clonogenic Assay. Following drug exposure, monolayer cell cultures were dissociated by addition of 0.05% trypsin for 5 min at 37° C., resuspended in complete media (RPMI 1640 containing 10% FBS), counted with a COULTER® Channelyzer, diluted and plated with 5 replicates per dilution. After 10 days incubation at 37° C., colonies were stained with crystal violet. Colonies (>50 cells) were counted and the concentration: needed to reduce clonogenic cells by 90% (i.e., the IC90) was determined.

BrdUrd Labeling of Asynchronously Growing Tumors. BrdUrd in sterile phosphate-buffered saline (PBS), pH 7.4, was administered by infusion via the tail vein to tumor bearing mice (100 mpk). Excised tumors (at 24 hr) were minced and dissociated with 0.025% collagenase and 0.04% DNase (Sigma Chemical Co., St Louis, Mo.), 0.05% pronase (Calbiochem, LaJolla, Calif.) and for 1 hr at 37° C. The dissociated cells were fixed with 75% methanol, washed and stained with anti-BrdUrd-FITC (10 μg/ml) (Boeringer Mannheim). After incubation, RNAse treatment and finally, propidium iodide staining (Sigma, 10 μg/ml), cells were analyzed by sorting with a FACSCalibur and data were analyzed with Tree Star's Flow Jo software. Detection of active caspase-3 was performed using a FITC conjugated antibody (cat#C-92-605, BD Biosciences) and p85 PARP cleavage with antibody (cat #G7341 from Promega Corporation, Madison, Wis.).

Enzyme Assays. Prenyltransferases and H-Ras processing inhibition were carried out as described earlier (Hunt et al., Rose et al., Lackner et al., Manne et al., and Lombardo et al).

### Example 2

### Method of Identifying the Mechanism of Action of BMS-214662 in Selectively Inducing Apoptosis in Quiescent Tumor Cells Using Affinity Chromatography and a Biotinylated Analogue of BMS-214662

Initial experiments designed to identify the mechanism of action of BMS-214662 consisted of the analysis of size fractionated, crosslinked material, that bound to a avidin affinity chromatography column and used a short biotinylated arm attached to BMS-214662.

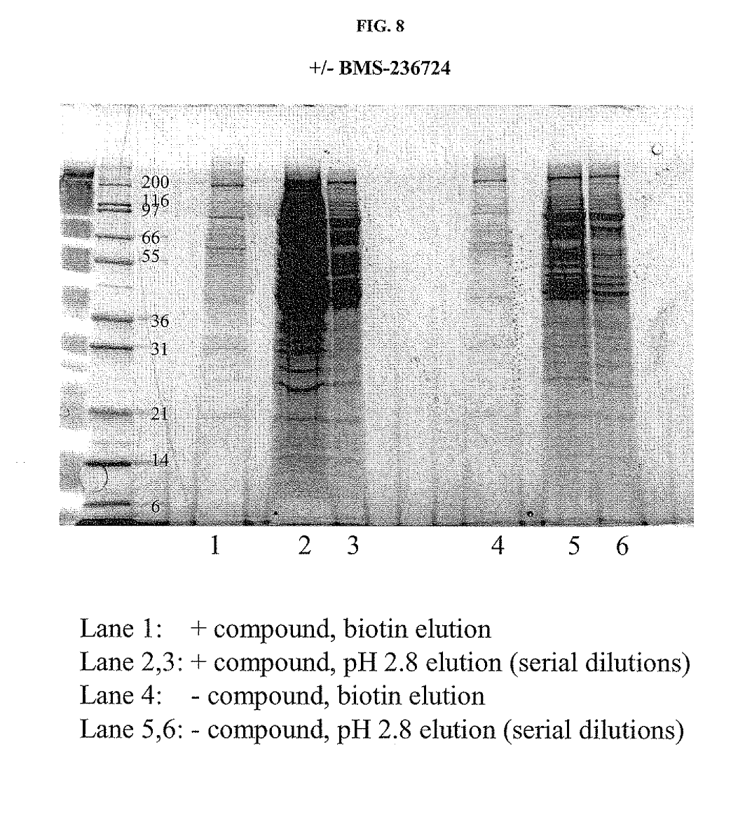

Using HCT116 quiescent cell extracts, affinity chromatography on monomeric avidin agarose columns was performed with the biotinylated compound. BMS-236724 shown below. This compound was inhibitory to farnesyl transferase and retained some of the proapoptotic activity of BMS-214662, considering that the much larger compound might have cell permeability issues. Moreover, this solid phase compound was able to bind and enrich for farnesyl transferase from a crude extract. An S100 extract from HCT116 quiescent cells was chromatographed on the agarose beads, incubated with or without BMS-236724 and eluted with biotin and also with pH 2.8 glycine buffer. The results are shown in FIG. 8. Individual bands specifically detected here have molecular weights of approximately 80 and 60 kDa (these results were compatible with the sizes of AIF identified in our more recent experiments outlined below). The structure of the biotinylated BMS-214662 derivative is provided below.

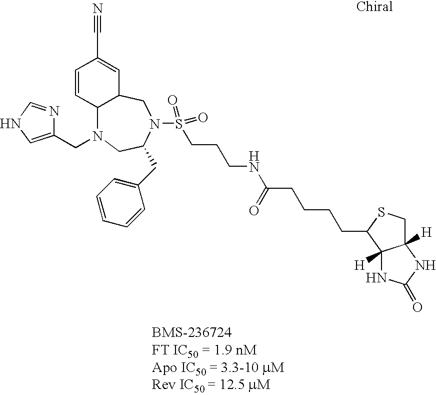

### Example 3

### Method of Identifying the Mechanism of Action of BMS-214662 in Selectively Inducing Apoptosis in Quiescent Tumor Cells Using Affinity Chromatography and Cross-Linking Compound(s)

Using affinity chromatography as described elsewhere herein, a cross-linking compound that was labeled with tritium allowed detection of a band approximately at 60-70 kDa on an SDS polyacrylamide gel, as shown in FIG. 7. The molecular weight of this band is compatible with the data the inventors obtained from the protein band B that bound to the biotinylated derivative BMS-844472 (this compound is identical to the BMS-236724 compound). The compounds utilized for the cross-linking studies are provided below.

### Crosslinking Analogues

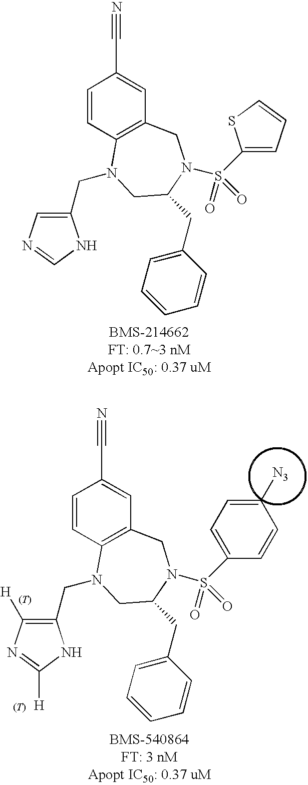 [0277] Protein bands pulled out with the help of BMS-540864

The bands containing the proteins corresponding to the indicated molecular weight were cut out from the gels and proteins identified by mass spectrometry of protease digests and shown in FIG. 7.

With the exception of AIF, no clear indication of a polypeptide that made sense to the pro-apoptotic biology was found at the time in this high molecular weight. However, it should be noted that the two major forms of AIF have a molecular weight of 75 and 61 kDa, which seem to agree with the crosslinked material detected here in the gel provided in FIG. 7. The smallest form of AIF (AIFsh) that may retain the binding pocket detected by crystallography has a MW of 35 Kda and a band at this point is in the gel provided in FIG. 7.

### Example 4

### Method of Identifying the Mechanism of Action of BMS-214662 in Selectively Inducing Apoptosis in Quiescent Tumor Cells Using Affinity Chromatography and Cross-Linking Compound(s) in Conjunction with Competition Experiments

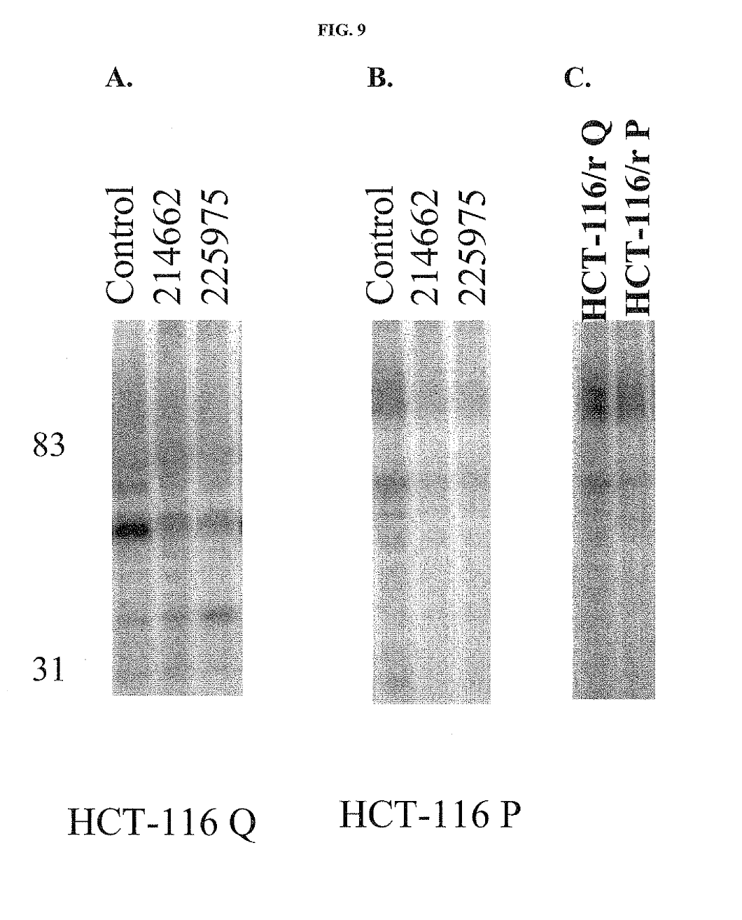

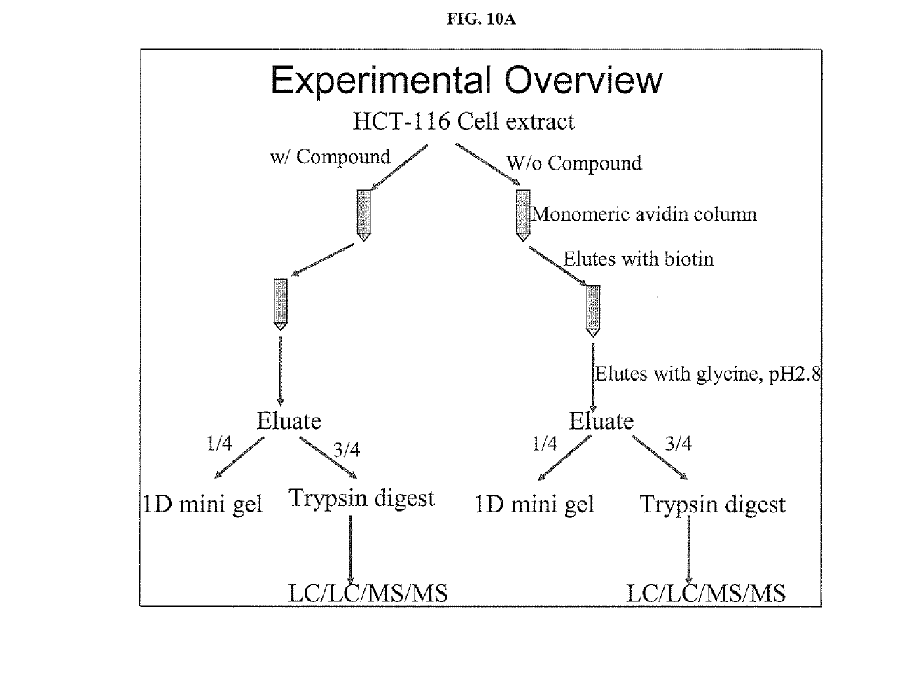

Competition experiments were also performed with the tritium labeled azido analogue BMS-540864 (shown above) and competed either with BMS-214662 or other analogues. Both compounds that have been modeled to fit into the AIF pocket, BMS-214662 and BMS-225975, are able to compete for the cross-linker site, but the functional consequence of this competition is not assessed in this kind of experiment. More importantly, these crosslinked proteins seem to be absent from the proliferating P cells, further supporting the connection between BMS-214662 affect on quiescent cells, its binding to AIF, and the subsequent induction of apoptosis. Equally significant, a HCT-116 variant selected from a tumor xenograft resistant to BMS-214662 (HCT-116/r) displayed a pattern of cross-linked proteins more similar to the HC-116 P, irrespective of whether these cells were proliferating or quiescent (see FIG. 9). The crosslinked proteins were not identified in this experiment, however. These results further support the connection between AIF and the pro-apoptotic effects of BMS-214662 because the AIF bands were not observed in either the resistant HCT-116 strain nor in the proliferating, BMS-214662 sensitive HCT-116 strain, but rather only in the quiescent HCT-116 strain.

Prior to the elucidation of AIF as being the target of BMS-214662 (i.e., the work first described herein), extensive transcriptional profiling had been performed on the P and Q HCT116 and K562 cells treated with BMS-214662 for different times and at different doses (data not shown). Very limited information on the potential target for BMS-214662 could be gleaned from that information, compatible with secondary protein modifications and or subcellular localization being part of the mode of action of the BMS-214662 compound.

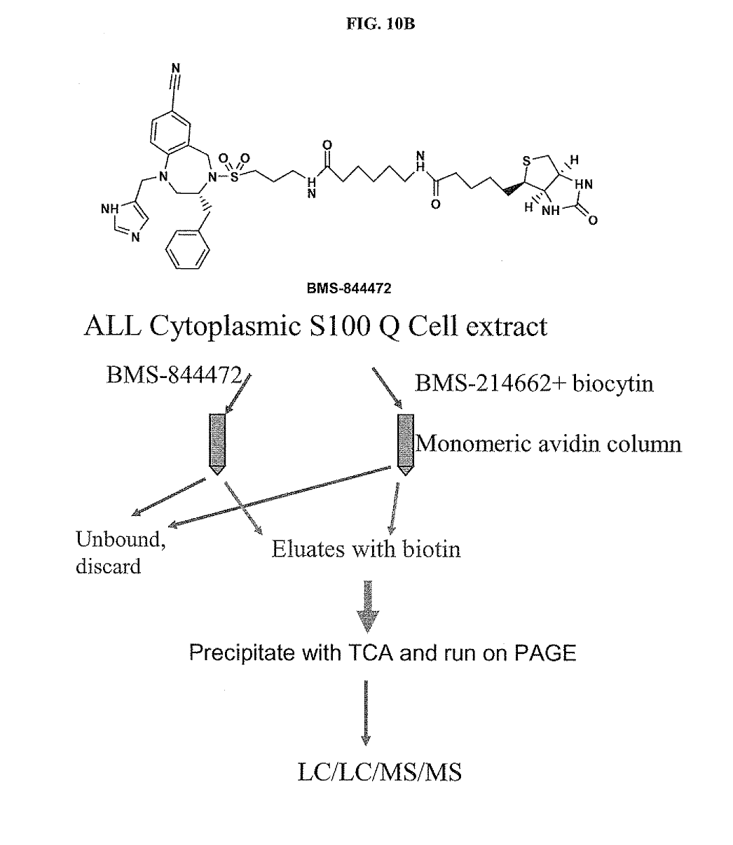

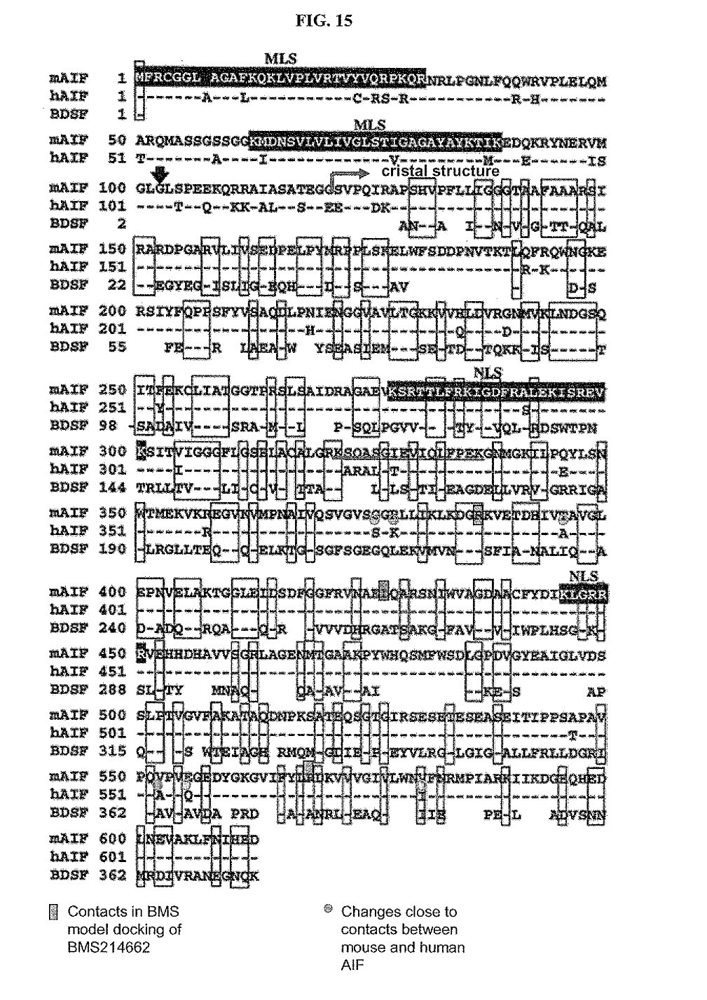

Although the early results described herein and illustrated in FIGS. 7, 8, and 9 were not conclusive, they are consistent with the new results described in FIGS. 10 to 14 which identify AIF as being the target for BMS-214662. During the repeat of several of the early experiments using the tritium labeled BMS-540864 analogue, it was noted that there was a much lower sensitivity to this compound in rodents vs. humans, and that may explain the good therapeutic window in animal models. A comparison of the mouse and human AIF sequences reveals very strong conservation, but there are some changes in the proximity of the predicted contact sites, which could lead to a difference in substrate specificity (see FIG. 15).

### Example

### Additional Methods of Identifying the Mechanism of Action of BMS-214662 in Selectively Inducing Apoptosis in Quiescent Tumor Cells Using a Biotinylated Analogues of BMS-214662

An important first step was to establish a source of cells that grew to high density and could remain viable even without replenishing the medium, so that they would be quiescent (Q). After several trials, the inventors settled on ALL cells. Nuclear and cytoplamic fractions were separated by low speed centrifugation after cell lysis with mild detergent. The nuclear fractions were treated with 0.5 M KCl and the chromatin spun out at 100,000 g for one hour. The cytoplasmic fraction was also spun at 100,000 g for one hour, glycerol was added to 20% and fractions frozen at −80 C. The next day the nuclear and cytoplasmic fractions were incubated either with biocytin and BMS-214662 (control) or BMS-844472 (shown below) for 1 hour at 4 C.

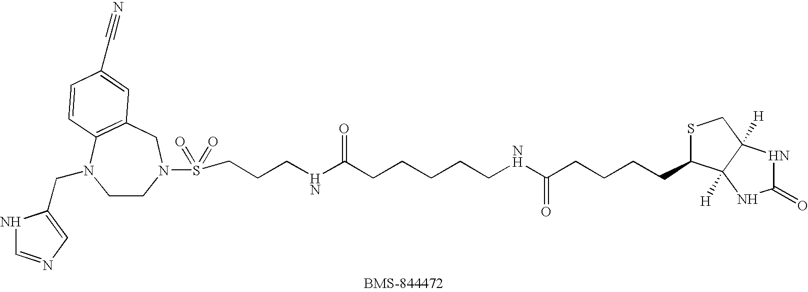

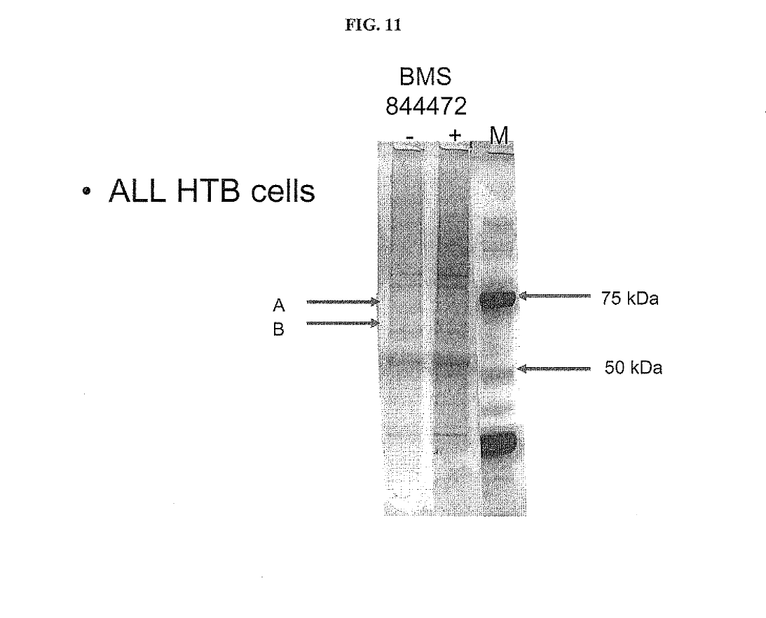

The fractions were loaded onto 1 ml columns of monomeric avidin beads that had been pretreated with biotin to block accessible sites and washed with pH 2.8 glycine buffer and the incubation buffer (see FIG. 10). Proteins were eluted from both columns with buffer containing 2 mM biotin, 10% TCA precipitated and analyzed on polyacrylamide gels. A representative silver stained gel is shown in FIG. 11.

Bands A and B from the 1 mm gel shown in FIG. 11 were excised out and submitted to the proteomics group for analysis. The results of our first successful analysis are summarized in Table 2.

TABLE 2

Peptide Hits

| Protein | Exp 1 | Exp 2 |
|---|---|---|
| Stress induced-phosphoprotein 1 | 15 | 18 |
| (Hsp70/Hsp90-organizing protein) | | |
| Protein phosphatase 1G (Homo sapiens) | 4 | 1 |
| Tubulin (several forms) | 10 | 1 |
| Programmed cell death (AIF) | 3 | 5 |

Two peptides were detected for AIF in band B of the BMS-844472 eluates that were absent from the control eluates. AIF appears in cells in several forms, the intact 613 precursor, a splice variant with apoptotic activity of 609 aa, a proteolyzed form with apoptotic activity of 512 aa, a short form with an alternative start site (AIFsh, 261aa) and two forms (AIFsh2 and AIFsh3, 324 and 237 aa, respectively) that are inactive in the apoptotic process. The molecular weight of the band from which the inventors detected the peptides seems to correspond to the molecular weight of the complete 613 aa inner mitochondrial membrane bound form or the 512 as form that transits to the nucleus. The only second protein represented by two polypeptides was eukaryotic translation elongation factor 1 alpha with no known role in apoptosis.

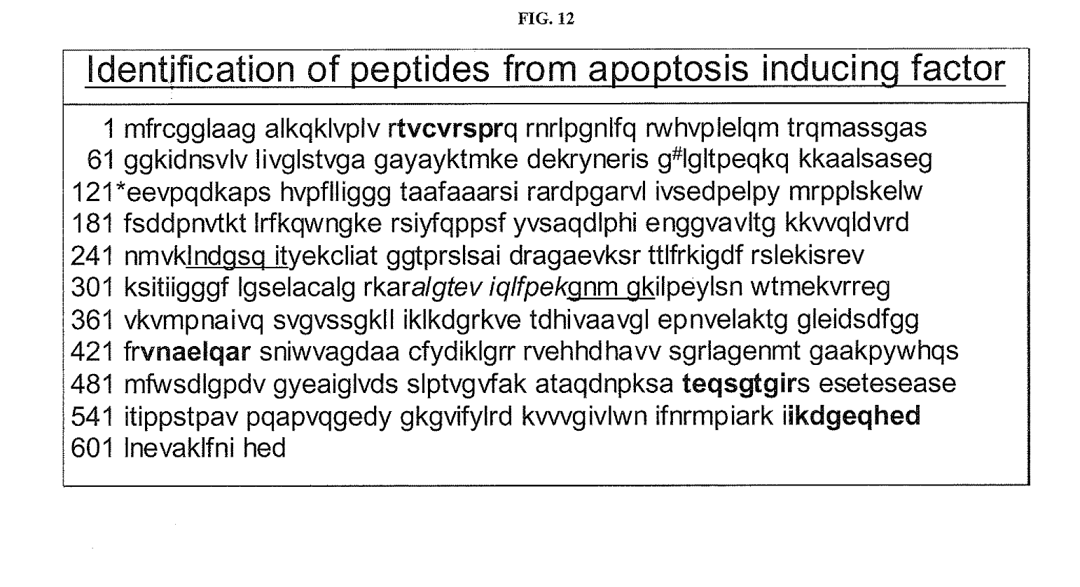

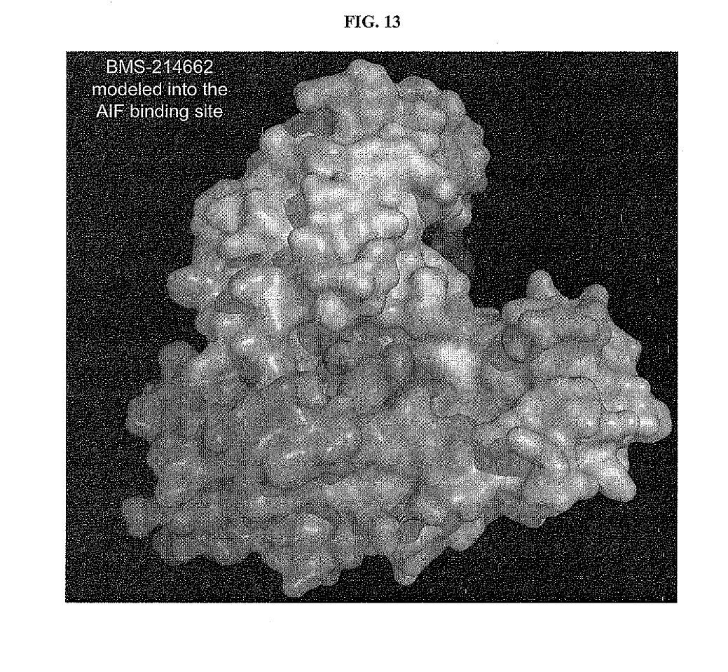

A second analysis of proteins from this cytoplasmic ALL fraction revealed 5 peptides corresponding to AIF. The peptides detected are indicated in the FIG. 12 coded with different colors. One peptide, in red was detected in both runs. This peptide was also detected in the run of size fractionated cytoplasmic S100 from Q HCT-116 cells run in earlier experiments, when the MS spectral data was reanalyzed with current software.

In view of this assignment of AIF as the target for BMS-214662, an effort was made to establish whether the binding of AIF with BMS-214662 could be predicted based upon in silica molecular modeling. Molecular modeling was performed and showed clearly the existence of a deep pocket on the AIF protein that could accommodate BMS-214662 (see FIG. 13). Three contacts appear to be established with BMS-214662.

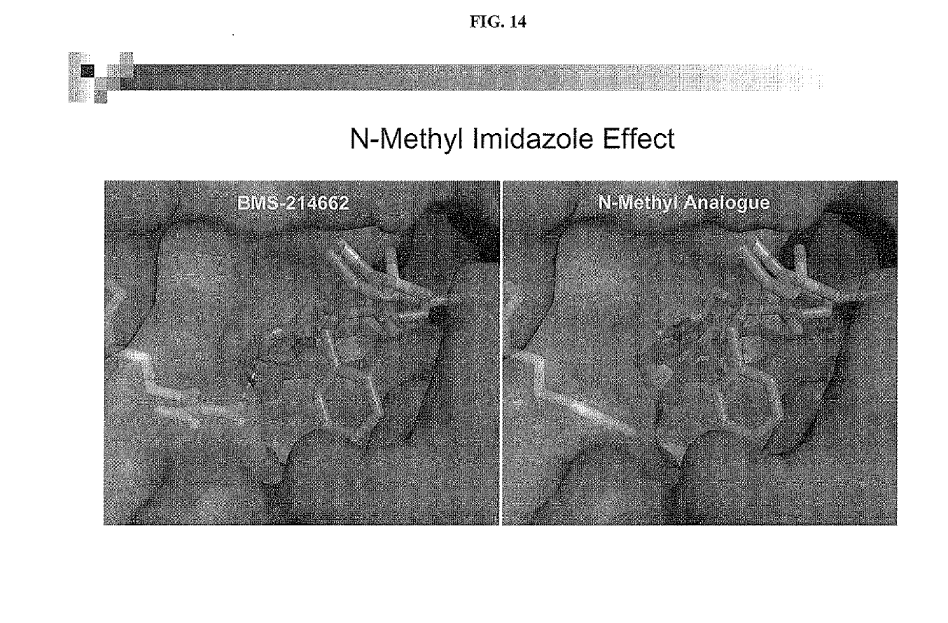

In support of this interaction, the inability of the BMS-225975 compound to induce apoptosis could also be explained by molecular modeling because the methyl on this compound was such that its binding within this pocket would be occluded, and also didn't permit forming contacts with the arginine at position 450 (see FIG. 14). Specifically, N-Methyl substitution favors an imidazole rotamer which moves the acceptor N away from Arg in order to avoid a steric clash between methyl and the fused ring system. This results in the loss of a potential key H-bond to Arg, an observation consistent with apoptotic SAR. These data were derived from the ATP structure available in the public domain.

In view of this data accumulating on the possible role of AIF in the MOA of BMS-214662, it was essential for us to determine that the data previously collected was compatible with the findings outlined supra for the BMS-214662, BMS-225975 and BMS-844472 compounds.

### Example 6

### Method of Determining the Identity of Proteins that Bound to BMS-214662 Using Mass Spectrometry

The identity of the proteins in the bands isolated from the gels shown in FIGS. 7 thru 9 were made using LC/LC/MS/MS. Prior to subjecting the samples to mass spectrometry, the bands were first subjected to a series of washing and destaining, reduction/alkylation, tryptic enzymatic digestion, and extraction steps designed to prepare the bands for mass spectrometry as outlined below.

As outlined herein, the SDS-PAGE gels were silver stained using the Silver Quest Silver Staining Kit (Invitrogen (Carlsbad, Calif.)). The following steps were performed to prepare the gel slices for analysis:

### Washing and Destaining

The gel slices were washed dd H₂O for 15 minutes, twice. Then the protein bands were excised from the gel and placed into a 1.5 ml snap-cap. The gel was destained with 150 μl Invitrogen Silver Quest destainer A and 150 μl destainer B for 15 minutes. Samples were washed with dd H₂O for a few minutes, twice. The gel slices were then cut into ˜1 mm cubes, and washed with 150 μl 50:50 H₂0: acetonitrile. The samples were then washed with 150 μl acetonitrile, and dried in a speed-vac until they were VERY dry.

### Reduction/Alkylation

50 μl of 10 mM DTT/100 mM NH₄HCO₃ was added to the samples and incubated at 56° C. for 45 minutes. Samples were allowed to cool to room temperature, and the solution was then pull-off and discarded. 50 Ξl of 50 mM iodoacetamide/100 mM NH₄HCO₃ was added, and the samples were allowed to incubate in the dark at room temperature for 30 minutes. The solution was then pulled off and discarded. The samples were then washed with 150 μl 100 mM NH₄HCO₃ for 5 minutes. 150 μl acetonitrile was added to each samples and incubated at room temperature for 15 minutes. The solution was then pulled off, discarded, and the samples were then dried in a speed-vac until they were VERY dry.

### Digestion/Extraction

40 μl of trypsin buffer solution (or enough to moisten the dried gel cubes) was added to each sample, and then allowed to incubate on ice for 45 minutes to allow the trypsin to be absorbed by the gel. The excess solution was pulled off and discarded. 40 μl (or enough to cover the pieces) of trypsin-free buffer was added and allowed to incubate 16 hours (overnight) at 37° C. The samples were then centrifuged to remove condensation from top of vial to bottom. The solution was pulled off and saved in 0.5 ml snap-cap vial (Eppendorf, Protein LoBind, stock #2243106-4). 50 μl 25 mM NH₄HCO₃ was added and incubated at room temperature for 15 minutes. 50 acetonitrile was added to each sample and incubated for further 15 minutes. The solution was pulled off and saved with above retained volume. 50 μl 5% formic acid was added and incubated at room temperature for 15 minutes. 50 μl acetonitrile was added and incubated for further 15 minutes. Solution was pulled off and saved with above retained volume. The last three steps were repeated, and then the samples were completely dried in a speed-vac.

The following stock and working solutions were used:

### Stock Solutions:

### 1 M NH₄HCO₃ (MW=79.06)

0.1 M DTT (dithiothreitol, C₄H₁₀O₂S₂, MW=154.25)

### 0.1M CaCl₂ (MW=111.0)

### Working Solutions:

50% acetonitrile/50% dd H₂O

### 100 mM NH₄HCO₃

### 25 mM NH₄HCO₃

### 10 mM DTT in 100 mM NH₄HCO₃

50 mM IAA (iodoacetamide, ICH₂CONH₂) in 100 mM NH₄HCO₃

### Trypsin-Free Buffer Solution:

### 50 mM NH₄HCO₃ (150 μl 1M)

### 5 mM CaCl₂ (15 μl 1.0 M)

### 2700 μl dd H₂O

### Trypsin Buffer Solution:

Dissolve 1 vial=20 μg of Sequencing Grade Modified Trypsin (Promega, stock #V5111) in 1.6 ml of trypsin-free buffer solution.
final concentration=12.5 ng/ul. (40 μl=>0.5 μg trypsin/sample)

### FTI/AIF Mass Spectrometry Instrument Method

Mass spectrometry analyses was performed on the samples on a Thermo Scientific (San Jose, Calif.) LTQ ion trap mass spectrometer equipped with an AGILENT® 1100 (Santa Clara, Calif.) liquid chromatography system configured for nanospray. Chromatographic separations were done on a PHENOMENEX® (Torrance, Calif.) C18-monolithic column (150 mm length×75p I.D.) Buffer A was 0.1% formic acid in water and buffer B was 0.1% formic acid in acetonitrile. All solutions were mass spectrometry grade quality and obtained from J. T. Baker (Phillipsburg, N.J.). The flow was 5 μl/min. with a 1:10 pre-column split before the manual injection valve (Upchurch Scientific, Oak Harbor, Wash.). The gradient used was as follows:

| Time | % A | % B |
|---|---|---|
| 0.0 | 98 | 2 |
| 5 | 98 | 2 |
| 53 | 50 | 50 |
| 58 | 2 | 98 |
| 63 | 2 | 98 |
| 63.1 | 98 | 2 |
| 85 | 98 | 2 |

Data acquisitions were done under collision induced disassociation (CID) conditions with seven product ion scans collected from the seven most abundant parent ions per cycle. Dynamic exclusion was used with a repeat count of 2 for 30 seconds. The collision energy was 30 eV and the activation time was 30 ms.

Data was processed using Sequest on a Sorcerer (Sage-N-Research, Saratoga, Calif.) system and reviewed with TPP (trans proteomic pipeline, Institute for Systems Biology, Seattle, Wash.).

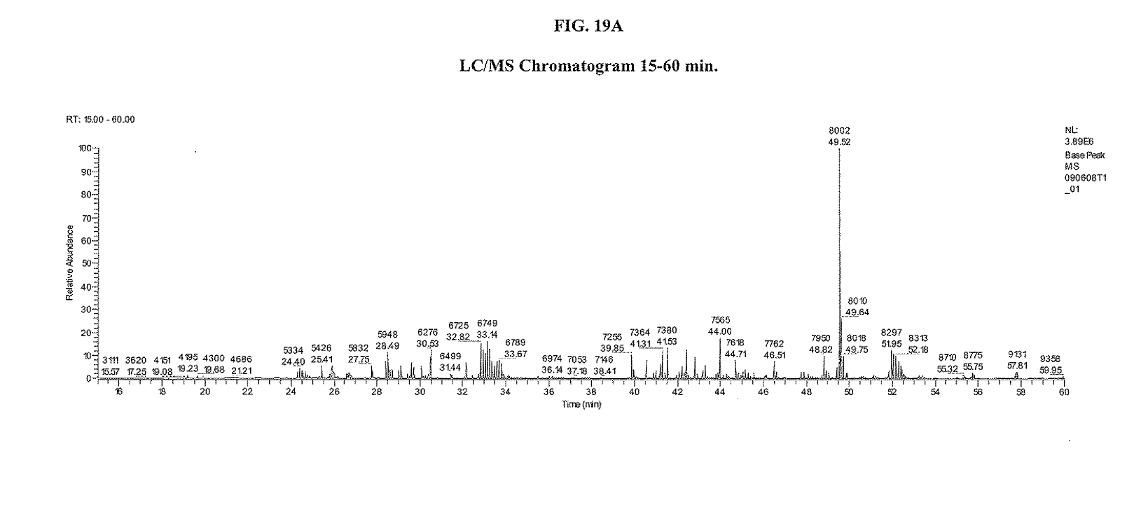

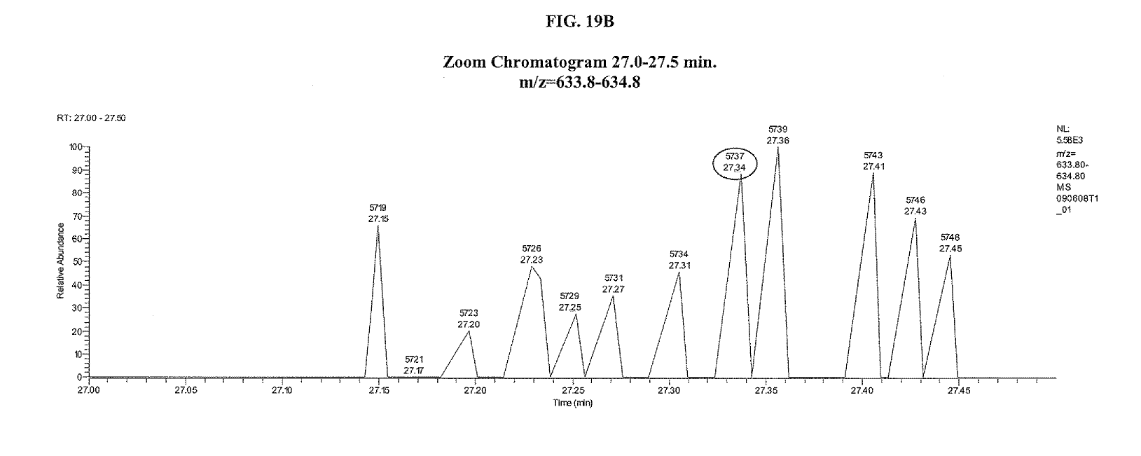

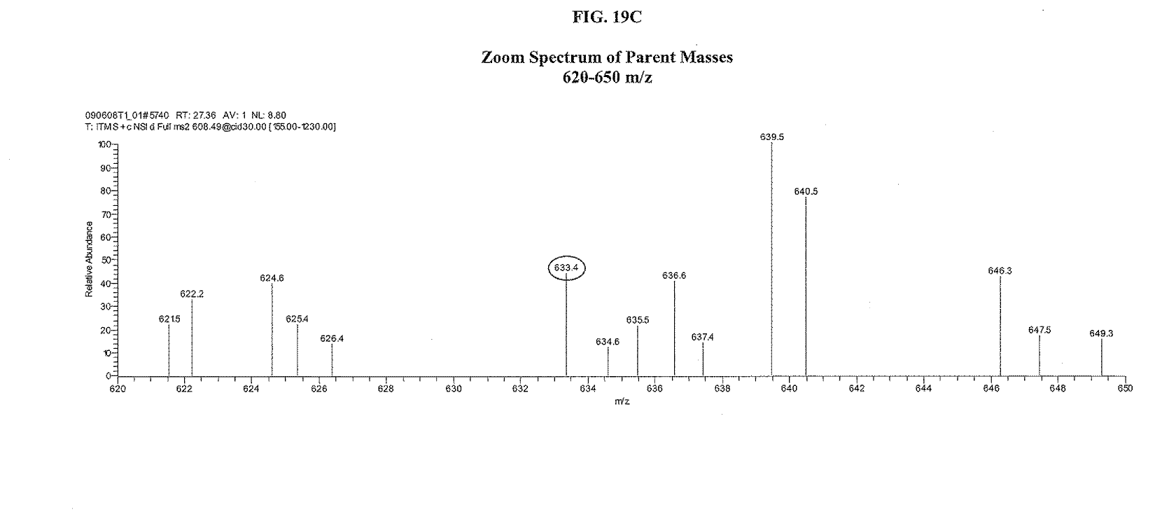

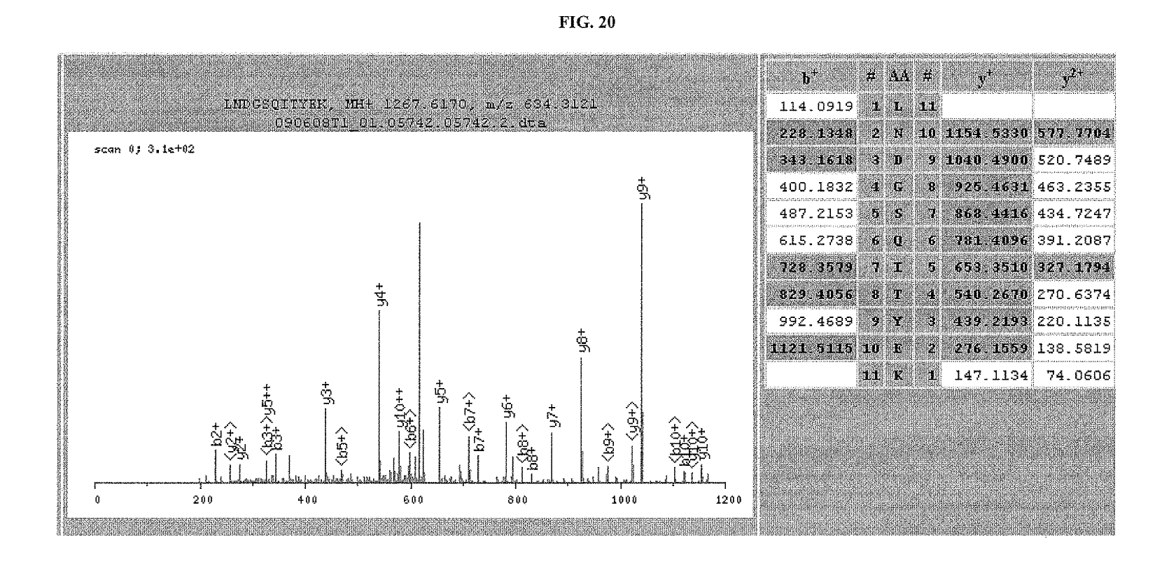

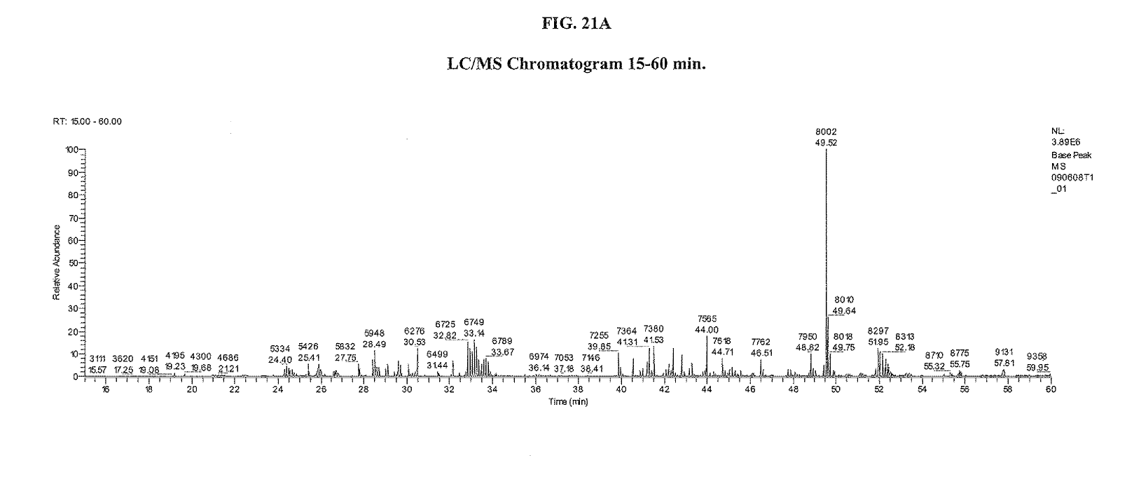

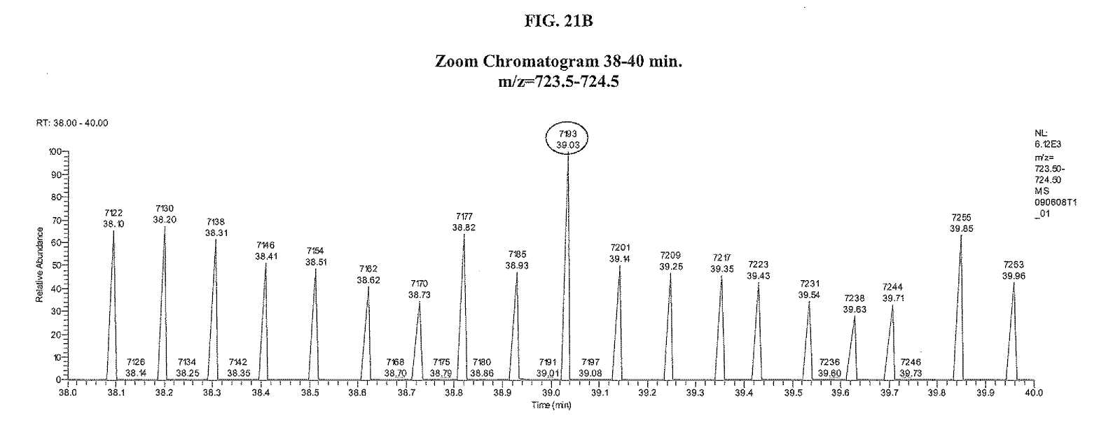

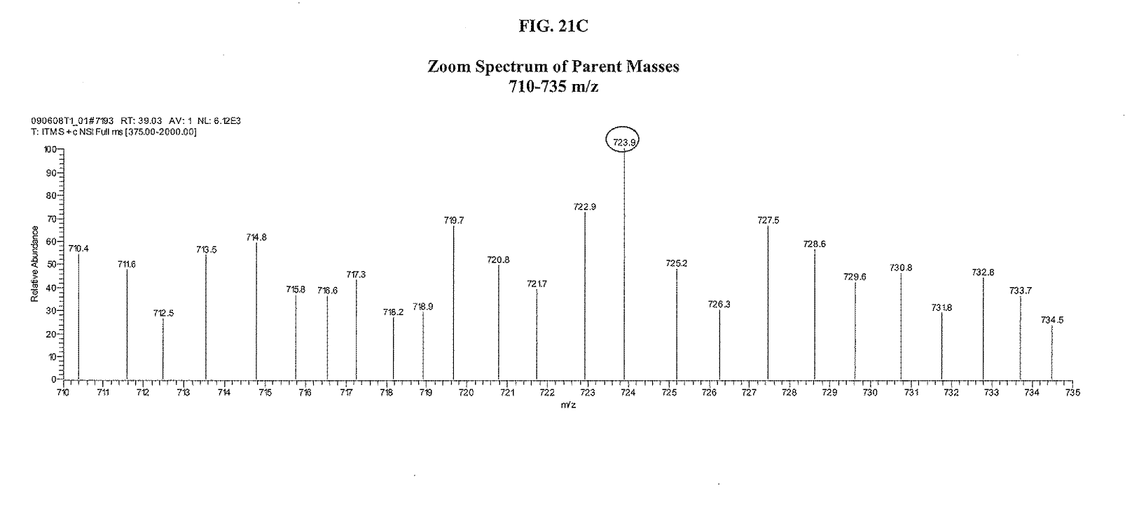

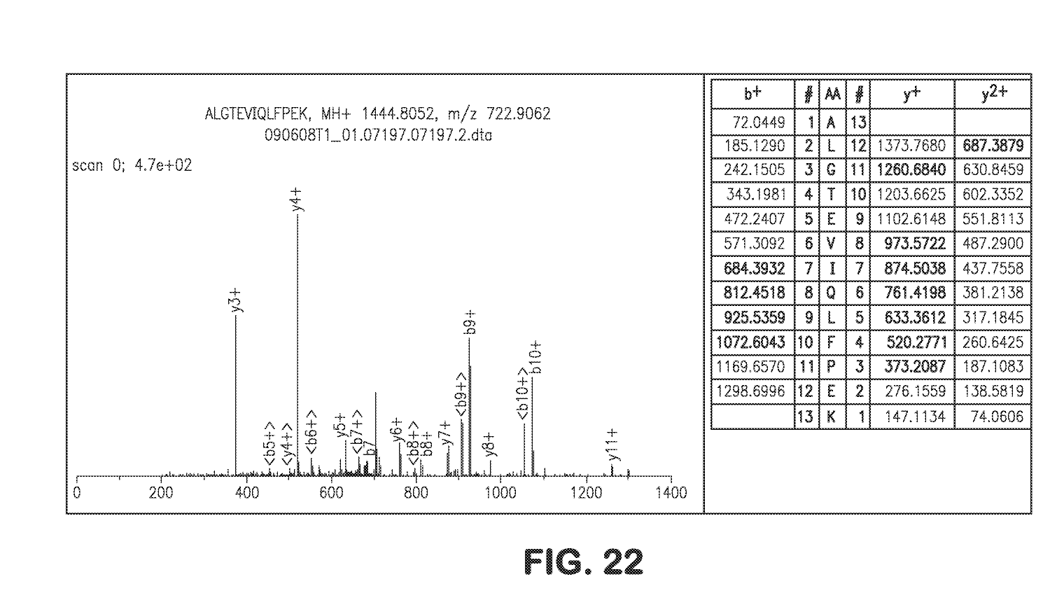

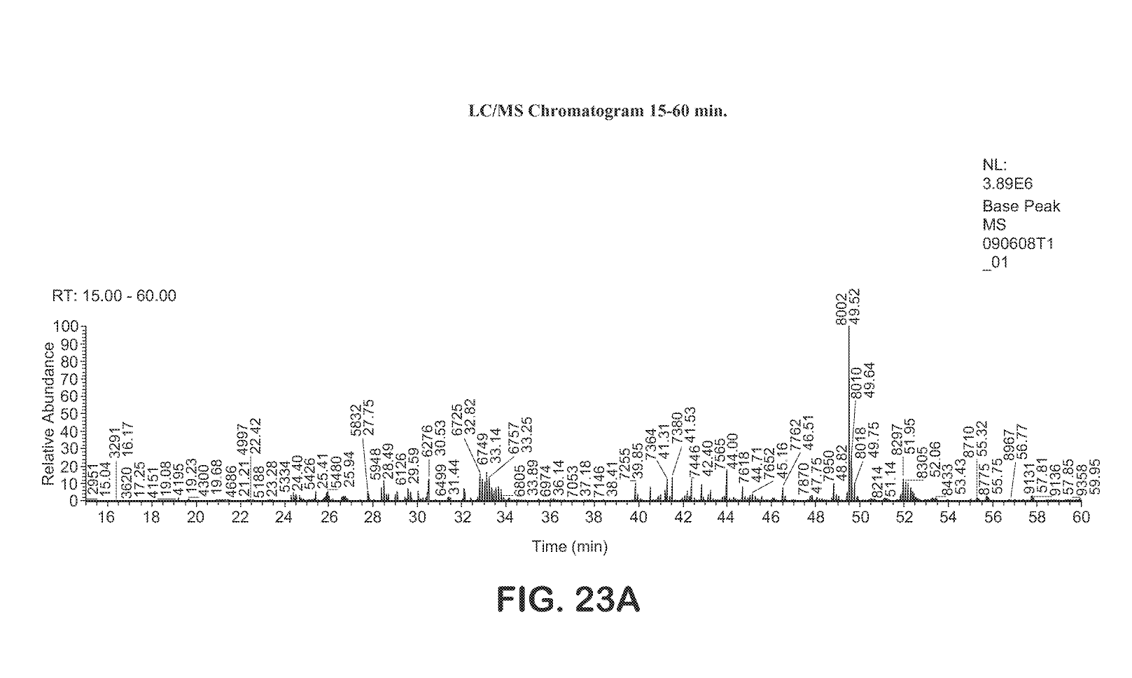

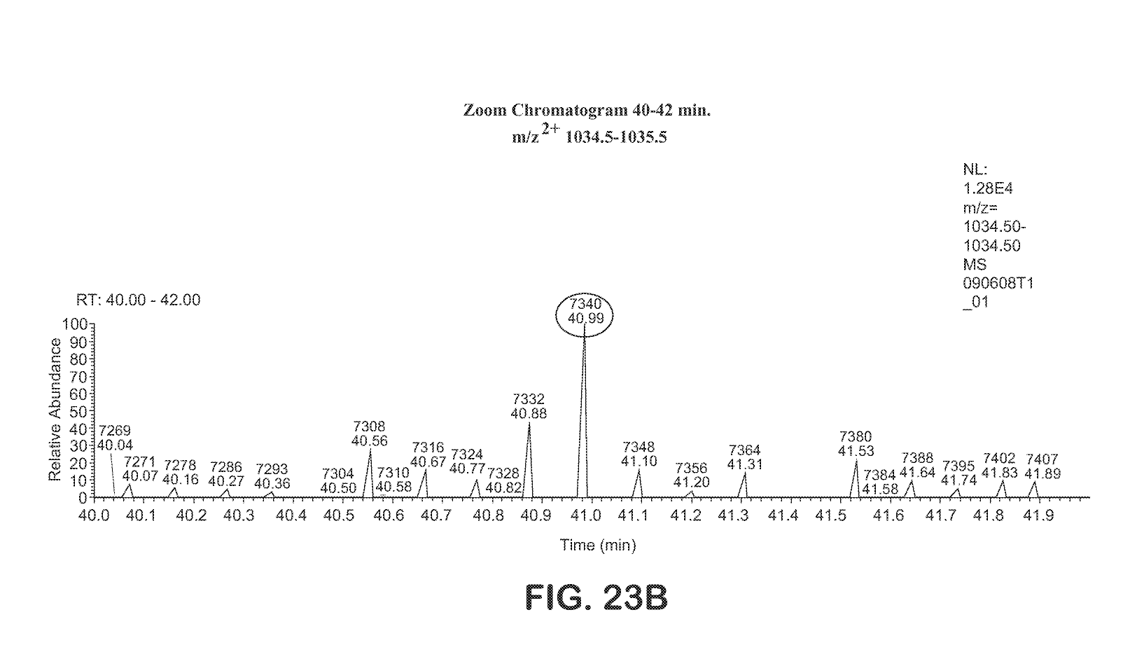

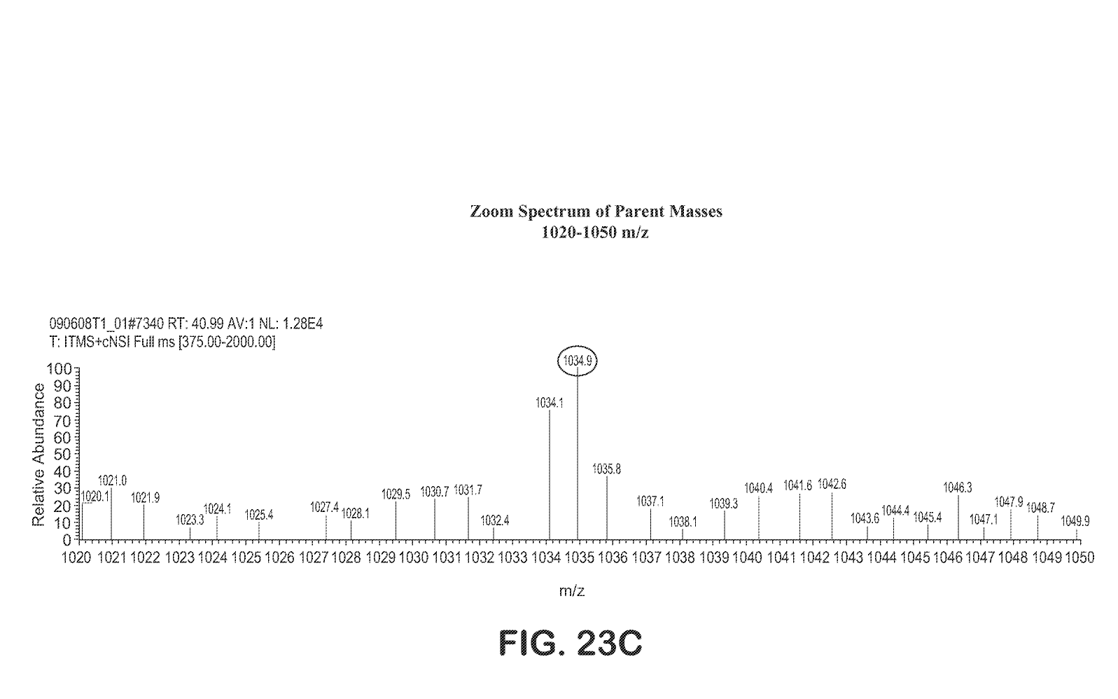

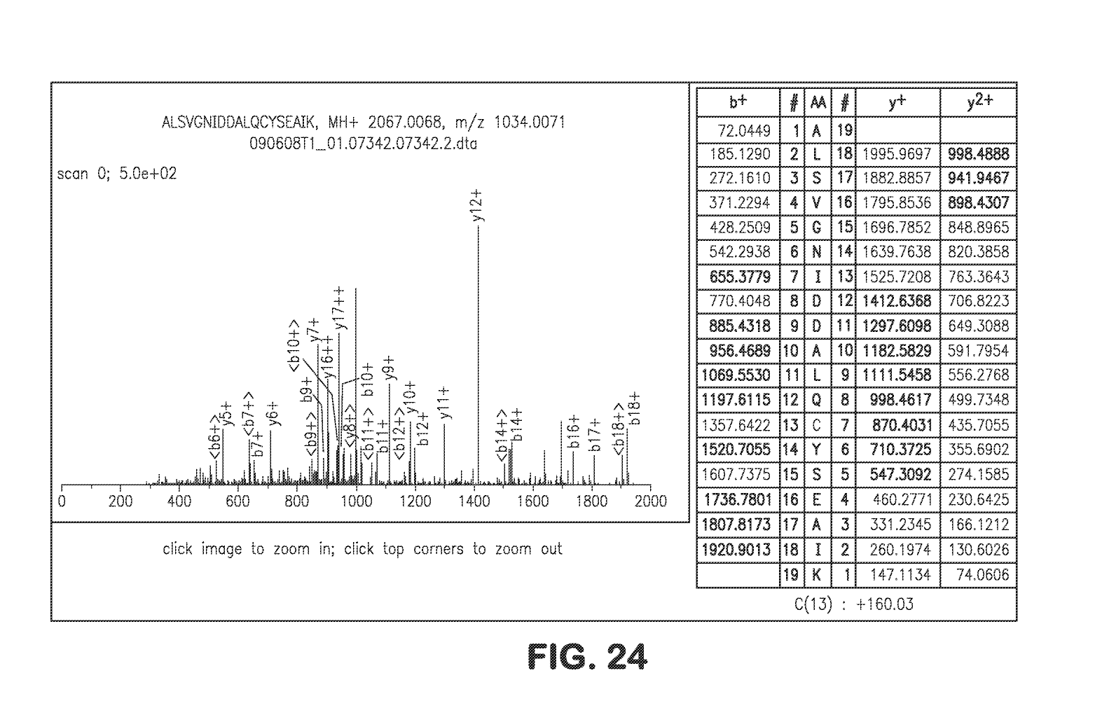

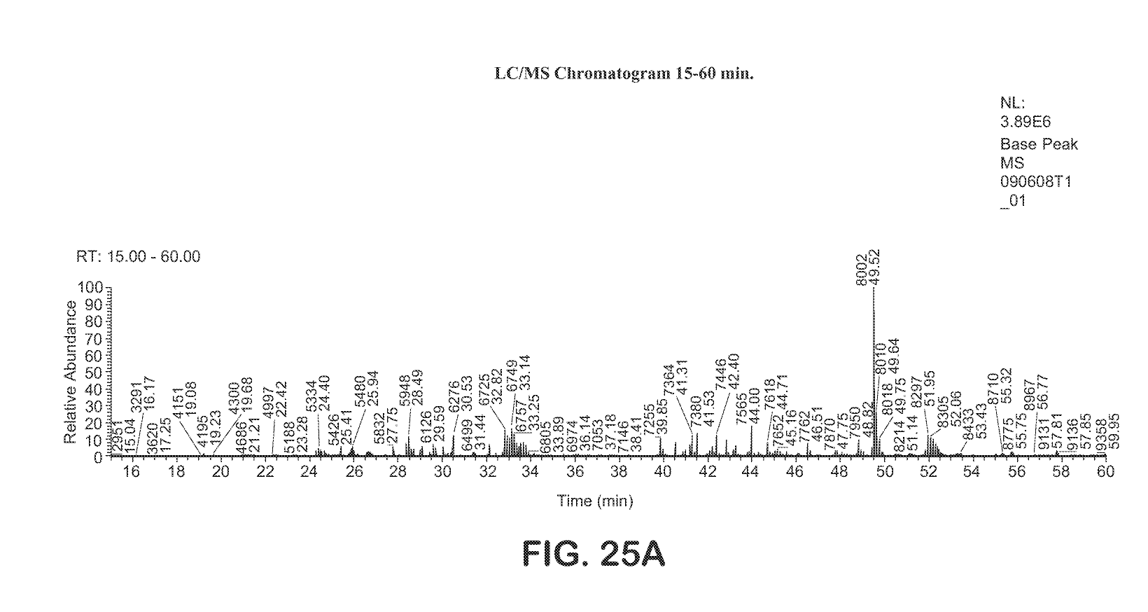

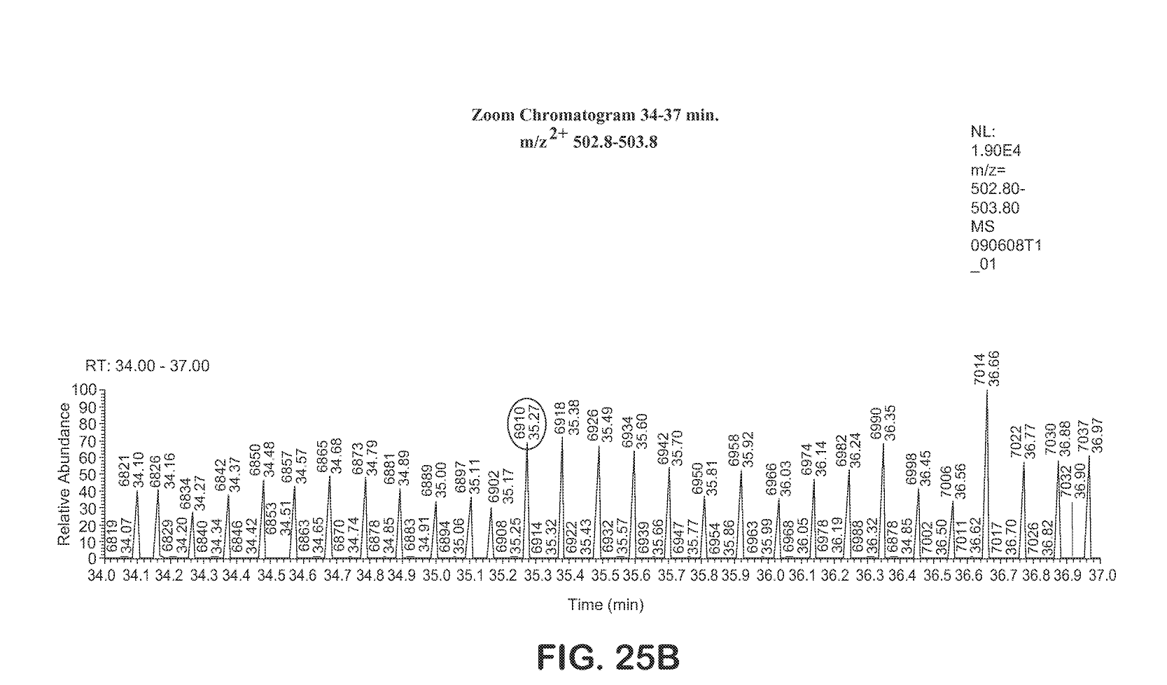

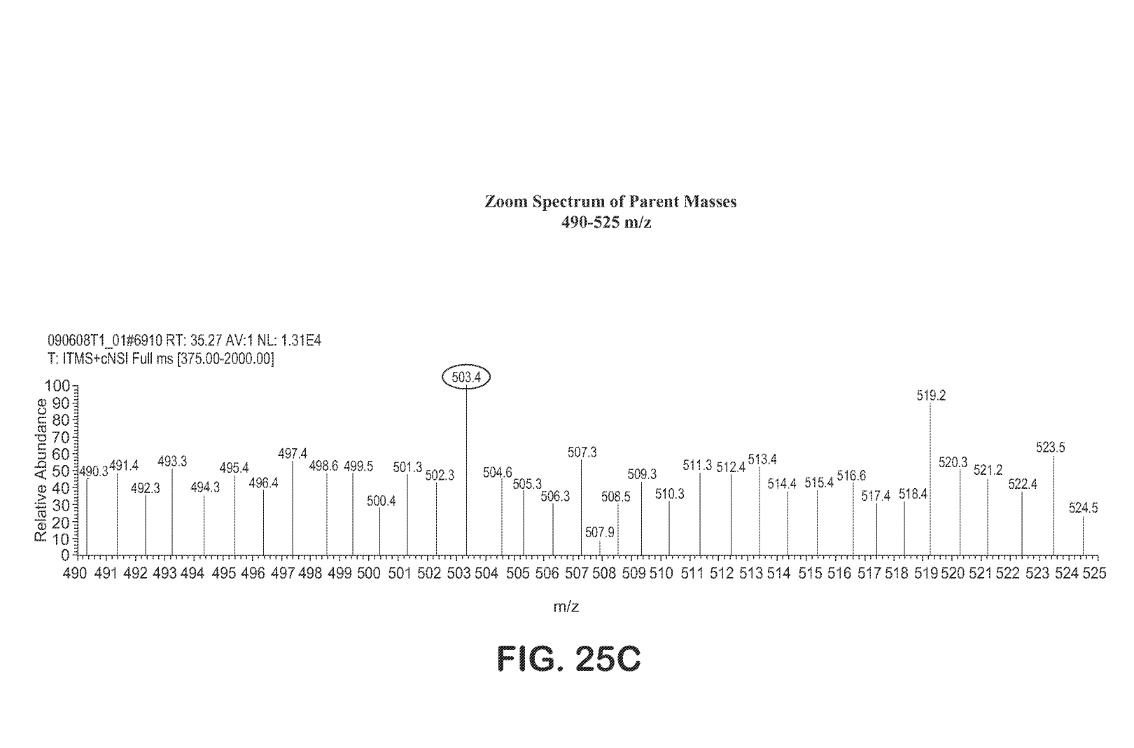

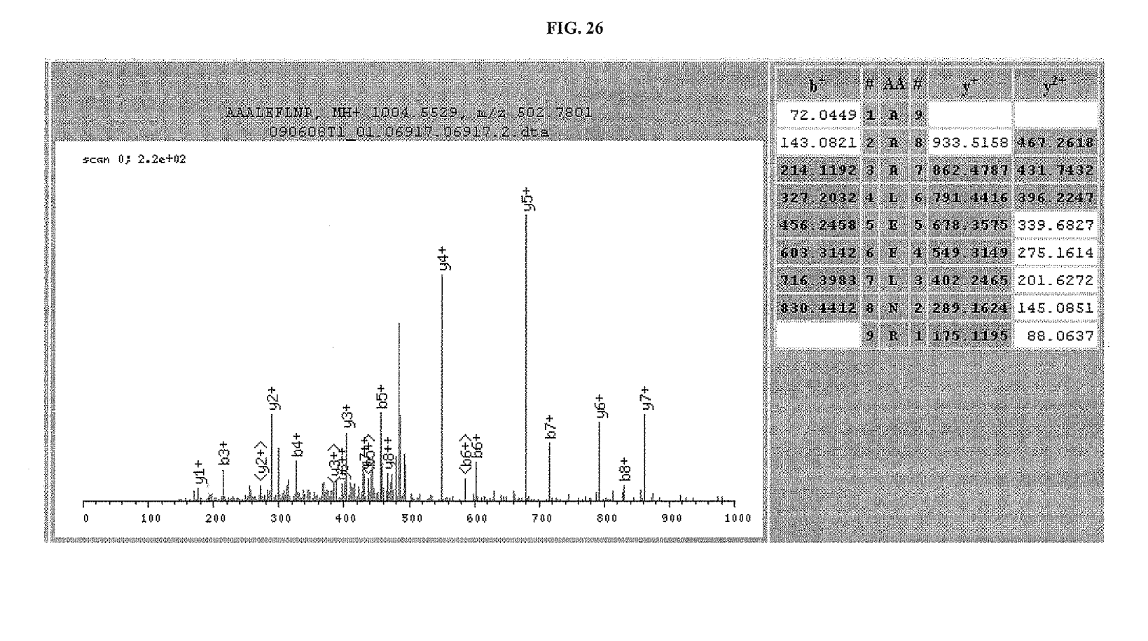

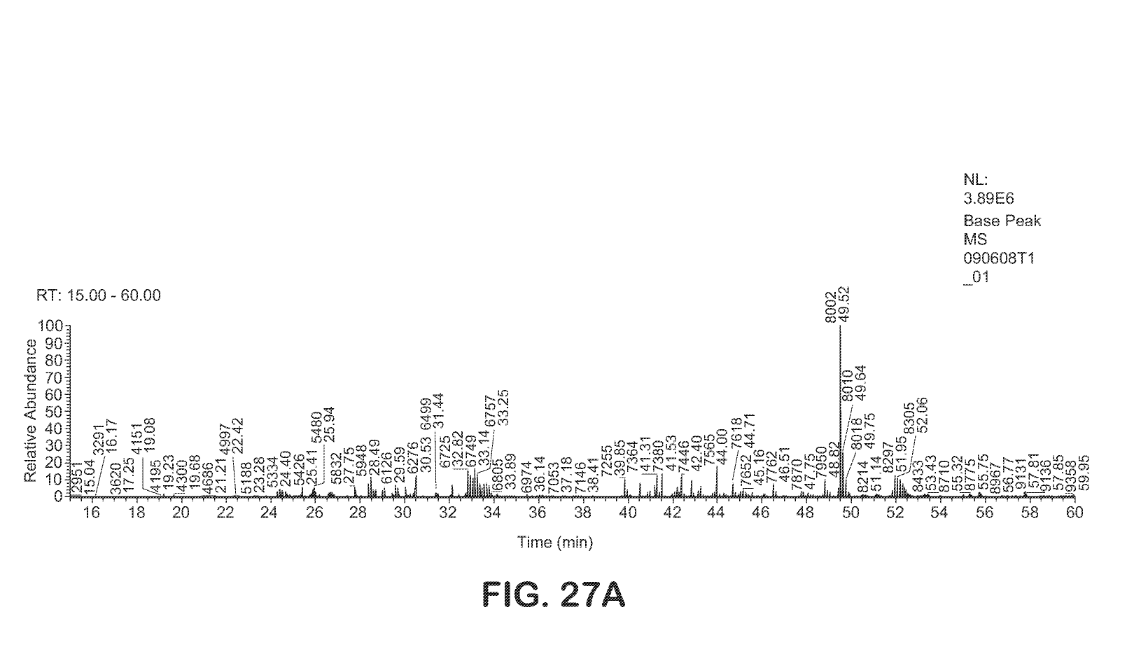

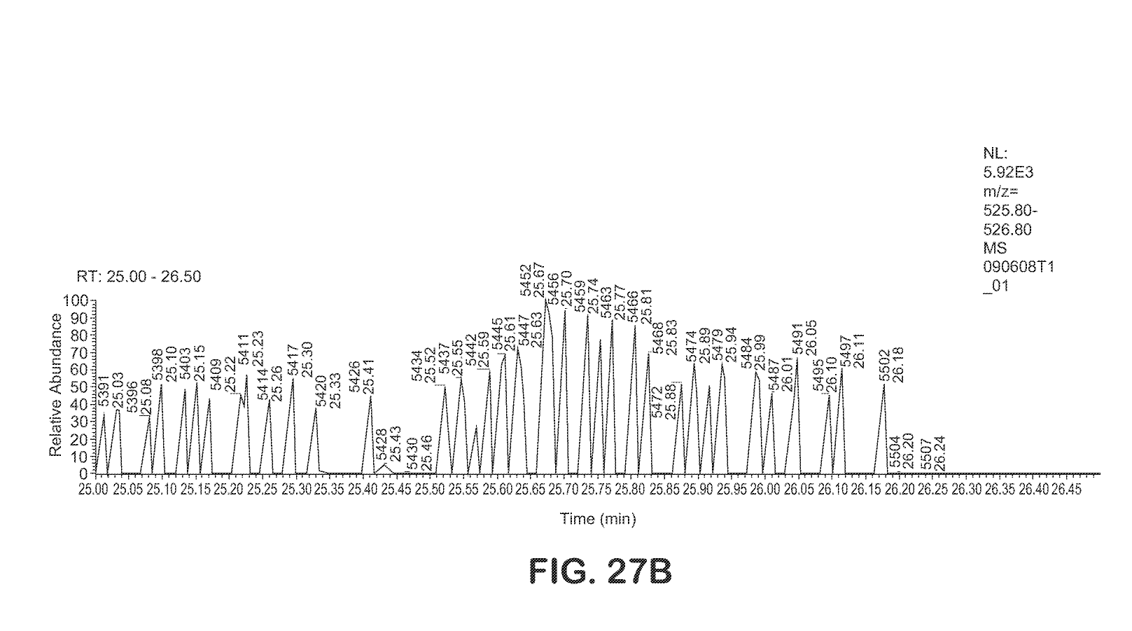

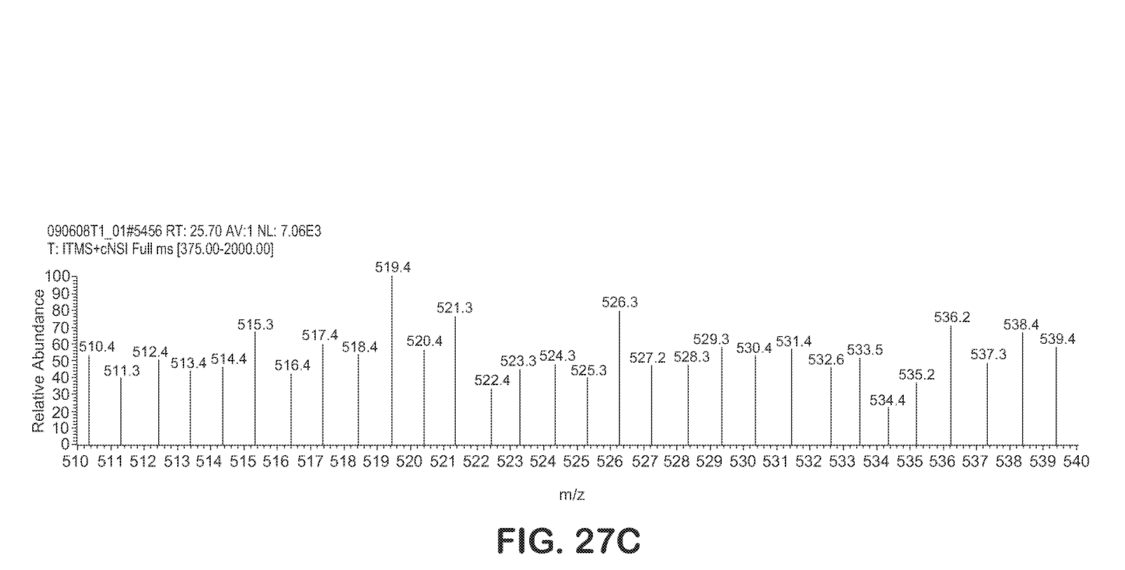

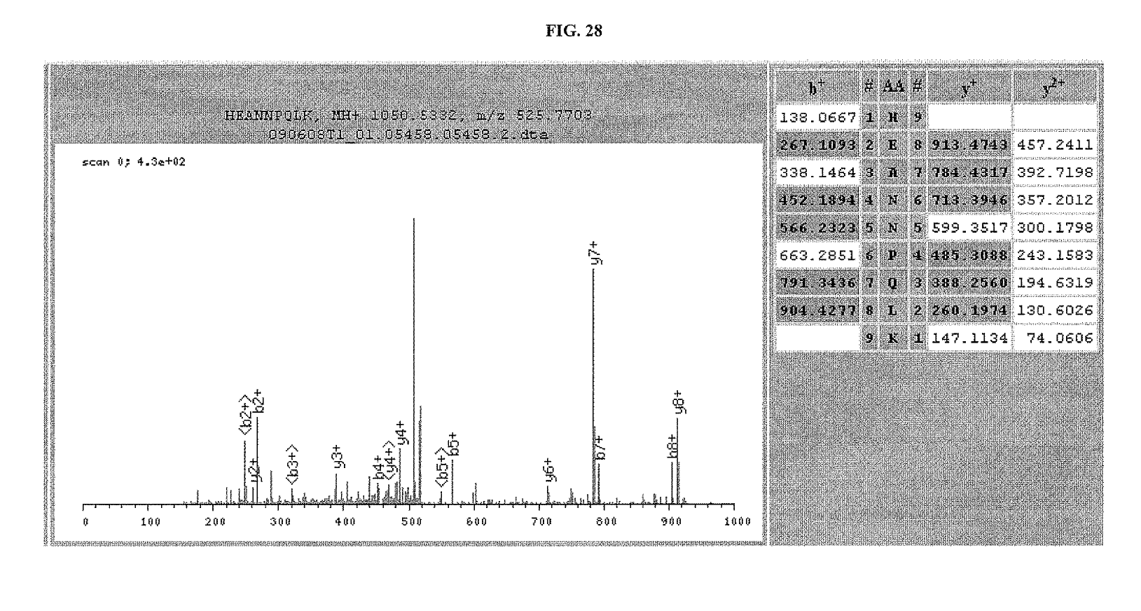

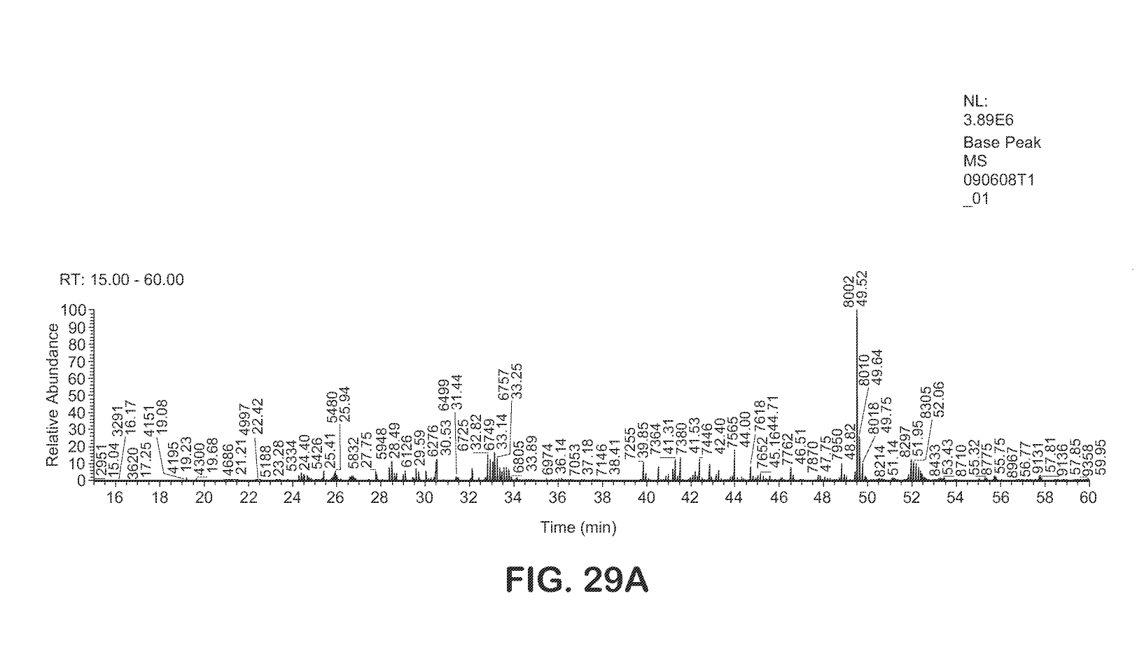

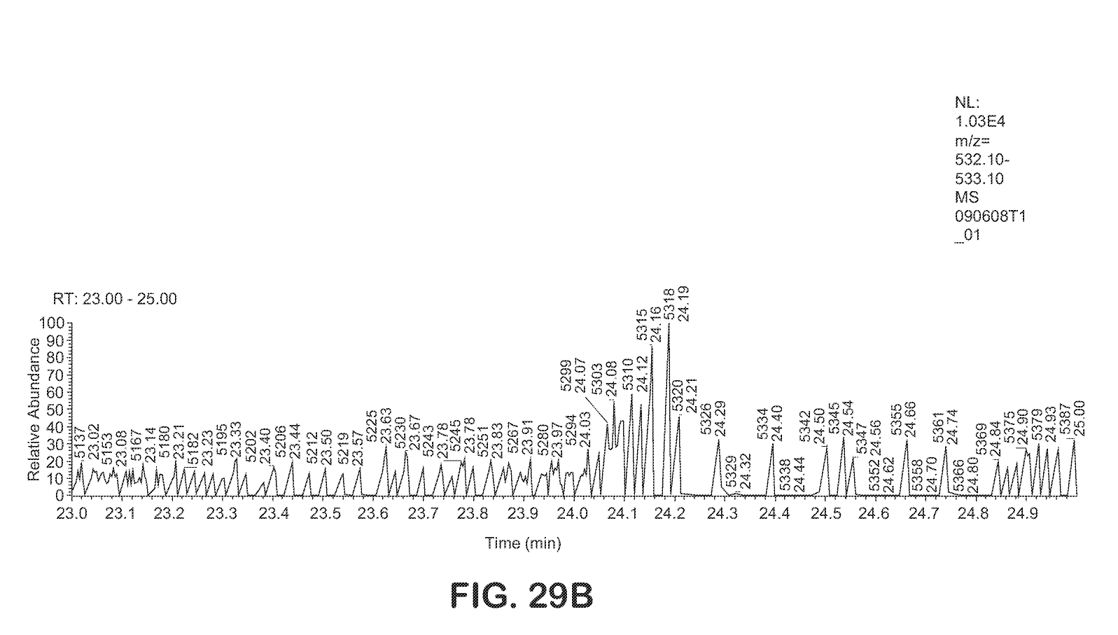

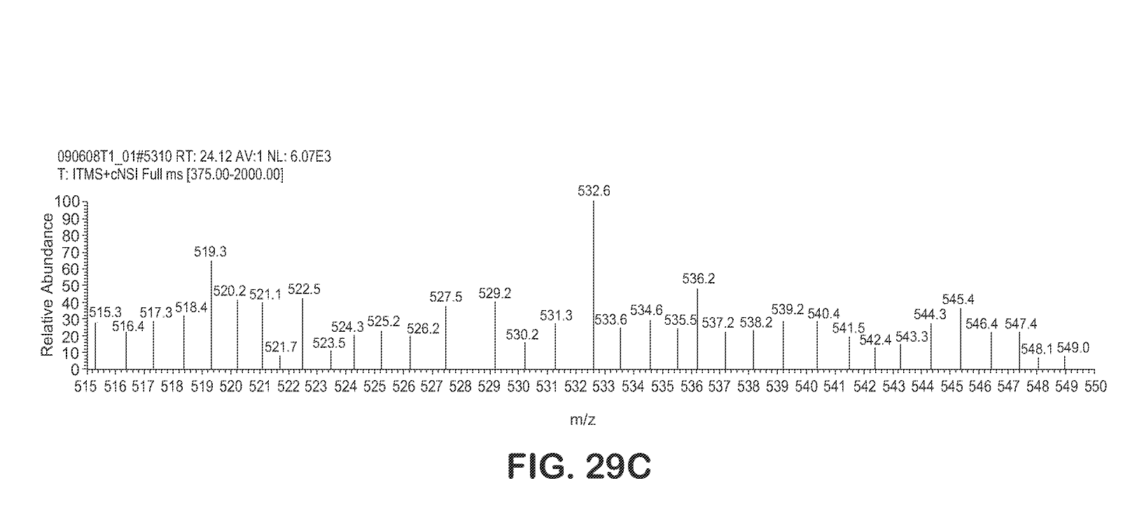

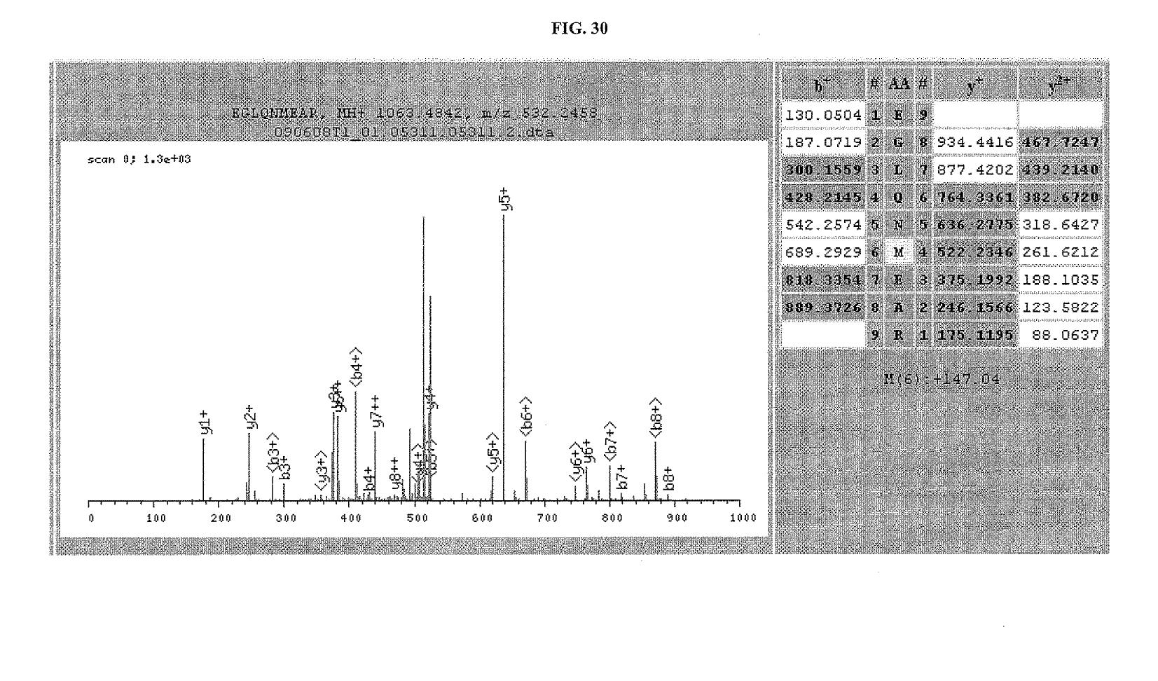

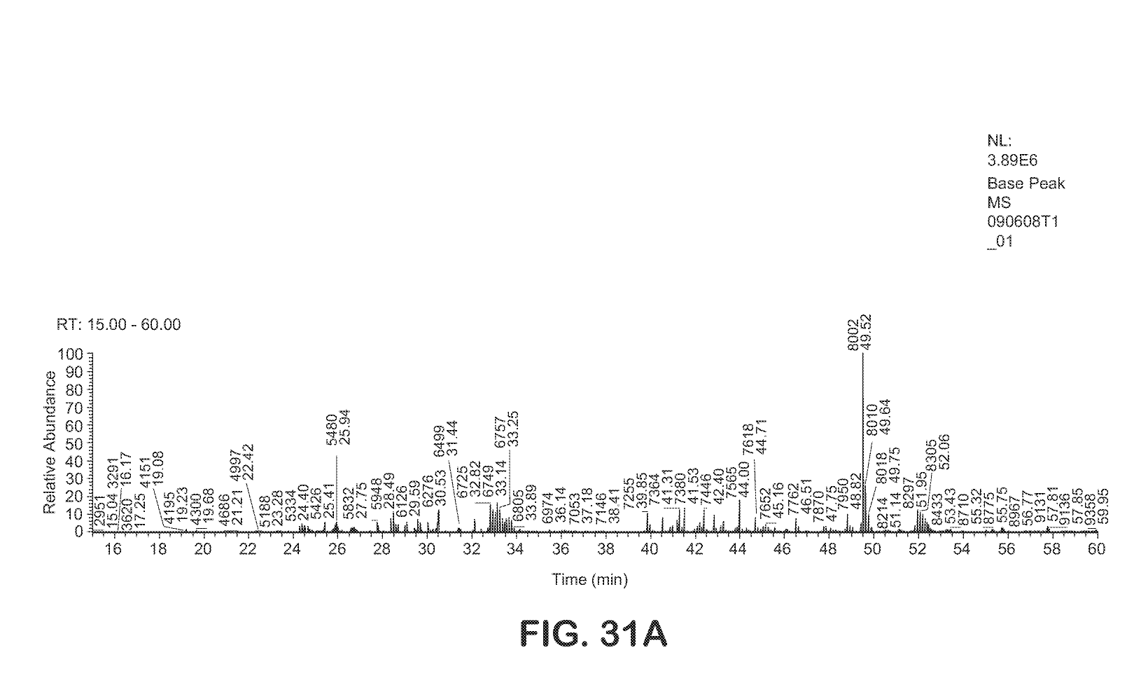

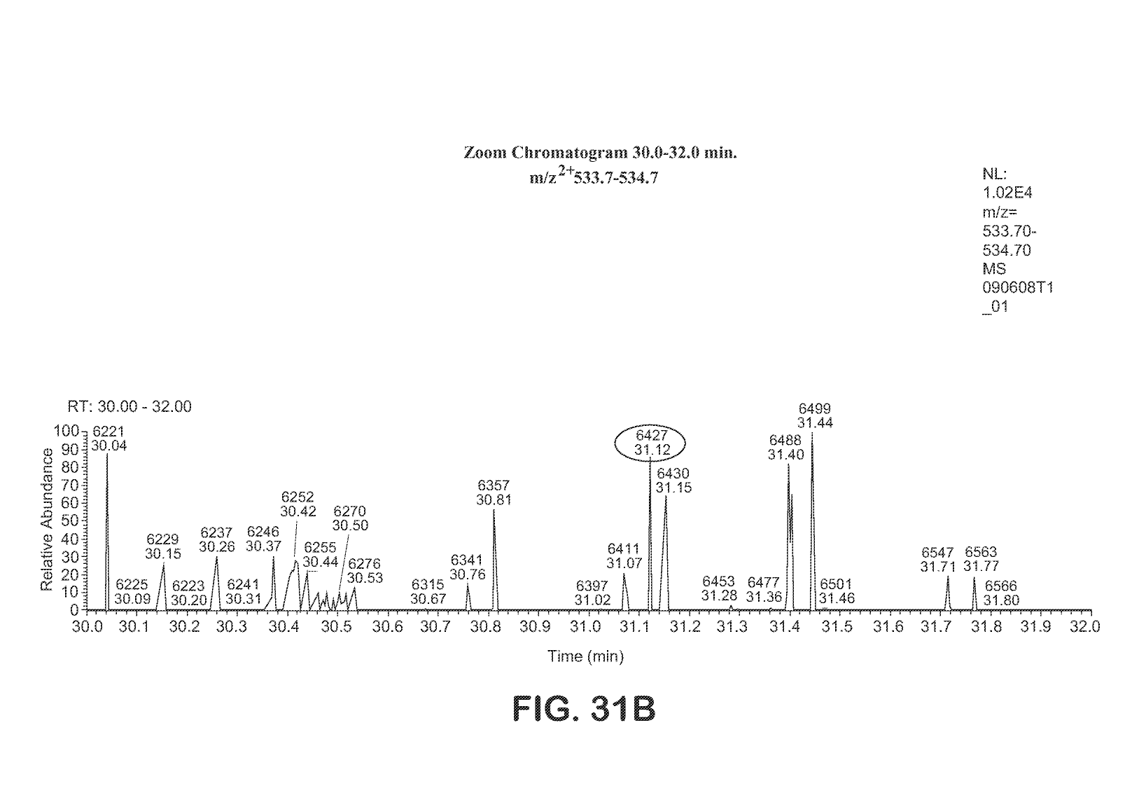

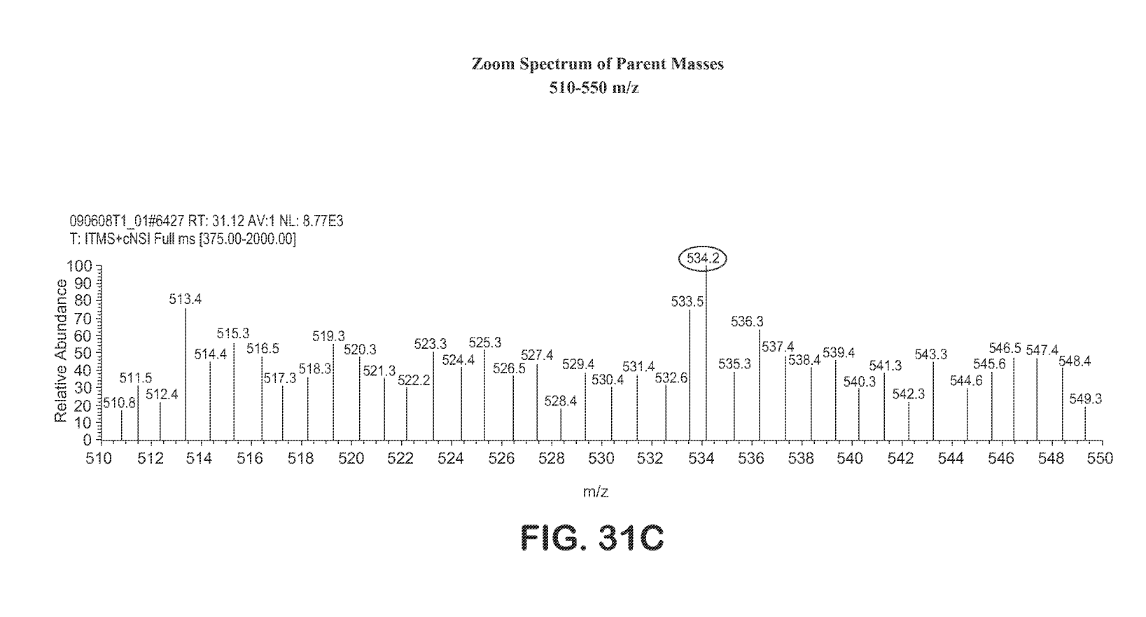

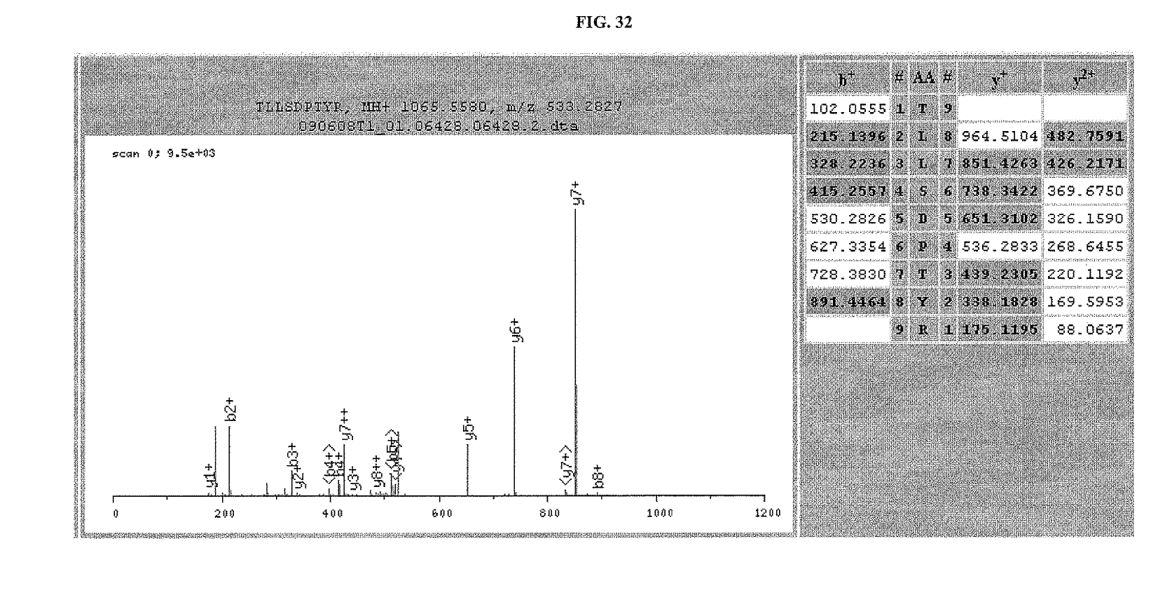

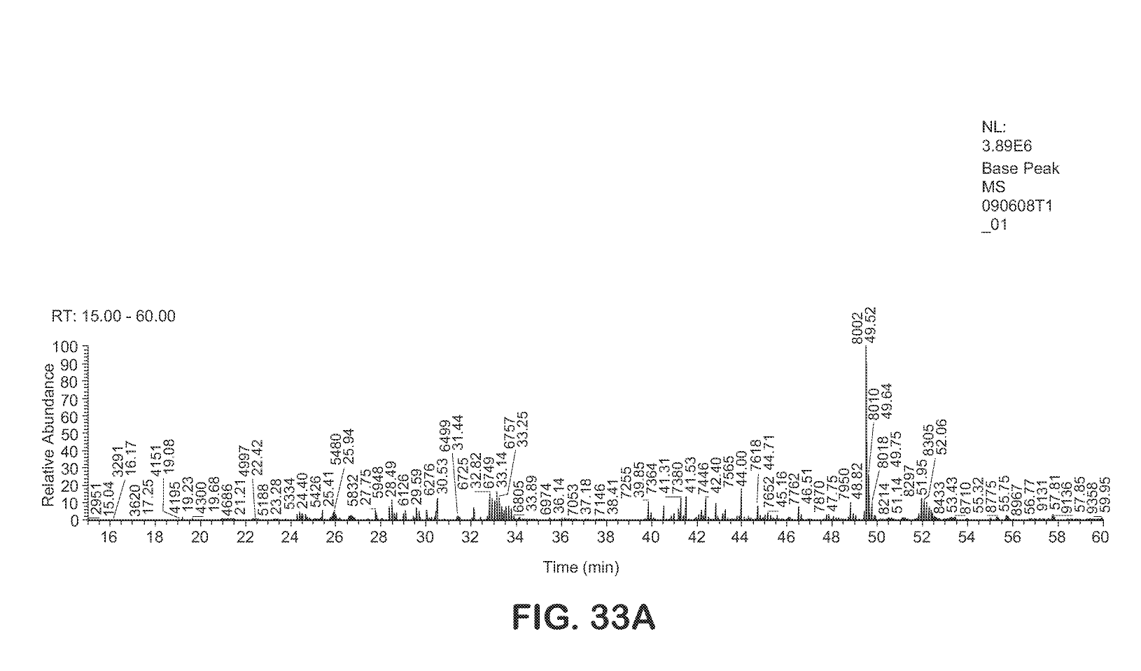

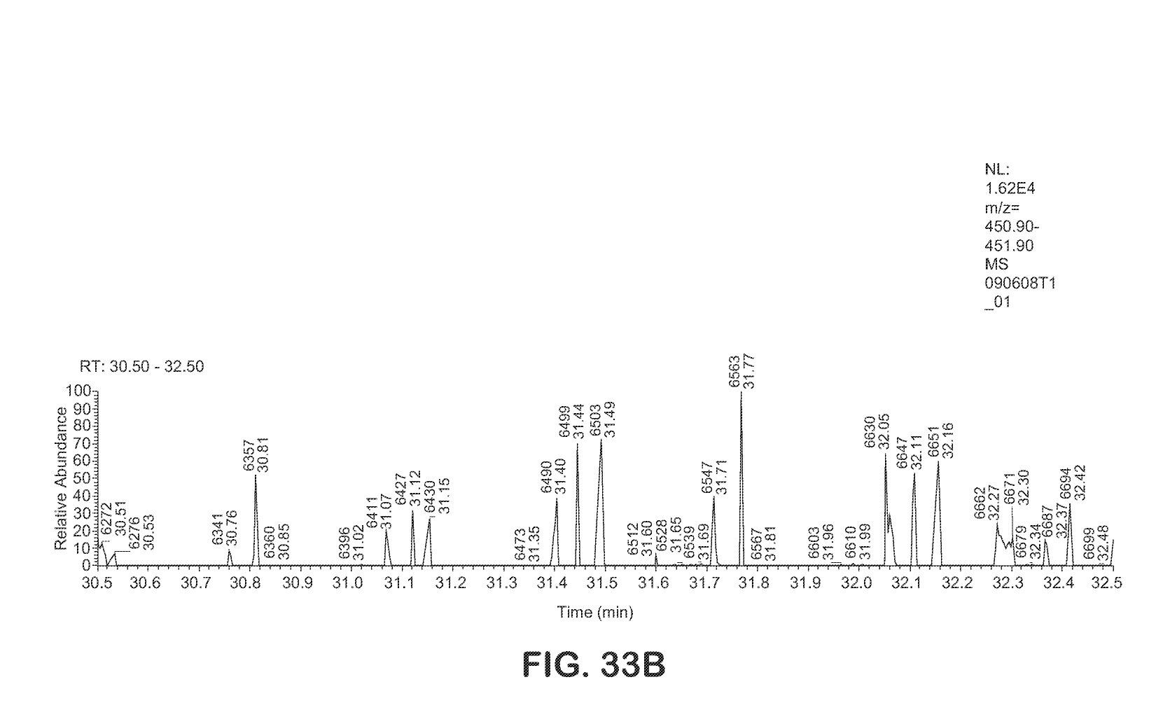

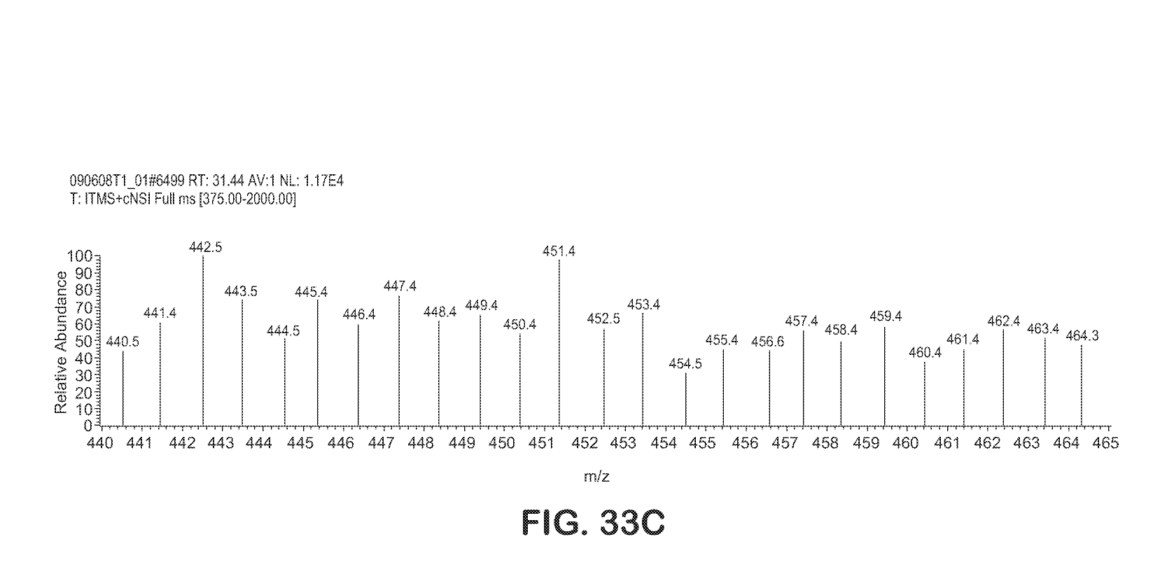

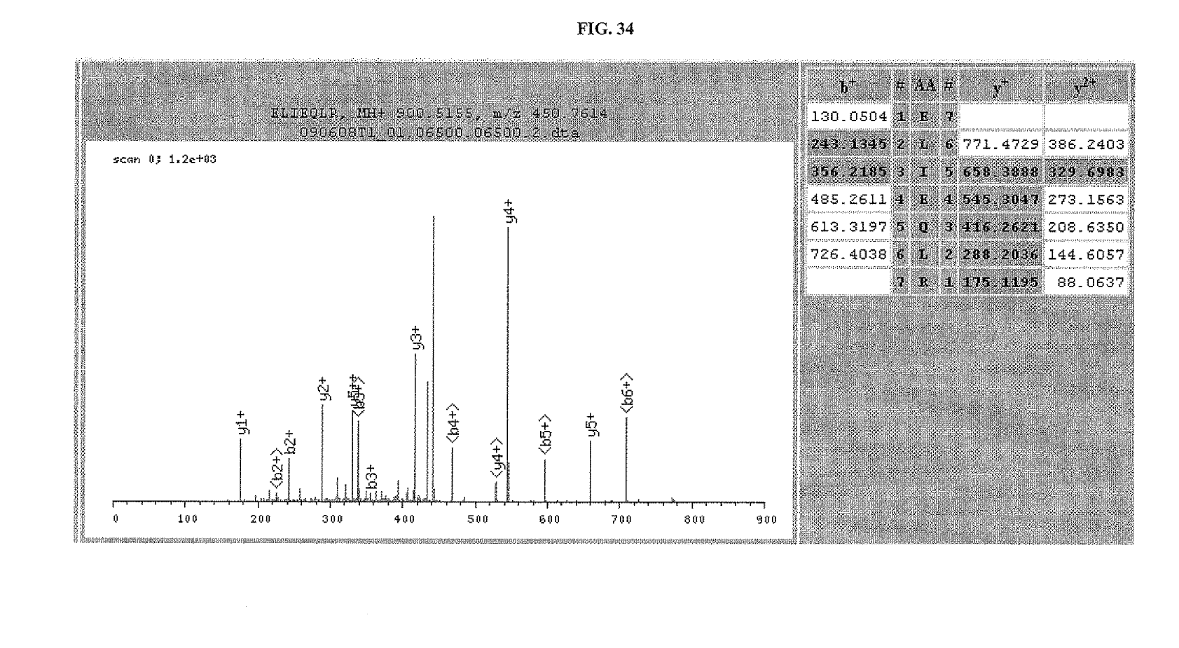

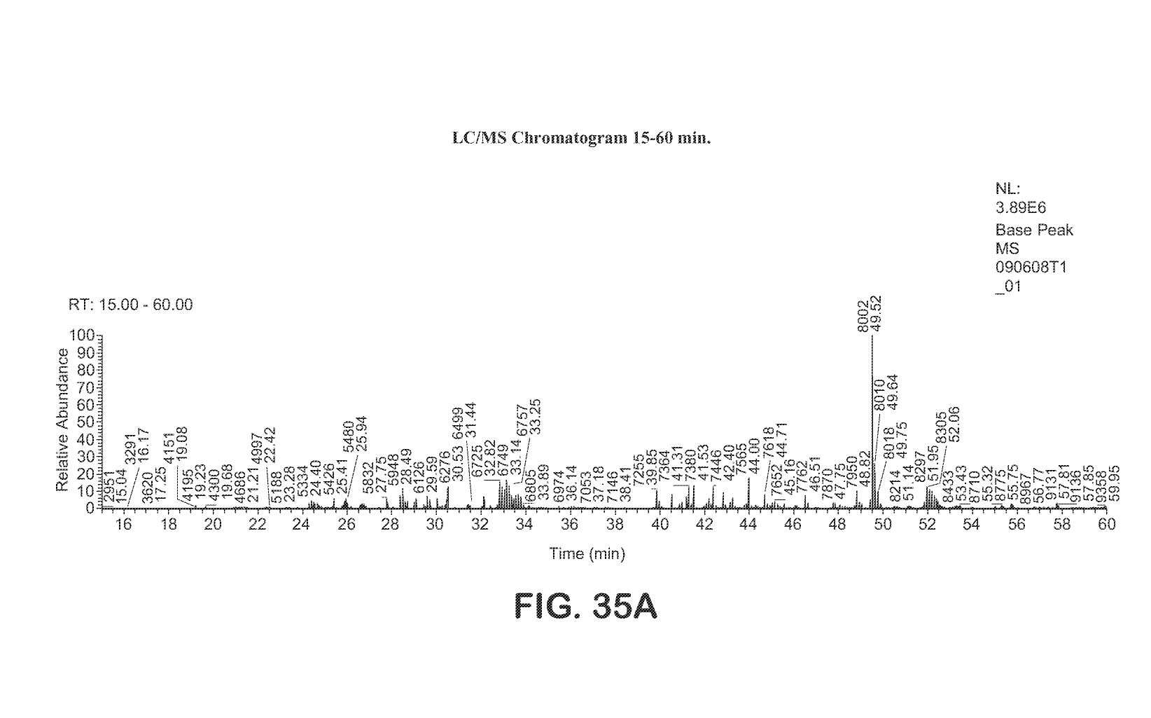

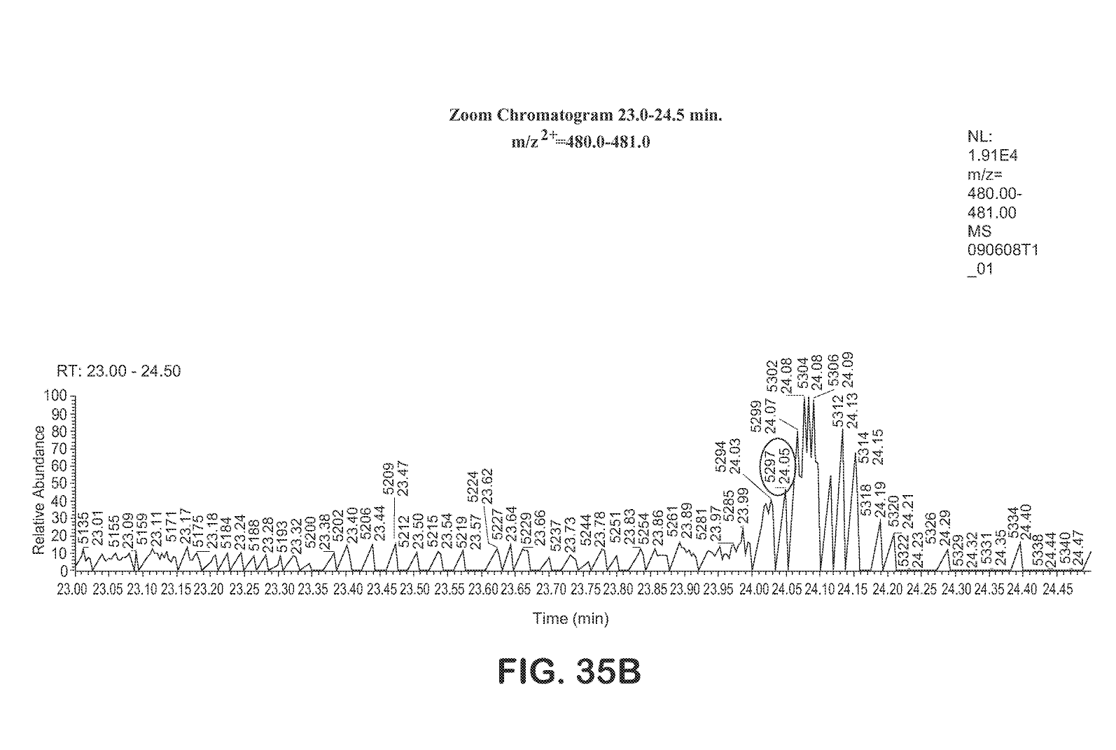

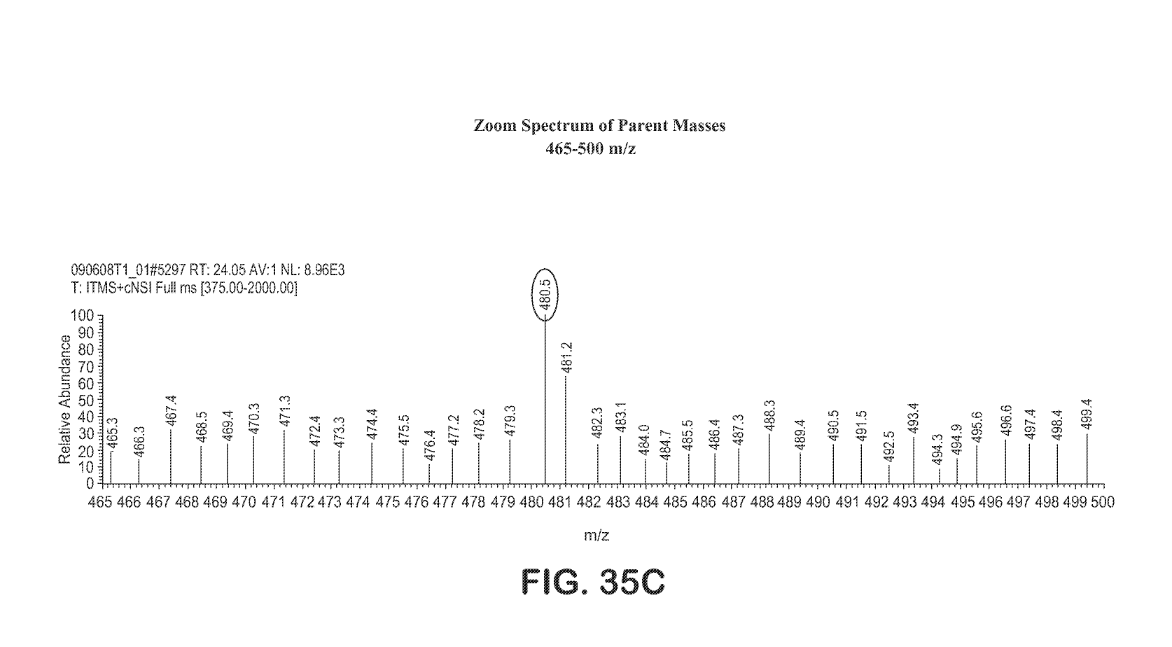

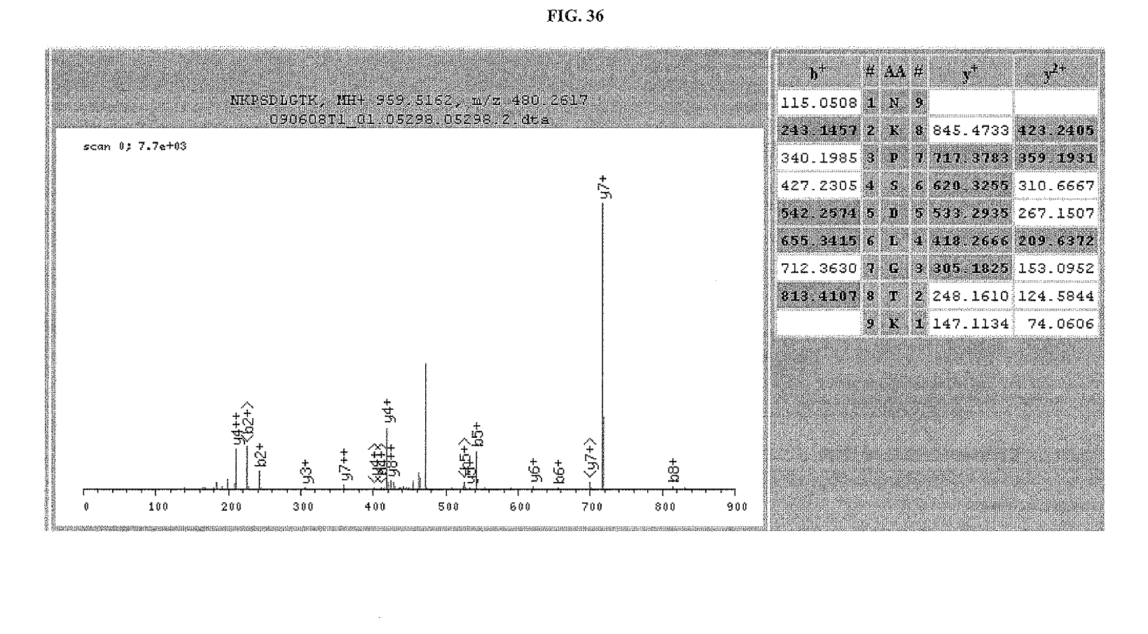

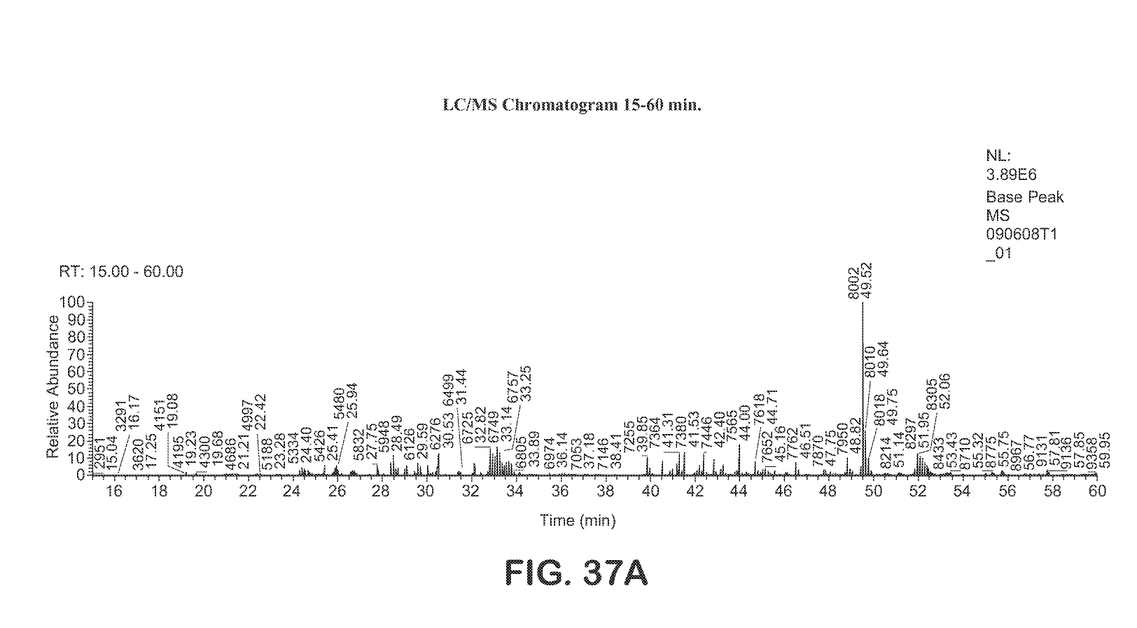

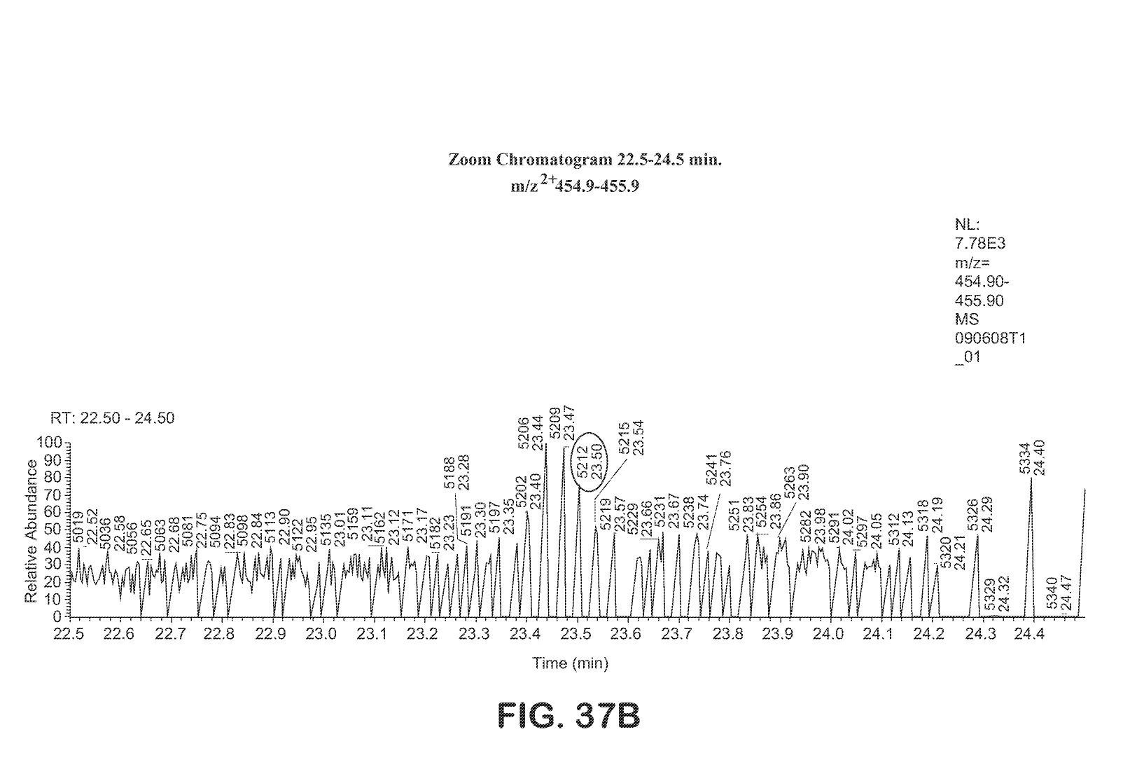

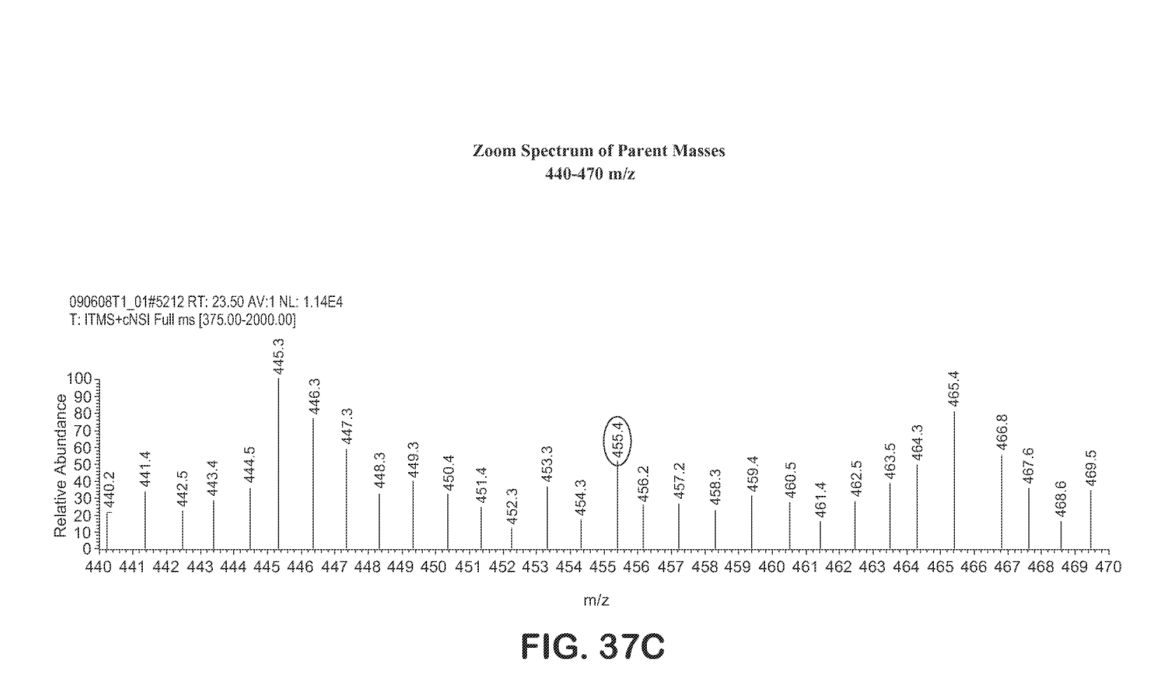

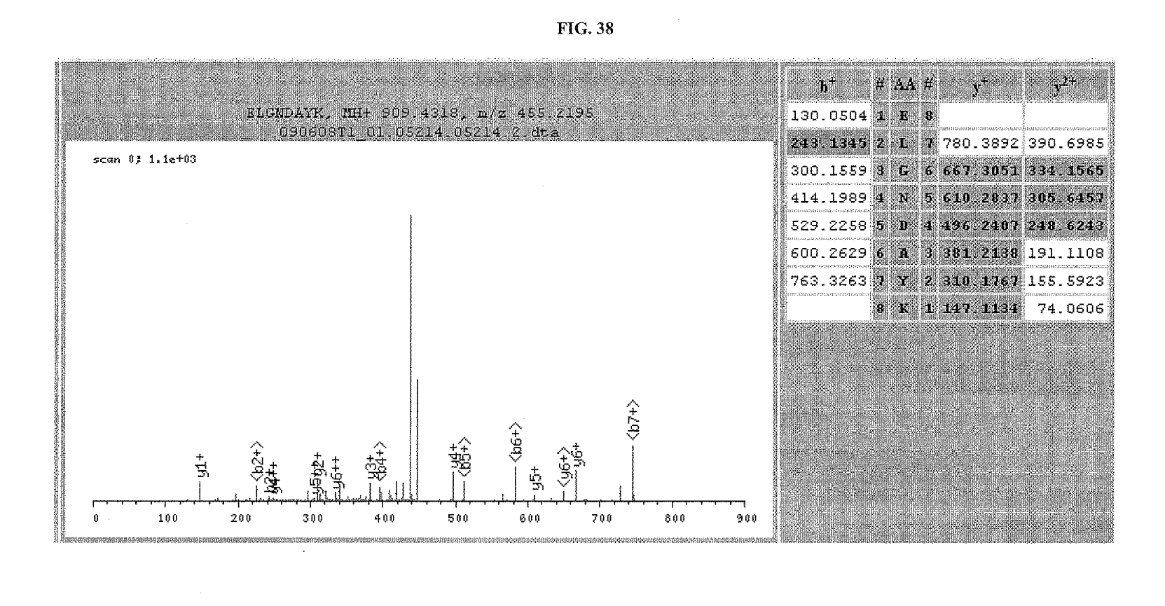

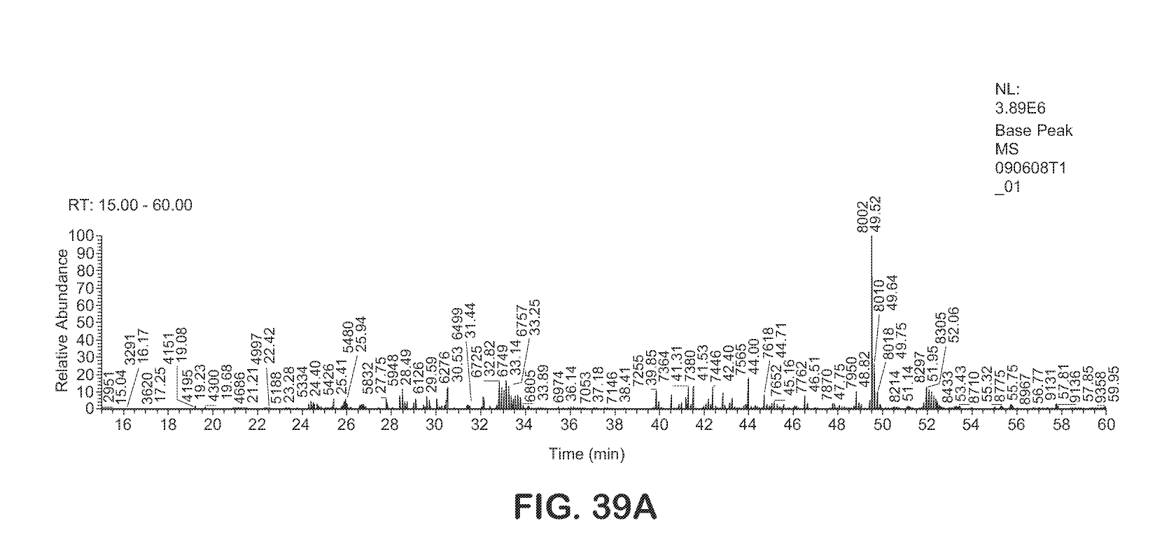

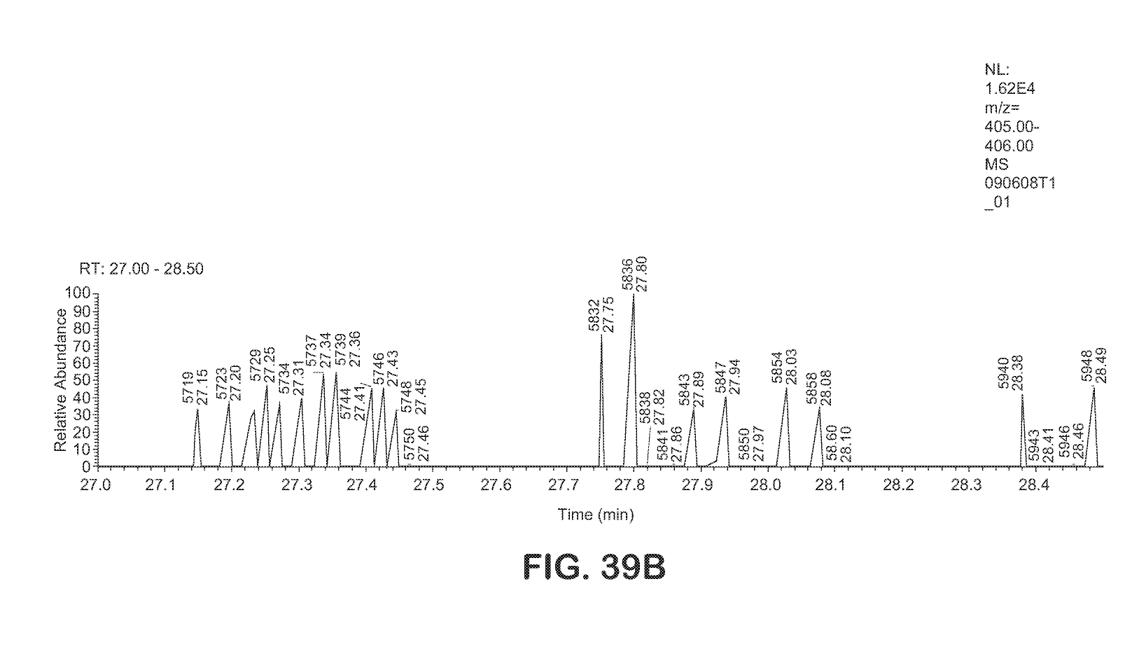

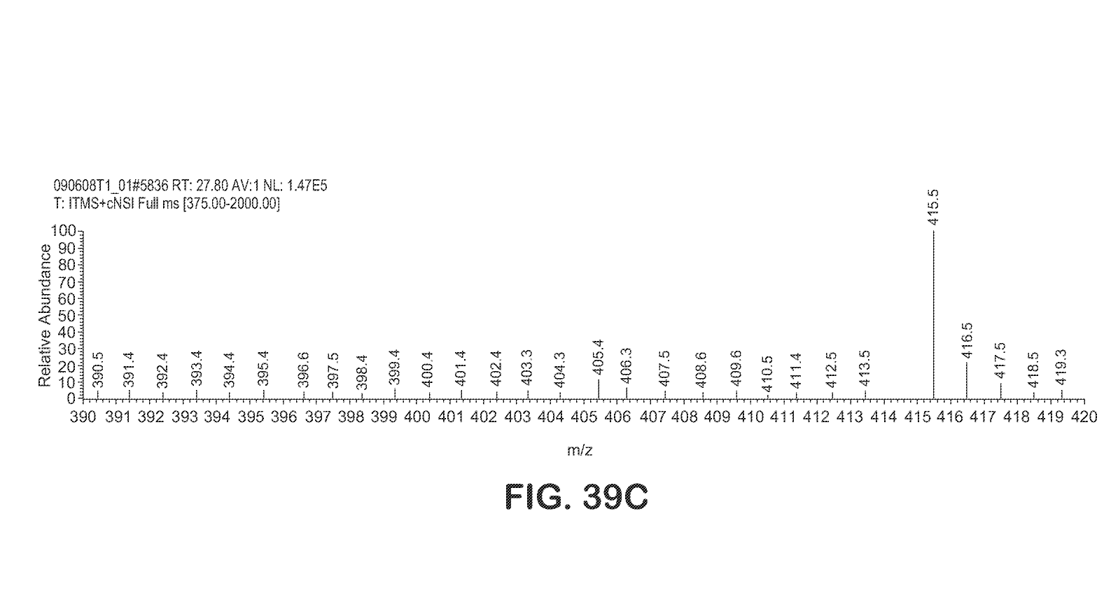

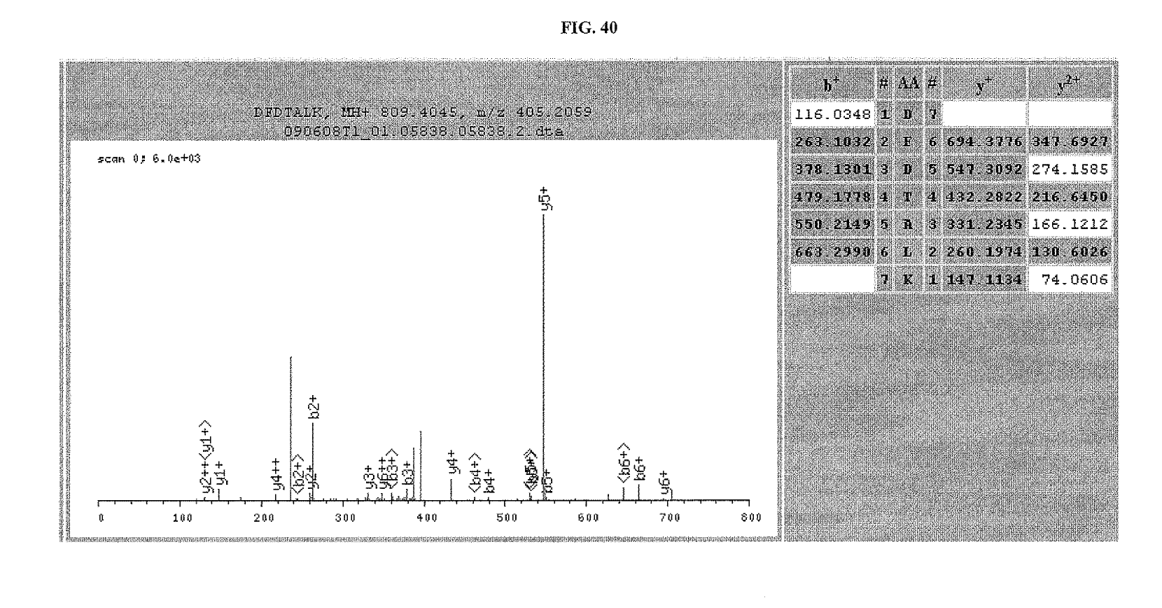

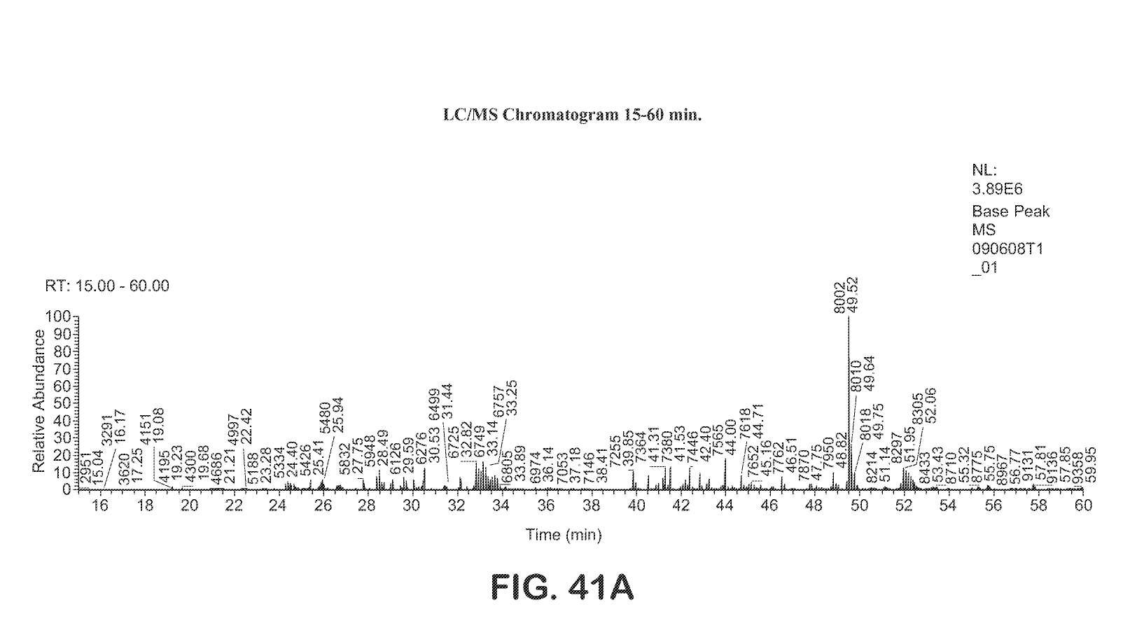

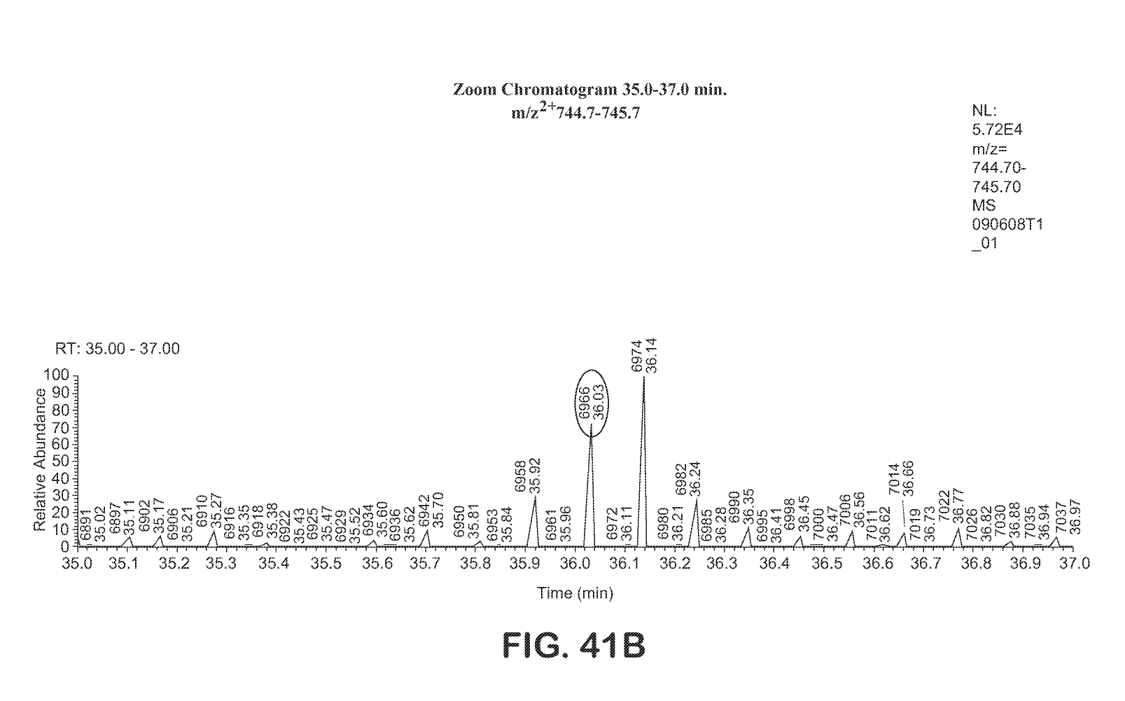

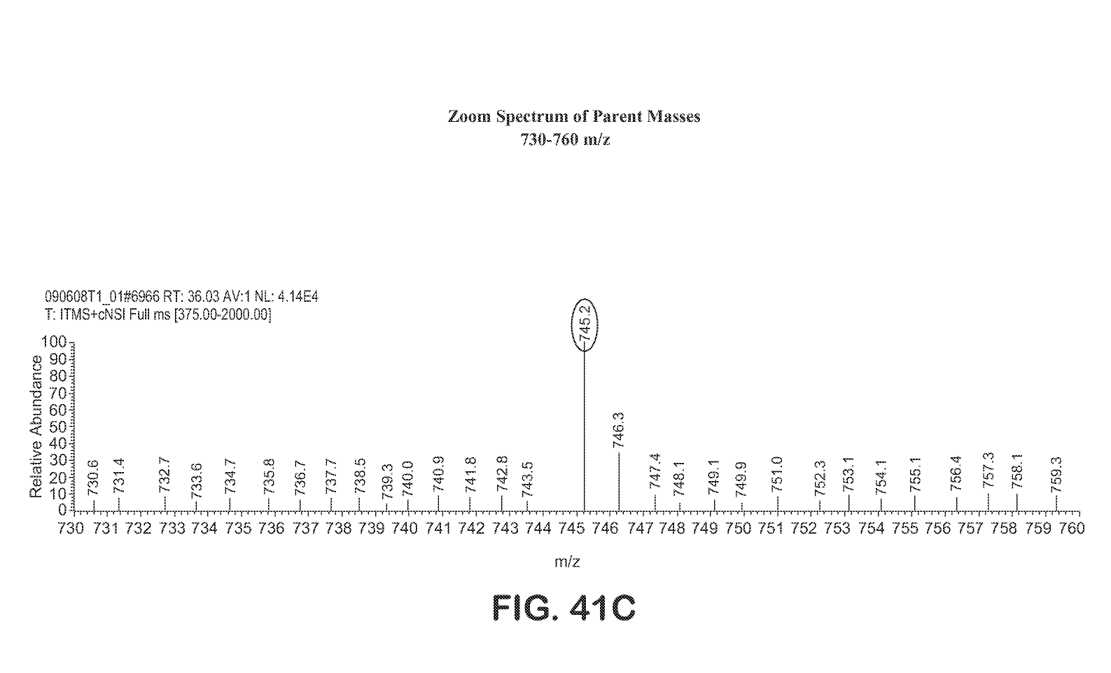

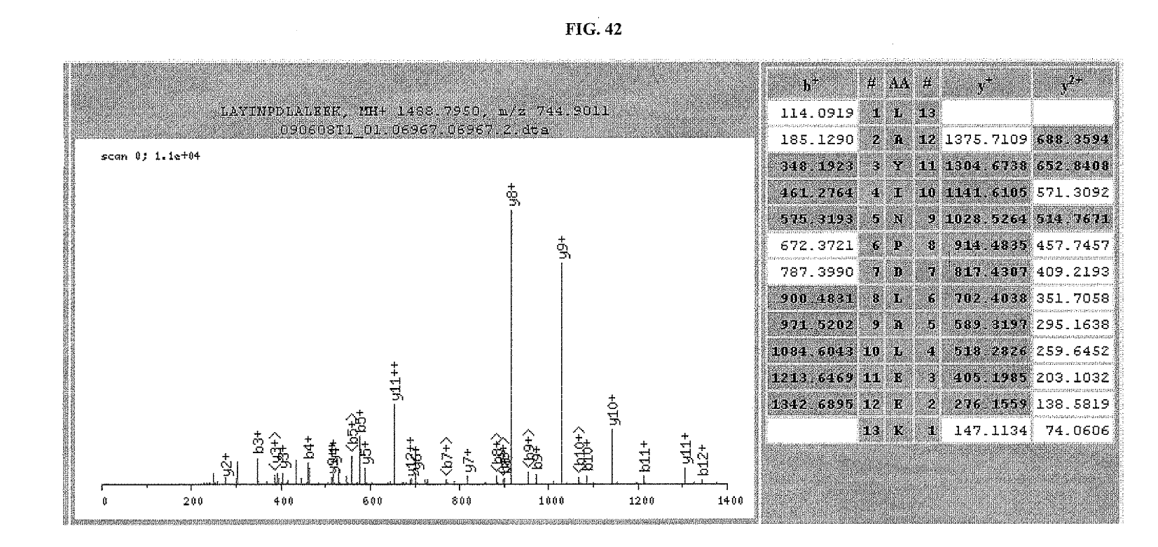

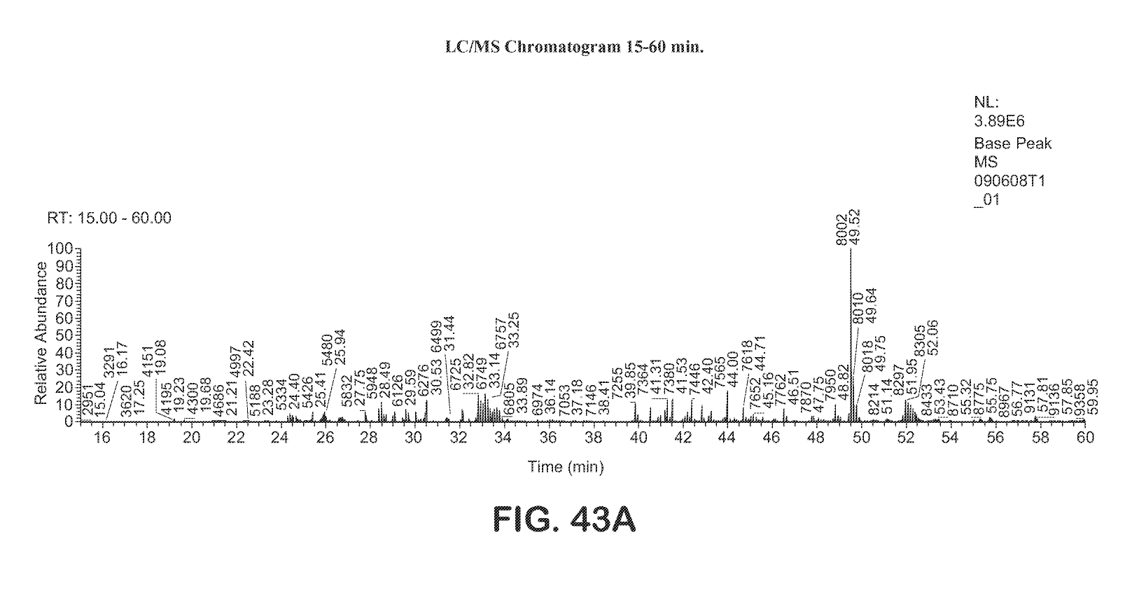

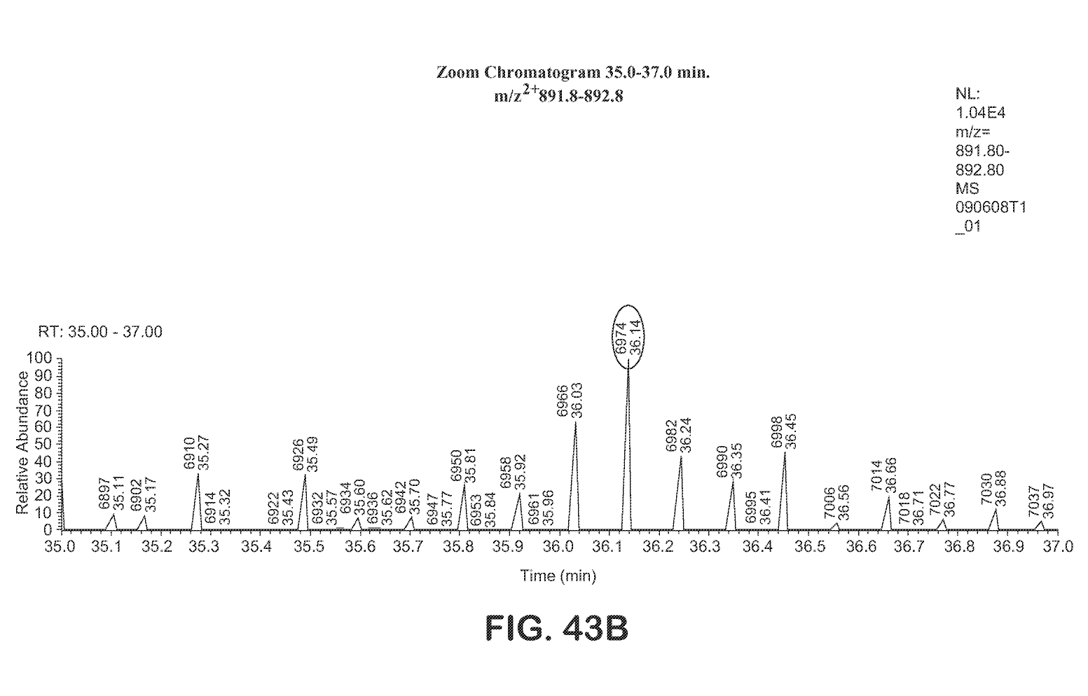

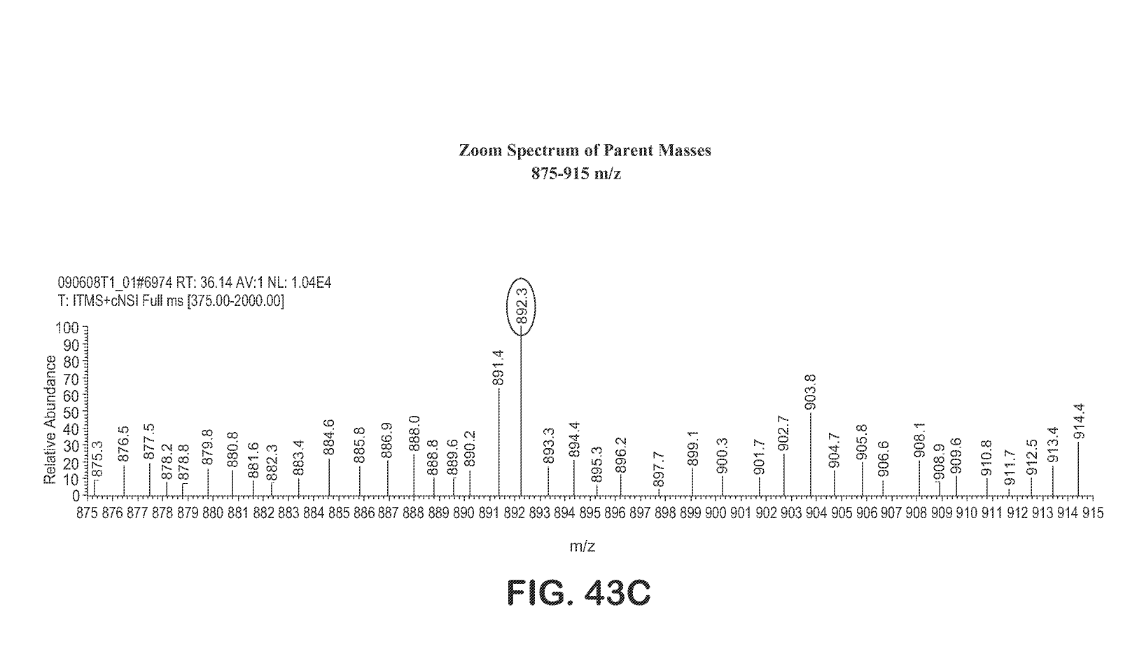

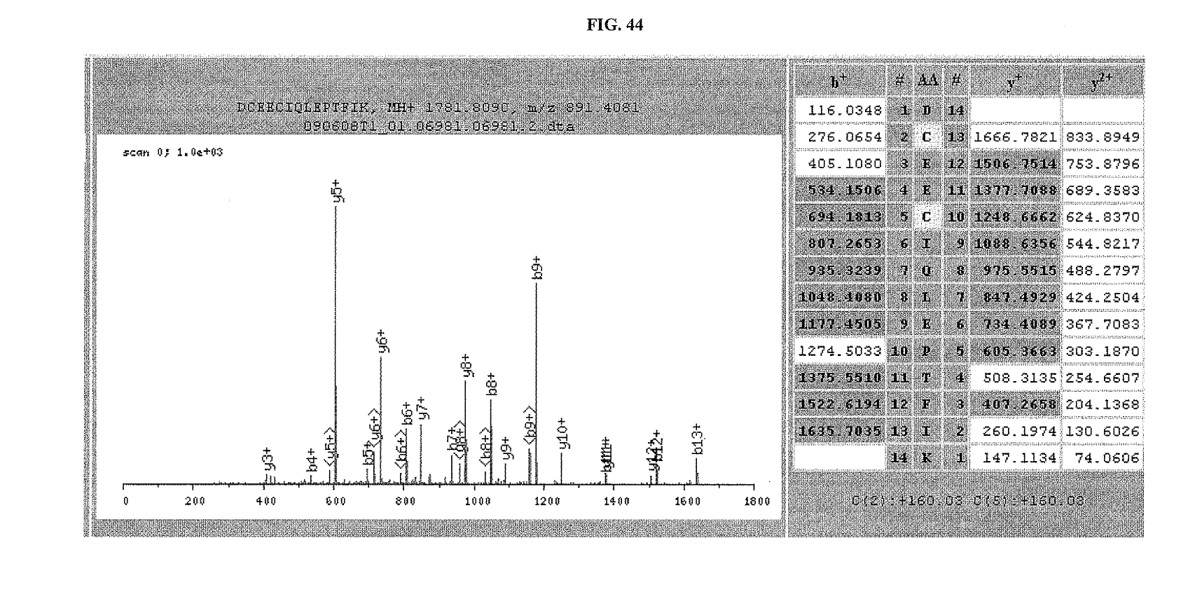

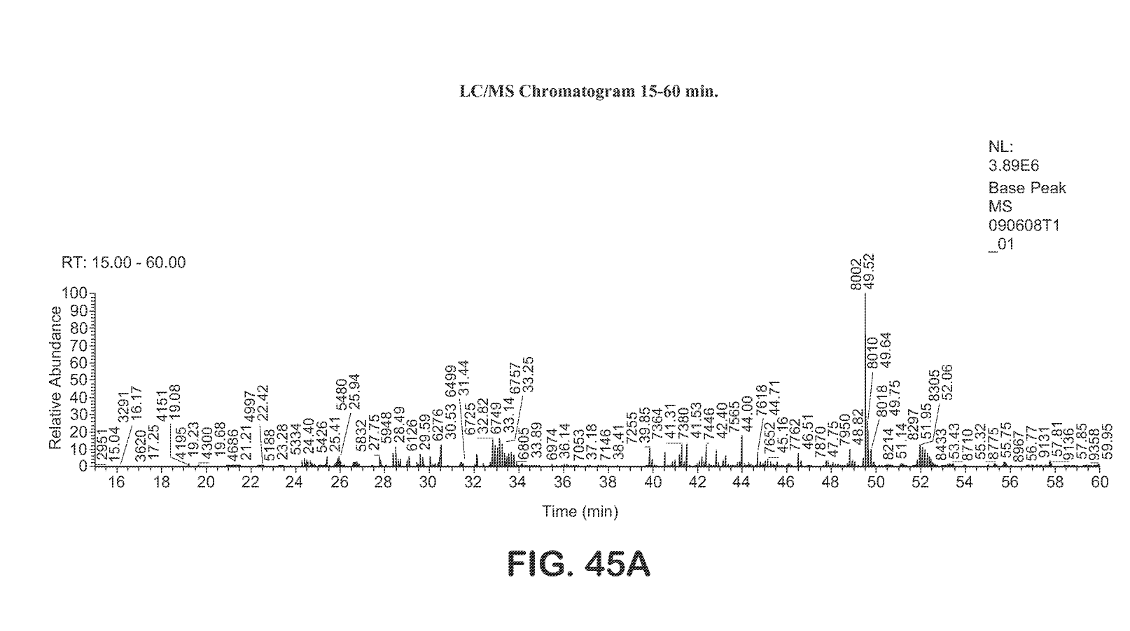

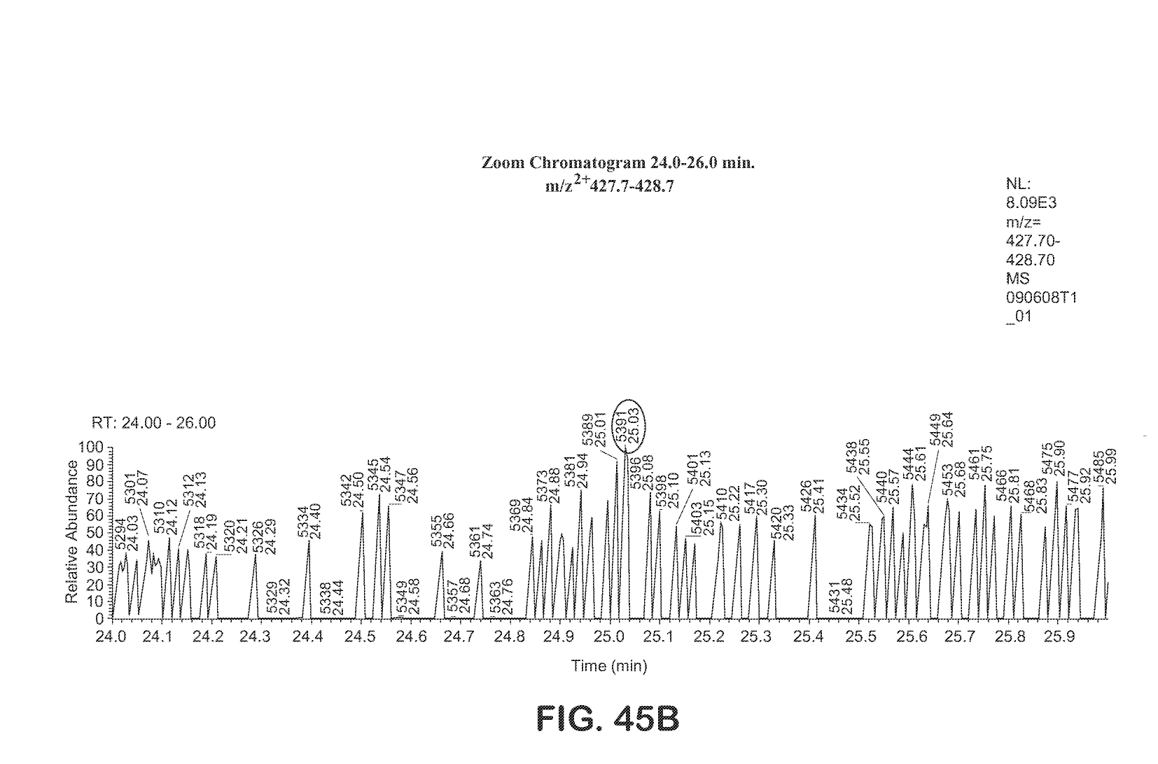

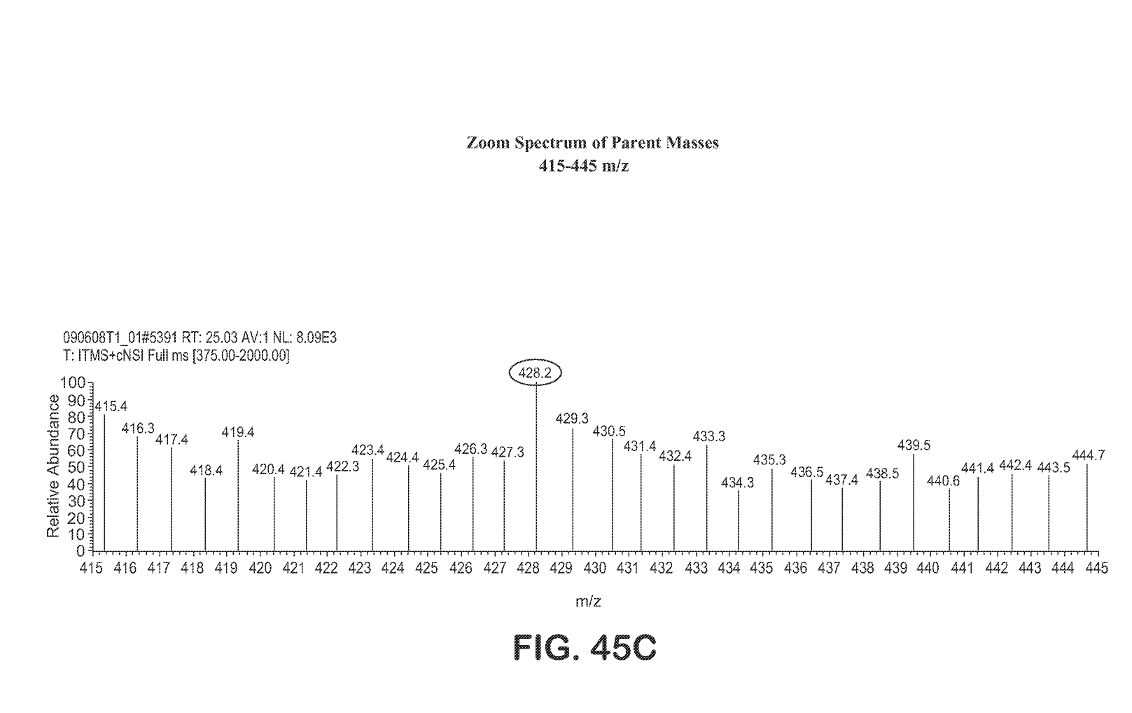

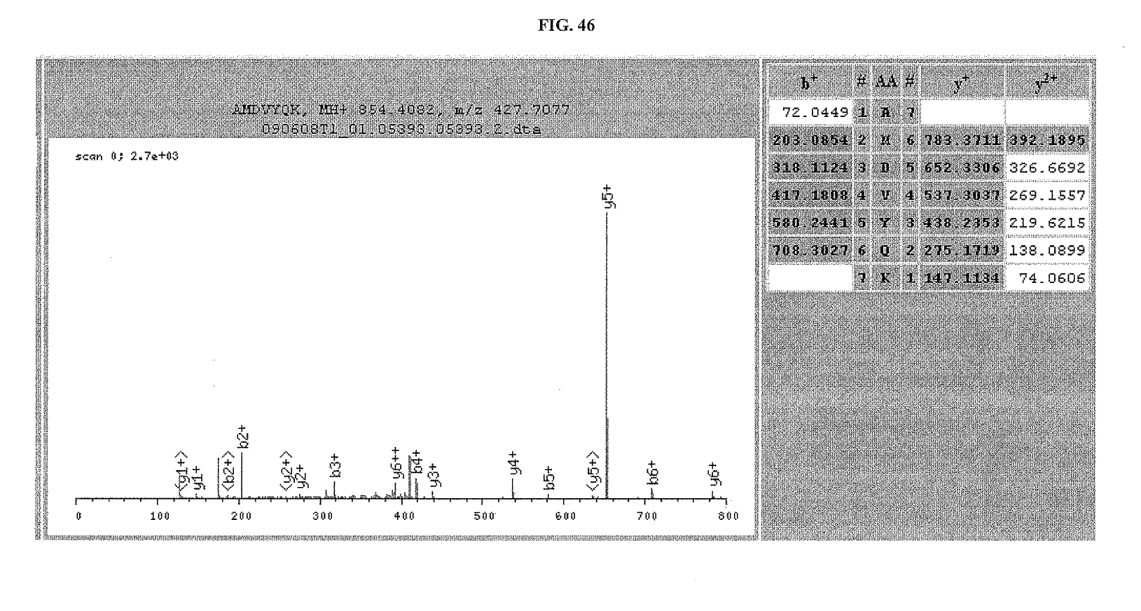

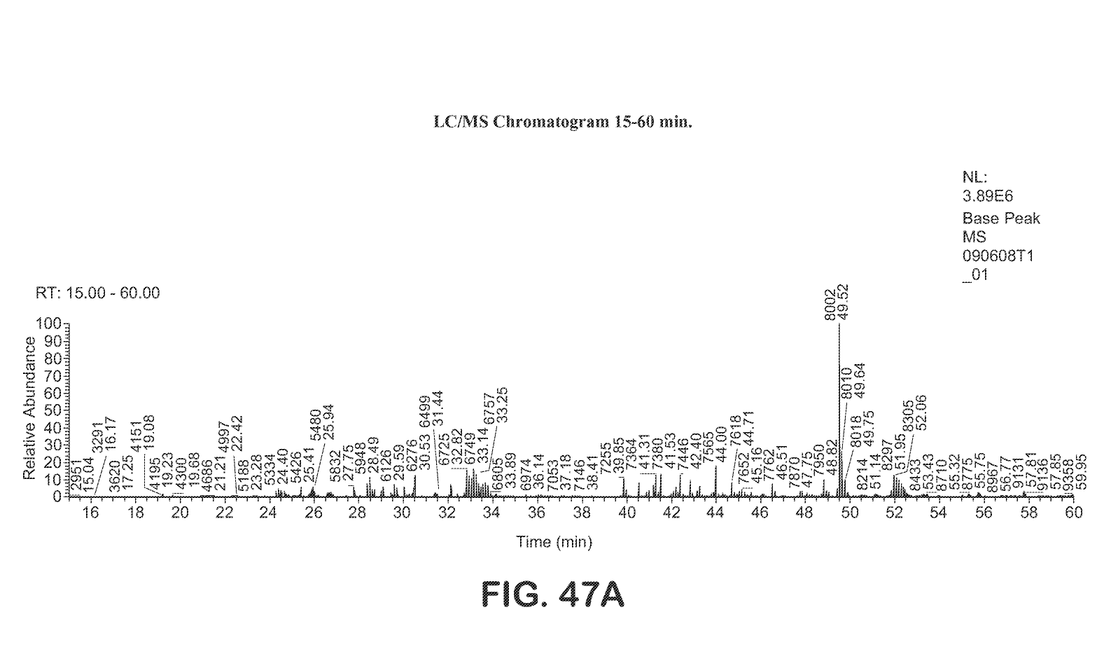

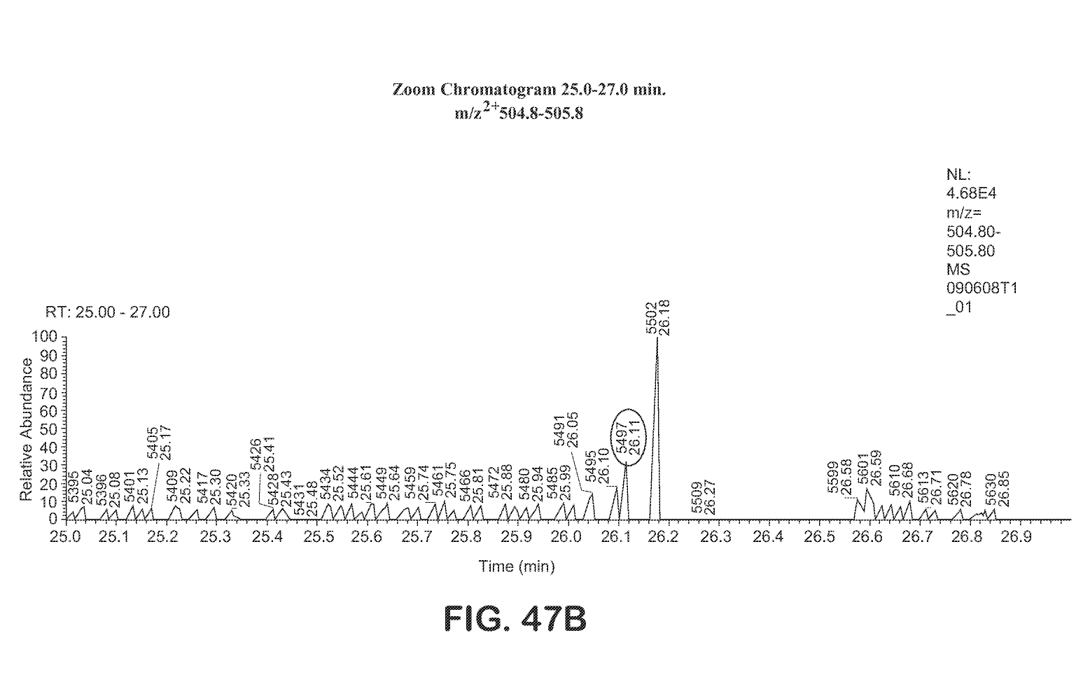

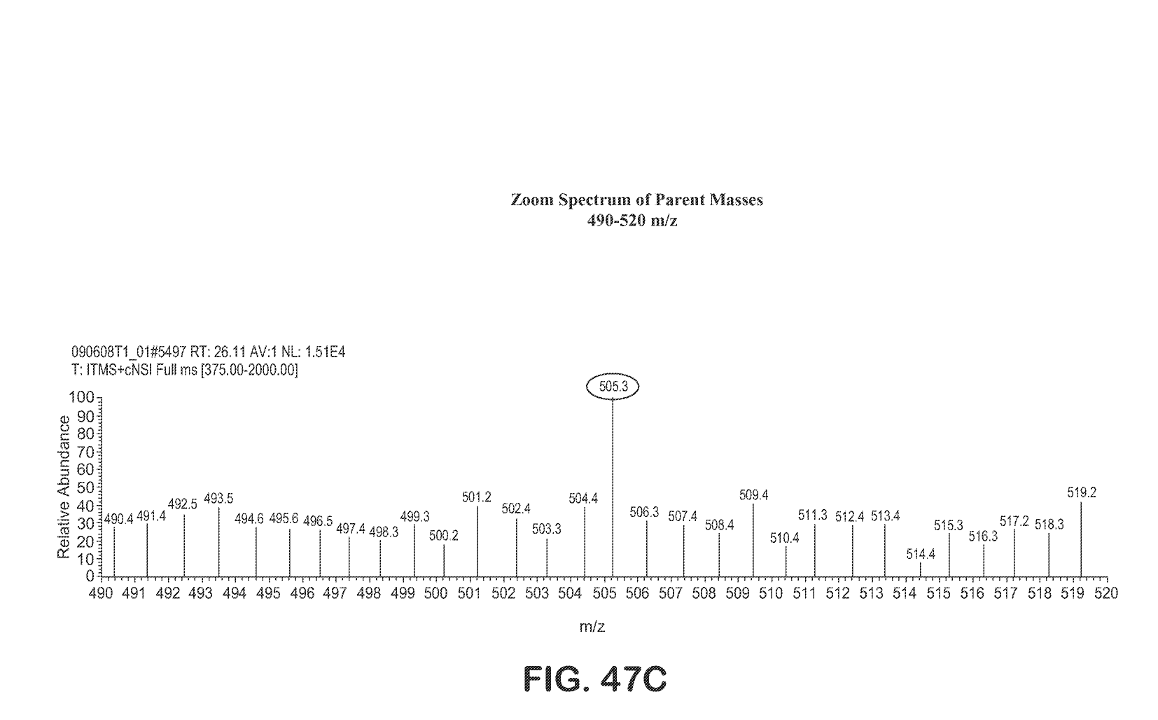

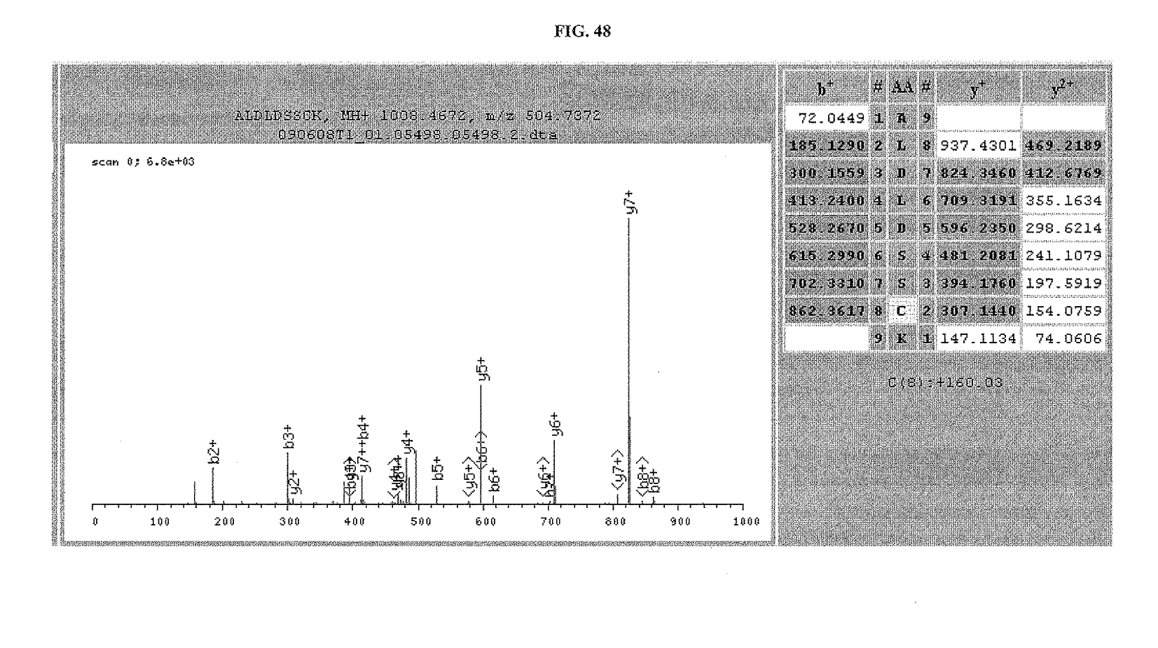

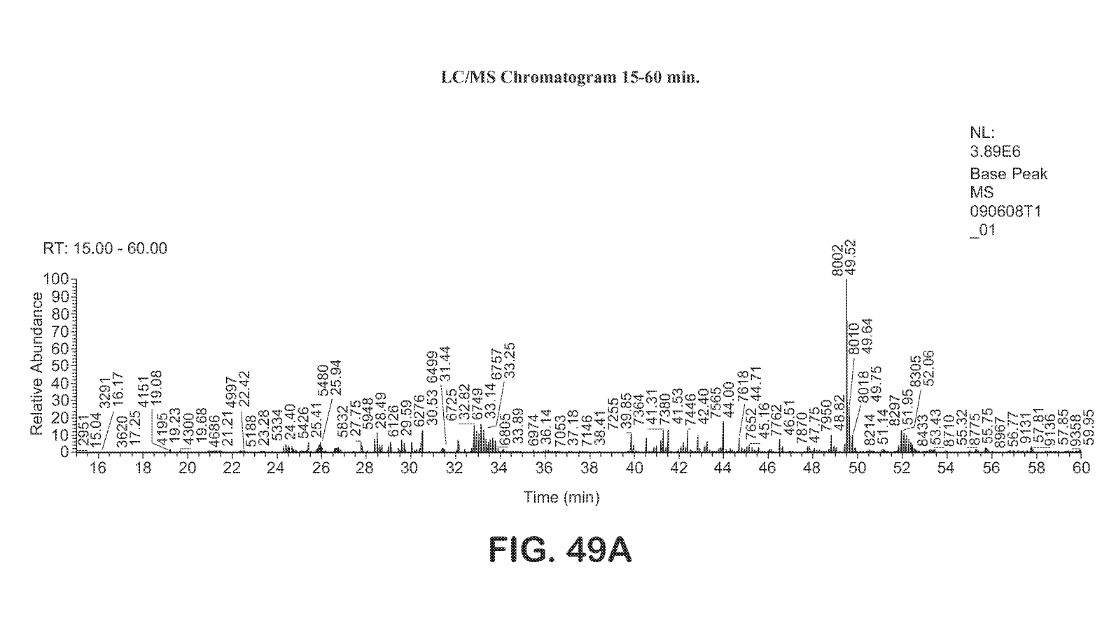

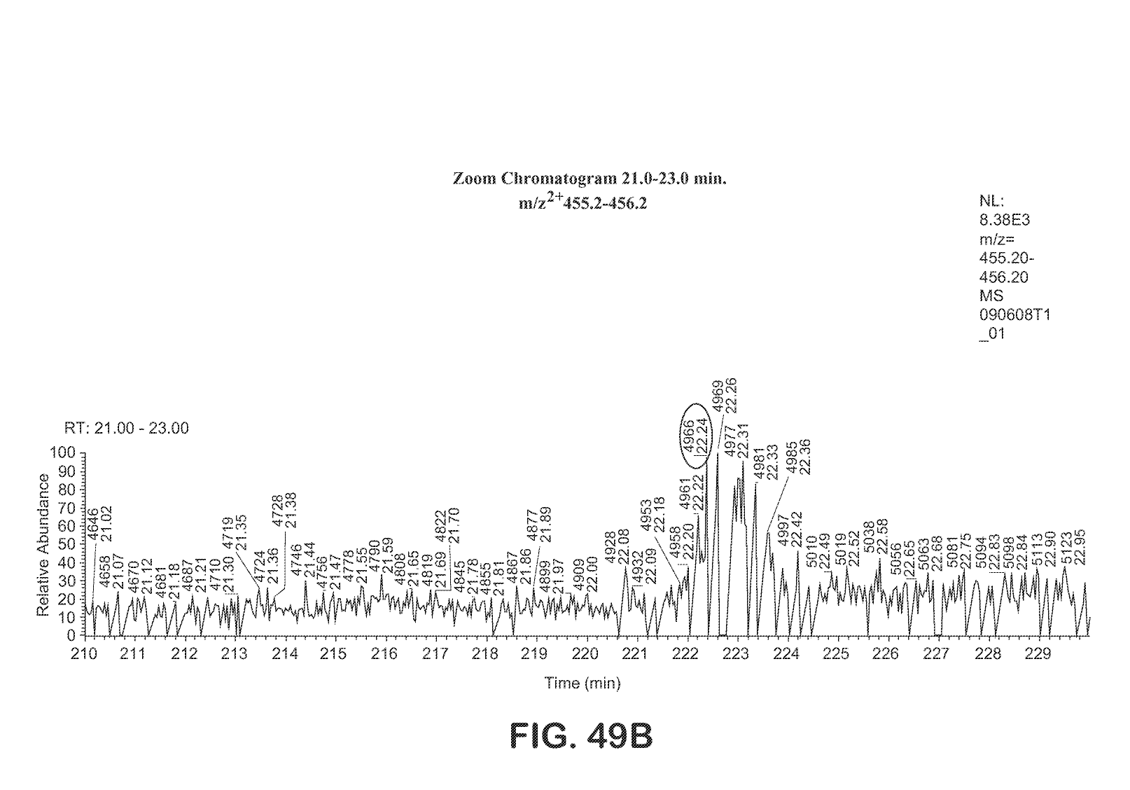

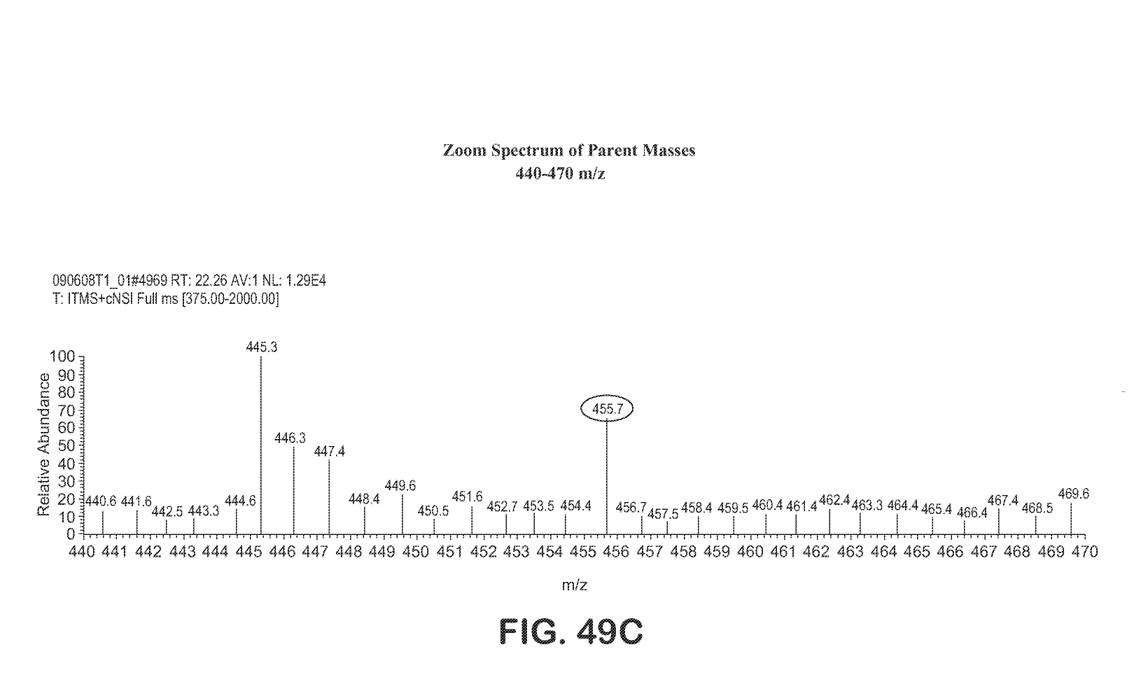

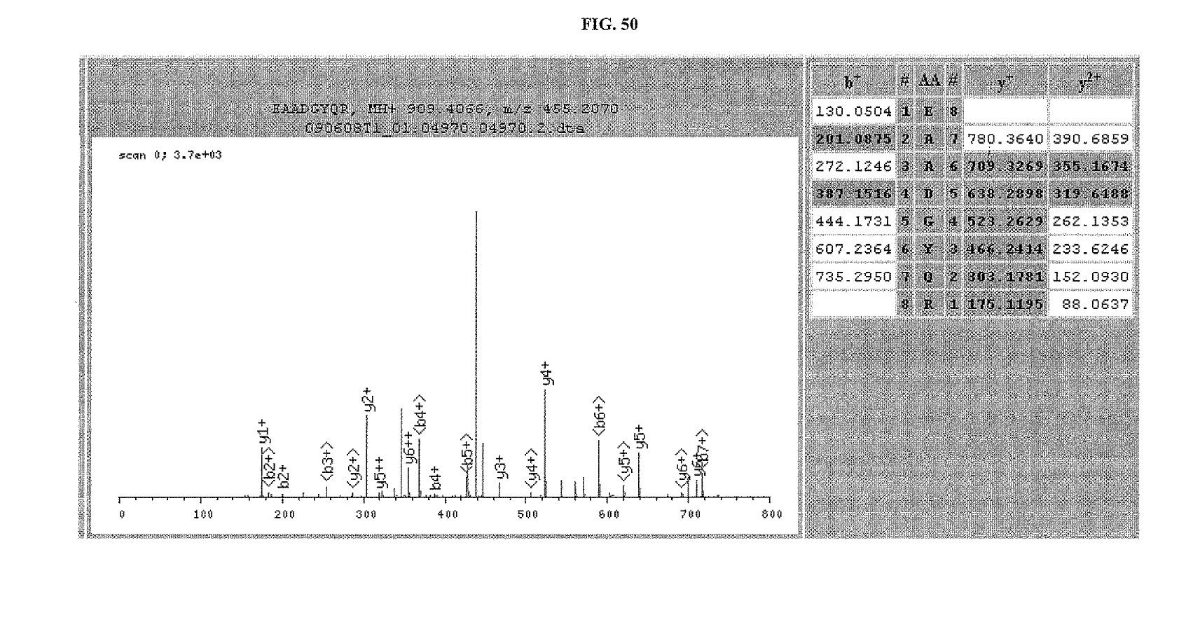

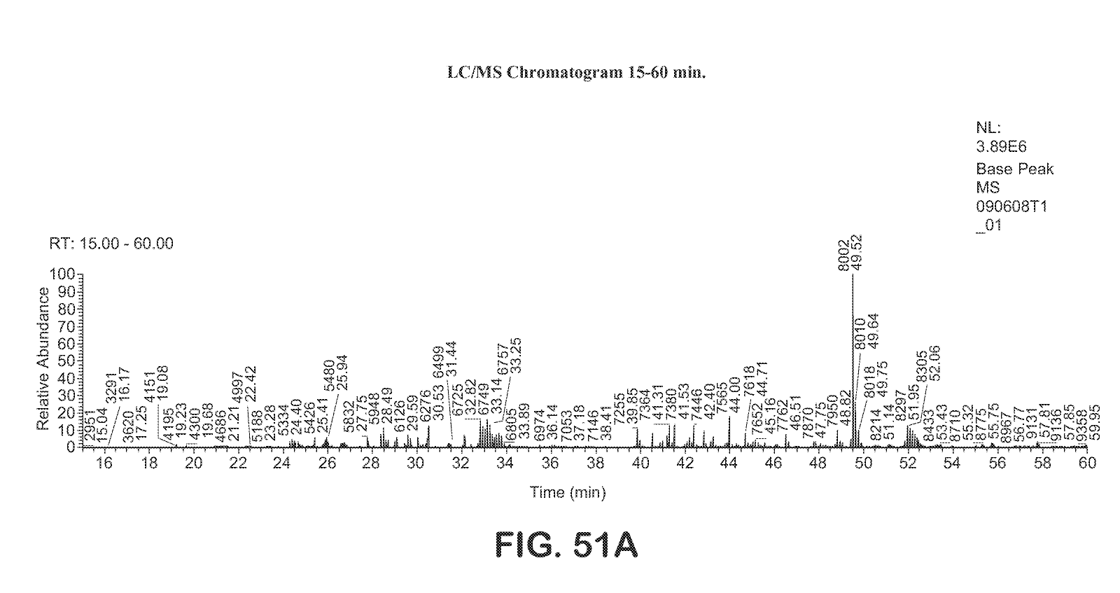

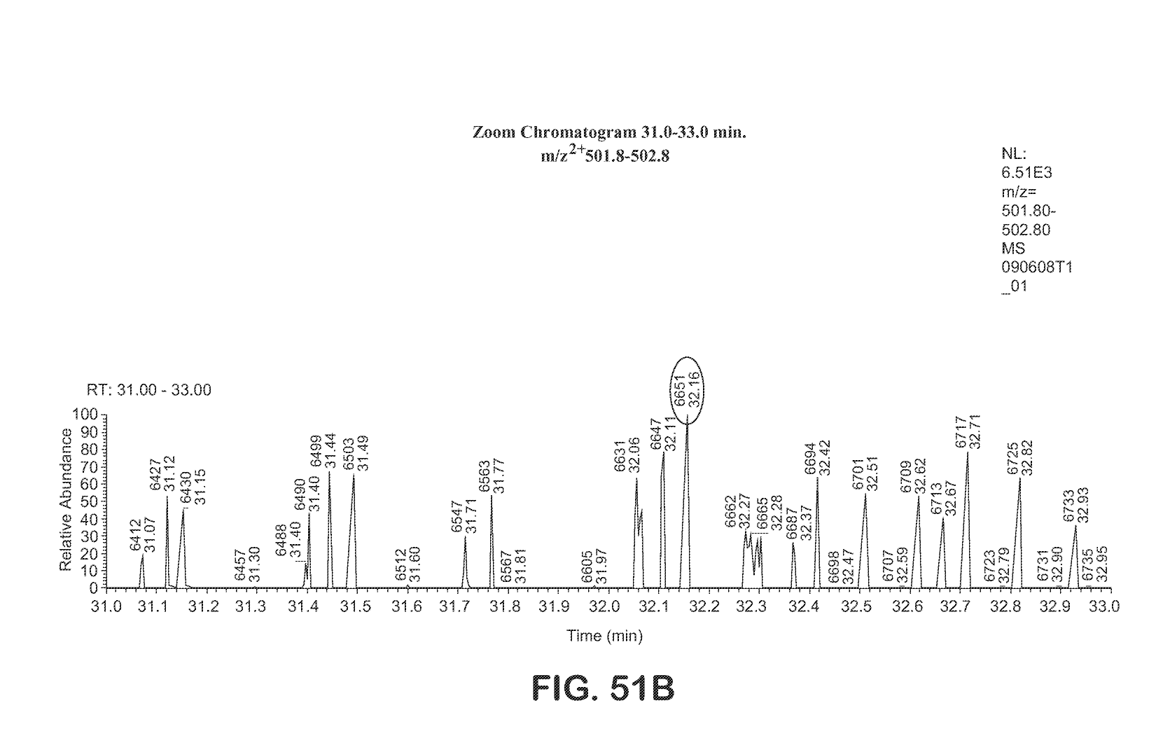

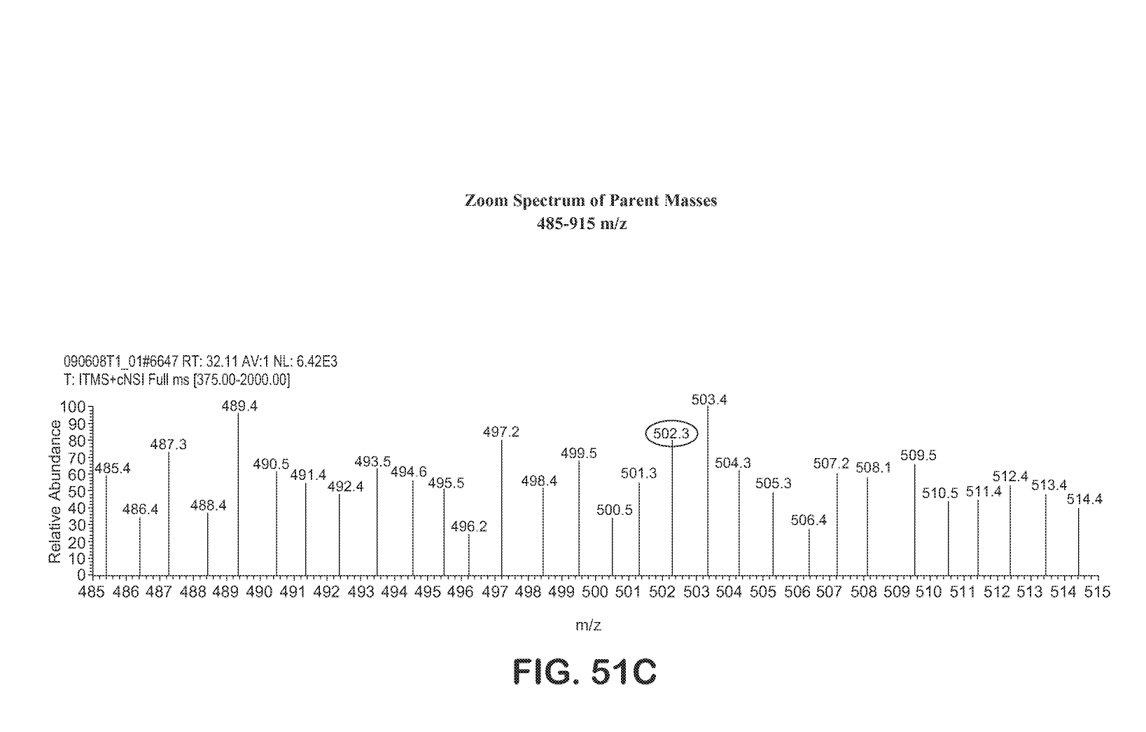

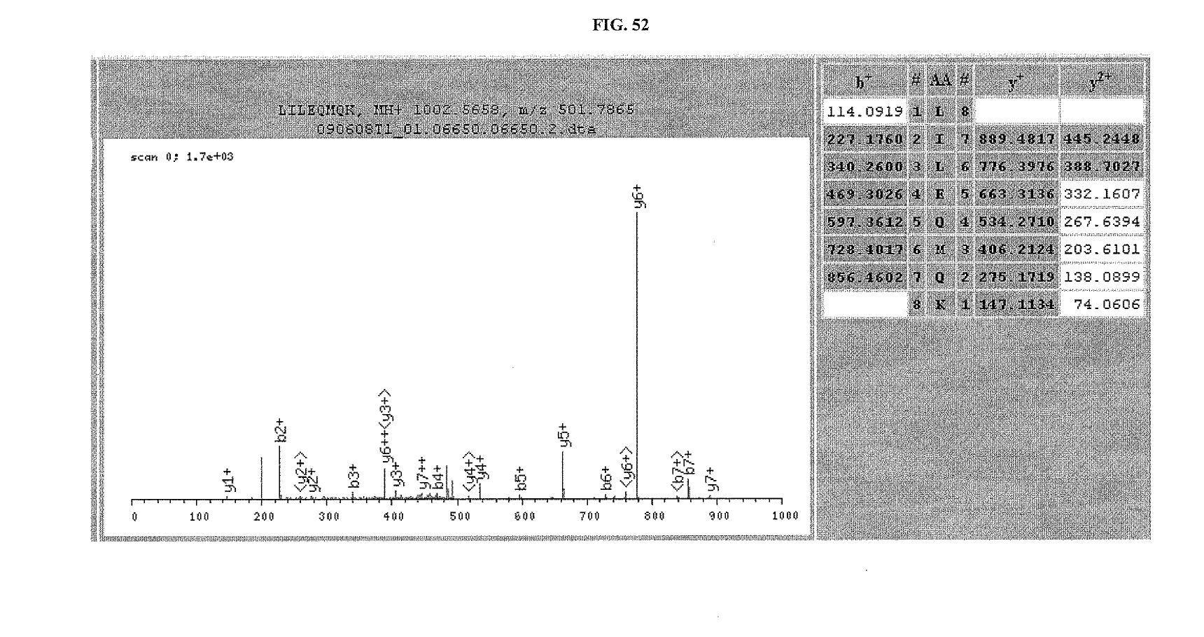

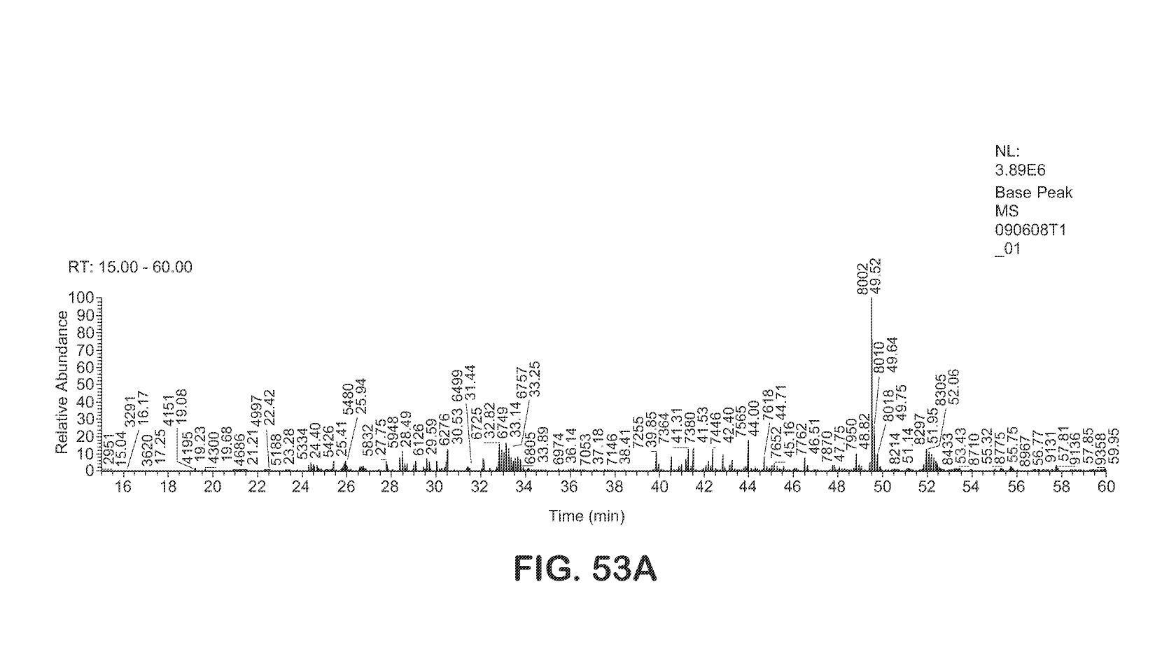

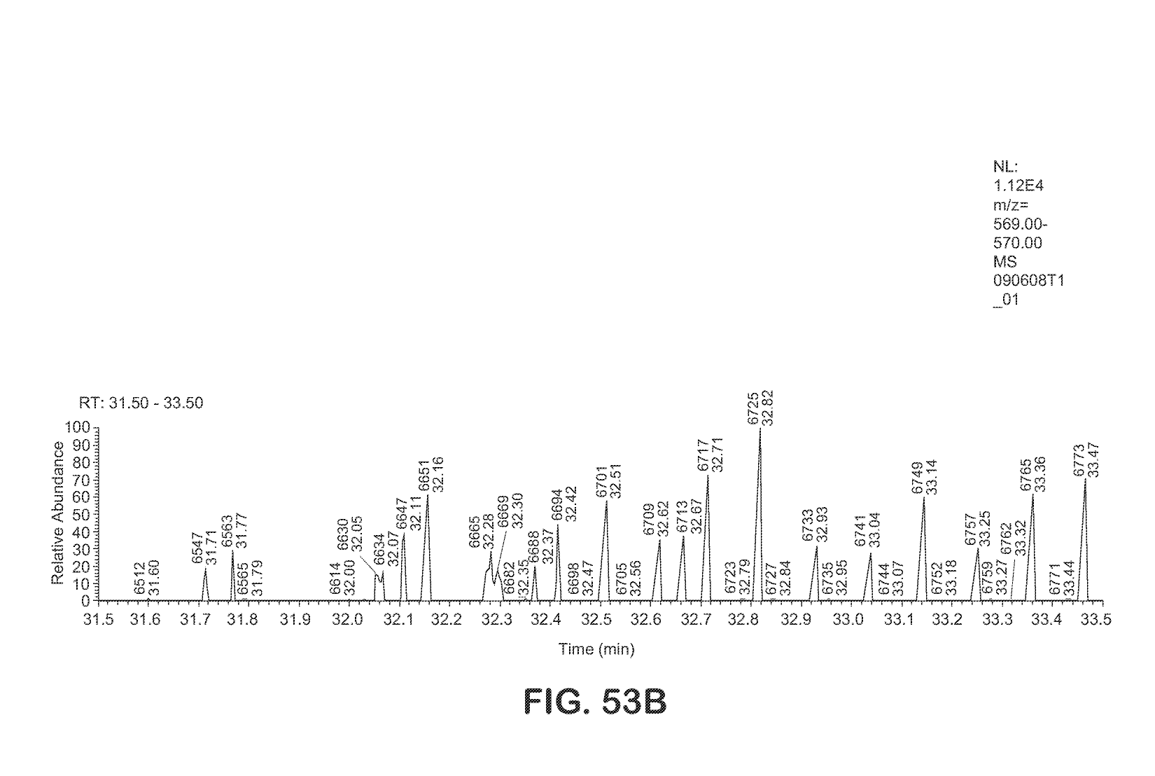

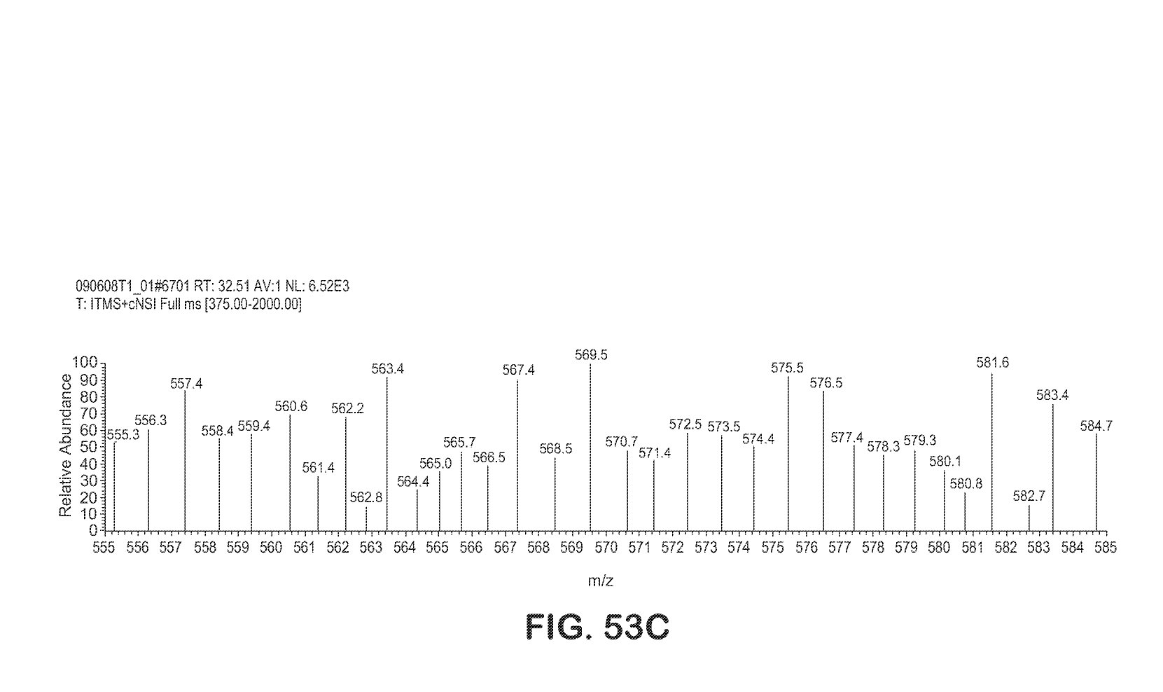

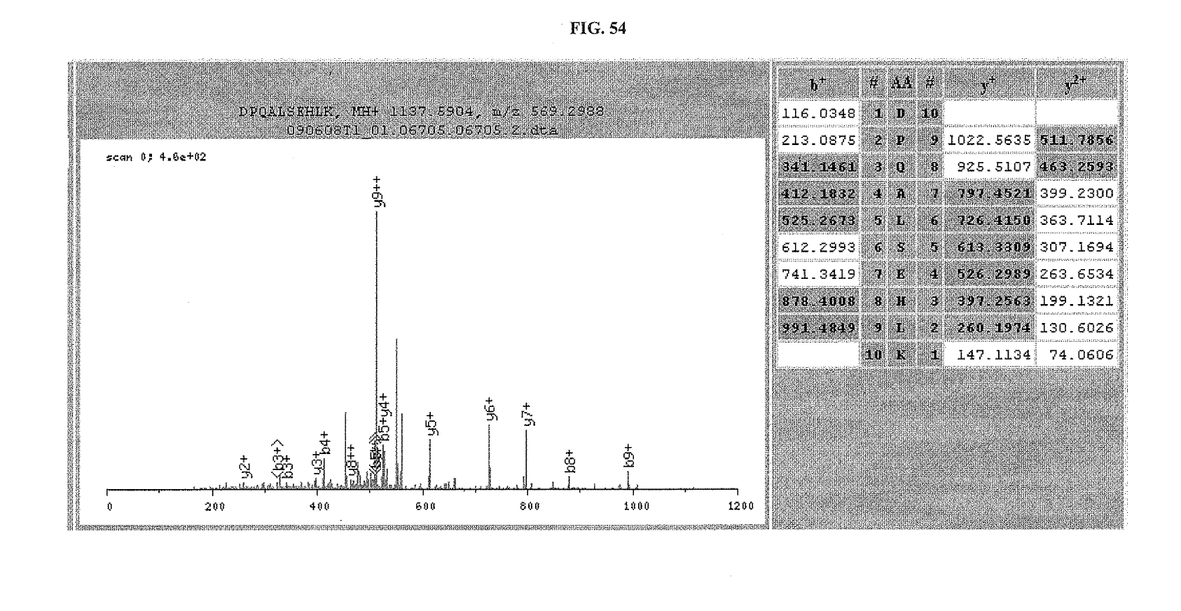

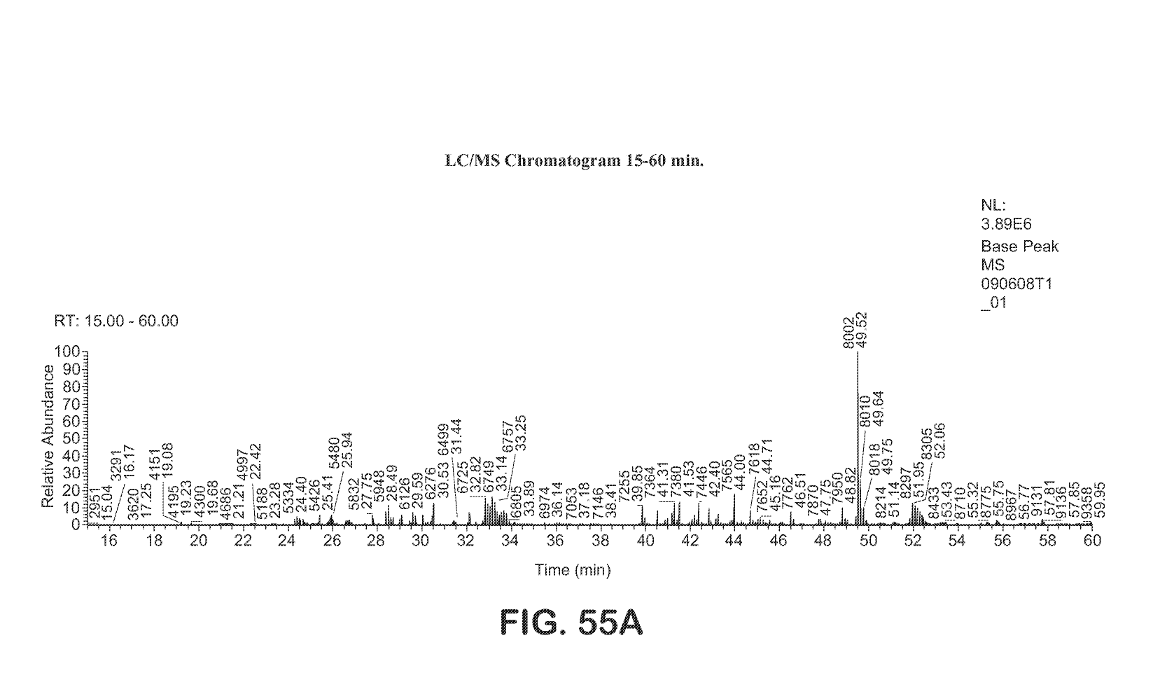

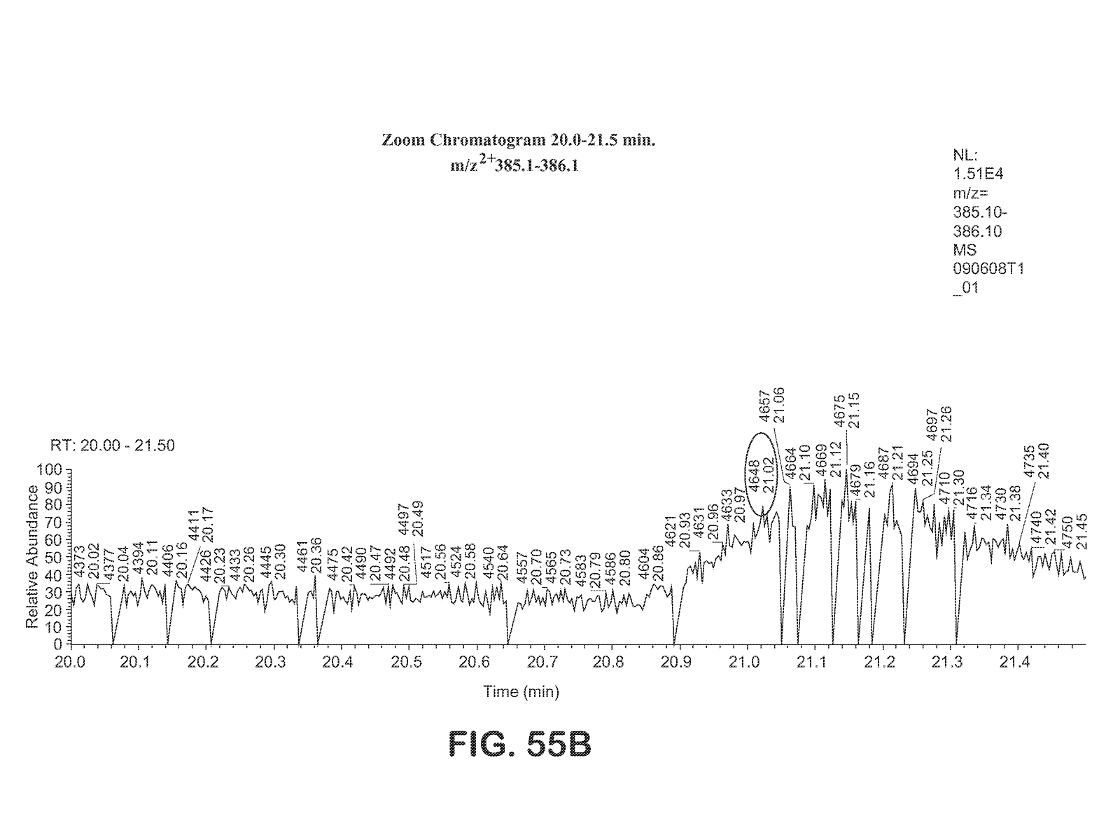

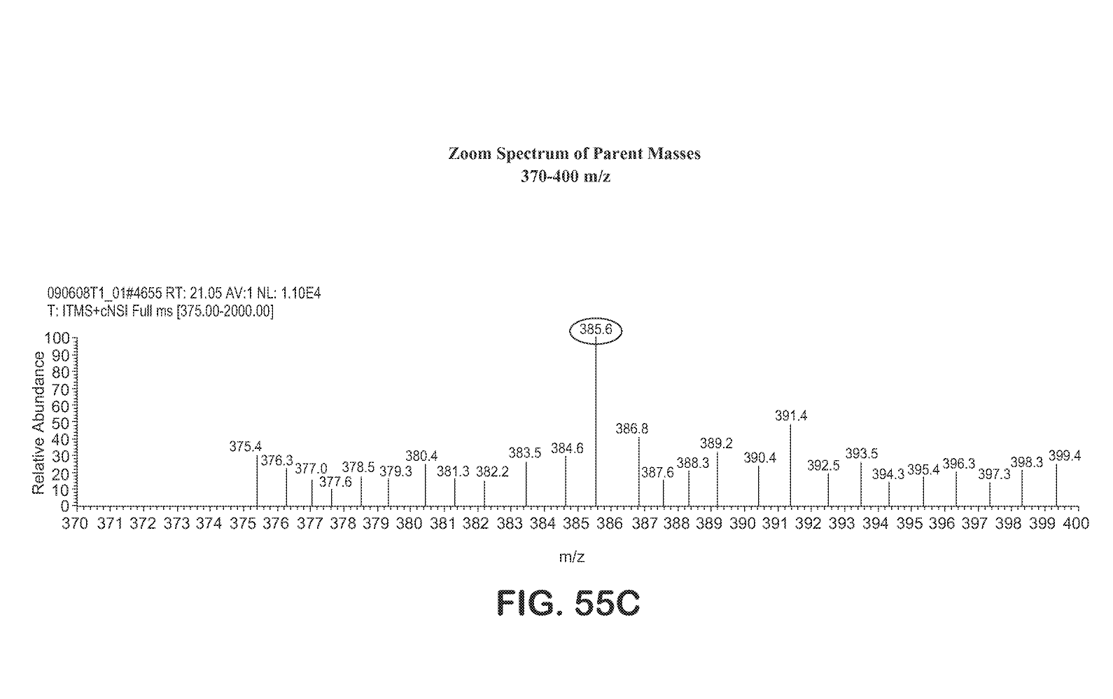

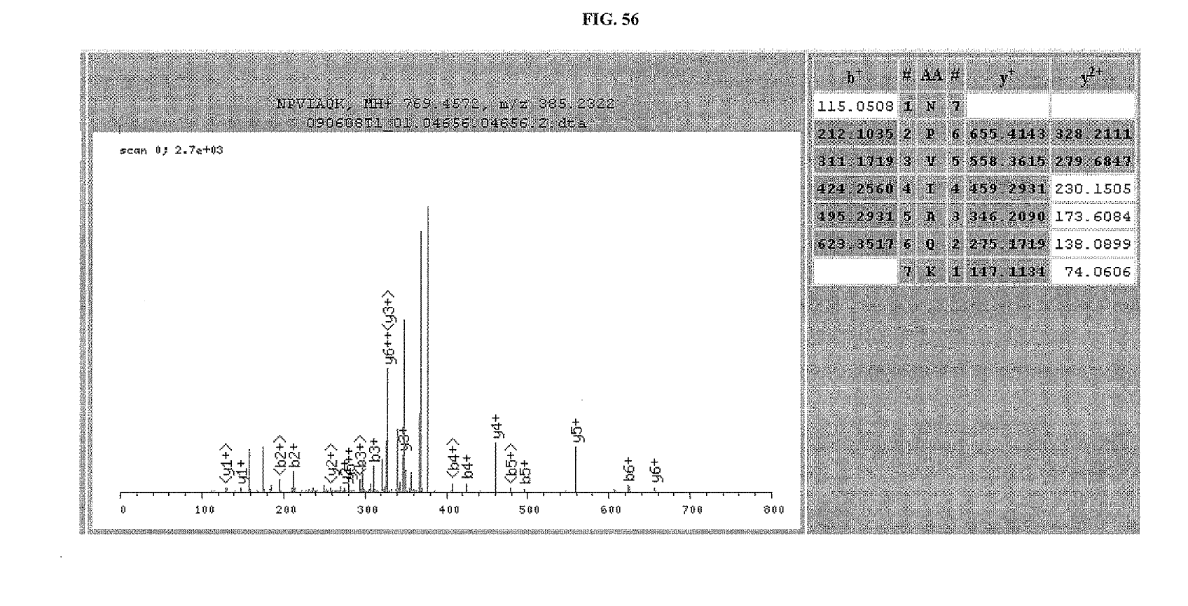

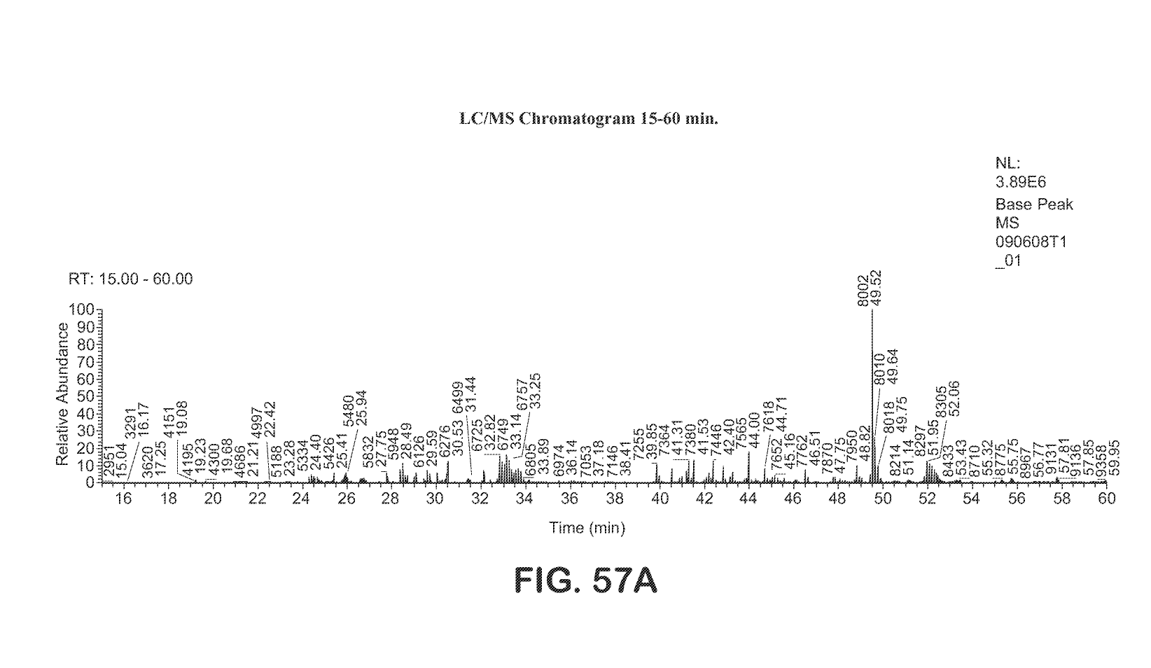

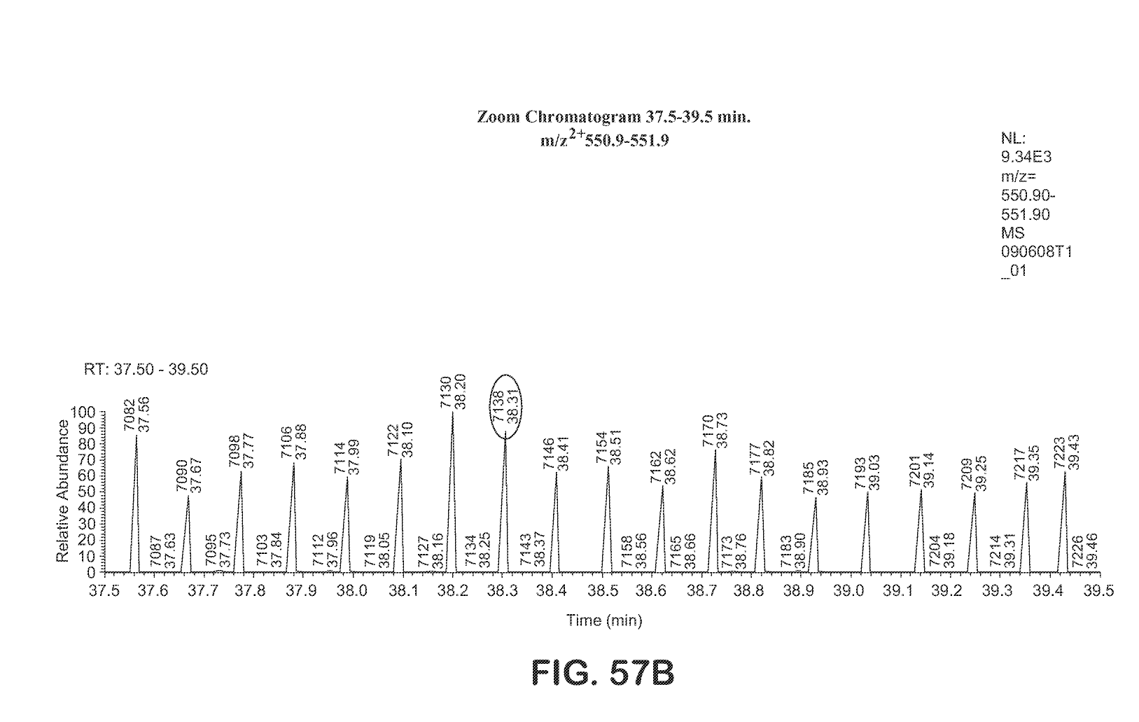

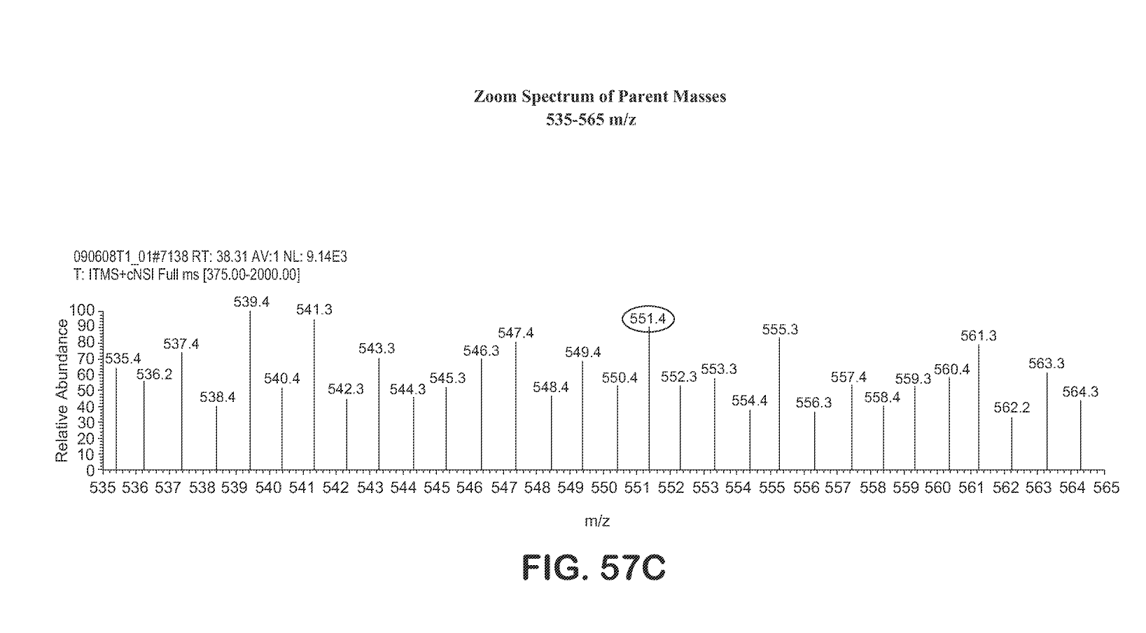

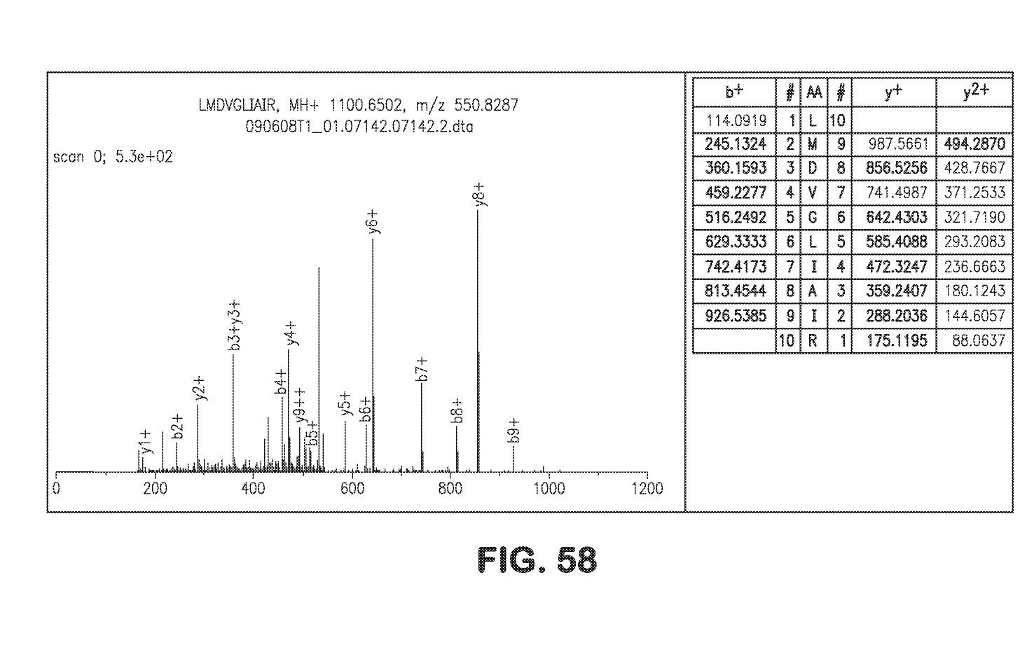

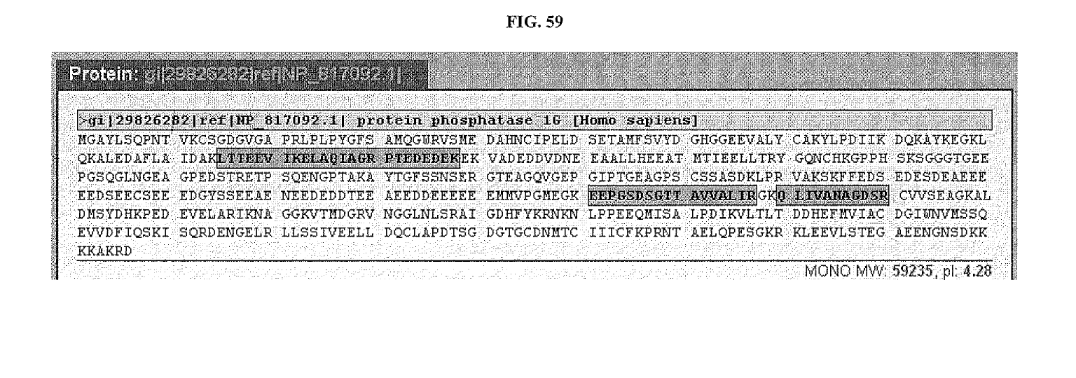

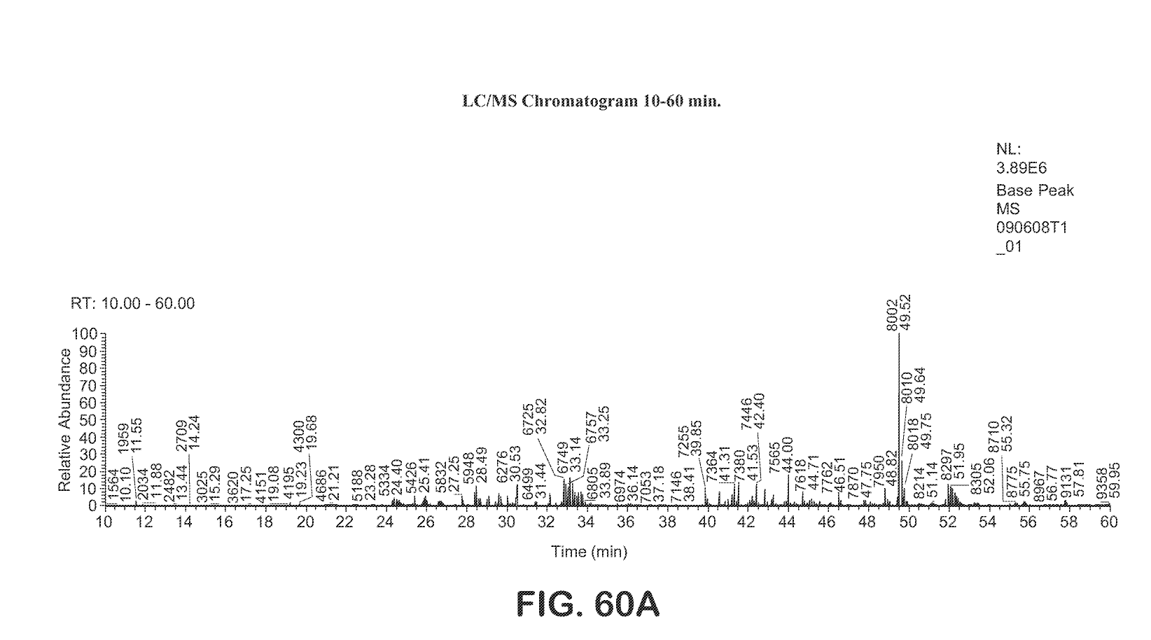

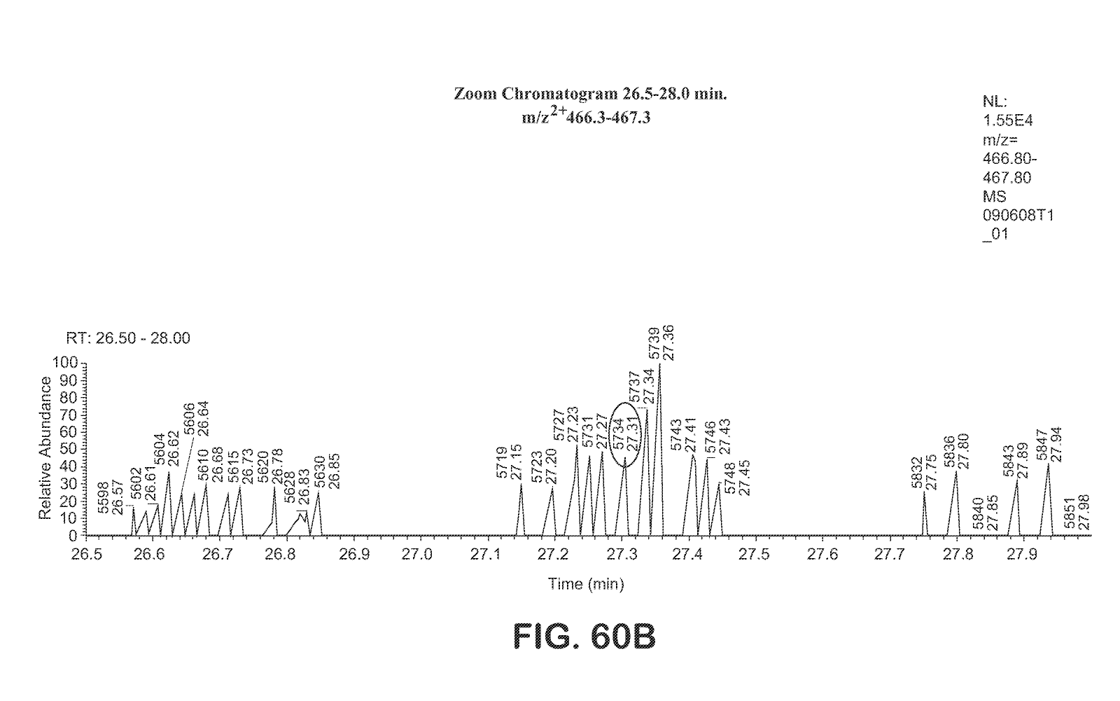

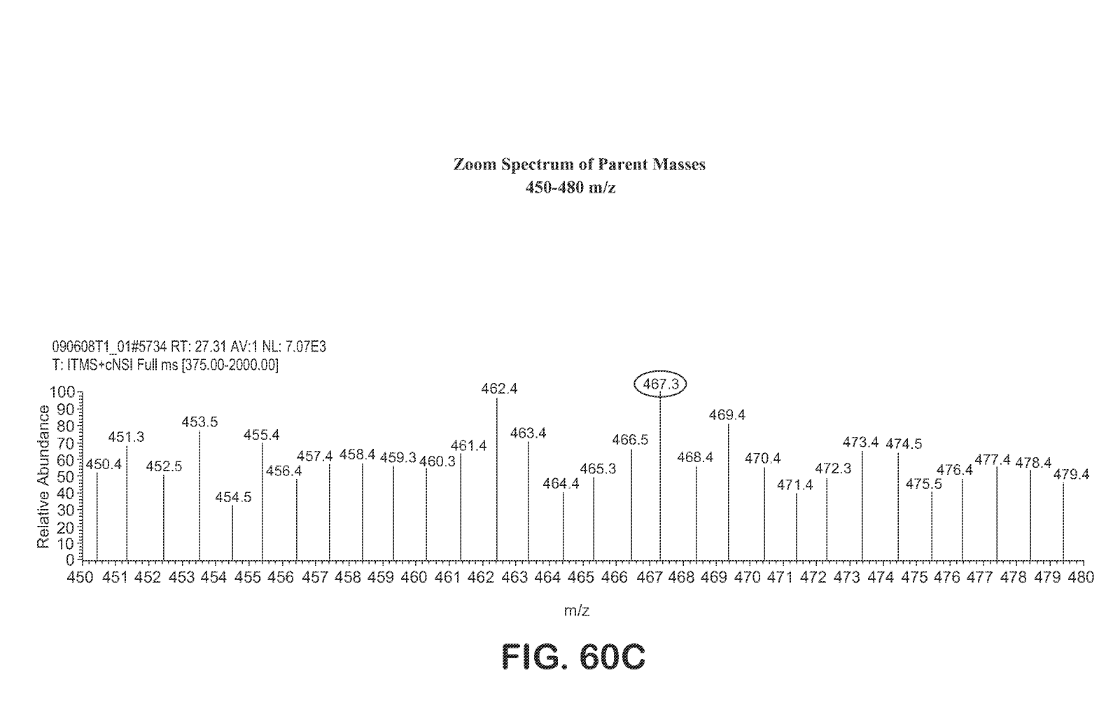

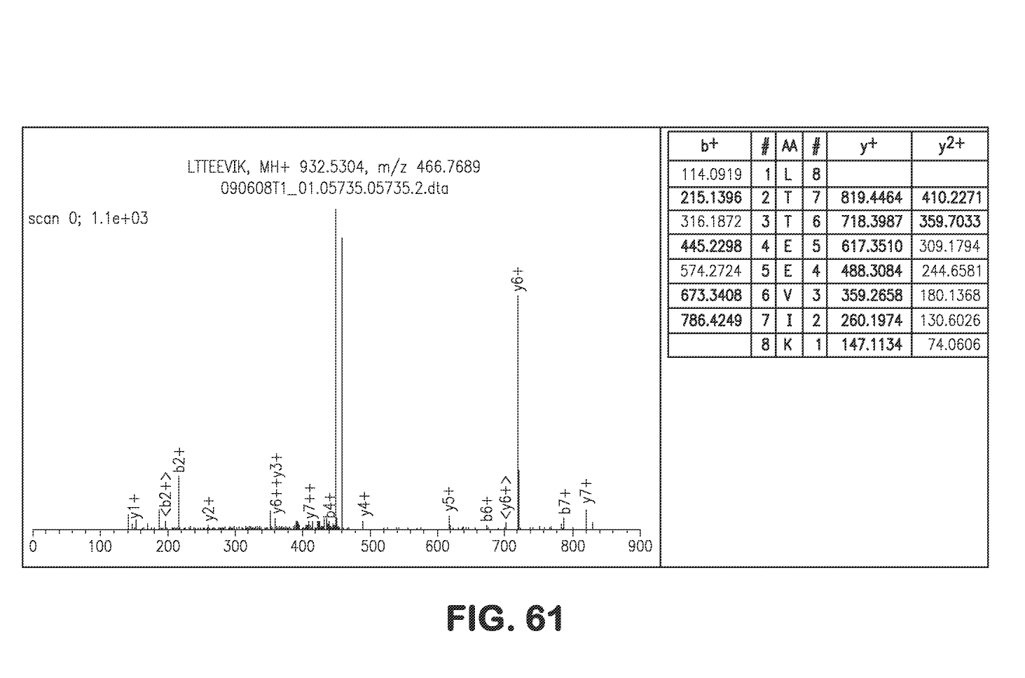

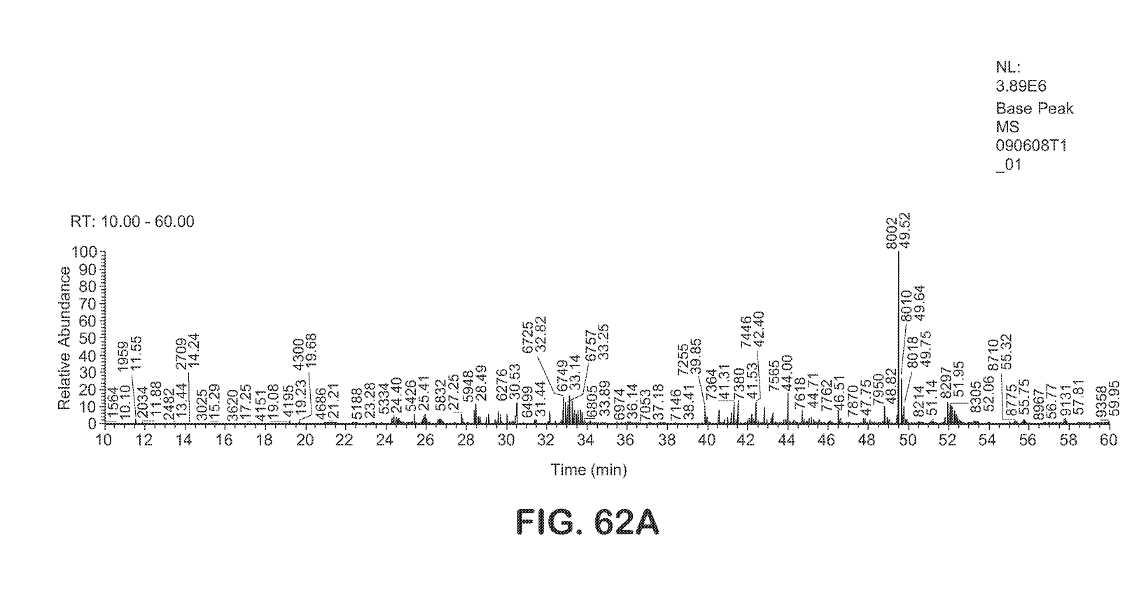

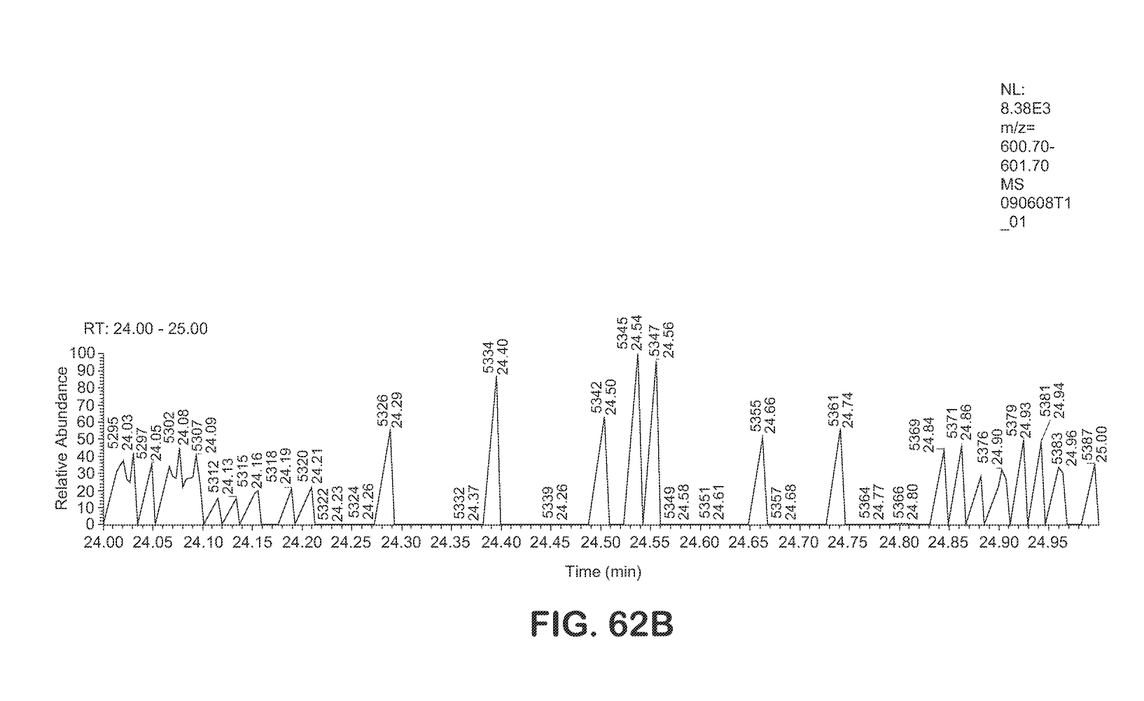

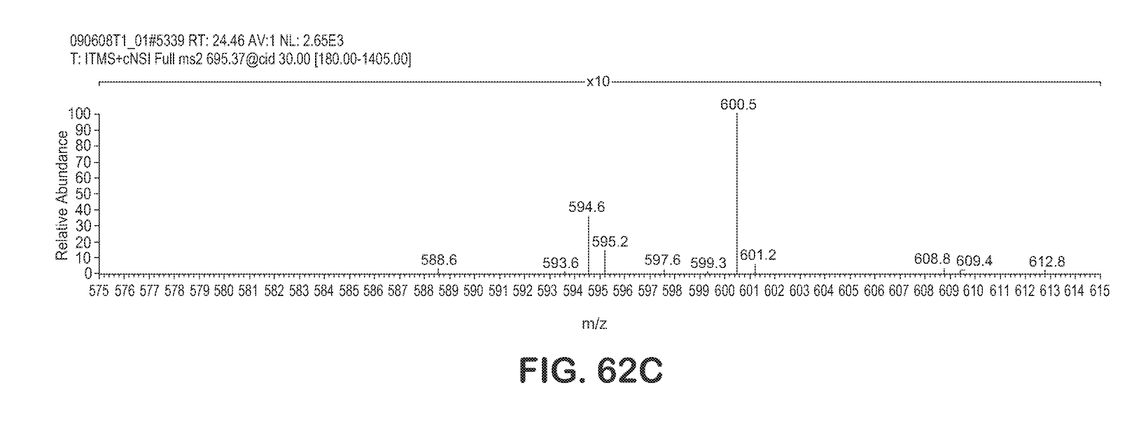

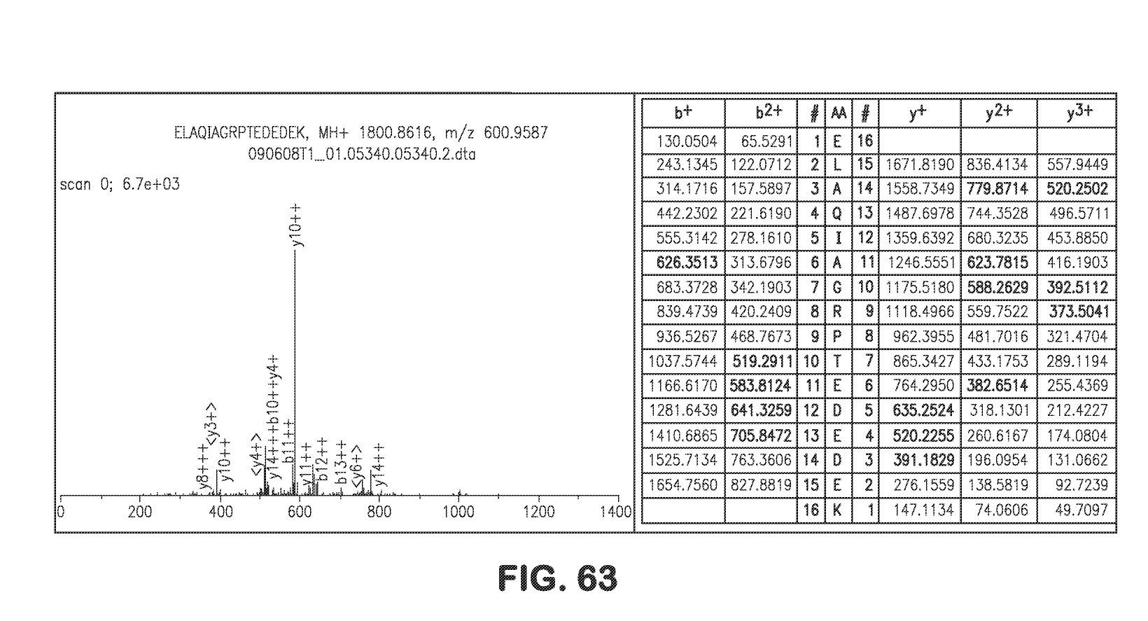

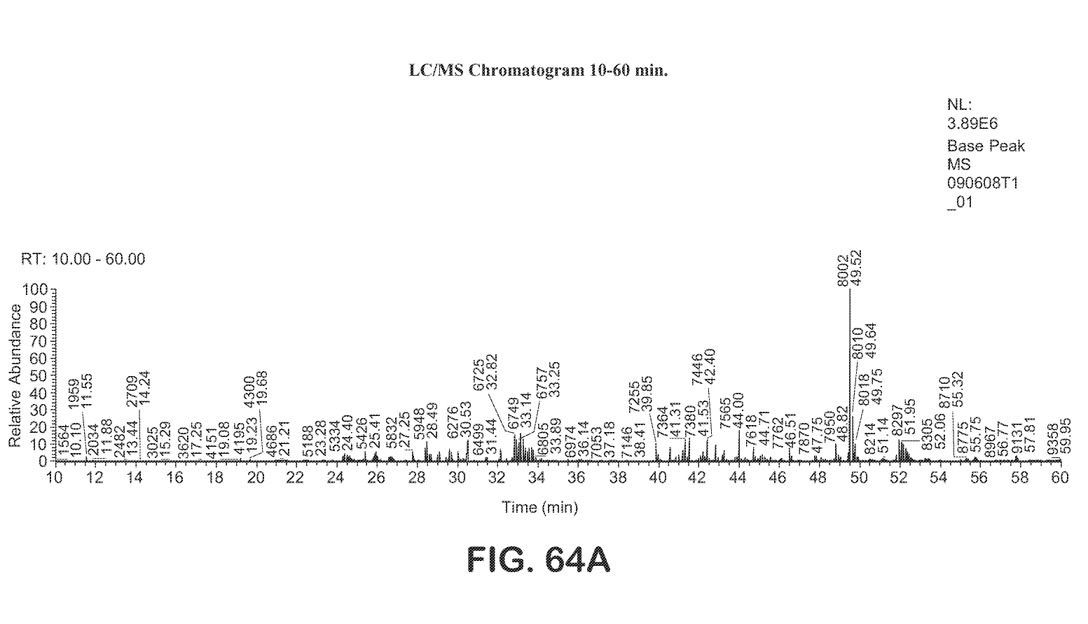

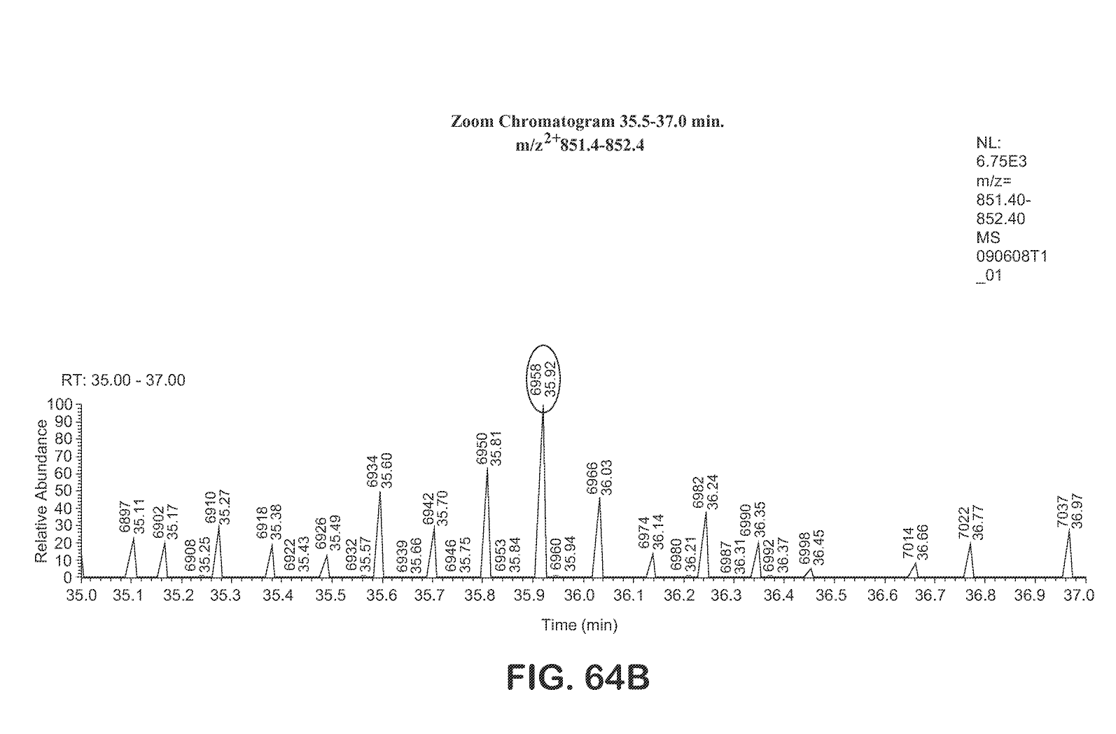

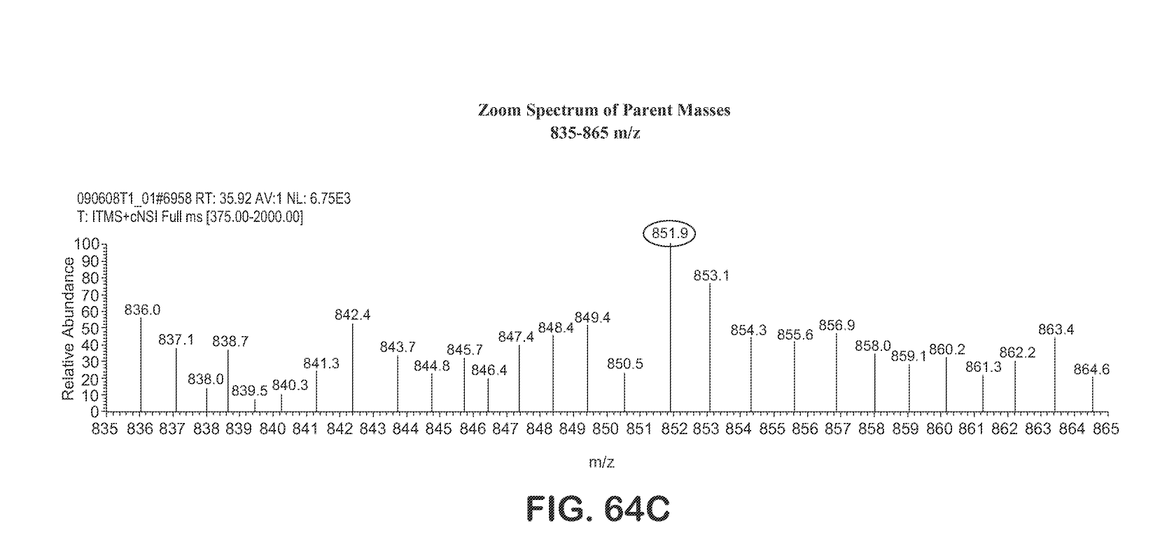

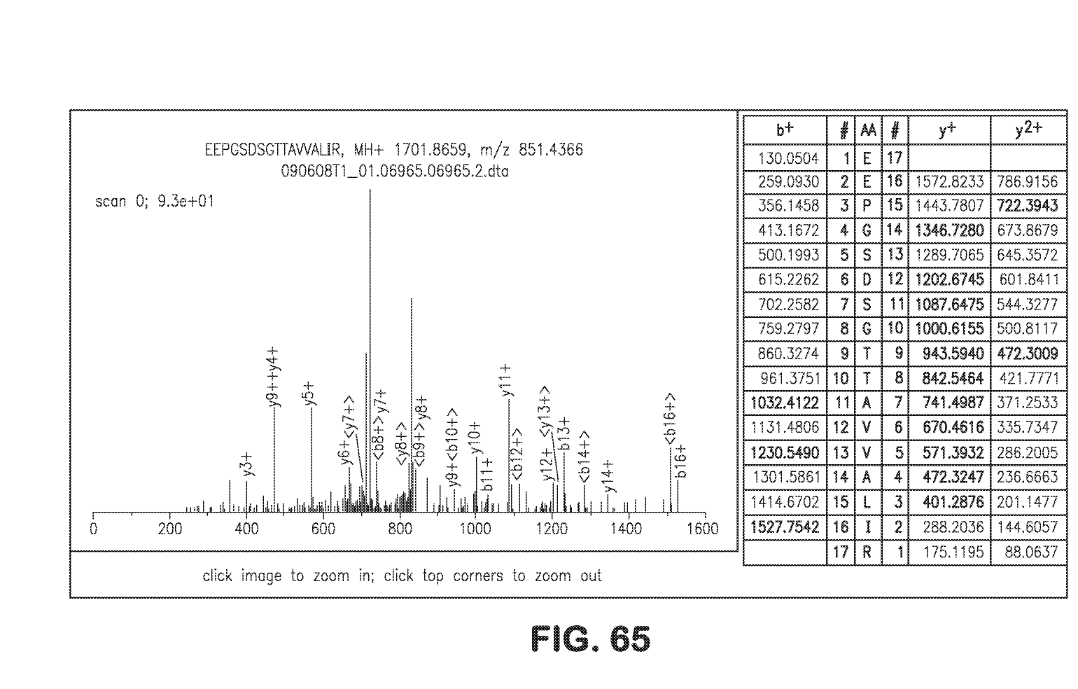

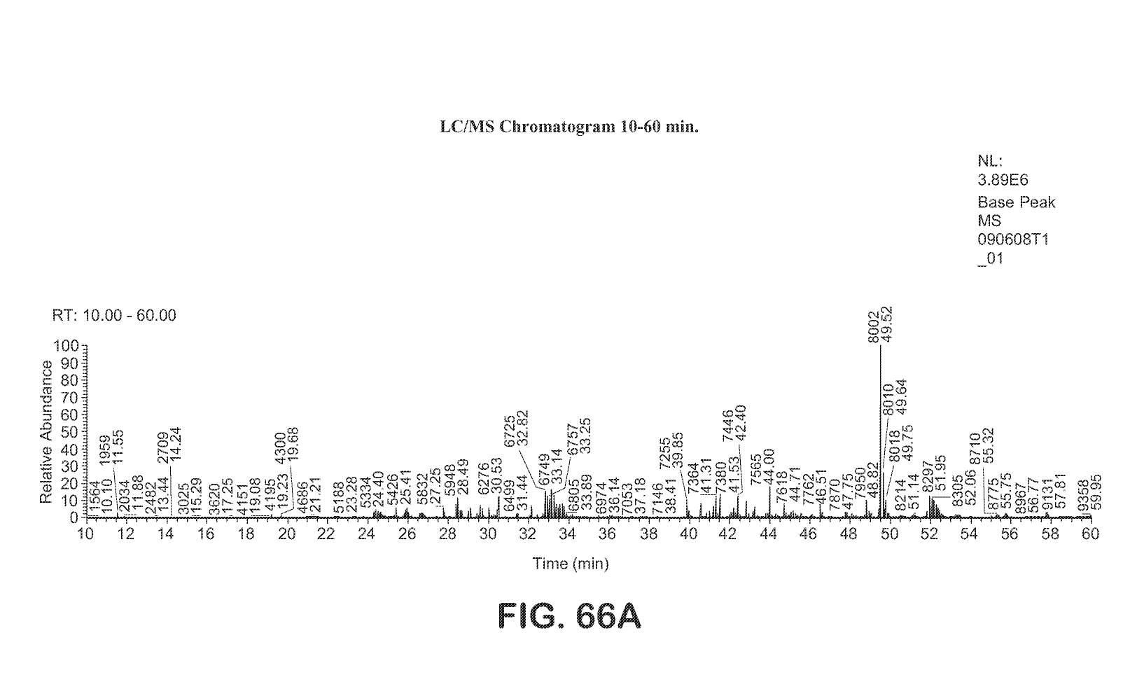

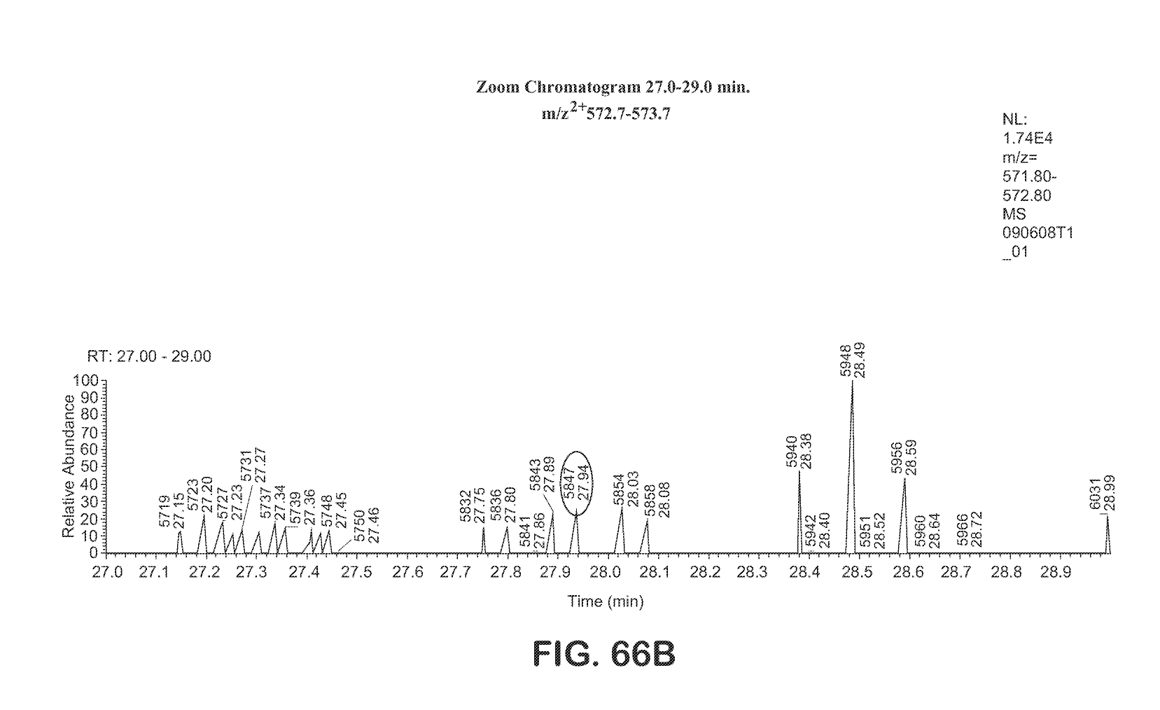

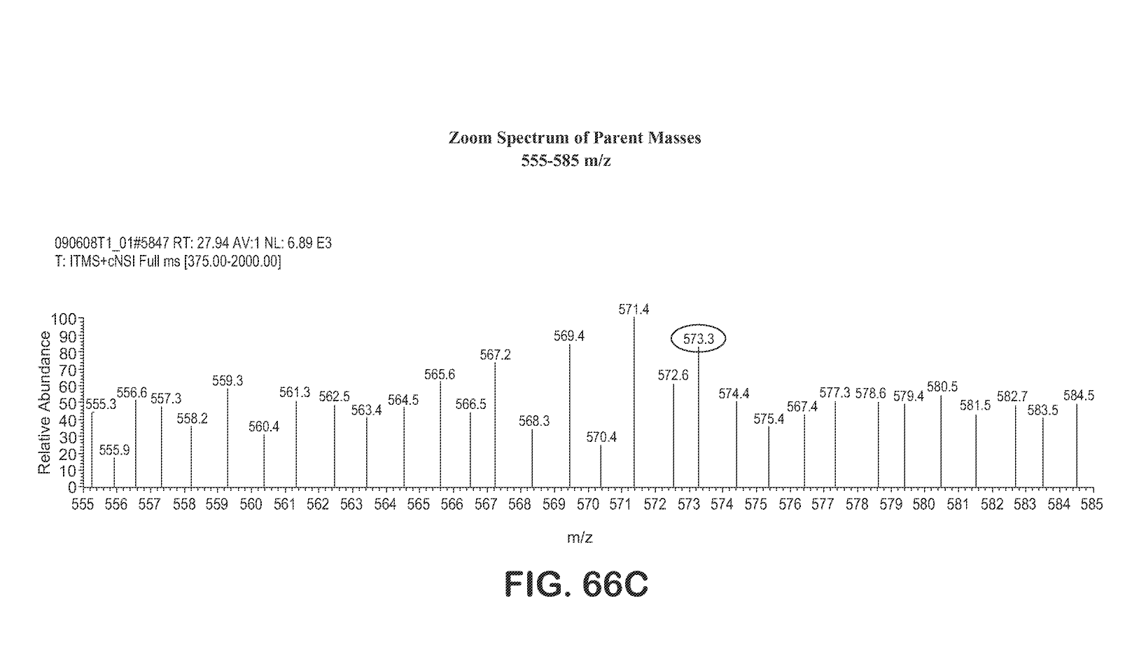

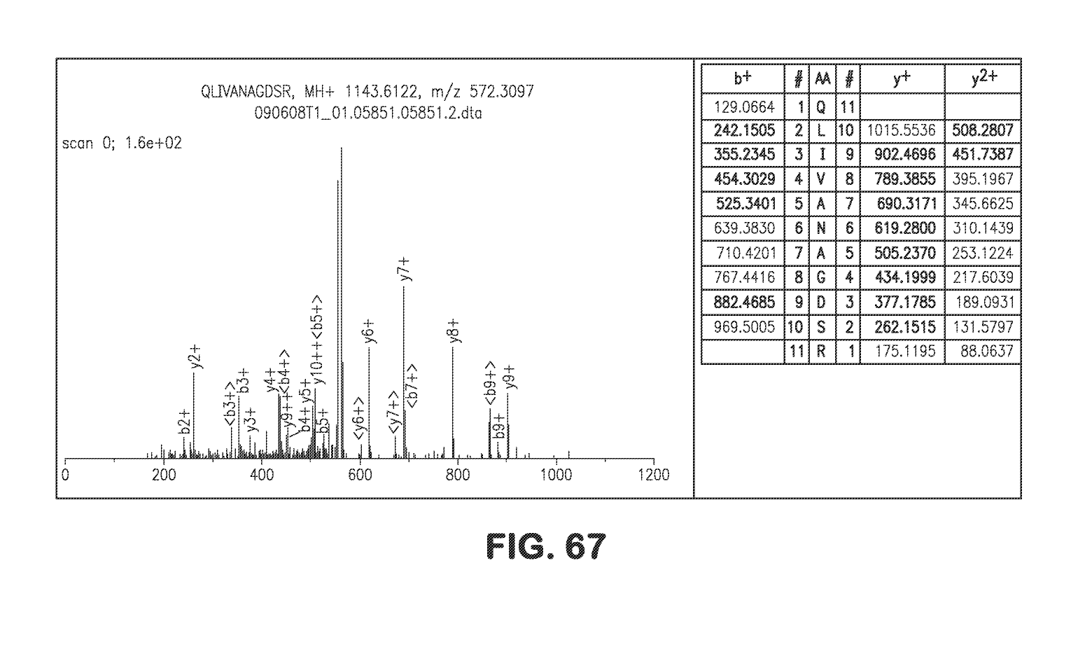

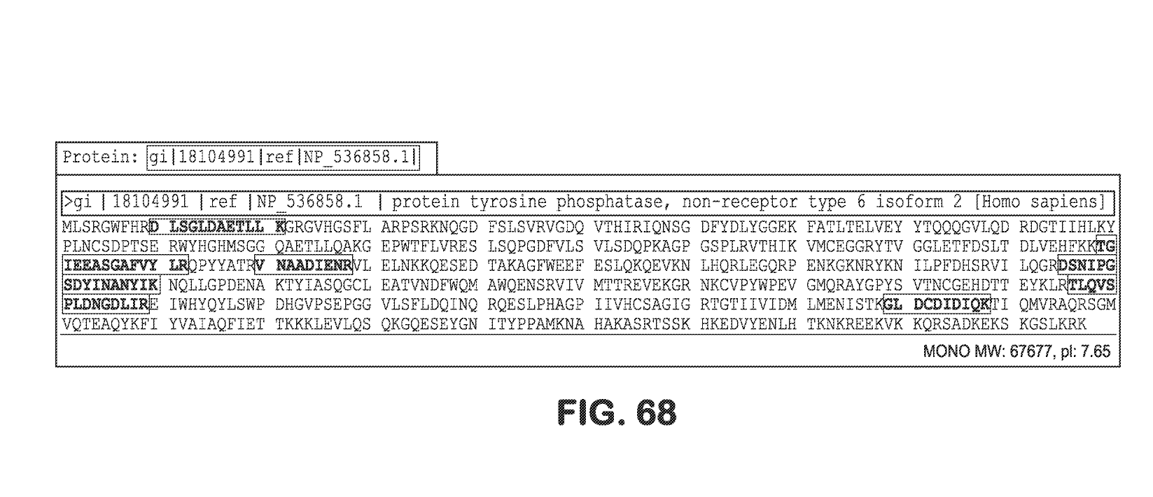

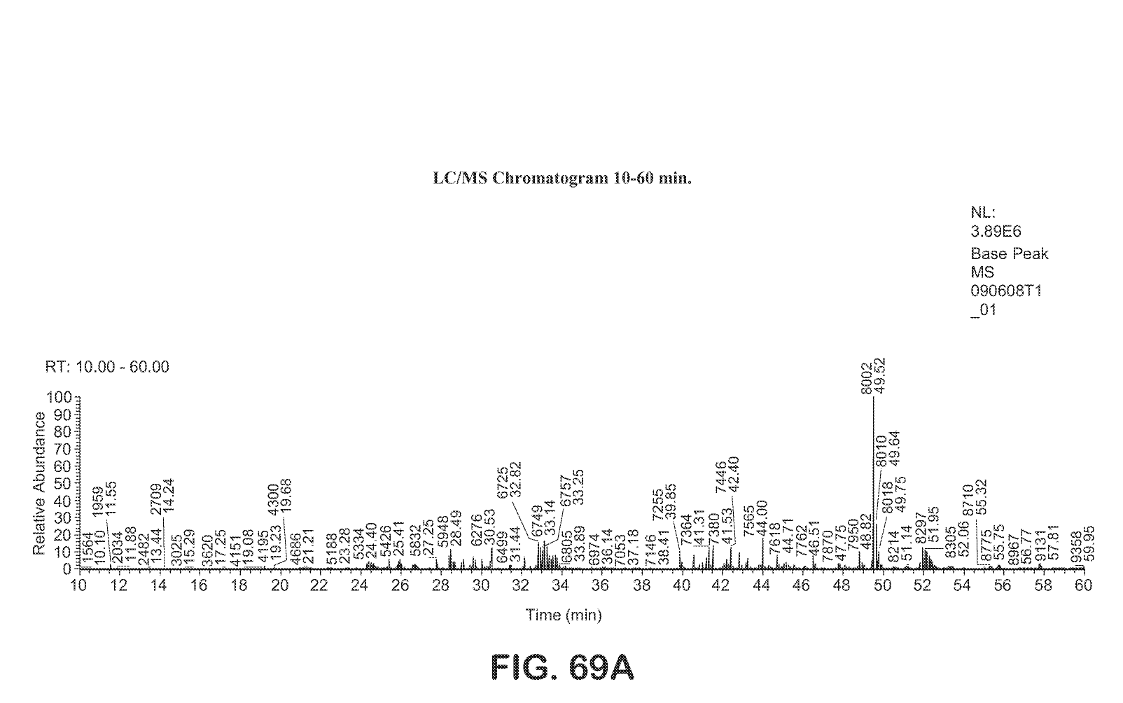

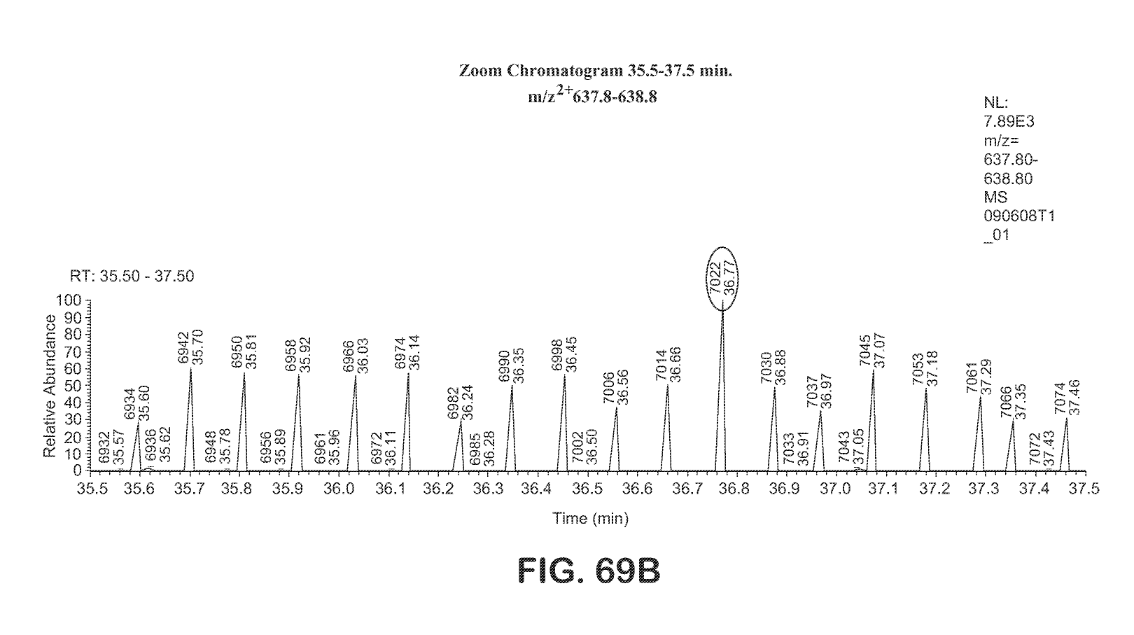

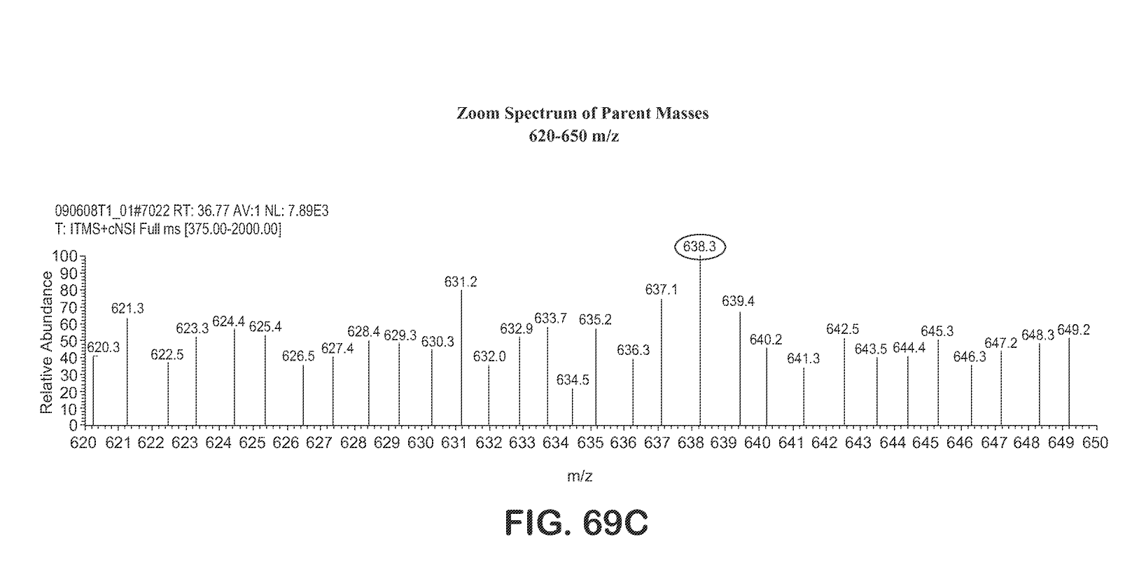

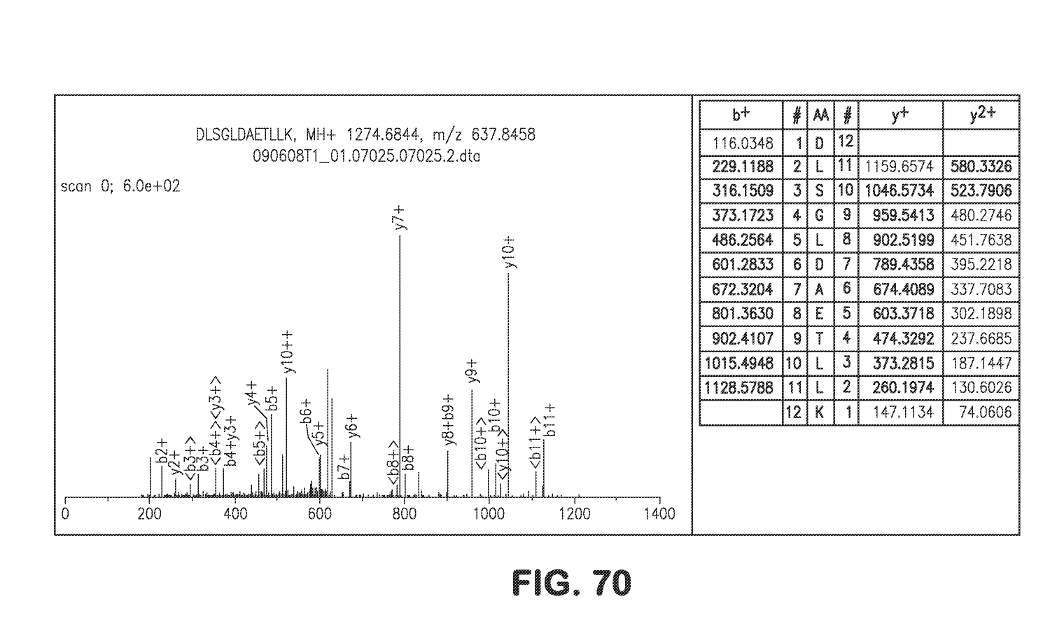

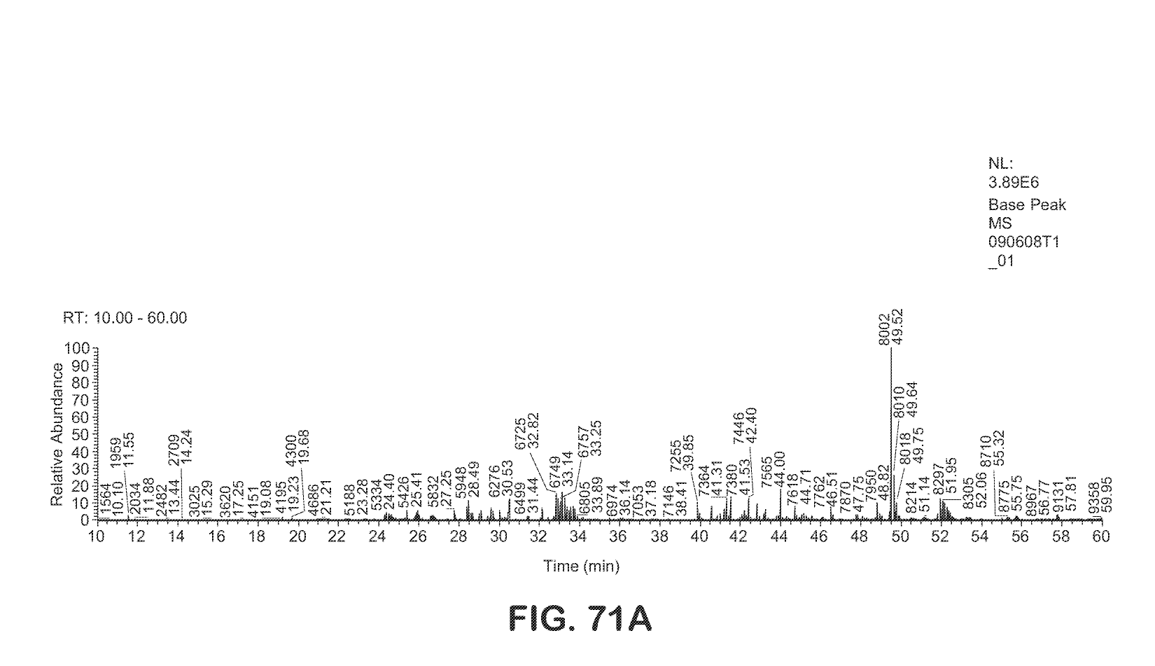

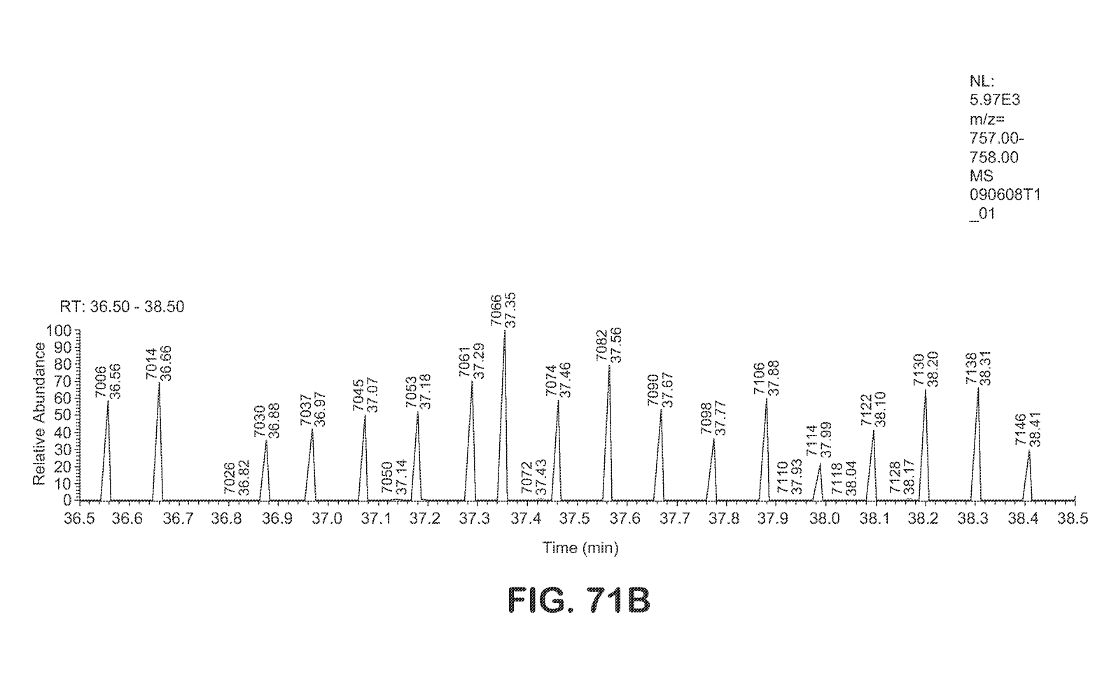

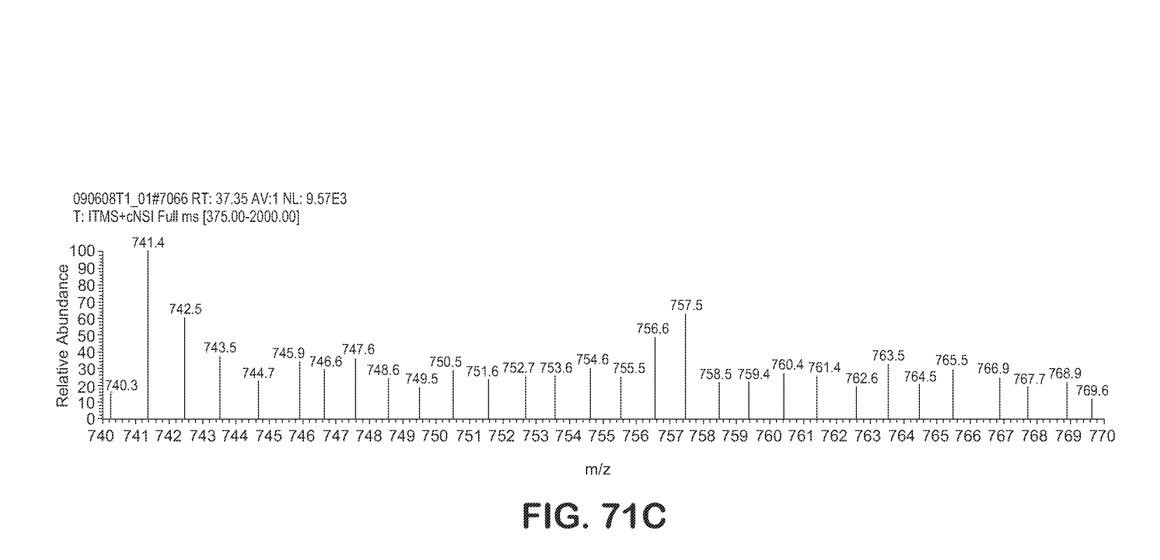

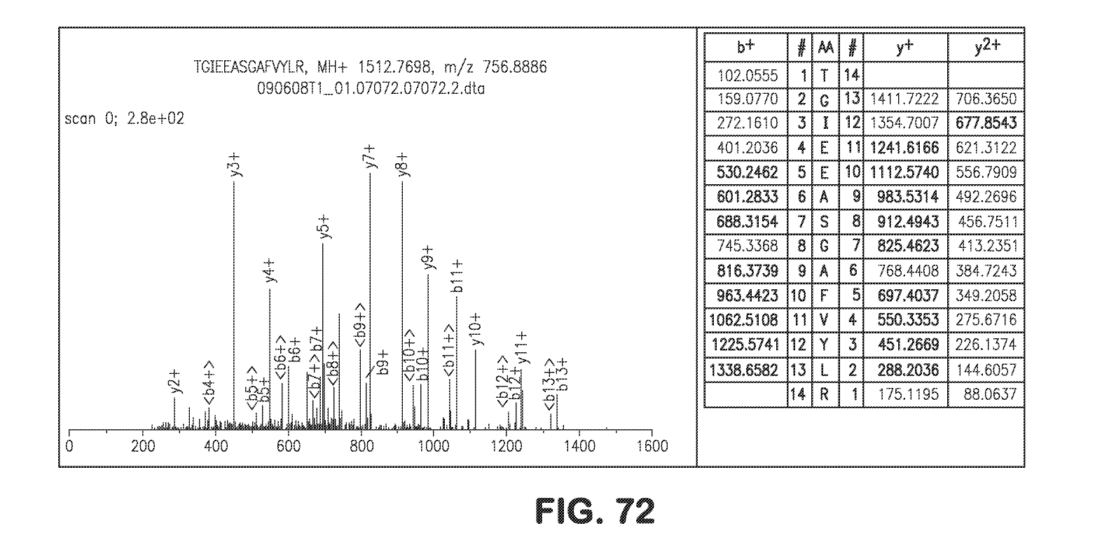

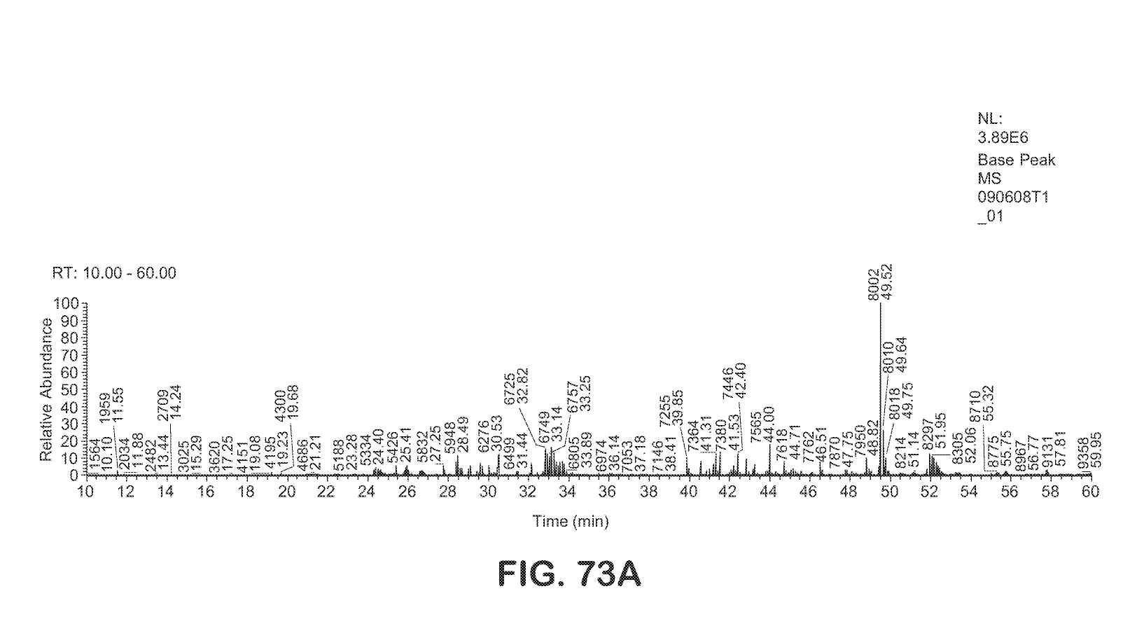

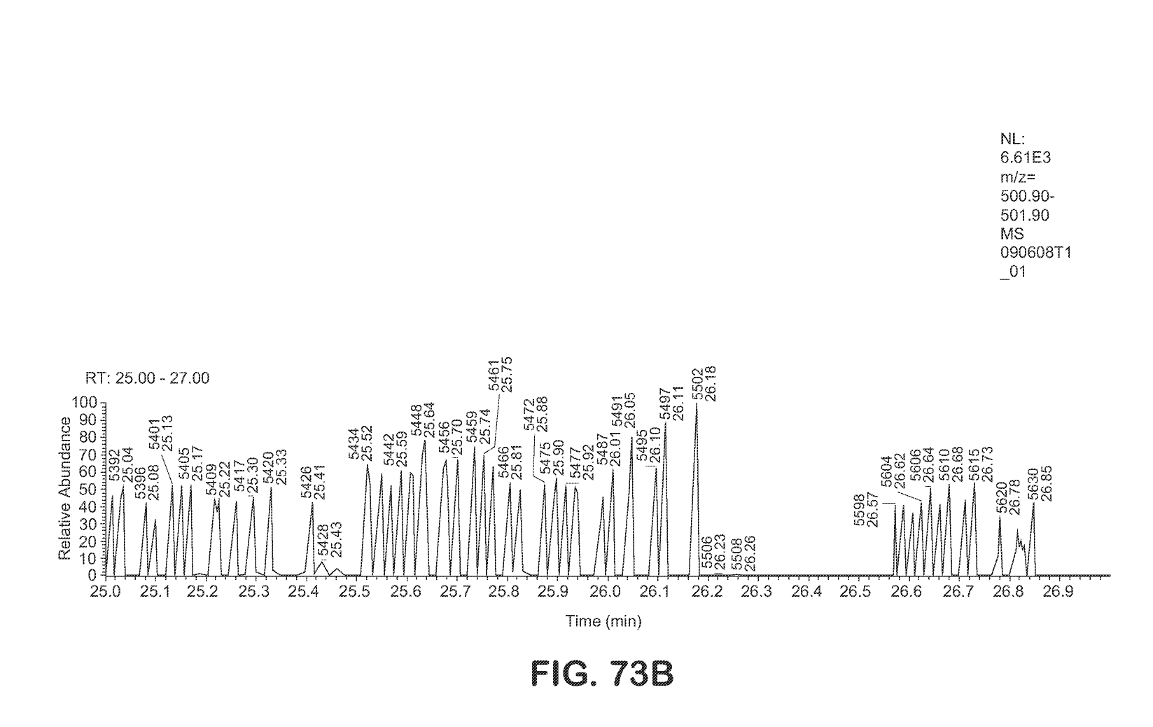

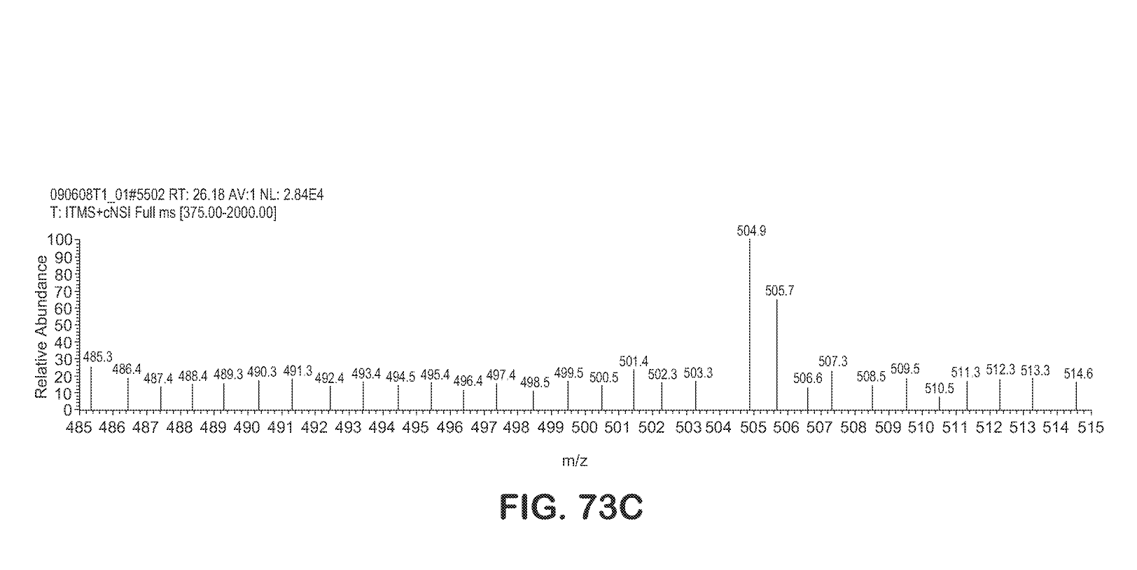

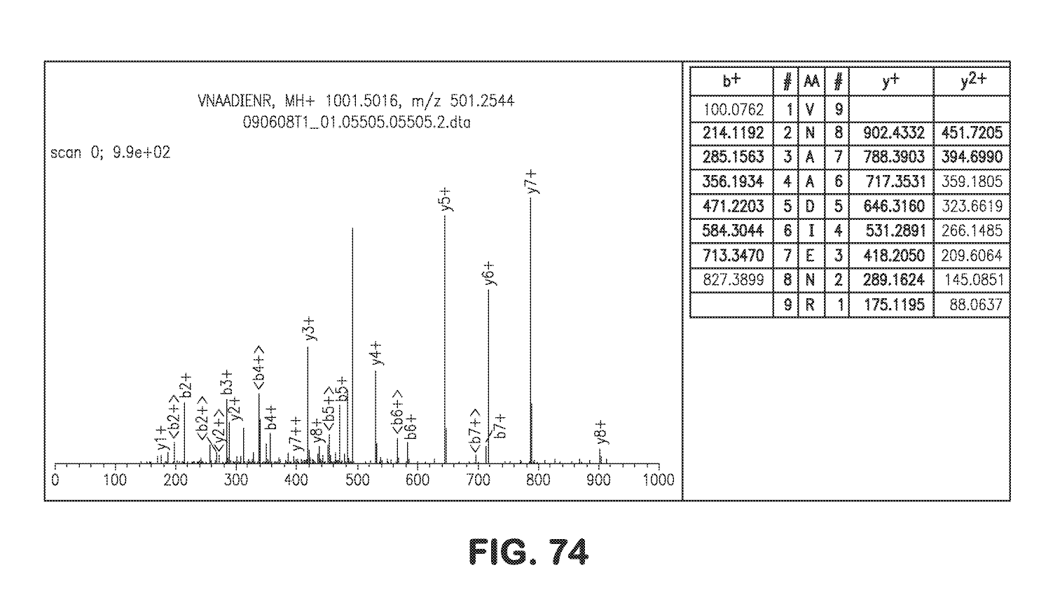

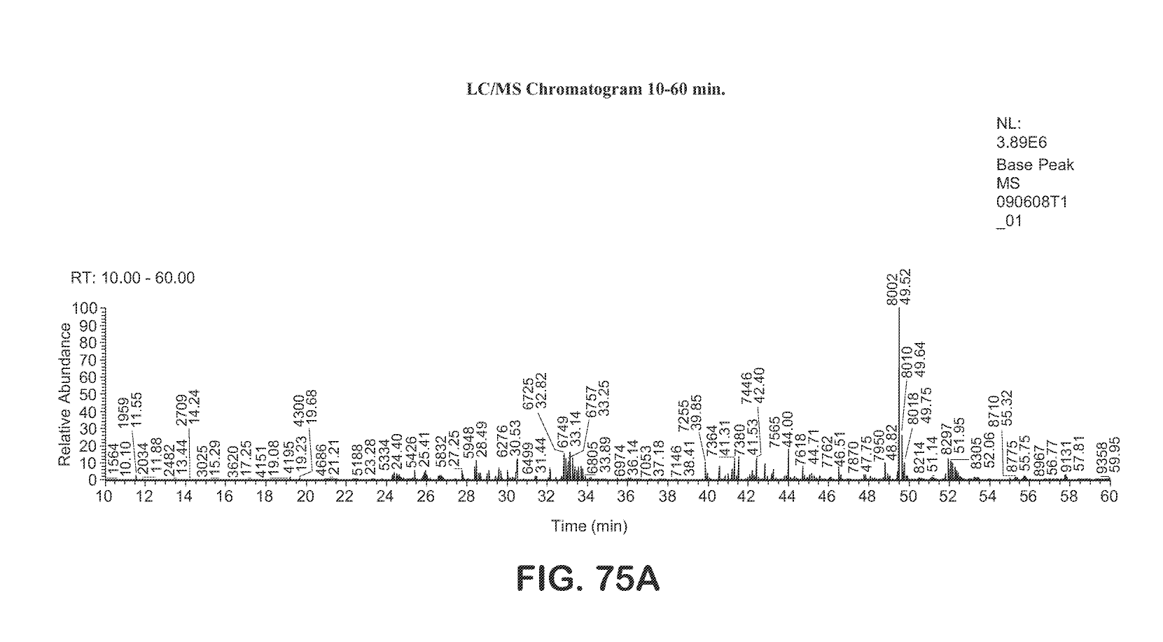

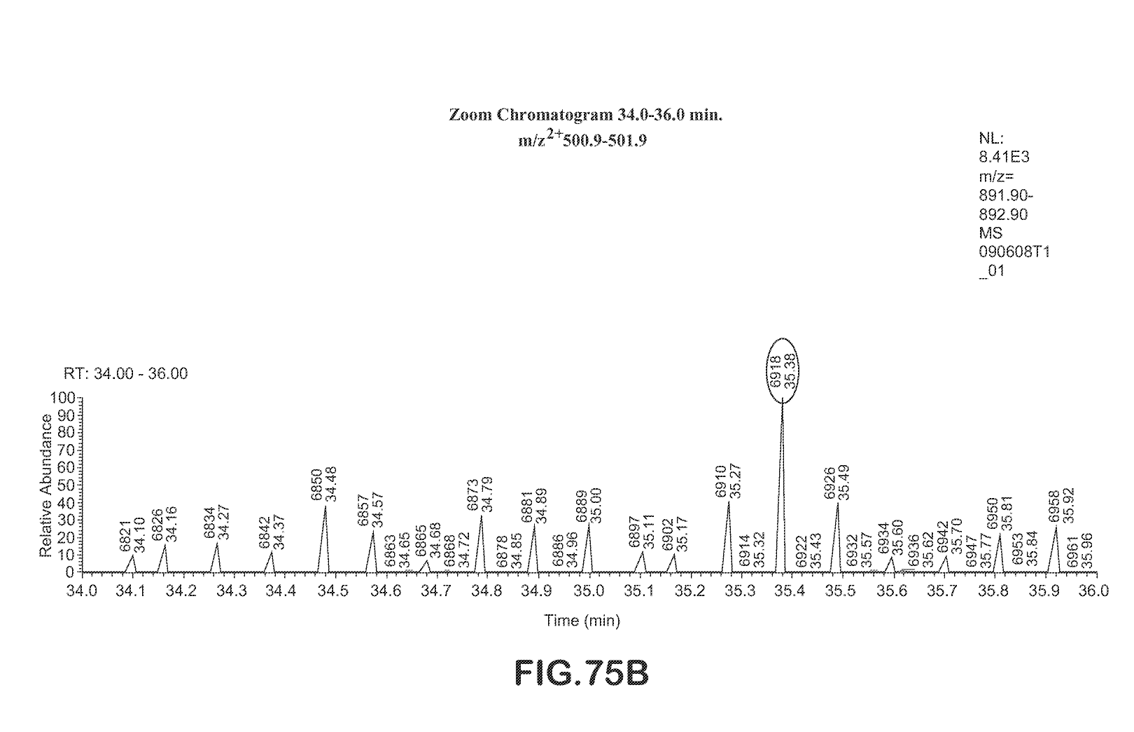

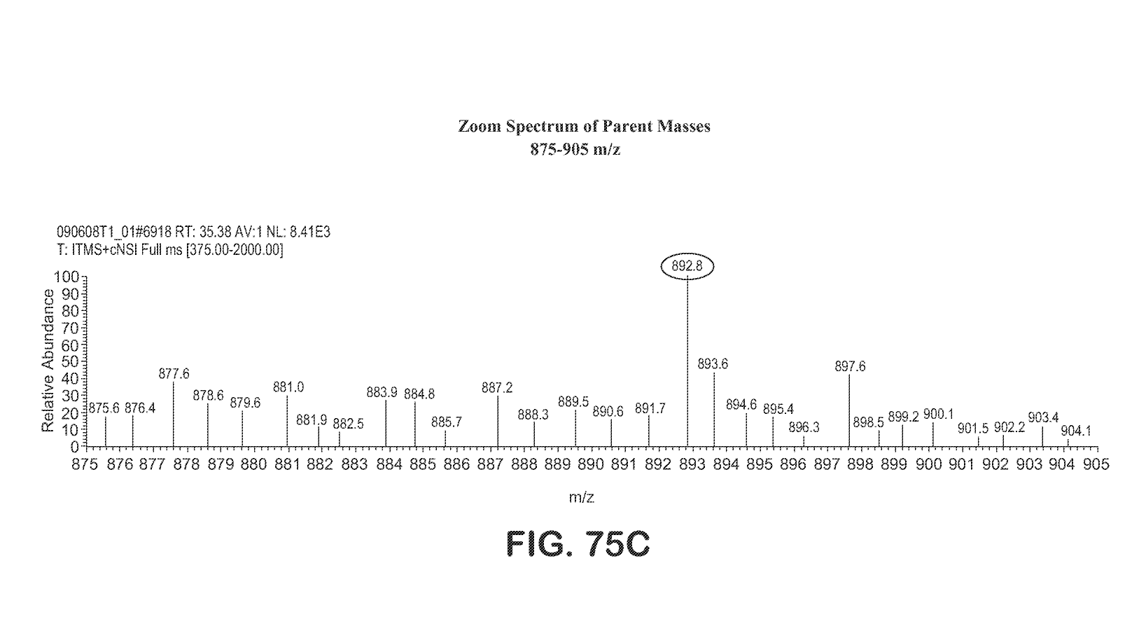

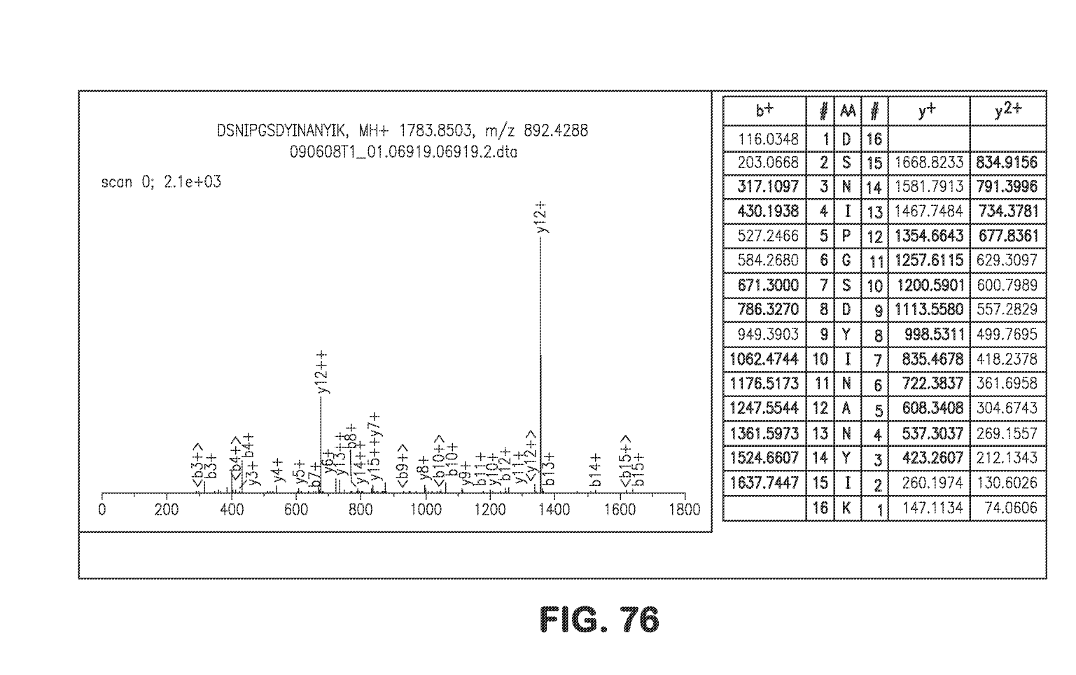

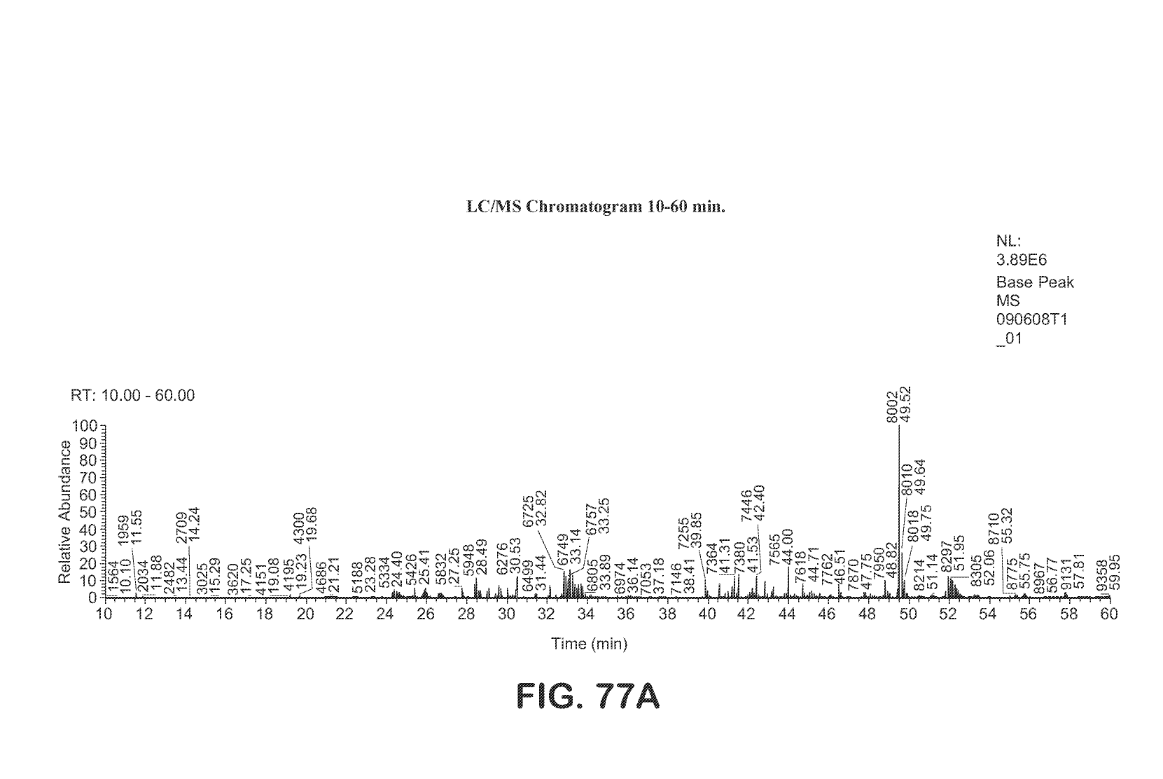

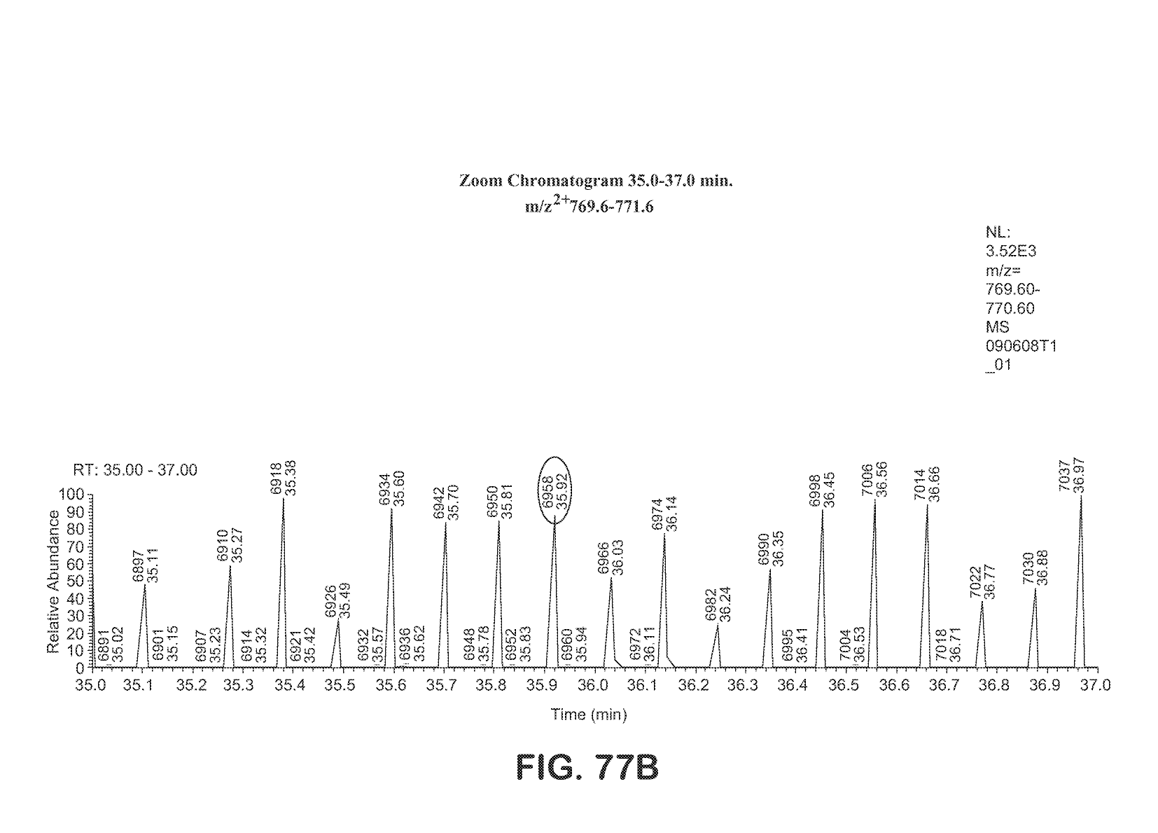

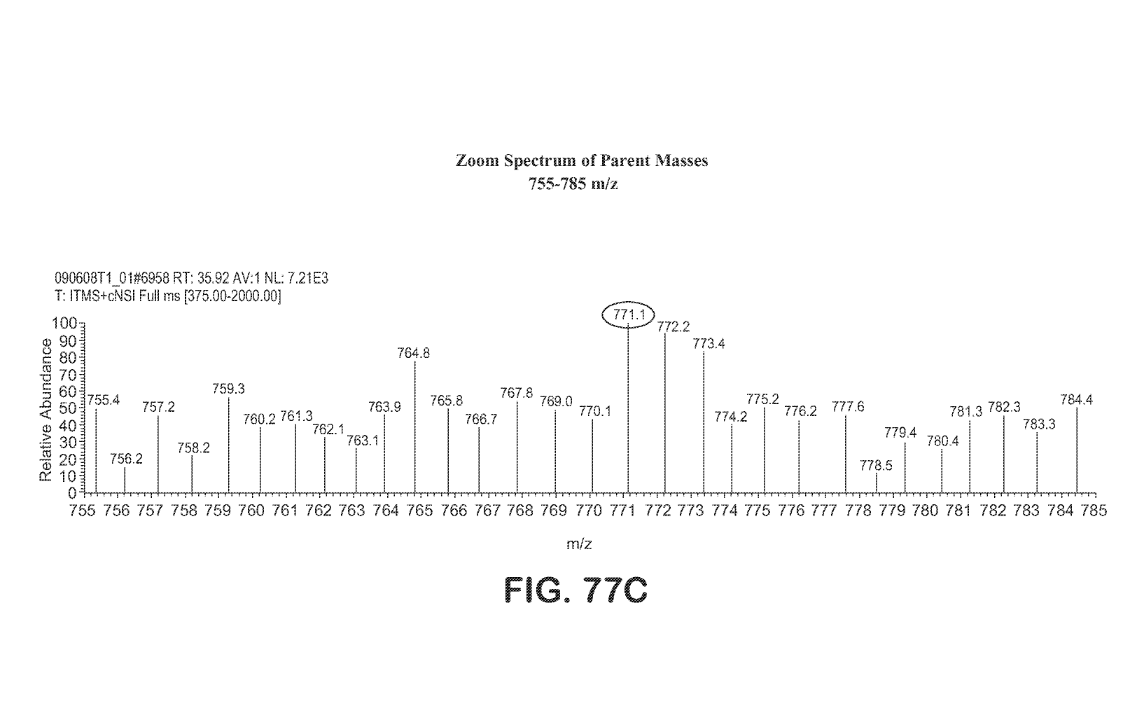

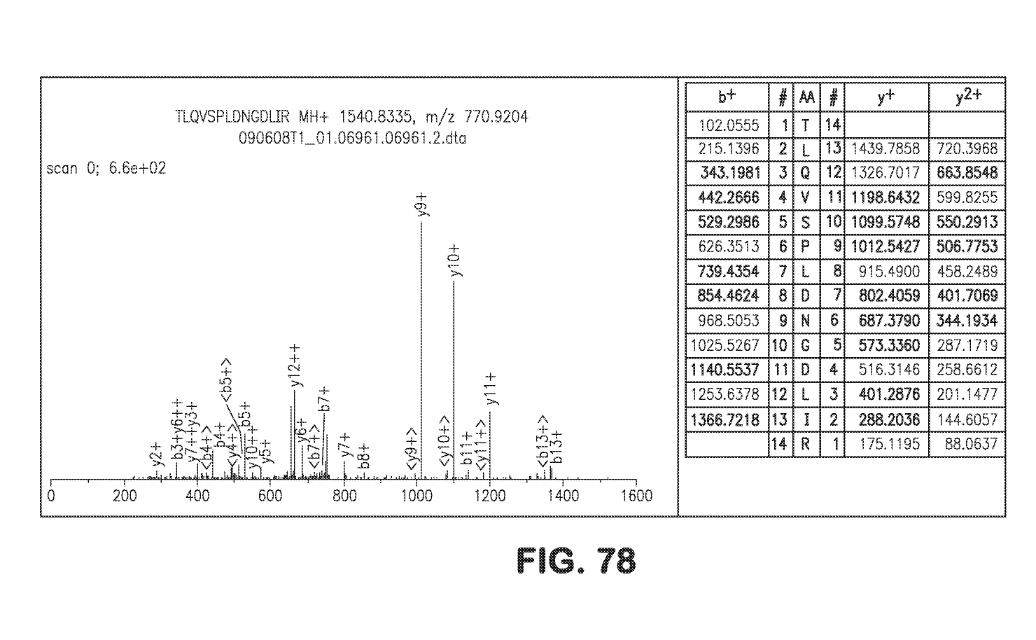

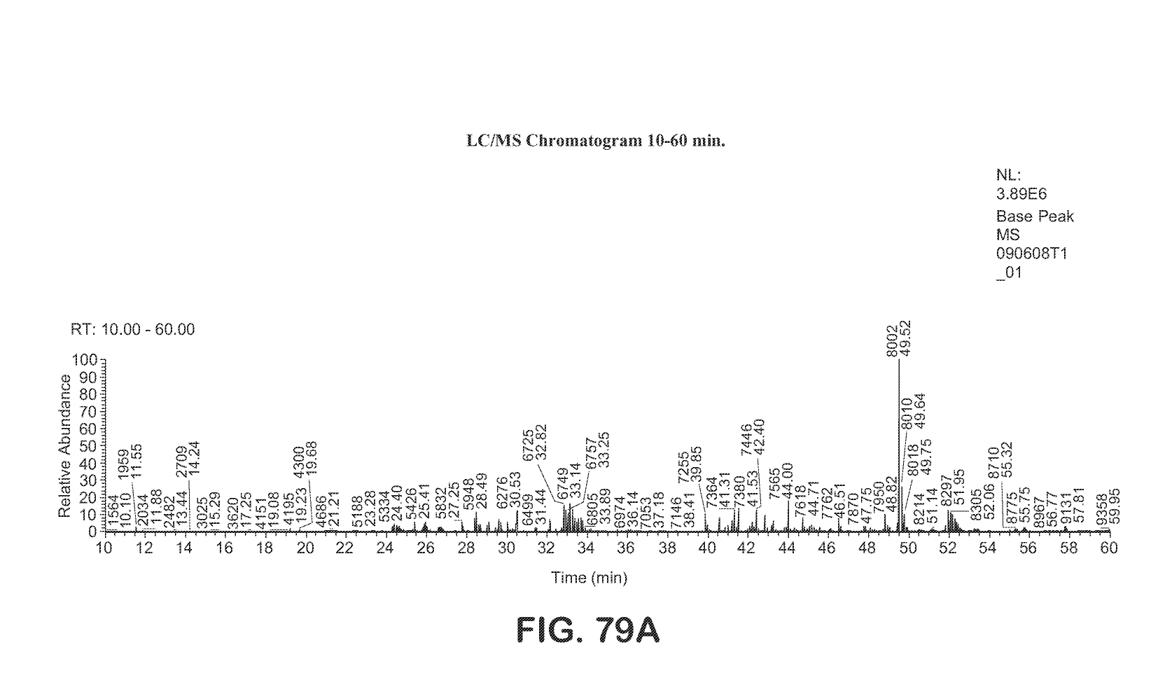

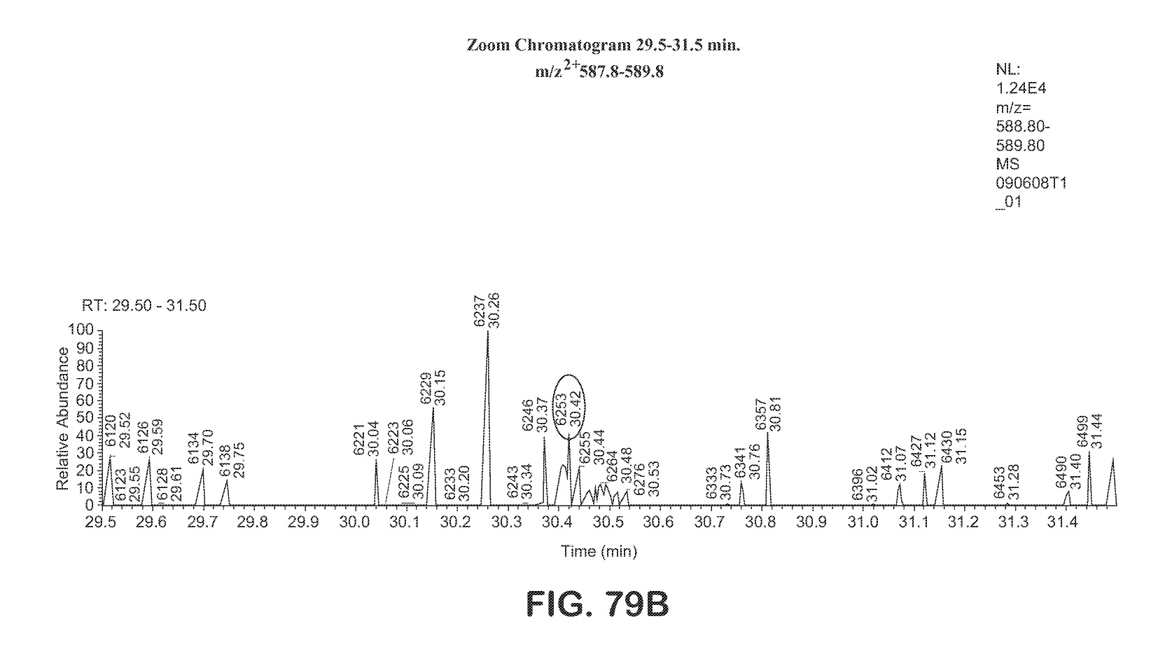

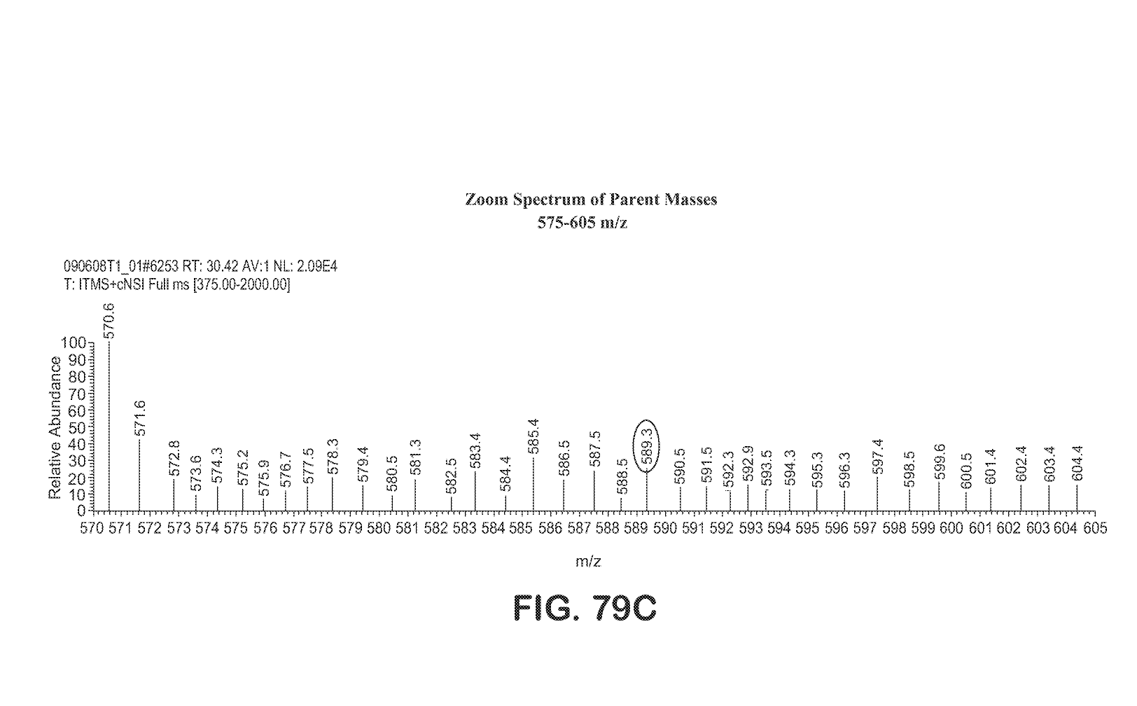

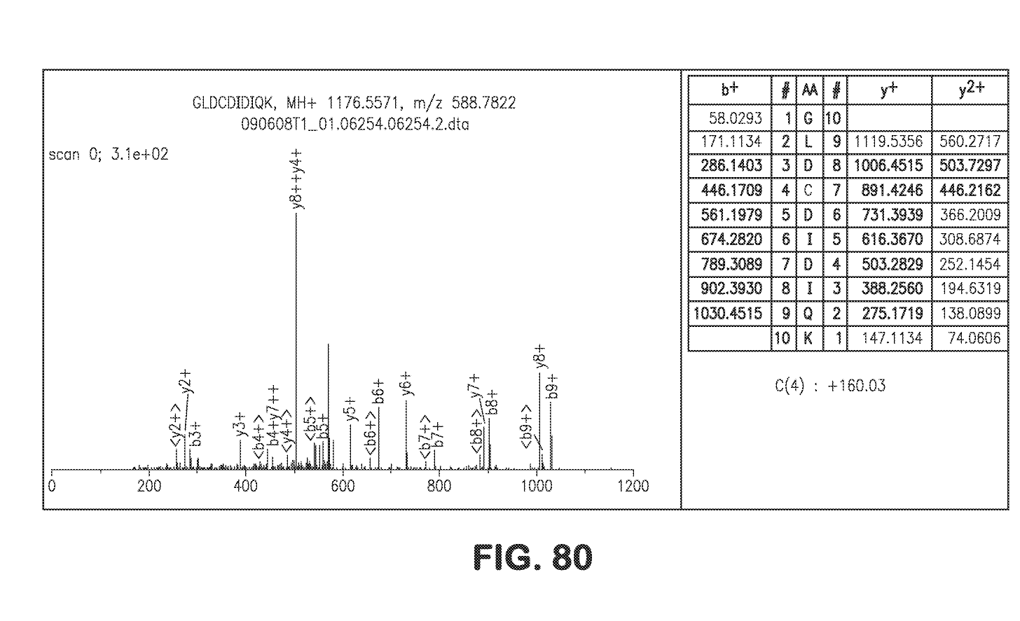

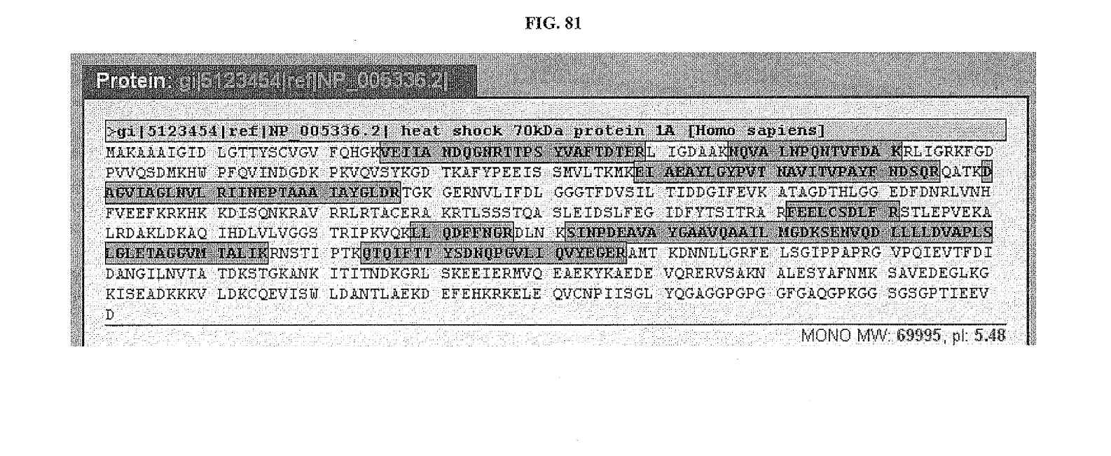

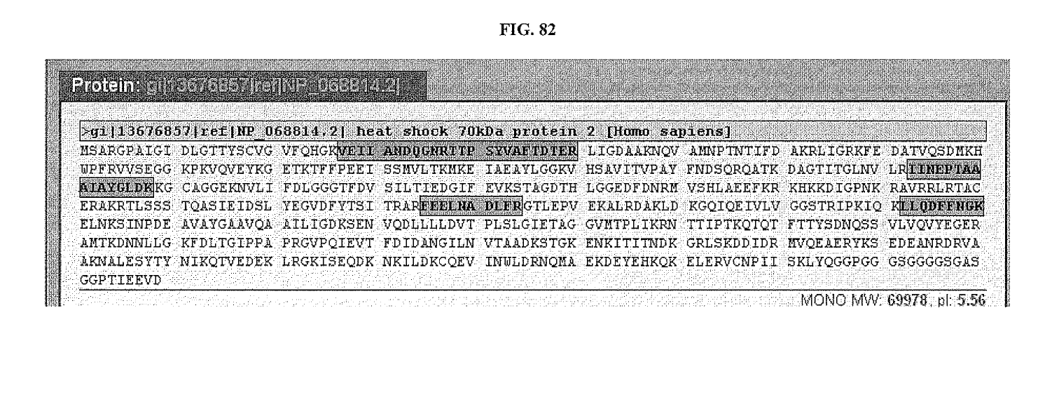

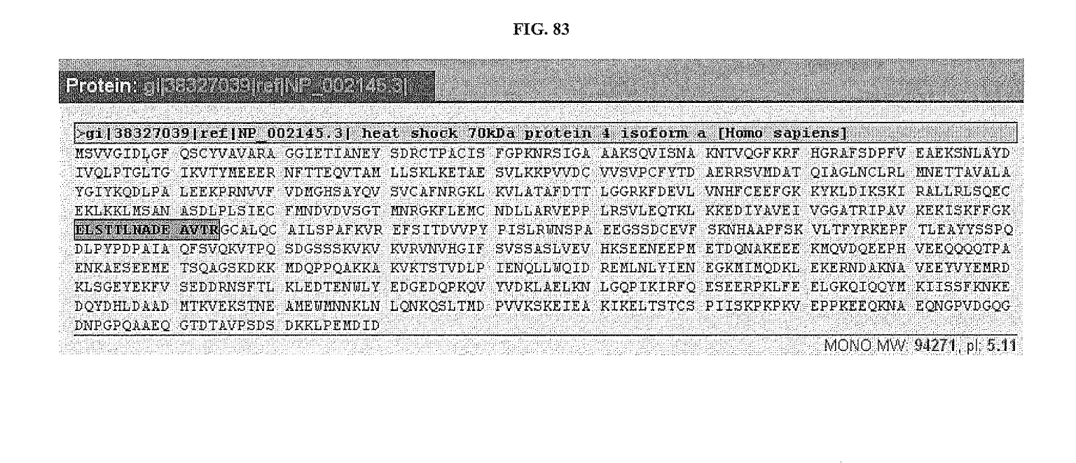

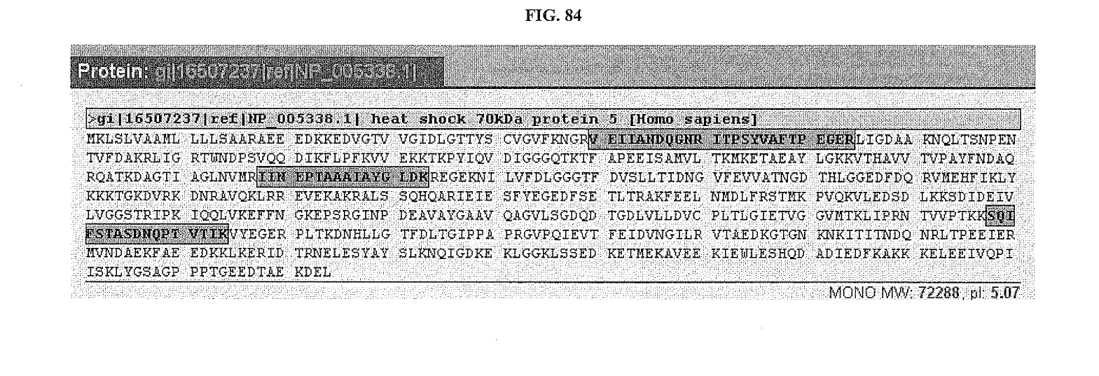

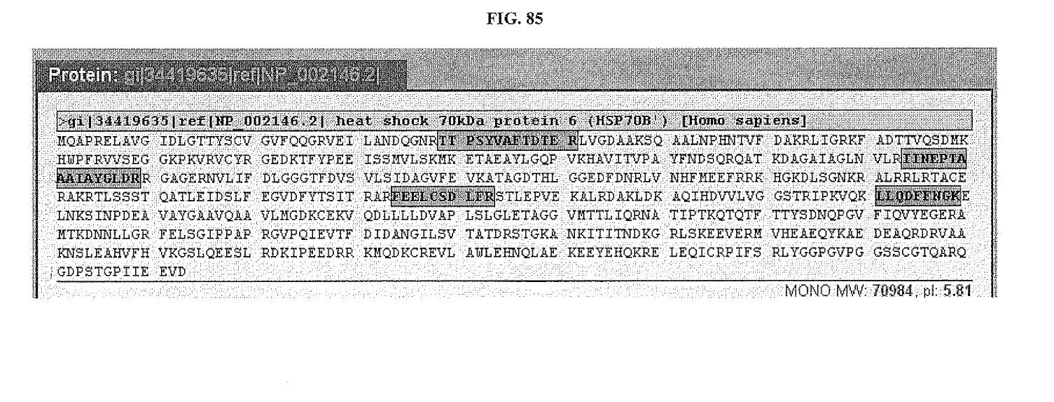

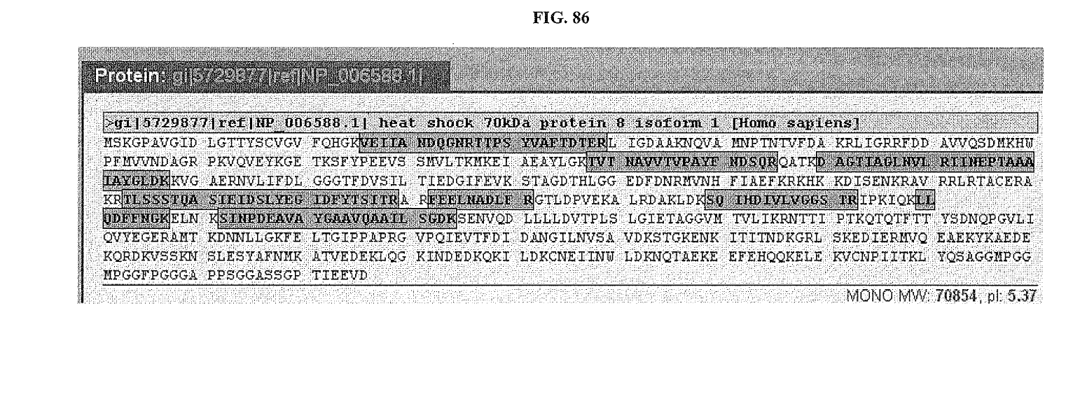

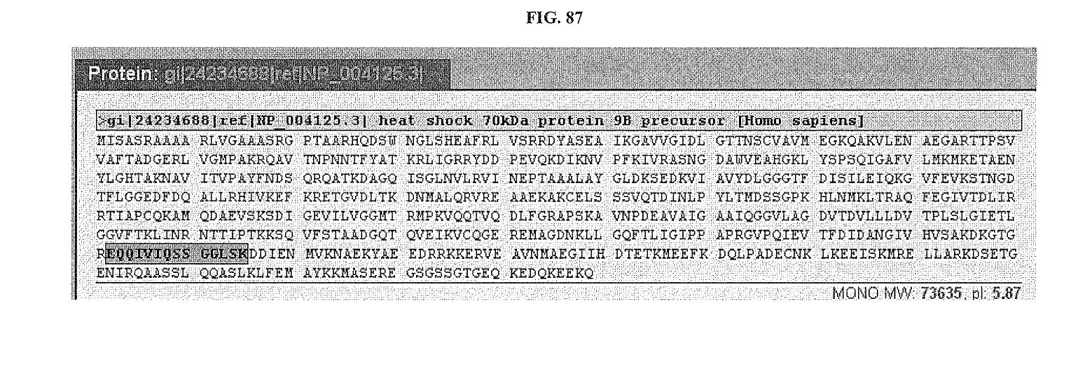

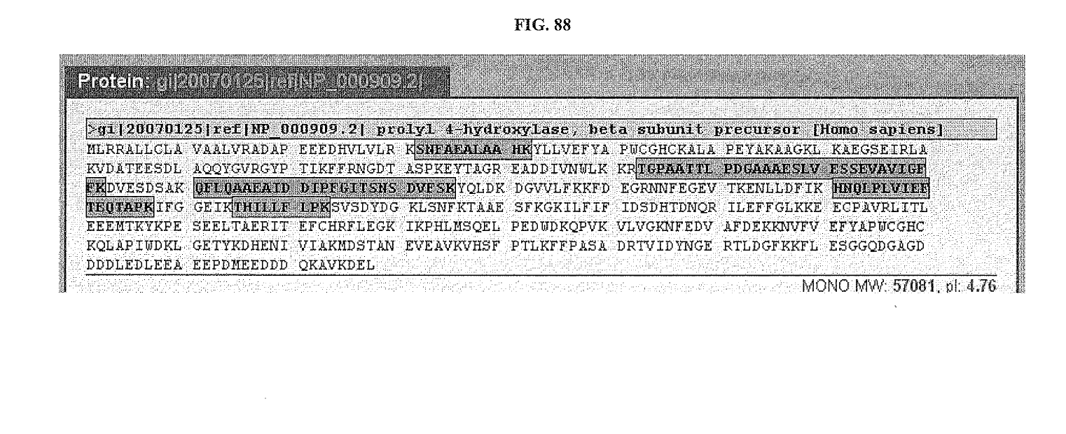

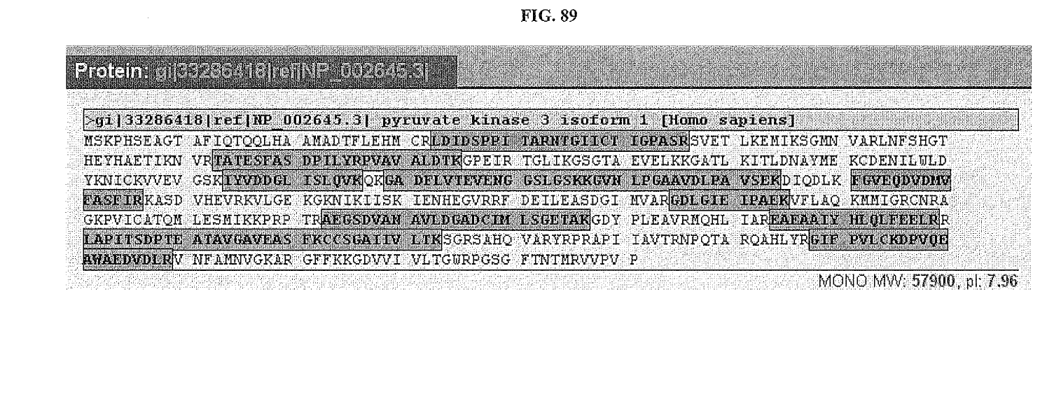

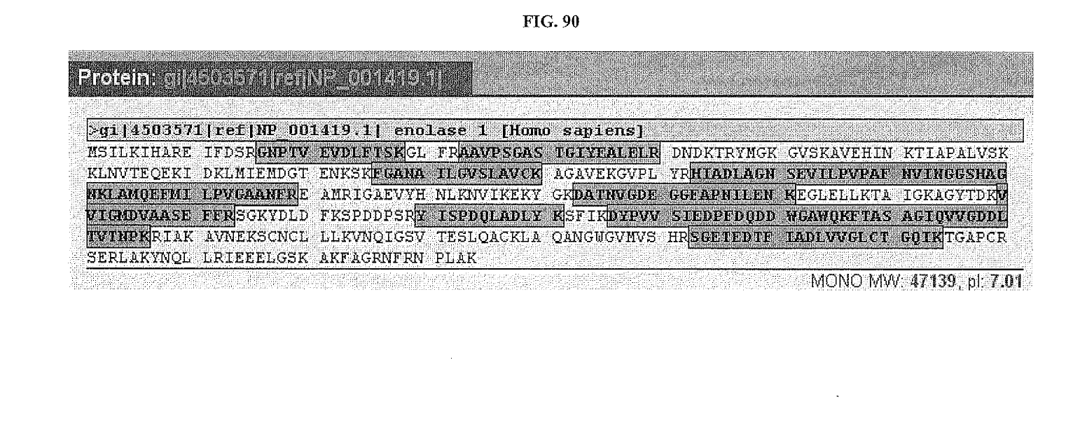

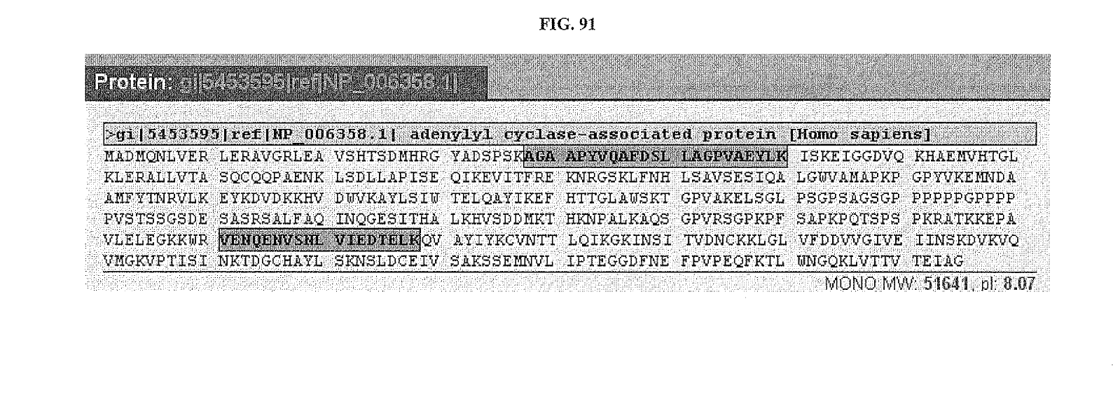

The observed peptide fragments, number of times the peptides were observed, and each fragments parent mass are provided infra in Tables 3 thru 6. The accompanying spectra for the detected peptides and summaries are shown in FIGS. 19 to 58, 60 to 67, and 69 to 80.

The isolation width was 2.0 amu. This value explains why some of the measured parent ions in Tables 3 thru 6 are different from the calculated values shown in the spectra in FIGS. 19 to 58, 60 to 67, and 69 to 80.

TABLE 3

AIF Peptides that Bound to BMS-214662 and Biotinylated
Analogues

| | | | SEQ | | | |
|---|---|---|---|---|---|---|
| | | | ID | Spectral | (m + 2H)2+ | Scan |
| Position | Mass | Peptide | NO: | Hits | Parent Mass | Number |
| 241-251 | 1266.6 | LNDGSQITYEK | 7 | 1 | 634.3 | 5742 |
| 321-333 | 1443.8 | ALGTEVIQLFPEK | 8 | 1 | 723.9 | 7197 |

| | |
|---|---|
| Total Hits = | 2 |
| Unique Hits = | 2 |

TABLE 4

HOP Peptides that Bound to BMS-214662 and Biotinylated
Analogues

| | | | | | | |
|---|---|---|---|---|---|---|
| | | | SEQ | | | |
| | | | ID | Spectral | (m + 2H)2+ | Scan |
| Position | Mass | Peptide | NO: | Hits | Parent Mass | Number |

| | | | | | | |
|---|---|---|---|---|---|---|
| 14-32 | 2009.0 | ALSVGNIDDALQCYSEAIK | 9 | 1 | 1034.9 | 7342 |
| 79-87 | 1003.6 | AAALEFLNR | 10 | 1 | 503.4 | 6917 |
| 101-109 | 1049.5 | HEANNPQLK | 11 | 1 | 526.3 | 5458 |
| 110-118 | 1046.5 | EGLQNMEAR | 12 | 4 | 532.6 | 5311 |
| 145-153 | 1064.6 | TLLSDPTYR | 13 | 2 | 534.2 | 6428 |
| 154-160 | 899.5 | ELIEQLR | 14 | 1 | 451.4 | 6500 |
| 161-169 | 958.5 | NKPSDLGTK | 15 | 2 | 480.5 | 5298 |
| 230-237 | 908.4 | ELGNDAYK | 16 | 3 | 455.4 | 5214 |
| 240-246 | 808.4 | DFDTALK | 17 | 1 | 405.5 | 5838 |
| 352-364 | 1487.8 | LAYINPDLALEEK | 18 | 2 | 745.2 | 6967 |
| 416-429 | 1666.8 | DCEECIQLEPTFIK | 19 | 1 | 892.3 | 6981 |
| 447-453 | 853.4 | AMDVYQK | 20 | 2 | 428.2 | 5393 |
| 454-462 | 950.4 | ALDLDSSCK | 21 | 2 | 505.3 | 5498 |
| 463-470 | 908.4 | EAADGYQR | 22 | 2 | 455.7 | 4970 |
| 506-513 | 1001.6 | LILEQMQK | 23 | 2 | 502.3 | 6650 |
| 514-523 | 1136.6 | DPQALSEHLK | 24 | 1 | 569.5 | 6705 |
| 524-530 | 768.5 | NPVIAQK | 25 | 1 | 385.6 | 4656 |
| 534-543 | 1099.6 | LMDVGLIAIR | 26 | 1 | 551.4 | 7142 |

| | |
|---|---|
| Total Hits = | 30 |
| Unique Hits = | 18 |

TABLE 5

Phosphatase 1G Peptides that Bound to BMS-214662 and
Biotinylated Analogues

| | | | | | | |
|---|---|---|---|---|---|---|
| | | | SEQ | | | |
| | | | ID | Spectral | (m + 2H)2+ | Scan |
| Position | Mass | Peptide | NO: | Hits | Parent Mass | Number |

| | | | | | | |
|---|---|---|---|---|---|---|
| 105-112 | 931.5 | LTTEEVIK | 27 | 2 | 467.3 | 5735 |
| 113-128 | 1799.9 | ELAQIAGRPTEDEDEK | 28 | 1 | 601.2* | 5340 |
| 321-337 | 1700.9 | EEPGSDSGTTAVVALIR | 29 | 1 | 851.9 | 6965 |
| 340-350 | 1142.6 | QLIVANAGDSR | 30 | 1 | 573.2 | 5851 |

| | |
|---|---|
| Total Hits = | 5 |
| Unique Hits = | 4 |

TABLE 6

PTPN6 Peptides that Bound to BMS-214662 and Biotinylated
Analogues

| | | | SEQ | | | |
|---|---|---|---|---|---|---|
| | | | ID | Spectral | (m + 2H)2+ | Scan |
| Position | Mass | Peptide | NO: | Hits | Parent Mass | Number |
| 10-21 | 1273.7 | DLSGLDAETLLK | 31 | 1 | 638.3 | 7025 |
| 199-212 | 1511.8 | TGIEEASGAFVYLR | 32 | 1 | 757.5 | 7072 |
| 220-228 | 1000.5 | VNAADIENR | 33 | 1 | 501.4 | 5505 |
| 295-310 | 1782.8 | DSNIPGSDYINANYIK | 34 | 1 | 892.8 | 6919 |
| 396-409 | 1539.8 | TLQVSPLDNGDLIR | 35 | 1 | 771.1 | 6961 |
| 479-488 | 1118.5 | GLDCDIDIQK | 36 | 1 | 589.3 | 6254 |

| | |
|---|---|
| Total Hits = | 6 |
| Unique Hits = | 6 |

TABLE 7

Hsp70 Peptides (from gi|NP_005336.2 and gi|NP_005337.1) that Bound to
BMS-214662 and Biotinylated Analogues

| | | | SEQ | | | |
|---|---|---|---|---|---|---|
| | | | ID | Spectral | (m + 2H)2+ | Scan |
| Position | Mass | Peptide | NO: | Hits | Parent Mass | Number |
| 26-36 | 1227.6 | VEIIANDQGNR | 38 | 1 | 614.8 | 28 |
| 37-49 | 1486.7 | TTPSYVAFTDTER | 39 | 1 | 744.3 | 291 |
| 57-71 | 1657.8 | NQVALNPQNTVFDAK | 40 | 1 | 829.9 | 312 |
| 129-155 | 3000.5 | EIAEAYLGYPVTNAVITVPAYFNDSQR | 41 | 1 | 150.3 | 630 |
| 160-171 | 1196.7 | DAGVIAGLNVLR | 42 | 1 | 599.3 | 407 |
| 172-187 | 1686.9 | IINEPTAAAIAYGLDR | 43 | 2 | 844.5, 554.0 | 452, 470 |
| 302-311 | 1257.6 | FEELCSDLFR | 44 | 1 | 658.3 | 462 |
| 349-357 | 1108.6 | LLQDFFNGR | 45 | 1 | 555.3 | 415 |
| 362-384 | 2303.2 | SINPDEAVAYGAAVQAAILMGDK | 46 | 2 | 1152.6, 768.7 | 686, 671 |
| 385-415 | 3179.7 | SENVQDLLLLDVAPLSLGLETAGGVMTALIK | 47 | 1 | 1060.9 | 887 |
| 424-447 | 2785.4 | QTQIFTTYSDNQPGVLIQVYEGER | 48 | 2 | 1393.7, 929.5 | 554, 559 |

| | |
|---|---|
| Total Hits = | 14 |
| Unique Hits = | 11 |

TABLE 8

Hsp70 Peptides (from gi|NP_068814.2) that Bound to
BMS-214662 and Biotinylated Analogues

| | | | SEQ | | | |
|---|---|---|---|---|---|---|
| | | | ID | Spectral | Parent | Scan |
| Position | Mass | Peptide | NO: | Hits | Mass | Number |
| 26-36 | 1227.6 | VEIIANDQGNR | 50 | 1 | 614.8 | 28 |
| 37-49 | 1486.7 | TTPSYVAFTDTER | 51 | 1 | 744.3 | 291 |
| 172-187 | 1686.9 | IINEPTAAAIAYGLDR | 52 | 2 | 844.5, 554.0 | 452, 470 |
| 302-311 | 1257.6 | FEELCSDLFR | 53 | 1 | 658.3 | 462 |
| 349-357 | 1108.6 | LLQDFFNGR | 54 | 1 | 555.3 | 415 |

| | |
|---|---|
| Total Hits = | 6 |
| Unique Hits = | 5 |

TABLE 9

Hsp70 Peptides (from gi|NP_002145.3) that Bound to
BMS-214662 and Biotinylated Analogues

| | | | SEQ | | | |
|---|---|---|---|---|---|---|
| | | | ID | Spectral | (m + 2H)2+ | Scan |
| Position | Mass | Peptide | NO: | Hits | Parent Mass | Number |
| 361-374 | 1518.8 | ELSTTLNADEAVTR | 56 | 1 | 725.9 | 583 |

| | |
|---|---|
| Total Hits = | 1 |
| Unique Hits = | 1 |

TABLE 10

Hsp70 Peptides (from gi|NP_005338.2) that Bound to
BMS-214662 and Biotinylated Analogues

| | | | SEQ | | | |
|---|---|---|---|---|---|---|
| | | | ID | Spectral | (m + 2H)2+ | Scan |
| Position | Mass | Peptide | NO: | Hits | Parent Mass | Number |
| 50-60 | 1227.6 | VEIIANDQGNR | 58 | 1 | 614.8 | 28 |
| 61-74 | 1565.8 | ITPSYVAFTPEGER | 59 | 1 | 783.9 | 360 |
| 198-213 | 1658.9 | IINEPTAAAIAYGLDK | 60 | 2 | 844.5, 554.0 | 452, 470 |
| 448-464 | 1835.9 | SQIFSTASDNQPTVTIK | 61 | 1 | 919.0 | 319 |

| | |
|---|---|
| Total Hits = | 5 |
| Unique Hits = | 4 |

TABLE 11

Hsp70 Peptides (from gi|NP_002146.2) that Bound to
BMS-214662 and Biotinylated Analogues

| | | | SEQ | | | |
|---|---|---|---|---|---|---|
| | | | ID | Spectral | (m + 2H)2+ | Scan |
| Position | Mass | Peptide | NO: | Hits | Parent Mass | Number |
| 39-51 | 1486.7 | TTPSYVAFTDTER | 63 | 1 | 744.3 | 291 |
| 174-189 | 1686.9 | IINEPTAAAIAYGLDR | 64 | 2 | 844.5, 554.0 | 452, 470 |
| 304-313 | 1257.6 | FEELCSDLFR | 65 | 1 | 658.3 | 462 |
| 351-359 | 1080.6 | LLQDFFNGK | 66 | 1 | 541.3 | 374 |

| | |
|---|---|
| Total Hits = | 5 |
| Unique Hits = | 4 |

TABLE 12

Hsp70 Peptides (from gi|NP_006588.1) that Bound to BMS-214662 and
Biotinylated Analogues

| | | | SEQ | | | |
|---|---|---|---|---|---|---|
| | | | ID | Spectral | (m + 2H)2+ | Scan |
| Position | Mass | Peptide | NO: | Hits | Parent Mass | Number |
| 26-36 | 1227.6 | VEIIANDQGNR | 68 | 1 | 614.8 | 28 |
| 37-49 | 1486.7 | TTPSYVAFTDTER | 69 | 1 | 744.3 | 291 |
| 138-155 | 1981,0 | TVTNAVVTVPAYFNDSQR | 70 | 1 | 661.3 | 431 |
| 160-171 | 1198.7 | DAGTIAGLNVLR | 71 | 1 | 600.3 | 334 |
| 172-187 | 1658.9 | IINEPTAAAIAYGLDK | 72 | 2 | 844.5, 554.0 | 452, 470 |
| 273-299 | 2996.5 | TLSSSTQASIEIDSLYEGIDFYTSITR | 73 | 1 | 1499.2 | 779 |
| 302-311 | 1252.6 | FEELNADLFR | 74 | 1 | 627.3 | 471 |
| 329-342 | 1480.8 | SQIHDIVLVGGSTR | 75 | 1 | 741.4 | 263 |
| 349-357 | 1080.6 | LLQDFFNGK | 76 | 1 | 541.3 | 374 |
| 362-384 | 2259.1 | SINPDEAVAYGAAVQAAILSGDK | 77 | 1 | 754.1 | 606 |

| | |
|---|---|
| Total Hits = | 11 |
| Unique Hits = | 10 |

TABLE 13

Hsp70 Peptides (from gi|NP_004125.3) that Bound to
BMS-214662 and Biotinylated Analogues

| | | | SEQ | | | |
|---|---|---|---|---|---|---|
| | | | ID | Spectral | (m + 2H)2+ | Scan |
| Position | Mass | Peptide | NO: | Hits | Parent Mass | Number |
| 542-555 | 1472.8 | EQQIVIQSSGGLSK | 77 | 1 | 737.4 | 183 |

| | |
|---|---|
| Total Hits = | 1 |
| Unique Hits = | 1 |

TABLE 14
Prolyl-4-Hydroxylase, Beta Peptides (from
gi|NP_000909.2) that Bound to BMS-214662 and
Biotinylated Analogues

| | | | SEQ |
|---|---|---|---|
| | | | ID |
| Position | Mass | Peptide | NO: |
| 32-42 | 1157.6 | SNFAEALAAHK | 79 |
| 133-162 | 2934.5 | TGPAATTLPDGAAAESLVESSEVAVIGFFK | 80 |
| 171-195 | 2712.3 | QFLQAAEAIDDIPFGITSNSDVFSK | 81 |
| 231-247 | 1964.0 | HNQLPLVIEFTEQTAPK | 82 |
| 255-263 | 1080.7 | THILLFLPK | 83 |

**Spectra, spectral hits, parent mass, and scan number were obtained but have not been provided.

TABLE 15
Pyruvate Kinase 3 Peptides (from
gi|NP_002645.3) that Bound to BMS-214662 and
Biotinylated Analogues.

| | | | SEQ ID |
|---|---|---|---|
| Position | Mass | Peptide | NO: |
| 33-43 | 1196.6 | LDIDSPPITAR | 85 |
| 44-56 | 1301.7 | NTGIICTIGPASR | 86 |
| 93-115 | 2464.3 | TATESFASDPILYRPVAVALDTK | 87 |
| 174-186 | 1461.8 | IYVDDGLISLQVK | 88 |
| 189-206 | 1778.9 | GADFLVTEVENGGSLGSK | 89 |
| 207-224 | 1764.0 | KGVNLPGAAVDLPAVSEK | 90 |
| 208-224 | 1635.9 | GVNLPGAAVDLPAVSEK | 91 |
| 231-246 | 1858.9 | FGVEQDVDMVFASFIR | 92 |
| 295-305 | 1140.6 | GDLGIEIPAEK | 93 |
| 343-367 | 2436.1 | AEGSDVANAVLDGADCIMLSGETAK | 94 |
| 384-399 | 1931.0 | EAEAAIYHLQLFEELR | 95 |
| 401-422 | 2174.1 | LAPITSDPTEATAVGAVEASFK | 96 |
| 423-433 | 1106.6 | CCSGAIIVLTK | 97 |
| 468-475 | 875.5 | GIFPVLCK | 98 |
| 476-489 | 1641.8 | DPVQEAWAEDVDLR | 99 |

**Spectra, spectral hits, parent mass, and scan number were obtained but have not been provided.

TABLE 16
Enolase 1 Peptides (from gi|NP_001419.1) that
Bound to BMS-214662 and Biotinylated Analogues.

| | | | SEQ |
|---|---|---|---|
| | | | ID |
| Position | Mass | Peptide | NO: |
| 16-28 | 1405.7 | GNPTVEVDLFTSK | 101 |
| 33-50 | 1803.9 | AAVPSGASTGIYEALELR | 102 |
| 106-120 | 1461.8 | FGANAILGVSLAVCK | 103 |
| 133-162 | 3010.6 | HIADLAGNSEVILPVPAFNVINGGSHAGNK | 104 |
| 163-179 | 1907.0 | LAMQEFMILPVGAANFR | 105 |
| 203-221 | 1959.9 | DATNVGDEGGFAPNILENK | 106 |
| 240-253 | 1539.8 | VVIGMDVAASEFFR | 107 |
| 270-281 | 1424.7 | YISPDQLADLYK | 108 |
| 286-306 | 2509.1 | DYPVVSIEDPFDQDDWGAWQK | 109 |
| 307-326 | 2032.1 | FTASAGIQVVGDDLTVTNPK | 110 |
| 373-394 | 2295.1 | SGETEDTFIADLVVGLCTGQIK | 111 |

**Spectra, spectral hits, parent mass, and scan number were obtained but have not been provided.

TABLE 17
Adenylate Cyclase Peptides (from
gi|NP_006358.1) that Bound to BMS-214662 and
Biotinylated Analogues.

| Position | Mass | Peptide | SEQ ID NO: |
|---|---|---|---|
| 38-60 | 2350.2 | AGAAPYVQAFDSLLAGPVAEYLK | 113 |
| 331-348 | 2072.0 | VENQENVSNLVIEDTELK | 114 |

**Spectra, spectral hits, parent mass, and scan number were obtained but have not been provided.

### Example 7

### Production of Antibodies Against Target Proteins

Antibodies against AIF, HOP, PTPN6, HSP70, Phosphatase I G, or other target proteins described elsewhere herein can be prepared by a variety of methods. For example, cells expressing a AIF, HOP, PTPN6, HSP70, Phosphatase 1G, or other target polypeptide can be administered to an animal to induce the production of sera containing polyclonal antibodies directed to the expressed polypeptides. In one aspect, the AIF, HOP, PTPN6, HSP70, Phosphatase 1G, or other target protein is prepared and isolated or otherwise purified to render it substantially free of natural contaminants, using techniques commonly practiced in the art. Such a preparation is then introduced into an animal in order to produce polyclonal antisera of greater specific activity for the expressed and isolated polypeptide.

In one aspect, the antibodies of the invention are monoclonal antibodies (or protein binding fragments thereof). Cells expressing the biomarker polypeptide can be cultured in any suitable tissue culture medium, however, it is preferable to culture cells in Earle's modified Eagle's medium supplemented to contain 10% fetal bovine serum (inactivated at about 56° C.), and supplemented to contain about 10 g/l nonessential amino acids, about 1.00 U/ml penicillin, and about 100 μg/ml streptomycin.

The splenocytes of immunized (and boosted) mice can be extracted and fused with a suitable myeloma cell line. Any suitable myeloma cell line can be employed in accordance with the invention, however, it is preferable to employ the parent myeloma cell line (SP2/0), available from the ATCC® (Manassas, Va.). After fusion, the resulting hybridoma cells are selectively maintained in HAT medium, and then cloned by limiting dilution as described by Wands et al. (Gastroenterology, 80:225-232 (1981)). The hybridoma cells obtained through such a selection are then assayed to identify those cell clones that secrete antibodies capable of binding to the polypeptide immunogen, or a portion thereof.

Alternatively, additional antibodies capable of binding to the AIF, HOP, PTPN6, HSP70, Phosphatase I G, or other target protein can be produced in a two-step procedure using anti-idiotypic antibodies. Such a method makes use of the fact that antibodies are themselves antigens and, therefore, it is possible to obtain an antibody that binds to a second antibody. In accordance with this method, protein specific antibodies can be used to immunize an animal, preferably a mouse. The splenocytes of such an immunized animal are then used to produce hybridoma cells, and the hybridoma cells are screened to identify clones that produce an antibody whose ability to bind to the protein-specific antibody can be blocked by the polypeptide. Such antibodies comprise anti-idiotypic antibodies to the protein-specific antibody and can be used to immunize an animal to induce the formation of further protein-specific antibodies.

The antibodies described herein can be labeled with a detectable moiety.

The detectable moiety should be capable of producing, either directly or indirectly, a detectable signal. For example, the detectable moiety may be a radioisotope, such as 2H, 14C, 32P, or 125I, a florescent or chemiluminescent compound, such as fluorescein isothiocyanate, rhodamine, or luciferin, or an enzyme, such as alkaline phosphatase, beta-galactosidase, green fluorescent protein, or horseradish peroxidase.

Any method known in the art for conjugating the antibody to the detectable moiety may be employed, including those methods described by Hunter et al., Nature, 144:945 (1962); David et al., Biochem., 13:1014 (1974); Pain et al., J. Immunol. Metho., 40:219 (1981); and Nygren, J. Histochem. Cytochem., 30:407 (1982).

The present invention encompasses antibodies that bind to at least onn epitope of the polypeptides disclosed herein, in particular to one or more of SEQ ID NOs:1-114. Such antibodies may be useful as therapeutics for inhibiting quiescent, non-proliferating cancer cells, or other cells described herein.

It will be clear that the invention may be practiced otherwise than as particularly described in the foregoing description and examples. Numerous modifications and variations of the present invention are possible in light of the above teachings and, therefore, are within the scope of the appended claims.

The entire disclosure of each document cited (including patents, patent applications, journal articles, abstracts, laboratory manuals, books, GENBANK® Accession numbers, SWISS-PROT® Accession numbers, or other disclosures) in the Background of the Invention, Detailed Description, Brief Description of the Figures, and Examples is hereby incorporated herein by reference in their entirety. Further, the Sequence Listing submitted herewith is incorporated herein by reference in its entirety.

What is claimed is:

1. A method of treating proliferative disease comprising the step of administering to a mammal in need thereof a therapeutically acceptable amount of a compound that induces caspase-independent apoptosis by agonizing the biological activity of AIF in a cell selected from the group consisting of: quiescent cells, quiescent tumor cells, tumor stem cells, and/or quiescent stem cells.

2. The method according to claim 1 wherein said compound induces apoptosis by agonizing the amount of unbound, AIF in said cell.

3. The method according to claim 1, wherein said compound induces apoptosis by increasing the ability of AIF to accumulate, localize, or translocate from the cytoplasm to the nucleus of said cell.

4. The method according to claim 1, wherein said compound induces apoptosis by selectively binding to a member of the group consisting of: AIF; HOP; phosphatase 1G; protein tyrosine phosphatase non-receptor type 6 isoform 2; Hsp70; tubulin, HSP60, adenyl cyclase AP, pyruvate kinase, alpha-enolase, 5′ methylthioadenosine phosphorylase, 14-3-3 Protein Sigma, Nit protein 2 (Nit-2), Prolyl-4-hydroxylase-beta, and eukaryotic translation elongation factor 1 alpha.

5. The method according to claim 1, wherein said proliferative disorder is cancer.

6. The method according to claim 1, wherein said mammal is a human, mouse, rat, or rabbit.

7. The method according to claim 2, wherein said agonizing of AIF is due to inhibiting the ability of Hsp70 to bind to and sequester the biological activity of AIF, wherein said inhibition results in AIF being free to accumulate, localize, or translocate into the nucleus.

8. The method according to claim 3, wherein said translocation of AIF is due to agonizing the ability of HOP to accumulate, localize, or translocate AIF into the nucleus.

9. The method according to claim 8, wherein said agonism of HOP results in an increased frequency to translocate AIF into the nucleus.

10. The method according to claim 1, wherein said compound is selected from the group consisting of small molecule, antibody, antisense molecule, RNAi molecule, adnectin, and domain antibody.

11. A method of screening to identify a compound useful for treating a proliferative disease comprising the steps of: (i) incubating quiescent cells, quiescent tumor cells, tumor stem cells, and/or quiescent stem cells with a test compound, (ii) determining whether apoptosis is induced in said cells, and (iii) confirming that said compound binds to a member of the group consisting of: AIF; HOP; phosphatase 1G; protein tyrosine phosphatase non-receptor type 6 isoform 2; Hsp70; tubulin, HSP60, adenyl cyclase AP, pyruvate kinase, alpha-enolase, 5′ methylthioadenosine phosphorylase, 14-3-3 Protein Sigma, Nit protein 2 (Nit-2), Prolyl-4-hydroxylase-beta, and eukaryotic translation elongation factor 1 alpha.

12. A method for identifying a compound useful for treatment of proliferative disease comprising the steps of (i) incubating a test compound with a target protein, (ii) identifying those compounds that bind to said target protein, (iii) and determining whether incubation of said target protein-binding compounds are capable of inducing apoptosis in quiescent cells, quiescent tumor cells, tumor stem cells, and/or quiescent stem cells with a test compound, wherein said target protein is selected from the group consisting of: binding AIF; HOP; phosphatase 1G; protein tyrosine phosphatase non-receptor type 6 isoform 2; Hsp70; tubulin, HSP60, adenyl cyclase AP, pyruvate kinase, alpha-enolase, 5′ methylthioadenosine phosphorylase, 14-3-3 Protein Sigma, Nit protein 2 (Nit-2), Prolyl-4-hydroxylase-beta, and eukaryotic translation elongation factor 1 alpha.

13. A method for identifying a compound useful for treatment of proliferative disease comprising the steps of: (i) incubating a cell with a compound, wherein said cell is capable of expressing a target protein either endogenously or recombinately, wherein said cell is further incubated with a labeled antibody specific to said target protein either prior to, during, or after incubation with said compound, and (ii) determining whether said compound increases the frequency or amount of AIF that is translocated to the nucleus, relative to a control cell that has not been exposed to said test compound, wherein said cells are quiescent cells, quiescent tumor cells, tumor stem cells, and quiescent stem cells, wherein said target protein is a member of the group consisting of: AIF; HOP; phosphatase 1G; protein tyrosine phosphatase non-receptor type 6 isoform 2; Hsp70; tubulin, HSP60, adenyl cyclase AP, pyruvate kinase, alpha-enolase, 5′ methylthioadenosine phosphorylase, 14-3-3 Protein Sigma, Nit protein 2 (Nit-2), Prolyl-4-hydroxylase-beta, and eukaryotic translation elongation factor 1 alpha.

14. A method of inducing apoptosis in a cell comprising administering a pharmaceutically acceptable amount of a compound according to formula I, 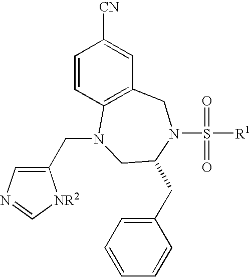
wherein R¹ is selected from the group consisting of: 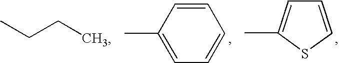
wherein R² is either H or CH₃, and wherein said cell is selected from the group consisting of: quiescent cells, quiescent tumor cells, tumor stem cells, and quiescent stem cells.


SEQUENCE LISTING (114 sequences)

SEQ ID NO: 1 - protein, 613 aa, Homo sapiens
MFRCGGLAAGALKQKLVPLVRTVCVRSPRQRNRLPGNLFQRWHVPLELQMTRQMASSGASGGKIDNSVLVLIVGLSTVGAGAYAYKTMKEDEKRYNERISGLGLTPEQKQKKAALSASEGEEVPQDKAPSHVPFLLIGGGTAAFAAARSIRARDPGARVLIVSEDPELPYMRPPLSKELWFSDDPNVTKTLRFKQWNGKERSIYFQPPSFYVSAQDLPHIENGGVAVLTGKKVVQLDVRDNMVKLNDGSQITYEKCLIATGGTPRSLSAIDRAGAEVKSRTTLFRKIGDFRSLEKISREVKSITIIGGGFLGSELACALGRKARALGTEVIQLFPEKGNMGKILPEYLSNWTMEKVRREGVKVMPNAIVQSVGVSSGKLLIKLKDGRKVETDHIVAAVGLEPNVELAKTGGLEIDSDFGGFRVNAELQARSNIWVAGDAACFYDIKLGRRRVEHHDHAVVSGRLAGENMTGAAKPYWHQSMFWSDLGPDVGYEAIGLVDSSLPTVGVFAKATAQDNPKSATEQSGTGIRSESETESEASEITIPPSTPAVPQAPVQGEDYGKGVIFYLRDKVVVGIVLWNIFNRMPIARKIIKDGEQHEDLNEVAKLFNIHED

SEQ ID NO: 2 - protein, 612 aa, Mus musculus
MFRCGGLAGAFKQKLVPLVRTVYVQRPKQRNRLPGNLFQQWRVPLELQMARQMASSGSSGGKMDNSVLVLIVGLSTIGAGAYAYKTIKEDQKRYNERVMGLGLSPEEKQRRAIASATEGGSVPQIRAPSHVPFLLIGGGTAAFAAARSIRARDPGARVLIVSEDPELPYMRPPLSKELWFSDDPNVTKTLQFRQWNGKERSIYFQPPSFYVSAQDLPNIENGGVAVLTGKKVVHLDVRGNMVKLNDGSQITFEKCLIATGGTPRSLSAIDRAGAEVKSRTTLFRKIGDFRALEKISREVKSITVIGGGFLGSELACALGRKSQASGIEVIQLFPEKGNMGKILPQYLSNWTMEKVKREGVKVMPNAIVQSVGVSGGRLLIKLKDGRKVETDHIVTAVGLEPNVELAKTGGLEIDSDFGGFRVNAELQARSNIWVAGDAACFYDIKLGRRRVEHHDHAVVSGRLAGENMTGAAKPYWHQSMFWSDLGPDVGYEAIGLVDSSLPTVGVFAKATAQDNPKSATEQSGTGIRSESETESEASEITIPPSAPAVPQVPVEGEDYGKGVIFYLRDKVVVGIVLWNVFNRMPIARKIIKDGEQHEDLNEVAKLFNIHED

SEQ ID NO: 3 - protein, 410 aa, Homo sapiens
MANHVAIIGNGVAGFTTAQALRAEGYEGRISLIGEEQHLPYDRPSLSKAVLDGSFEQPPRLAEADWYSEASIEMLTGSEVTDLDTQKKMISLNDGSTISADAIVIATGSRARMLSLPGSQLPGVVTLRTYGDVQLLRDSWTPNTRLLIVGGGLIGCEVATTARKLGLSVTILEAGDELLVRVLGRRIGAWLRGLLTEQGVQVELKTGVSGFSGEGQLEKVMVNDGRSFIADNALICVGADPADQLARQAGLECDRGVVVDHRGATSAKGIFAVGDVATWPLHSGGKRSLETYMNAQRQATAVAKAILGKEVSAPQLPVSWTEIAGHRMQMAGDIEGPGEYVLRGTLGIGSALLFRLLDGRIQAVVAVDAPRDFALANRLVEAQVIIEPEKLADVSNNMRDIVRANEGNQK

SEQ ID NO: 4 - protein, 543 aa, Homo sapiens
MEQVNELKEKGNKALSVGNIDDALQCYSEAIKLDPHNHVLYSNRSAAYAKKGDYQKAYEDGCKTVDLKPDWGKGYSRKAAALEFLNRFEEAKRTYEEGLKHEANNPQLKEGLQNMEARLAERKFMNPFNMPNLYQKLESDPRTRTLLSDPTYRELIEQLRNKPSDLGTKLQDPRIMTTLSVLLGVDLGSMDEEEEIATPPPPPPPKKETKPEPMEEDLPENKKQALKEKELGNDAYKKKDFDTALKHYDKAKELDPTNMTYITNQAAVYFEKGDYNKCRELCEKAIEVGRENREDYRQIAKAYARIGNSYFKEEKYKDAIHFYNKSLAEHRTPDVLKKCQQAEKILKEQERLAYINPDLALEEKNKGNECFQKGDYPQAMKHYTEAIKRNPKDAKLYSNRAACYTKLLEFQLALKDCEECIQLEPTFIKGYTRKAAALEAMKDYTKAMDVYQKALDLDSSCKEAADGYQRCMMAQYNRHDSPEDVKRRAMADPEVQQIMSDPAMRLILEQMQKDPQALSEHLKNPVIAQKIQKLMDVGLIAIR

SEQ ID NO: 5 - protein, 546 aa, Homo sapiens
MGAYLSQPNTVKCSGDGVGAPRLPLPYGFSAMQGWRVSMEDAHNCIPELDSETAMFSVYDGHGGEEVALYCAKYLPDIIKDQKAYKEGKLQKALEDAFLAIDAKLTTEEVIKELAQIAGRPTEDEDEKEKVADEDDVDNEEAALLHEEATMTIEELLTRYGQNCHKGPPHSKSGGGTGEEPGSQGLNGEAGPEDSTRETPSQENGPTAKAYTGFSSNSERGTEAGQVGEPGIPTGEAGPSCSSASDKLPRVAKSKFFEDSEDESDEAEEEEEDSEECSEEEDGYSSEEAENEEDEDDTEEAEEDDEEEEEEMMVPGMEGKEEPGSDSGTTAVVALIRGKQLIVANAGDSRCVVSEAGKALDMSYDHKPEDEVELARIKNAGGKVTMDGRVNGGLNLSRAIGDHFYKRNKNLPPEEQMISALPDIKVLTLTDDHEFMVIACDGIWNVMSSQEVVDFIQSKISQRDENGELRLLSSIVEELLDQCLAPDTSGDGTGCDNMTCIIICFKPRNTAELQPESGKRKLEEVLSTEGAEENGNSDKKKKAKRD

SEQ ID NO: 6 - protein, 597 aa, Homo sapiens
MLSRGWFHRDLSGLDAETLLKGRGVHGSFLARPSRKNQGDFSLSVRVGDQVTHIRIQNSGDFYDLYGGEKFATLTELVEYYTQQQGVLQDRDGTIIHLKYPLNCSDPTSERWYHGHMSGGQAETLLQAKGEPWTFLVRESLSQPGDFVLSVLSDQPKAGPGSPLRVTHIKVMCEGGRYTVGGLETFDSLTDLVEHFKKTGIEEASGAFVYLRQPYYATRVNAADIENRVLELNKKQESEDTAKAGFWEEFESLQKQEVKNLHQRLEGQRPENKGKNRYKNILPFDHSRVILQGRDSNIPGSDYINANYIKNQLLGPDENAKTYIASQGCLEATVNDFWQMAWQENSRVIVMTTREVEKGRNKCVPYWPEVGMQRAYGPYSVTNCGEHDTTEYKLRTLQVSPLDNGDLIREIWHYQYLSWPDHGVPSEPGGVLSFLDQINQRQESLPHAGPIIVHCSAGIGRTGTIIVIDMLMENISTKGLDCDIDIQKTIQMVRAQRSGMVQTEAQYKFIYVAIAQFIETTKKKLEVLQSQKGQESEYGNITYPPAMKNAHAKASRTSSKHKEDVYENLHTKNKREEKVKKQRSADKEKSKGSLKRK

SEQ ID NO: 7 - protein, 11 aa, Homo sapiens
LNDGSQITYEK

SEQ ID NO: 8 - protein, 13 aa, Homo sapiens
ALGTEVIQLFPEK

SEQ ID NO: 9 - protein, 19 aa, Homo sapiens
ALSVGNIDDALQCYSEAIK

SEQ ID NO: 10 - protein, 9 aa, Homo sapiens
AAALEFLNR

SEQ ID NO: 11 - protein, 9 aa, Homo sapiens
HEANNPQLK

SEQ ID NO: 12 - protein, 9 aa, Homo sapiens
EGLQNMEAR

SEQ ID NO: 13 - protein, 9 aa, Homo sapiens
TLLSDPTYR

SEQ ID NO: 14 - protein, 7 aa, Homo sapiens
ELIEQLR

SEQ ID NO: 15 - protein, 9 aa, Homo sapiens
NKPSDLGTK

SEQ ID NO: 16 - protein, 8 aa, Homo sapiens
ELGNDAYK

SEQ ID NO: 17 - protein, 7 aa, Homo sapiens
DFDTALK

SEQ ID NO: 18 - protein, 13 aa, Homo sapiens
LAYINPDLALEEK

SEQ ID NO: 19 - protein, 14 aa, Homo sapiens
DCEECIQLEPTFIK

SEQ ID NO: 20 - protein, 7 aa, Homo sapiens
AMDVYQK

SEQ ID NO: 21 - protein, 9 aa, Homo sapiens
ALDLDSSCK

SEQ ID NO: 22 - protein, 8 aa, Homo sapiens
EAADGYQR

SEQ ID NO: 23 - protein, 8 aa, Homo sapiens
LILEQMQK

SEQ ID NO: 24 - protein, 10 aa, Homo sapiens
DPQALSEHLK

SEQ ID NO: 25 - protein, 7 aa, Homo sapiens
NPVIAQK

SEQ ID NO: 26 - protein, 10 aa, Homo sapiens
LMDVGLIAIR

SEQ ID NO: 27 - protein, 8 aa, Homo sapiens
LTTEEVIK

SEQ ID NO: 28 - protein, 16 aa, Homo sapiens
ELAQIAGRPTEDEDEK

SEQ ID NO: 29 - protein, 17 aa, Homo sapiens
EEPGSDSGTTAVVALIR

SEQ ID NO: 30 - protein, 11 aa, Homo sapiens
QLIVANAGDSR

SEQ ID NO: 31 - protein, 12 aa, Homo sapiens
DLSGLDAETLLK

SEQ ID NO: 32 - protein, 14 aa, Homo sapiens
TGIEEASGAFVYLR

SEQ ID NO: 33 - protein, 9 aa, Homo sapiens
VNAADIENR

SEQ ID NO: 34 - protein, 16 aa, Homo sapiens
DSNIPGSDYINANYIK

SEQ ID NO: 35 - protein, 14 aa, Homo sapiens
TLQVSPLDNGDLIR

SEQ ID NO: 36 - protein, 10 aa, Homo sapiens
GLDCDIDIQK

SEQ ID NO: 37 - protein, 641 aa, Homo sapiens
MAKAAAIGIDLGTTYSCVGVFQHGKVEIIANDQGNRTTPSYVAFTDTERLIGDAAKNQVALNPQNTVFDAKRLIGRKFGDPVVQSDMKHWPFQVINDGDKPKVQVSYKGDTKAFYPEEISSMVLTKMKEIAEAYLGYPVTNAVITVPAYFNDSQRQATKDAGVIAGLNVLRIINEPTAAAIAYGLDRTGKGERNVLIFDLGGGTFDVSILTIDDGIFEVKATAGDTHLGGEDFDNRLVNHFVEEFKRKHKKDISQNKRAVRRLRTACERAKRTLSSSTQASLEIDSLFEGIDFYTSITRARFEELCSDLFRSTLEPVEKALRDAKLDKAQIHDLVLVGGSTRIPKVQKLLQDFFNGRDLNKSINPDEAVAYGAAVQAAILMGDKSENVQDLLLLDVAPLSLGLETAGGVMTALIKRNSTIPTKQTQIFTTYSDNQPGVLIQVYEGERAMTKDNNLLGRFELSGIPPAPRGVPQIEVTFDIDANGILNVTATDKSTGKANKITITNDKGRLSKEEIERMVQEAEKYKAEDEVQRERVSAKNALESYAFNMKSAVEDEGLKGKISEADKKKVLDKCQEVISWLDANTLAEKDEFEHKRKELEQVCNPIISGLYQGAGGPGPGGFGAQGPKGGSGSGPTIEEVD

SEQ ID NO: 38 - protein, 11 aa, Homo sapiens
VEIIANDQGNR

SEQ ID NO: 39 - protein, 13 aa, Homo sapiens
TTPSYVAFTDTER

SEQ ID NO: 40 - protein, 15 aa, Homo sapiens
NQVALNPQNTVFDAK

SEQ ID NO: 41 - protein, 27 aa, Homo sapiens
EIAEAYLGYPVTNAVITVPAYFNDSQR

SEQ ID NO: 42 - protein, 12 aa, Homo sapiens
DAGVIAGLNVLR

SEQ ID NO: 43 - protein, 16 aa, Homo sapiens
IINEPTAAAIAYGLDR

SEQ ID NO: 44 - protein, 10 aa, Homo sapiens
FEELCSDLFR

SEQ ID NO: 45 - protein, 9 aa, Homo sapiens
LLQDFFNGR

SEQ ID NO: 46 - protein, 23 aa, Homo sapiens
SINPDEAVAYGAAVQAAILMGDK

SEQ ID NO: 47 - protein, 31 aa, Homo sapiens
SENVQDLLLLDVAPLSLGLETAGGVMTALIK

SEQ ID NO: 48 - protein, 24 aa, Homo sapiens
QTQIFTTYSDNQPGVLIQVYEGER

SEQ ID NO: 49 - protein, 639 aa, Homo sapiens
MSARGPAIGIDLGTTYSCVGVFQHGKVEIIANDQGNRTTPSYVAFTDTERLIGDAAKNQVAMNPTNTIFDAKRLIGRKFEDATVQSDMKHWPFRVVSEGGKPKVQVEYKGETKTFFPEEISSMVLTKMKEIAEAYLGGKVHSAVITVPAYFNDSQRQATKDAGTITGLNVLRIINEPTAAAIAYGLDKKGCAGGEKNVLIFDLGGGTFDVSILTIEDGIFEVKSTAGDTHLGGEDFDNRMVSHLAEEFKRKHKKDIGPNKRAVRRLRTACERAKRTLSSSTQASIEIDSLYEGVDFYTSITRARFEELNADLFRGTLEPVEKALRDAKLDKGQIQEIVLVGGSTRIPKIQKLLQDFFNGKELNKSINPDEAVAYGAAVQAAILIGDKSENVQDLLLLDVTPLSLGIETAGGVMTPLIKRNTTIPTKQTQTFTTYSDNQSSVLVQVYEGERAMTKDNNLLGKFDLTGIPPAPRGVPQIEVTFDIDANGILNVTAADKSTGKENKITITNDKGRLSKDDIDRMVQEAERYKSEDEANRDRVAAKNALESYTYNIKQTVEDEKLRGKISEQDKNKILDKCQEVINWLDRNQMAEKDEYEHKQKELERVCNPIISKLYQGGPGGGSGGGGSGASGGPTIEEVD

SEQ ID NO: 50 - protein, 11 aa, Homo sapiens
VEIIANDQGNR

SEQ ID NO: 51 - protein, 13 aa, Homo sapiens
TTPSYVAFTDTER

SEQ ID NO: 52 - protein, 16 aa, Homo sapiens
IINEPTAAAIAYGLDR

SEQ ID NO: 53 - protein, 10 aa, Homo sapiens
FEELCDSLFR

SEQ ID NO: 54 - protein, 9 aa, Homo sapiens
LLQDFFNGR

SEQ ID NO: 55 - protein, 840 aa, Homo sapiens
MSVVGIDLGFQSCYVAVARAGGIETIANEYSDRCTPACISFGPKNRSIGAAAKSQVISNAKNTVQGFKRFHGRAFSDPFVEAEKSNLAYDIVQLPTGLTGIKVTYMEEERNFTTEQVTAMLLSKLKETAESVLKKPVVDCVVSVPCFYTDAERRSVMDATQIAGLNCLRLMNETTAVALAYGIYKQDLPALEEKPRNVVFVDMGHSAYQVSVCAFNRGKLKVLATAFDTTLGGRKFDEVLVNHFCEEFGKKYKLDIKSKIRALLRLSQECEKLKKLMSANASDLPLSIECFMNDVDVSGTMNRGKFLEMCNDLLARVEPPLRSVLEQTKLKKEDIYAVEIVGGATRIPAVKEKISKFFGKELSTTLNADEAVTRGCALQCAILSPAFKVREFSITDVVPYPISLRWNSPAEEGSSDCEVFSKNHAAPFSKVLTFYRKEPFTLEAYYSSPQDLPYPDPAIAQFSVQKVTPQSDGSSSKVKVKVRVNVHGIFSVSSASLVEVHKSEENEEPMETDQNAKEEEKMQVDQEEPHVEEQQQQTPAENKAESEEMETSQAGSKDKKMDQPPQAKKAKVKTSTVDLPIENQLLWQIDREMLNLYIENEGKMIMQDKLEKERNDAKNAVEEYVYEMRDKLSGEYEKFVSEDDRNSFTLKLEDTENWLYEDGEDQPKQVYVDKLAELKNLGQPIKIRFQESEERPKLFEELGKQIQQYMKIISSFKNKEDQYDHLDAADMTKVEKSTNEAMEWMNNKLNLQNKQSLTMDPVVKSKEIEAKIKELTSTCSPIISKPKPKVEPPKEEQKNAEQNGPVDGQGDNPGPQAAEQGTDTAVPSDSDKKLPEMDID

SEQ ID NO: 56 - protein, 14 aa, Homo sapiens
ELSTTLNADEAVTR

SEQ ID NO: 57 - protein, 654 aa, Homo sapiens
MKLSLVAAMLLLLSAARAEEEDKKEDVGTVVGIDLGTTYSCVGVFKNGRVEIIANDQGNRITPSYVAFTPEGERLIGDAAKNQLTSNPENTVFDAKRLIGRTWNDPSVQQDIKFLPFKVVEKKTKPYIQVDIGGGQTKTFAPEEISAMVLTKMKETAEAYLGKKVTHAVVTVPAYFNDAQRQATKDAGTIAGLNVMRIINEPTAAAIAYGLDKREGEKNILVFDLGGGTFDVSLLTIDNGVFEVVATNGDTHLGGEDFDQRVMEHFIKLYKKKTGKDVRKDNRAVQKLRREVEKAKRALSSQHQARIEIESFYEGEDFSETLTRAKFEELNMDLFRSTMKPVQKVLEDSDLKKSDIDEIVLVGGSTRIPKIQQLVKEFFNGKEPSRGINPDEAVAYGAAVQAGVLSGDQDTGDLVLLDVCPLTLGIETVGGVMTKLIPRNTVVPTKKSQIFSTASDNQPTVTIKVYEGERPLTKDNHLLGTFDLTGIPPAPRGVPQIEVTFEIDVNGILRVTAEDKGTGNKNKITITNDQNRLTPEEIERMVNDAEKFAEEDKKLKERIDTRNELESYAYSLKNQIGDKEKLGGKLSSEDKETMEKAVEEKIEWLESHQDADIEDFKAKKKELEEIVQPIISKLYGSAGPPPTGEEDTAEKDEL

SEQ ID NO: 58 - protein, 11 aa, Homo sapiens
VEIIANDQGNR

SEQ ID NO: 59 - protein, 14 aa, Homo sapiens
ITPSYVAFTPEGER

SEQ ID NO: 60 - protein, 16 aa, Homo sapiens
IINEPTAAAIAYGLDK

SEQ ID NO: 61 - protein, 17 aa, Homo sapiens
SQIFSTASDNQPTVTIK

SEQ ID NO: 62 - protein, 643 aa, Homo sapiens
MQAPRELAVGIDLGTTYSCVGVFQQGRVEILANDQGNRTTPSYVAFTDTERLVGDAAKSQAALNPHNTVFDAKRLIGRKFADTTVQSDMKHWPFRVVSEGGKPKVRVCYRGEDKTFYPEEISSMVLSKMKETAEAYLGQPVKHAVITVPAYFNDSQRQATKDAGAIAGLNVLRIINEPTAAAIAYGLDRRGAGERNVLIFDLGGGTFDVSVLSIDAGVFEVKATAGDTHLGGEDFDNRLVNHFMEEFRRKHGKDLSGNKRALRRLRTACERAKRTLSSSTQATLEIDSLFEGVDFYTSITRARFEELCSDLFRSTLEPVEKALRDAKLDKAQIHDVVLVGGSTRIPKVQKLLQDFFNGKELNKSINPDEAVAYGAAVQAAVLMGDKCEKVQDLLLLDVAPLSLGLETAGGVMTTLIQRNATIPTKQTQTFTTYSDNQPGVFIQVYEGERAMTKDNNLLGRFELSGIPPAPRGVPQIEVTFDIDANGILSVTATDRSTGKANKITITNDKGRLSKEEVERMVHEAEQYKAEDEAQRDRVAAKNSLEAHVFHVKGSLQEESLRDKIPEEDRRKMQDKCREVLAWLEHNQLAEKEEYEHQKRELEQICRPIFSRLYGGPGVPGGSSCGTQARQGDPSTGPIIEEVD

SEQ ID NO: 63 - protein, 13 aa, Homo sapiens
TTPSYVAFTDTER

SEQ ID NO: 64 - protein, 16 aa, Homo sapiens
IINEPTAAAIAYGLDR

SEQ ID NO: 65 - protein, 10 aa, Homo sapiens
FEELCSDLFR

SEQ ID NO: 66 - protein, 9 aa, Homo sapiens
LLQDFFNGK

SEQ ID NO: 67 - protein, 646 aa, Homo sapiens
MSKGPAVGIDLGTTYSCVGVFQHGKVEIIANDQGNRTTPSYVAFTDTERLIGDAAKNQVAMNPTNTVFDAKRLIGRRFDDAVVQSDMKHWPFMVVNDAGRPKVQVEYKGETKSFYPEEVSSMVLTKMKEIAEAYLGKTVTNAVVTVPAYFNDSQRQATKDAGTIAGLNVLRIINEPTAAAIAYGLDKKVGAERNVLIFDLGGGTFDVSILTIEDGIFEVKSTAGDTHLGGEDFDNRMVNHFIAEFKRKHKKDISENKRAVRRLRTACERAKRTLSSSTQASIEIDSLYEGIDFYTSITRARFEELNADLFRGTLDPVEKALRDAKLDKSQIHDIVLVGGSTRIPKIQKLLQDFFNGKELNKSINPDEAVAYGAAVQAAILSGDKSENVQDLLLLDVTPLSLGIETAGGVMTVLIKRNTTIPTKQTQTFTTYSDNQPGVLIQVYEGERAMTKDNNLLGKFELTGIPPAPRGVPQIEVTFDIDANGILNVSAVDKSTGKENKITITNDKGRLSKEDIERMVQEAEKYKAEDEKQRDKVSSKNSLESYAFNMKATVEDEKLQGKINDEDKQKILDKCNEIINWLDKNQTAEKEEFEHQQKELEKVCNPIITKLYQSAGGMPGGMPGGFPGGGAPPSGGASSGPTIEEVD

SEQ ID NO: 68 - protein, 11 aa, Homo sapiens
VEIIANDQGNR

SEQ ID NO: 69 - protein, 13 aa, Homo sapiens
TTPSYVAFTDTER

SEQ ID NO: 70 - protein, 18 aa, Homo sapiens
TVTNAVVTNPAYFNDSQR

SEQ ID NO: 71 - protein, 12 aa, Homo sapiens
DAGTIAGLNVLR

SEQ ID NO: 72 - protein, 15 aa, Homo sapiens
IIEPTAAAIAYGLDK

SEQ ID NO: 73 - protein, 27 aa, Homo sapiens
TLSSSTQASIEIDSLYEFIDFYTSITR

SEQ ID NO: 74 - protein, 10 aa, Homo sapiens
FEELNADLFR

SEQ ID NO: 75 - protein, 14 aa, Homo sapiens
SQIHDIVLVGGSTR

SEQ ID NO: 76 - protein, 679 aa, Homo sapiens
MISASRAAAARLVGAAASRGPTAARHQDSWNGLSHEAFRLVSRRDYASEAIKGAVVGIDLGTTNSCVAVMEGKQAKVLENAEGARTTPSVVAFTADGERLVGMPAKRQAVTNPNNTFYATKRLIGRRYDDPEVQKDIKNVPFKIVRASNGDAWVEAHGKLYSPSQIGAFVLMKMKETAENYLGHTAKNAVITVPAYFNDSQRQATKDAGQISGLNVLRVINEPTAAALAYGLDKSEDKVIAVYDLGGGTFDISILEIQKGVFEVKSTNGDTFLGGEDFDQALLRHIVKEFKRETGVDLTKDNMALQRVREAAEKAKCELSSSVQTDINLPYLTMDSSGPKHLNMKLTRAQFEGIVTDLIRRTIAPCQKAMQDAEVSKSDIGEVILVGGMTRMPKVQQTVQDLFGRAPSKAVNPDEAVAIGAAIQGGVLAGDVTDVLLLDVTPLSLGIETLGGVFTKLINRNTTIPTKKSQVFSTAADGQTQVEIKVCQGEREMAGDNKLLGQFTLIGIPPAPRGVPQIEVTFDIDANGIVHVSAKDKGTGREQQIVIQSSGGLSKDDIENMVKNAEKYAEEDRRKKERVEAVNMAEGIIHDTETKMEEFKDQLPADECNKLKEEISKMRELLARKDSETGENIRQAASSLQQASLKLFEMAYKKMASEREGSGSSGTGEQKEDQKEEKQ

SEQ ID NO: 77 - protein, 14 aa, Homo sapiens
EQQIVIQSSGGLSK

SEQ ID NO: 78 - protein, 508 aa, Homo sapiens
MLRRALLCLAVAALVRADAPEEEDHVLVLRKSNFAEALAAHKYLLVEFYAPWCGHCKALAPEYAKAAGKLKAEGSEIRLAKVDATEESDLAQQYGVRGYPTIKFFRNGDTASPKEYTAGREADDIVNWLKKRTGPAATTLPDGAAAESLVESSEVAVIGFFKDVESDSAKQFLQAAEAIDDIPFGITSNSDVFSKYQLDKDGVVLFKKFDEGRNNFEGEVTKENLLDFIKHNQLPLVIEFTEQTAPKIFGGEIKTHILLFLPKSVSDYDGKLSNFKTAAESFKGKILFIFIDSDHTDNQRILEFFGLKKEECPAVRLITLEEEMTKYKPESEELTAERITEFCHRFLEGKIKPHLMSQELPEDWDKQPVKVLVGKNFEDVAFDEKKNVFVEFYAPWCGHCKQLAPIWDKLGETYKDHENIVIAKMDSTANEVEAVKVHSFPTLKFFPASADRTVIDYNGERTLDGFKKFLESGGQDGAGDDDDLEDLEEAEEPDMEEDDDQKAVKDEL

SEQ ID NO: 79 - protein, 11 aa, Homo sapiens
SNFAEALAAHK

SEQ ID NO: 80 - protein, 30 aa, Homo sapiens
TGPAATTLPDGAAAESLVESSEVAVIGFFK

SEQ ID NO: 81 - protein, 25 aa, Homo sapiens
QFLQAAEAIDDIPFGITSNSDVFSK

SEQ ID NO: 82 - protein, 17 aa, Homo sapiens
HNQLPLVIEFTEQTAPK

SEQ ID NO: 83 - protein, 9 aa, Homo sapiens
THILLFLPK

SEQ ID NO: 84 - protein, 531 aa, Homo sapiens
MSKPHSEAGTAFIQTQQLHAAMADTFLEHMCRLDIDSPPITARNTGIICTIGPASRSVETLKEMIKSGMNVARLNFSHGTHEYHAETIKNVRTATESFASDPILYRPVAVALDTKGPEIRTGLIKGSGTAEVELKKGATLKITLDNAYMEKCDENILWLDYKNICKVVEVGSKIYVDDGLISLQVKQKGADFLVTEVENGGSLGSKKGVNLPGAAVDLPAVSEKDIQDLKFGVEQDVDMVFASFIRKASDVHEVRKVLGEKGKNIKIISKIENHEGVRRFDEILEASDGIMVARGDLGIEIPAEKVFLAQKMMIGRCNRAGKPVICATQMLESMIKKPRPTRAEGSDVANAVLDGADCIMLSGETAKGDYPLEAVRMQHLIAREAEAAIYHLQLFEELRRLAPITSDPTEATAVGAVEASFKCCSGAIIVLTKSGRSAHQVARYRPRAPIIAVTRNPQTARQAHLYRGIFPVLCKDPVQEAWAEDVDLRVNFAMNVGKARGFFKKGDVVIVLTGWRPGSGFTNTMRVVPVP

SEQ ID NO: 85 - protein, 11 aa, Homo sapiens
LDIDSPPITAR

SEQ ID NO: 86 - protein, 13 aa, Homo sapiens
NTGIICTIGPASR

SEQ ID NO: 87 - protein, 23 aa, Homo sapiens
TATESFASDPILYRPVAVALDTK

SEQ ID NO: 88 - protein, 13 aa, Homo sapiens
IYVDDGLISLQVK

SEQ ID NO: 89 - protein, 18 aa, Homo sapiens
GADFLVTEVENGGSLGSK

SEQ ID NO: 90 - protein, 18 aa, Homo sapiens
KGVNLPGAAVDLPAVSEK

SEQ ID NO: 91 - protein, 17 aa, Homo sapiens
GVNLPGAAVDLPAVSEK

SEQ ID NO: 92 - protein, 16 aa, Homo sapiens
FGVEQDVDMVFASFIR

SEQ ID NO: 93 - protein, 11 aa, Homo sapiens
GDLGIEIPAEK

SEQ ID NO: 94 - protein, 25 aa, Homo sapiens
AEGSDVANAVLDGADCIMLSGETAK

SEQ ID NO: 95 - protein, 16 aa, Homo sapiens
EAEAAIYHLQLFEELR

SEQ ID NO: 96 - protein, 22 aa, Homo sapiens
LAPITSDPTEATAVGAVEASFK

SEQ ID NO: 97 - protein, 11 aa, Homo sapiens
CCSGAIIVLTK

SEQ ID NO: 98 - protein, 8 aa, Homo sapiens
GIFPVLCK

SEQ ID NO: 99 - protein, 14 aa, Homo sapiens
DPVQEAWAEDVDLR

SEQ ID NO: 100 - protein, 434 aa, Homo sapiens
MSILKIHAREIFDSRGNPTVEVDLFTSKGLFRAAVPSGASTGIYEALELRDNDKTRYMGKGVSKAVEHINKTIAPALVSKKLNVTEQEKIDKLMIEMDGTENKSKFGANAILGVSLAVCKAGAVEKGVPLYRHIADLAGNSEVILPVPAFNVINGGSHAGNKLAMQEFMILPVGAANFREAMRIGAEVYHNLKNVIKEKYGKDATNVGDEGGFAPNILENKEGLELLKTAIGKAGYTDKVVIGMDVAASEFFRSGKYDLDFKSPDDPSRYISPDQLADLYKSFIKDYPVVSIEDPFDQDDWGAWQKFTASAGIQVVGDDLTVTNPKRIAKAVNEKSCNCLLLKVNQIGSVTESLQACKLAQANGWGVMVSHRSGETEDTFIADLVVGLCTGQIKTGAPCRSERLAKYNQLLRIEEELGSKAKFAGRNFRNPLAK

SEQ ID NO: 101 - protein, 13 aa, Homo sapiens
GNPTVEVDLFTSK

SEQ ID NO: 102 - protein, 18 aa, Homo sapiens
AAVPSGASTGIYEALELR

SEQ ID NO: 103 - protein, 15 aa, Homo sapiens
FGANAILGVSLAVCK

SEQ ID NO: 104 - protein, 30 aa, Homo sapiens
HIADLAGNSEVILPVPAFNVINGGSHAGNK

SEQ ID NO: 105 - protein, 17 aa, Homo sapiens
LAMQEFMILPVGAANFR

SEQ ID NO: 106 - protein, 19 aa, Homo sapiens
DATNVGDEGGFAPNILENK

SEQ ID NO: 107 - protein, 14 aa, Homo sapiens
VVIGMDVAASEFFR

SEQ ID NO: 108 - protein, 12 aa, Homo sapiens
YISPDQLADLYK

SEQ ID NO: 109 - protein, 21 aa, Homo sapiens
DYPVVSIEDPFDQDDWGAWQK

SEQ ID NO: 110 - protein, 20 aa, Homo sapiens
FTASAGIQVVGDDLTVTNPK

SEQ ID NO: 111 - protein, 22 aa, Homo sapiens
SGETEDTFIADLVVGLCTGQIK

SEQ ID NO: 112 - protein, 679 aa, Homo sapiens; identical to SEQ ID NO: 76

SEQ ID NO: 113 - protein, 23 aa, Homo sapiens
AGAAPYVQAFDSLLAGPVAEYLK

SEQ ID NO: 114 - protein, 18 aa, Homo sapiens
VENQENVSNLVIEDTELK